(19) United States
(12) Patent Application Publication
(10) Pub. No.: US 2019/0161523 A1
(43) Pub. Date: May 30, 2019

(54) METHODS OF TREATMENT USING CHLOROTOXIN CONJUGATES

(71) Applicant: BLAZE BiOSCIENCE, INC., Seattle, WA (US)

(72) Inventors: Dennis M. Miller, Seattle, WA (US); Kelly Byrnes-Blake, Seattle, WA (US)

(21) Appl. No.: 16/091,692

(22) PCT Filed: Apr. 12, 2017
(86) PCT No.: PCT/US17/27276

§ 371 Date: Oct. 5, 2018

Related U.S. Application Data
(60) Provisional application No. 62/321,646, filed on Apr. 12, 2016.
(60) Provisional application No. 62/484,818, filed on Apr. 12, 2017.

(51) Int. Cl. C07K 14/435; A61K 49/00; A61K 9/00; A61P 35/00

(52) U.S. Cl. CPC C07K 14/43522; A61K 49/0032; A61K 38/00; A61K 9/0019; A61P 35/00; A61K 49/0056

(57) ABSTRACT

Compositions, formulations, and kits comprising chlorotoxin conjugate compounds are provided, including native and modified variants of chlorotoxin peptide conjugated to reporter molecules including fluorescent dyes or conjugated to cytotoxic agents. Dosing and pharmacokinetic profiles for therapeutic and diagnostic applications using chlorotoxin conjugate compounds are provided.

### CROSS-REFERENCE

This application claims the benefit of U.S. Provisional Application No. 62/321,646, filed Apr. 12, 2016, and U.S. Provisional Application No. 62/484,818, filed Apr. 12, 2017, which are incorporated herein by reference in their entireties for all purposes.

### SEQUENCE LISTING

The instant application contains a Sequence Listing which has been submitted electronically in ASCII format and is hereby incorporated by reference in its entirety. Said ASCII copy, created on Apr. 12, 2017, is named 45639-708_601_SL.txt and is 245,130 bytes in size.

### BACKGROUND

For many types of cancer, the precision of surgical resection directly influences patient prognosis. Unfortunately, intra-operative identification of tumor margins or small foci of cancer cells remains imprecise or depends on surgical judgment. Thus, the extent of surgical resection is constrained by the requirement to avoid harming vital structures.

Despite the advances in the development of probes for targeting and imaging tumors, there exists a need for a probe that allows for intra-operative visualization of cancerous tissues and cells. Systemic delivery of imaging probes has the advantage of delivering drug to wherever the tumor is, including tumor that has spread locally or to adjacent lymph nodes.

Intravenous dosing often provides the fastest and most predictable systemic exposure to imaging drugs. It is generally used as a reference data set by which drug exposure from other routes of administration, such as subcutaneous or oral, are compared, using pharmacokinetic measures such as initial peak concentration (C₀) or Area under the concentration curve (AUC). Changing the rate of intravenous administration from bolus to infusion is expected to influence the peak concentration values, but not the AUC nor other dose-independent pharmacokinetic parameters, such as clearance or half-life. Only a few chemicals have been reported to have “context-sensitive” half-life, in which the rate of input or injection/infusion influences the rate of output or clearance/half-life. For an imaging agent or imaging probe a “context-sensitive” half-life indicates the dose and the rate of administration influences the systemic exposure and the imaging performance of the agent. This is particularly important in their application to many human disease conditions, such as intra-operative visualization of cancerous tissues and cells.

### SUMMARY

The present disclosure provides peptides or peptide conjugates that give rise to a pharmacokinetic profile when administered intravenously to a human subject. Following administration of the peptides or peptide conjugates described herein, the conjugates can bind selectively to cancer cells. The cancer cells can then be detected, for example, by imaging or other visualization or method suitable for detecting, visualizing, or observing the peptide conjugated to a label or the cancer cells can be treated by the peptides or peptides conjugated to a therapeutic agent. Furthermore, the present disclosure provides peptides or peptide conjugates that at the same dosage produce pharmacokinetic profiles that vary according to the rate of administration of the compound. Additionally, the present disclosure provides peptides or peptide conjugates that at increasing dosages produce pharmacokinetic profiles that vary according to the increase in dosage.

In various aspects, the present disclosure provides a method of administering a composition to a human subject, the method comprising intravenously administering to the human subject a compound comprising a polypeptide having at least 80%, at least 85%, at least 90%, or at least 95% sequence identity with MCMPCFTTDHQMARRCDDCCGGRGRGKCYGPQCLCR (SEQ ID NO: 9) or a fragment thereof, wherein the compound is administered at a dosage within a range from about 1 mg to about 100 mg over a time period within a range from about 1 minute to about 120 minutes; and producing an average maximum blood plasma concentration (average Cₘₐₓ) in the human subject within a range from about 15 ng/mL to about 600 ng/mL per each 1 mg dosage of the compound administered.

In other aspects, the present disclosure provides a method of administering a composition to a human subject, the method comprising intravenously administering to the human subject a compound comprising a polypeptide having at least 80%, at least 85%, at least 90%, or at least 95% sequence identity with any one of SEQ ID NO: 1-SEQ ID NO: 481 or a fragment thereof, wherein the compound is administered at a dosage within a range from about 1 mg to about 100 mg over a time period within a range from about 1 minute to about 120 minutes; and producing an average maximum blood plasma concentration (average Cₘₐₓ) in the human subject within a range from about 15 ng/mL to about 600 ng/mL per each 1 mg dosage of the compound administered.

In further aspects, the present disclosure provides a method of administering a composition to a human subject, the method comprising intravenously administering to the human subject a compound comprising a polypeptide of any one of SEQ ID NO: 482-SEQ ID NO: 485 or a fragment thereof, wherein the compound is administered at a dosage within a range from about 1 mg to about 100 mg over a time period within a range from about 1 minute to about 120 minutes; and producing an average maximum blood plasma concentration (average Cₘₐₓ) in the human subject within a range from about 15 ng/mL to about 600 ng/mL per each 1 mg dosage of the compound administered.

In some aspects, the time period of any of the methods is greater than or equal to about 5 minutes, greater than or equal to about 10 minutes, greater than or equal to about 15 minutes, greater than or equal to about 20 minutes, greater than or equal to about 25 minutes, greater than or equal to about 30 minutes, greater than or equal to about 40 minutes, greater than or equal to about 50 minutes, greater than or equal to about 60 minutes, greater than or equal to about 70 minutes, greater than or equal to about 80 minutes, greater than or equal to about 90 minutes, greater than or equal to about 100 minutes, or greater than or equal to about 110 minutes. In other aspects, the time period for any of the methods is less than or equal to about 5 minutes, less than or equal to about 10 minutes, less than or equal to about 15 minutes, less than or equal to about 20 minutes, less than or equal to about 25 minutes, less than or equal to about 30 minutes, less than or equal to about 40 minutes, less than or equal to about 50 minutes, less than or equal to about 60 minutes, less than or equal to about 70 minutes, less than or equal to about 80 minutes, less than or equal to about 90 minutes, less than or equal to about 100 minutes, or less than or equal to about 110 minutes. In further aspects, the time period of any of the methods is within a range from about 1 minute to about 2 minutes, within range from about 2 minutes to about 5 minutes, or within a range from about 5 minutes to about 120 minutes.

In some aspects, the average Cₘₐₓ per each 1 mg dosage of the compound administered of any of the methods is greater than or equal to about 20 ng/mL, greater than or equal to about 30 ng/mL, greater than or equal to about 40 ng/mL, greater than or equal to about 50 ng/mL, greater than or equal to about 60 ng/mL, greater than or equal to about 70 ng/mL, greater than or equal to about 80 ng/mL, greater than or equal to about 90 ng/mL, greater than or equal to about 100 ng/mL, greater than or equal to about 150 ng/mL, greater than or equal to about 200 ng/mL, greater than or equal to about 250 ng/mL, greater than or equal to about 300 ng/mL, greater than or equal to about 350 ng/mL, greater than or equal to about 400 ng/mL, greater than or equal to about 450 ng/mL, greater than or equal to about 500 ng/mL, or greater than or equal to about 550 ng/mL. In further aspects, the average Cₘₐₓ per each 1 mg dosage of the compound administered of any of the methods is less than or equal to about 20 ng/mL, less than or equal to about 30 ng/mL, less than or equal to about 40 ng/mL, less than or equal to about 50 ng/mL, less than or equal to about 60 ng/mL, less than or equal to about 70 ng/mL, less than or equal to about 80 ng/mL, less than or equal to about 90 ng/mL, less than or equal to about 100 ng/mL, less than or equal to about 150 ng/mL, less than or equal to about 200 ng/mL, less than or equal to about 250 ng/mL, less than or equal to about 300 ng/mL, less than or equal to about 350 ng/mL, less than or equal to about 400 ng/mL, less than or equal to about 450 ng/mL, less than or equal to about 500 ng/mL, or less than or equal to about 550 ng/mL. In additional aspects, the average Cₘₐₓ per each 1 mg dosage of the compound administered of any of the methods is within a range from about 50 ng/mL to about 300 ng/mL.

In some aspects, the average time (average Tₘₐₓ) of any of the methods at which the average Cₘₐₓ is reached is within a range from about 0.5 min to about 120 min following administration of the compound.

In some aspects, the average Cₘₐₓ of any of the methods increases non-linearly with increasing dosage.

In other aspects, the average Cₘₐₓ/mg of the compound administered of any of the methods for dosages greater than 3 mg to 10 mg is up to 2 times, up to 3 times, up to 4 times, up to 5 times, up to 6 times, up to 7 times, up to 8 times, up to 9 times, or up to 10 times greater than the average Cₘₐₓ/mg of the compound administered for dosages of 0.1 mg to 3 mg.

In some aspects, the average Cₘₐₓ of any of the methods varies based on a rate of administration of the compound. In further aspects, the average Cₘₐₓ for any of the methods decreases non-linearly as the rate of administration of the compound decreases. In other aspects, the average Cₘₐₓ per each 1 mg dosage of the compound of any of the methods administered at a rate of greater than 0.2 mg/min to 120 mg/min is up to 1.5 times, up to 2 times, up to 2.5 times, or up to 3 times greater than the average Cₘₐₓ per each 1 mg dosage of the compound administered at a rate of 0.0007 mg/min to 0.2 mg/min.

In certain aspects, any of the methods further comprise producing an average area under the curve (average AUC) in the subject within a range from about 10 hr*ng/mL to about 750 hr*ng/mL per each 1 mg dosage of the compound administered.

In some aspects, the average AUC per each 1 mg dosage of the compound administered of any of the methods is greater than or equal to about 20 hr*ng/mL, greater than or equal to about 30 hr*ng/mL, greater than or equal to about 40 hr*ng/mL, greater than or equal to about 50 hr*ng/mL, greater than or equal to about 60 hr*ng/mL, greater than or equal to about 70 hr*ng/mL, greater than or equal to about 80 hr*ng/mL, greater than or equal to about 90 hr*ng/mL, greater than or equal to about 100 hr*ng/mL, greater than or equal to about 150 hr*ng/mL, greater than or equal to about 200 hr*ng/mL, greater than or equal to about 250 hr*ng/mL, greater than or equal to about 300 hr*ng/mL, greater than or equal to about 350 hr*ng/mL, greater than or equal to about 400 hr*ng/mL, greater than or equal to about 450 hr*ng/mL, greater than or equal to about 500 hr*ng/mL, greater than or equal to about 550 hr*ng/mL, greater than or equal to about 600 hr*ng/mL, greater than or equal to about 650 hr*ng/mL, or greater than or equal to about 700 hr*ng/mL. In other aspects, the average AUC per each 1 mg dosage of the compound administered of any of the methods is less than or equal to about 20 hr*ng/mL, less than or equal to about 30 hr*ng/mL, less than or equal to about 40 hr*ng/mL, less than or equal to about 50 hr*ng/mL, less than or equal to about 60 hr*ng/mL, less than or equal to about 70 hr*ng/mL, less than or equal to about 80 hr*ng/mL, less than or equal to about 90 hr*ng/mL, less than or equal to about 100 hr*ng/mL, less than or equal to about 150 hr*ng/mL, less than or equal to about 200 hr*ng/mL, less than or equal to about 250 hr*ng/mL, less than or equal to about 300 hr*ng/mL, less than or equal to about 350 hr*ng/mL, less than or equal to about 400 hr*ng/mL, less than or equal to about 450 hr*ng/mL, less than or equal to about 500 hr*ng/mL, less than or equal to about 550 hr*ng/mL, less than or equal to about 600 hr*ng/mL, less than or equal to about 650 hr*ng/mL, or less than or equal to about 700 hr*ng/mL.

In some aspects, the average AUC per each 1 mg dosage of the compound administered of any of the methods is within a range from about 15 hr*ng/mL to about 400 hr*ng/mL.

In other aspects, the average AUC of any of the methods increases non-linearly with increasing dosage.

In some aspects, the average AUC/mg of the compound administered for dosages greater than 3 mg to 100 mg of any of the methods is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average AUC/mg of the compound administered for dosages of 0.1 mg to 3 mg.

In other aspects, the average AUC of any of the methods varies based on a rate of administration of the compound. In further aspects, the average AUC of any of the methods increases non-linearly as the rate of administration of the compound decreases.

In some aspects, the average AUC of any of the methods per each 1 mg dosage of the compound administered at a rate of 0.0007 mg/min to 0.2 mg/min is up to 2 times, up to 3 times, up to 4 times, or up to 5 times greater than the average AUC per each 1 mg dosage of the compound administered at a rate of greater than 0.2 mg/min to 120 mg/min.

In certain aspects, the compound of any of the methods has an average elimination half-life (average t_{1/2}) in the human subject within a range from about 0.1 hr to about 10 hr. In further aspects, the average t_{1/2} of any of the methods is greater than or equal to about 0.2 hr, greater than or equal to about 0.3 hr, greater than or equal to about 0.4 hr, greater than or equal to about 0.5 hr, greater than or equal to about 0.6 hr, greater than or equal to about 0.7 hr, greater than or equal to about 0.8 hr, greater than or equal to about 0.9 hr, greater than or equal to about 1 hr, greater than or equal to about 1.5 hr, greater than or equal to about 2 hr, or greater than or equal to about 2.5 hr. In further aspects, the average t_{1/2} of any of the methods is less than or equal to about 0.2 hr, less than or equal to about 0.3 hr, less than or equal to about 0.4 hr, less than or equal to about 0.5 hr, less than or equal to about 0.6 hr, less than or equal to about 0.7 hr, less than or equal to about 0.8 hr, less than or equal to about 0.9 hr, less than or equal to about 1 hr, less than or equal to about 1.5 hr, less than or equal to about 2 hr, or less than or equal to about 2.5 hr.

In some aspects, the average t_{1/2} of any of the methods is within a range from about 0.15 hr to about 3 hr.

In other aspects, the average t_{1/2} of any of the methods increases non-linearly with increasing dosage. In further aspects, the average t_{1/2} of any of methods for dosages greater than 3 mg to 100 mg is up to 2 times, up to 3 times, up to 4 times, up to 5 times, up to 6 times, up to 7 times, up to 8 times, up to 9 times, up to 10 times, up to 15 times, or up to 20 times greater than the average t_{1/2} for dosages of 0.1 mg to 3 mg.

In some aspects, the average t_{1/2} of any of the methods varies based on a rate of administration of the compound.

In other aspects, the average t_{1/2} of any of the methods increases non-linearly as the rate of administration of the compound decreases. In some aspects, the average t_{1/2} of the compound of any of the methods administered at a rate of 0.007 mg/min to 0.2 mg/min is up to 2 times, up to 3 times, up to 4 times, up to 5 times, up to 6 times, up to 7 times, up to 8 times, up to 9 times, or up to 10 times greater than the average t_{1/2} of a compound administered at a rate of greater than 0.2 mg/min to 120 mg/min.

In some aspects, any of the methods further comprises producing an average clearance (average CL) in the subject within a range from about 2,000 mL/hr to about 100,000 mL/hr. the average CL of the compound administered is greater than or equal to 2,000 mL/hr, 4,000 mL/hr, 6,000 mL/hr, 8,000 mL/hr, 10,000 mL/hour, 15,000 mL/hr, 20,000 mL/hr, 25,000 mL/hr, 30,000 mL/hr, 35,000 mL/hr, 40,000 mL/hr, 45,000 mL/hr, or 50,000 mL/hr. In other aspects, the average CL per each 1 mg dosage of the compound administered of any of the methods is less than or equal to 60,000 mL/hr, 70,000 mL/hr, 80,000 mL/hr, 90,000 mL/hr, or 100,000 mL/hr. In further aspects, the average CL of the compound administered for any of the methods is within a range from 4,000 mL/hr to 46,000 mL/hr.

In some aspects, the average CL of any of the methods decreases non-linearly with increasing dosage. In other aspects, the average CL of the compound administered of any of the methods for dosages of 0.1 mg to 3 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average CL of the compound administered for dosages greater than 3 mg to 100 mg.

In some aspects, the average CL of any of the methods varies based on a rate of administration of the compound.

In other aspects, the average CL of any of the methods decreases non-linearly as the rate of administration of the compound decreases. In further aspects, the average CL of the compound of any of the methods administered at a rate of greater than 0.2 mg/min to 120 mg/min is up to 2 times, up to 3 times, up to 4 times, or up to 5 times greater than the average CL of the compound administered at a rate of 0.0007 mg/min to 0.2 mg/min.

In some aspects, any of the methods further comprise producing an average volume of distribution (average V_{d}) in the subject within a range from about 200 mL to about 20,000 mL.

In other aspects, the average V_{d} of the compound administered of any of the methods is greater than or equal to 200 mL, 300 mL, 400 mL, 500 mL, 1,000 mL, 1,500 mL, 2,000 mL, 2,500 mL, 3,000 mL, 4,000 mL, 5,000 mL, 6,000 mL, 7,000 mL, 8,000 mL, 9,000 mL, or 10,000 mL. In some aspects, the average V_{d} of the compound administered of any of the methods is less than or equal to 11,000 mL, 12,000 mL, 13,000 mL, 14,000 mL, 15,000 mL, 16,000 mL, 17,000 mL, 18,000 mL, 19,000 mL, or 20,000 mL. In further aspects, the average V_{d} of the compound administered of any of the methods is within a range from 3,000 mL to 10,000 mL.

In some aspects, the average V_{d} of any of the methods increases non-linearly with increasing dosage.

In other aspects, the average V_{d} of the compound of any of the methods administered for dosages greater than 3 to 100 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average V_{d} of the compound administered for dosages of 0.1 mg to 3 mg.

In some aspects, the average V_{d} of any of the methods varies based on a rate of administration of the compound.

In certain aspects, the average V_{d} of any of the methods decreases non-linearly as the rate of administration of the compound decreases.

In other aspects, the average V_{d} decreases as the rate of administration of the compound decreases. In further aspects, the average V_{d} of the compound of any of the methods administered at a rate of greater than 0.2 mg/min to 120 mg/min is up to 2 times, up to 3 times, up to 4 times, or up to 5 times greater than the average V_{d} of the compound administered at a rate of 0.0007 mg/min to 0.2 mg/min.

In certain aspects, the polypeptide of any of methods has at least 80%, at least 85%, at least 90%, or at least 95% sequence identity with MCMPCFTTDHQMARRCDDCCGGRGRGKCYGPQCLCR (SEQ ID NO: 9) or a fragment thereof. In other aspects, the polypeptide of any of methods has at least 80%, at least 85%, at least 90%, or at least 95% sequence identity with any one of SEQ ID NO: 1-SEQ ID NO: 481 or a fragment thereof. In further aspects, the polypeptide of any of methods is SEQ ID NO: 482-SEQ ID NO: 485 or a fragment thereof.

In other aspects, the fragment of the polypeptide of any of the methods has a length of at least 25 residues. In further aspects, each amino acid of the polypeptide of any of the methods is independently selected as an L- or D-enantiomer. In some aspects, the polypeptide of any of the methods contains no lysine residues. In other aspects, the polypeptide of any of the methods contains a single lysine residue. In further aspects, the single lysine residue of any of the methods is located at a position corresponding to K-27 of native chlorotoxin, K-23 of native chlorotoxin, or K-15 of native chlorotoxin. In some aspects, one, two, or three methionine residues of the polypeptide of any of the methods are replaced with other amino acids.

In other aspects, the N-terminus of the polypeptide of any of the methods is blocked by acetylation or cyclization.

In certain aspects, the polypeptide of any of the methods comprises at least 1, at least 2, at least 3, at least 4, at least 5, or at least 6 disulfide bonds.

In some aspects, the polypeptide of any of the methods comprises an isoelectric point of at least 6.0, at least 6.5, at least 7.0, at least 7.5, at least 8.0, at least 8.5, or at least 9.0.

In other aspects, the compound of any of the methods further comprises an agent.

In some aspects, the polypeptide of any of the methods is conjugated to the agent. In further aspects, the polypeptide of any of the methods comprises a single lysine residue and the agent is conjugated to the polypeptide at the single lysine residue. In other aspects, the polypeptide of any of the methods comprises no lysine residues and the agent is conjugated to the polypeptide at the N-terminus of the polypeptide.

In some aspects, the compound of any of the methods has the structure of Formula (IV), or a pharmaceutically acceptable salt thereof:

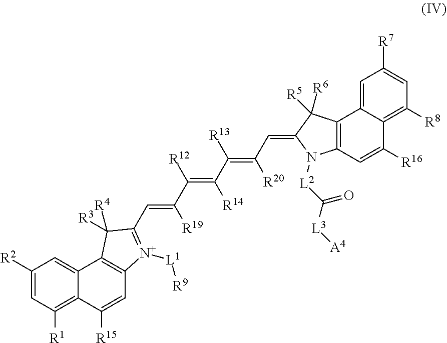

wherein:
R¹, R², R³, R⁴, R⁵, R⁶, R⁷, R⁸, R¹⁵, and R¹⁶ are each independently selected from hydrogen, C₁-C₆ alkyl, C₁-C₆ alkylene-COOH, sulfonate, C₁-C₆ alkylene-sulfonate, —COOH, —SO₂—NH₂, or C₁-C₆ alkoxy;
R⁹ is hydrogen, sulfonate, amine, or —COOH;
L¹ is C₃-C₆ alkylene;
L² is C₁-C₁₀ alkylene;
L³ is a bond, —O—, —NR¹⁰—, —NR¹⁰—C₁-C₆ alkylene-, —O—NR¹⁰—, —NR¹⁰—C₁-C₆ alkylene-(O—C₁-C₆ alkylene)ₙ-, —NR¹⁰-L⁴-, —NR¹⁰—C₁-C₆ alkylene-NR¹¹—(C(═O)—C₁-C₆ alkylene-O—)ₘ—, or —NR¹⁰—C₁-C₆ alkylene-NR¹⁰—C₁-C₆ alkylene-NR¹⁰—C₁-C₆ alkylene-;
L⁴ is a bond, -heterocyclyl-, or -heterocyclyl-C₁-C₆ alkylene-;
R¹⁰ is hydrogen or C₁-C₆ alkyl;
R¹¹ is hydrogen or C₁-C₆ alkyl;
R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;
R¹⁴ is hydrogen or C₁-C₆ alkylene, -(L⁵)-aryl, -(L⁵)-aryl-R²¹, -(L⁵)-heteroaryl, -(L⁵)-heteroaryl-R²¹, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
L⁵ is a bond, C₁-C₁₀ alkylene, —O—, —NR¹⁰—;
R¹⁷ and R¹⁸ are each independently hydrogen or aryl;
R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
R²¹ is hydrogen, sulfonate, or —COOH;
n is 0, 1, 2, or 3;
m is 0, 1, 2, or 3;
p is 0, 1, 2, or 3;
q is 0, 1, 2, or 3; and
A⁴ is the polypeptide.

In further aspects, for any of the methods,
R³, R⁴, R⁵, R⁶ are each independently methyl;
R¹, R², R⁷, R, R¹⁵, and R¹⁶ are each independently hydrogen;
R¹², R³, R¹⁴, R¹⁹, and R²⁰ are each independently hydrogen;
R⁹ is sulfonate;
R¹⁰ is hydrogen;
L¹ is butylene;
L² is pentylene; or
L³ is selected from a bond, —O—, —NR¹⁰—, —NR¹⁰—C₁-C₆ alkylene-, —O—NR¹⁰—, or —NR¹⁰-L⁴-.

In other aspects, the compound of any of the methods has the structure of any one of Formulas (IX), (X), (XI), (XII), (XIII), (XIV), (XV), or (XVI), wherein A⁴ is the polypeptide:

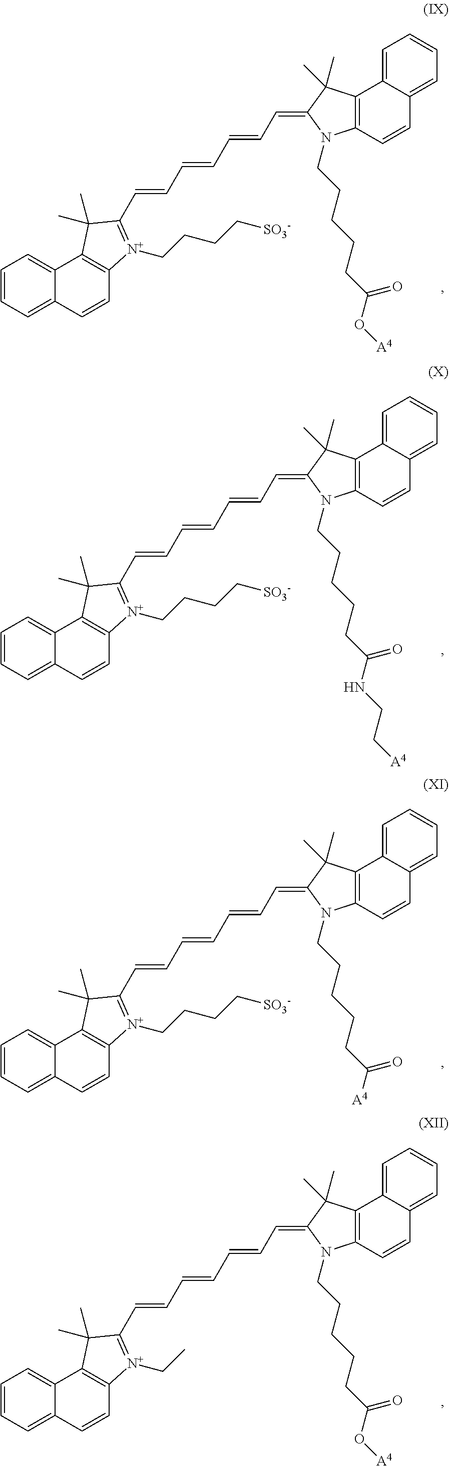

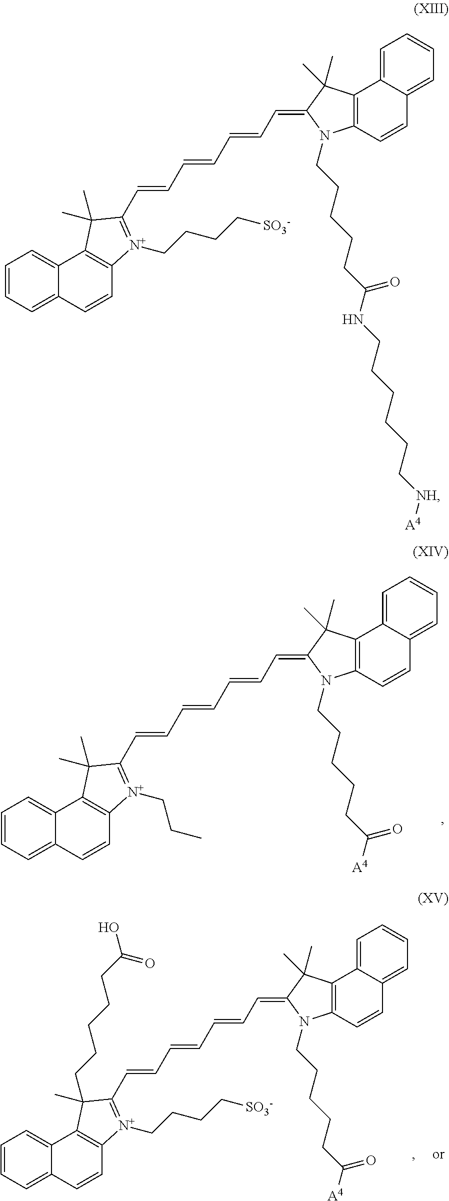

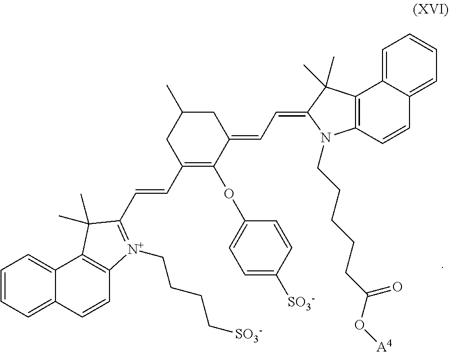

In other aspects, the compound of any of the methods comprises a detectable agent. In further aspects, the compound of any of the methods is conjugated to the detectable agent. In still further aspects, the detectable agent of any of the methods comprises a dye, a fluorophore, a fluorescent biotin compound, a luminescent compound, a chemiluminescent compound, a radioisotope, a paramagnetic metal ion, or a combination thereof.

In some aspects, the compound of any of the methods comprises a therapeutic agent. In further aspects, the polypeptide of any of the methods is conjugated to the therapeutic agent. In still further aspects, the therapeutic agent of any of the methods comprises a radioisotope, toxin, enzyme, sensitizing drug, radiosensitizer, nucleic acid, interfering RNA, antibody, antibody fragment, aptamer, anti-angiogenic agent, cisplatin, carboplatin, oxaliplatin, anti-metabolite, mitotic inhibitor, growth factor inhibitor, cytotoxin, microtubule disrupting agent, DNA modifying agent, maytansine derivative, auristatin derivative, dolostatin derivative, monomethyl auristatin E, monomethyl auristatin F, DM1, calicheamicin, duocarmycin derivative, campthotecin, pyrrolobenzodiazepine, paclitaxel, cyclophosphamide, chlorambucil, melphlan, bufulfan, carmustine, ifosfamide, temozolomide, topotecan, fluorouracil, vincristine, vinblastine, procarbazine, dacarbazine, altretamine, methotrexate, pemetrexed, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane, amifostine, lenalidomide, imatinib, abiraterone, erlotinib, enzalutimide, everolimus palbociclib, pomalidomide, sutininib, sorafenib, imatinib, gefitinib, afatinib, axitinib, crizotinib, vismoegib, dabrefenib, vemurafenib, or a combination thereof.

In other aspects, intravenously administering the compound of any of the methods comprises intravenously administering a composition comprising the compound and a pharmaceutically acceptable carrier.

In another aspect, the composition of any of the methods comprises a pH within a range from about 6 to about 7.5.

In other aspects, the composition of any of the methods comprises an ionic strength less than or equal to about 50 mM.

In some aspects, the composition of any of the methods further comprises a buffer comprising histidine, tris, HEPES, ethylene diamine, or a combination thereof.

In other aspects, the composition of any of the methods further comprises a sugar alcohol.

In some aspects, the composition of any of the methods comprises from about 0 mM to about 50 mM histidine, from about 0 mM to about 20 mM tris, about 20 mM methionine, from about 3% to about 10% sugar alcohol, and a pH within a range from about 6 to about 7.5.

In other aspects, any of the methods further comprises detecting the presence or absence of the compound in a tissue or cell, wherein the presence of the compound in the tissue or cell indicates the presence of a cancerous tissue or cancer cell.

In some aspects, the cancerous tissue or cancer cell of any of the methods is associated with one or more of: brain cancer, glioma, astrocytoma, medulloblastoma, oligiodendroglioma, choroids plexus carcinoma, ependymoma, pituitary cancer, neuroblastoma, basal cell carcinoma, cutaneous squamous cell carcinoma, melanoma, head and neck cancer, lung cancer, small cell lung cancer, non-small cell lung cancer, breast cancer, ductal carcinoma in situ, intestinal cancer, pancreatic cancer, liver cancer, kidney cancer, bladder cancer, carcinoma of unknown primary, sarcoma, osteosarcoma, rhabdomyosarcoma, Ewing's sarcoma, gastrointestinal stromal tumors, melanoma, ovarian cancer, cervical cancer, lymphoma, Hodgkin's lymphoma, non-Hodgkin's lymphoma, thyroid cancer, anal cancer, colo-rectal cancer, endometrial cancer, laryngeal cancer, multiple myeloma, prostate cancer, retinoblastoma, gastric cancer, esophageal cancer, testicular cancer, or Wilm's tumor.

In certain aspects, the compound of any of the methods binds the cancerous tissue or cancer cell.

In other aspects, the detecting of any of the methods is performed using fluorescence imaging.

In some aspects, any of the methods further comprises surgically removing the cancerous tissue or cancer cell from the human subject.

In other aspects, the compound of any of the methods is administered at a dosage sufficient to treat cancer in the human subject.

In some aspects, the cancer of any of the methods comprises one or more of: brain cancer, glioma, astrocytoma, medulloblastoma, oligiodendroglioma, choroids plexus carcinoma, ependymoma, pituitary cancer, neuroblastoma, basal cell carcinoma, cutaneous squamous cell carcinoma, melanoma, head and neck cancer, lung cancer, small cell lung cancer, non-small cell lung cancer, breast cancer, ductal carcinoma in situ, intestinal cancer, pancreatic cancer, liver cancer, kidney cancer, bladder cancer, carcinoma of unknown primary, sarcoma, osteosarcoma, rhabdomyosarcoma, Ewing's sarcoma, gastrointestinal stromal tumors, melanoma, ovarian cancer, cervical cancer, lymphoma, Hodgkin's lymphoma, non-Hodgkin's lymphoma, thyroid cancer, anal cancer, colo-rectal cancer, endometrial cancer, laryngeal cancer, multiple myeloma, prostate cancer, retinoblastoma, gastric cancer, esophageal cancer, testicular cancer, or Wilm's tumor.

In certain aspects, the compound of any of the methods binds a cancerous tissue or cancer cell.

In other aspects, the compound of any of the methods is intravenously administered about 1 hr, about 2 hrs, about 3 hrs, about 4 hrs, about 5 hrs, about 6 hrs, about 7 hrs, about 8 hrs, about 9 hrs, about 10 hrs, about 11 hrs, about 12 hrs, about 13 hrs, about 14 hrs, about 15 hrs, about 16 hrs, about 17 hrs, about 18 hrs, about 19 hrs, about 20 hrs, about 21 hrs, about 22 hrs, about 23 hrs, about 24 hrs, about 36 hrs, about 48 hrs, about 60 hrs, or about 72 hrs prior to performing surgery on the human subject.

In some aspects, a method of administering a composition to a human subject comprises determining a rate of administration of a compound to a human subject, the compound comprising a polypeptide having at least 80% sequence identity with MCMPCFTTDHQMARRCDDCCGGRGRGKCYGPQCLCR (SEQ ID NO: 9) or a fragment thereof, wherein a pharmacokinetic profile of the compound in the human subject varies according to the rate of administration of the compound; and intravenously administering the compound to the human subject at the determined rate.

In other aspects, a method of administering a composition to a human subject comprises determining a rate of administration of a compound to a human subject, the compound comprising a polypeptide having at least 80% sequence identity with any one of SEQ ID NO: 1-SEQ ID NO: 481 or a fragment thereof, wherein a pharmacokinetic profile of the compound in the human subject varies according to the rate of administration of the compound; and intravenously administering the compound to the human subject at the determined rate.

In further aspects, a method of administering a composition to a human subject comprises determining a rate of administration of a compound to a human subject, the compound comprising a polypeptide any one of SEQ ID NO: 482-SEQ ID NO: 485 or a fragment thereof, wherein a pharmacokinetic profile of the compound in the human subject varies according to the rate of administration of the compound; and intravenously administering the compound to the human subject at the determined rate.

In other aspects, the rate of administration per 1 mg dosage of any of the methods is selected from 120 mg/min to 0.5 mg/min, 0.5 mg/min to 0.2 mg/min, or 0.2 mg/min to 0.0007 mg/min.

In other aspects, the determining the rate of administration of any of the methods comprises determining a time period over which a predetermined dosage is to be intravenously administered to the human subject. In further aspects, the predetermined dosage of any of the methods is within a range from about 0.1 mg to about 100 mg.

In other aspects, the time period of any of the methods is selected from: less than or equal to about 2 minutes, within a range from about 2 minutes to about 5 minutes, or greater than or equal to about 5 minutes.

In some aspects, the rate of administration of any of the methods is determined based on one or more characteristics of a cancer in the human subject.

In other aspects, the cancer of any of the methods comprises one or more of: brain cancer, glioma, astrocytoma, medulloblastoma, oligiodendroglioma, choroids plexus carcinoma, ependymoma, pituitary cancer, neuroblastoma, basal cell carcinoma, cutaneous squamous cell carcinoma, melanoma, head and neck cancer, lung cancer, small cell lung cancer, non-small cell lung cancer, breast cancer, ductal carcinoma in situ, intestinal cancer, pancreatic cancer, liver cancer, kidney cancer, bladder cancer, carcinoma of unknown primary, sarcoma, osteosarcoma, rhabdomyosarcoma, Ewing's sarcoma, gastrointestinal stromal tumors, melanoma, ovarian cancer, cervical cancer, lymphoma, Hodgkin's lymphoma, non-Hodgkin's lymphoma, thyroid cancer, anal cancer, colo-rectal cancer, endometrial cancer, laryngeal cancer, multiple myeloma, prostate cancer, retinoblastoma, gastric cancer, esophageal cancer, testicular cancer, or Wilm's tumor.

In certain aspects, the one or more characteristics of any of the methods comprise a type of the cancer. In further aspects, the one or more characteristics of any of the methods comprise an aggressiveness of the cancer.

In other aspects, the determined rate of administration of any of the methods is higher when the cancer is more aggressive and lower when the cancer is less aggressive.

In some aspects, the one or more characteristics of any of the methods comprise a location of the cancer.

In other aspects, the determined rate of administration of any of the methods is lower when the cancer is located in the brain and higher when the cancer is not located in the brain.

In some aspects, the one or more characteristics of any of the methods comprise a rate of uptake of the compound by cancerous tissue or cancer cells.

In other aspects, the determined rate of administration of any of the methods is higher when the rate of uptake is higher and lower when the rate of uptake is lower.

In some aspects, the rate of administration of any of the methods is determined based on an amount of time between the administration of the compound and performing of a surgical procedure on the human subject. In further aspects, the determined rate of any of the methods is higher when the amount of time is shorter and lower when the amount of time is longer.

In other aspects, the rate of administration of any of the methods is determined based on a type of a surgical procedure to be performed on the human subject following the administration of the compound.

In some aspects, any of the methods further comprises performing the surgical procedure on the human subject, wherein the determined rate of administration results in an average blood plasma concentration of the compound greater than about 10 ng/mL when the surgical procedure is performed. In further aspects, the surgical procedure of any of the methods is performed to remove cancerous tissue or cancer cells from the human subject.

In other aspects, the rate of administration of any of the methods is determined based on a therapeutic usage of the compound.

In certain aspects, any of the methods further comprises producing a pharmacokinetic profile in the human subject.

In other aspects, the pharmacokinetic profile of any of the methods comprises an average maximum blood plasma concentration (average Cₘₐₓ) in the human subject within a range from about 15 ng/mL to about 600 ng/mL per each 1 mg dosage of the compound administered.

In some aspects, the average Cₘₐₓ per each 1 mg dosage of the compound administered of any of the methods is greater than or equal to about 20 ng/mL, greater than or equal to about 30 ng/mL, greater than or equal to about 40 ng/mL, greater than or equal to about 50 ng/mL, greater than or equal to about 60 ng/mL, greater than or equal to about 70 ng/mL, greater than or equal to about 80 ng/mL, greater than or equal to about 90 ng/mL, greater than or equal to about 100 ng/mL, greater than or equal to about 150 ng/mL, greater than or equal to about 200 ng/mL, greater than or equal to about 250 ng/mL, greater than or equal to about 300 ng/mL, greater than or equal to about 350 ng/mL, greater than or equal to about 400 ng/mL, greater than or equal to about 450 ng/mL, greater than or equal to about 500 ng/mL, or greater than or equal to about 550 ng/mL. In other aspects, the average Cₘₐₓ per each 1 mg dosage of the compound administered of any of the methods is less than or equal to about 20 ng/mL, less than or equal to about 30 ng/mL, less than or equal to about 40 ng/mL, less than or equal to about 50 ng/mL, less than or equal to about 60 ng/mL, less than or equal to about 70 ng/mL, less than or equal to about 80 ng/mL, less than or equal to about 90 ng/mL, less than or equal to about 100 ng/mL, less than or equal to about 150 ng/mL, less than or equal to about 200 ng/mL, less than or equal to about 250 ng/mL, less than or equal to about 300 ng/mL, less than or equal to about 350 ng/mL, less than or equal to about 400 ng/mL, less than or equal to about 450 ng/mL, less than or equal to about 500 ng/mL, or less than or equal to about 550 ng/mL.

In other aspects, the average Cₘₐₓ per each 1 mg dosage of the compound administered of any of the methods is within a range from about 50 ng/mL to about 300 ng/mL.

In some aspects, the average time (average Tₘₐₓ) of any of the methods at which the average Cₘₐₓ is reached is within a range from about 0.5 min to about 120 min following administration of the compound.

In other aspects, the average Cₘₐₓ of any of the methods increases non-linearly with increasing dosage. In some methods, the average Cₘₐₓ/mg of the compound administered of any of the methods for dosages greater than 3 mg to 100 mg is up to 2 times, up to 3 times, up to 4 times, up to 5 times, up to 6 times, up to 7 times, up to 8 times, up to 9 times, or up to 10 times greater than the average Cₘₐₓ/mg of the compound administered for dosages of 0.1 mg to 3 mg.

In other aspects, the average Cₘₐₓ of any of the methods varies based on a rate of administration of the compound.

In some aspects, the average Cₘₐₓ of any of the methods decreases non-linearly as the rate of administration of the compound decreases.

In other aspects, the average Cₘₐₓ per each 1 mg dosage of the compound of any of the methods administered at a rate of greater than 0.2 mg/min to 120 mg/min is up to 1.5 times, up to 2 times, up to 2.5 times, or up to 3 times greater than the average Cₘₐₓ per each 1 mg dosage of the compound administered at a rate of 0.0007 mg/min to 0.2 mg/min.

In some aspects, the pharmacokinetic profile of any of the methods comprises an average area under the curve (average AUC) in the subject within a range from about 10 hr*ng/mL to about 750 hr*ng/mL per each 1 mg dosage of the compound administered. In further aspects, the average AUC per each 1 mg dosage of the compound administered of any of the methods is greater than or equal to about 20 hr*ng/mL, greater than or equal to about 30 hr*ng/mL, greater than or equal to about 40 hr*ng/mL, greater than or equal to about 50 hr*ng/mL, greater than or equal to about 60 hr*ng/mL, greater than or equal to about 70 hr*ng/mL, greater than or equal to about 80 hr*ng/mL, greater than or equal to about 90 hr*ng/mL, greater than or equal to about 100 hr*ng/mL, greater than or equal to about 150 hr*ng/mL, greater than or equal to about 200 hr*ng/mL, greater than or equal to about 250 hr*ng/mL, greater than or equal to about 300 hr*ng/mL, greater than or equal to about 350 hr*ng/mL, greater than or equal to about 400 hr*ng/mL, greater than or equal to about 450 hr*ng/mL, greater than or equal to about 500 hr*ng/mL, greater than or equal to about 550 hr*ng/mL, greater than or equal to about 600 hr*ng/mL, greater than or equal to about 650 hr*ng/mL, or greater than or equal to about 700 hr*ng/mL. In other aspects, the average AUC per each 1 mg dosage of the compound administered of any of the methods is less than or equal to about 20 hr*ng/mL, less than or equal to about 30 hr*ng/mL, less than or equal to about 40 hr*ng/mL, less than or equal to about 50 hr*ng/mL, less than or equal to about 60 hr*ng/mL, less than or equal to about 70 hr*ng/mL, less than or equal to about 80 hr*ng/mL, less than or equal to about 90 hr*ng/mL, less than or equal to about 100 hr*ng/mL, less than or equal to about 150 hr*ng/mL, less than or equal to about 200 hr*ng/mL, less than or equal to about 250 hr*ng/mL, less than or equal to about 300 hr*ng/mL, less than or equal to about 350 hr*ng/mL, less than or equal to about 400 hr*ng/mL, less than or equal to about 450 hr*ng/mL, less than or equal to about 500 hr*ng/mL, less than or equal to about 550 hr*ng/mL, less than or equal to about 600 hr*ng/mL, less than or equal to about 650 hr*ng/mL, or less than or equal to about 700. In certain aspects, the average AUC per each 1 mg dosage of the compound administered of any of the methods is within a range from about 15 hr*ng/mL to about 400 hr*ng/mL.

In other aspects, the average AUC of any of the methods increases non-linearly with increasing dosage. In certain aspects, the average AUC/mg of the compound of any of the methods administered for dosages greater than 3 mg to 100 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average AUC/mg of the compound administered for dosages of 0.1 mg to 3 mg.

In some aspects, the average AUC of any of the methods varies based on the rate of administration of the compound.

In other aspects, the average AUC of any of the methods increases non-linearly as the rate of administration of the compound decreases. In further aspects, the average AUC per each 1 mg dosage of the compound of any of the methods administered at a rate of 0.0007 mg/min to 0.2 mg/min is up to 2 times, up to 3 times, up to 4 times, or up to 5 times greater than the average AUC per each 1 mg dosage of the compound administered at a rate of greater than 0.2 mg/min to 120 mg/min.

In some aspects, the pharmacokinetic profile of any of the methods comprises an average elimination half-life (average t_{1/2}) in the human subject within a range from about 0.1 hr to about 10 hr.

In other aspects, the average t_{1/2} of any of the methods is greater than or equal to about 0.2 hr, greater than or equal to about 0.3 hr, greater than or equal to about 0.4 hr, greater than or equal to about 0.5 hr, greater than or equal to about 0.6 hr, greater than or equal to about 0.7 hr, greater than or equal to about 0.8 hr, greater than or equal to about 0.9 hr, greater than or equal to about 1 hr, greater than or equal to about 1.5 hr, greater than or equal to about 2 hr, or greater than or equal to about 2.5 hr. In further aspects, the average t_{1/2} of any of the methods is less than or equal to about 0.2 hr, less than or equal to about 0.3 hr, less than or equal to about 0.4 hr, less than or equal to about 0.5 hr, less than or equal to about 0.6 hr, less than or equal to about 0.7 hr, less than or equal to about 0.8 hr, less than or equal to about 0.9 hr, less than or equal to about 1 hr, less than or equal to about 1.5 hr, less than or equal to about 2 hr, or less than or equal to about 2.5 hr. In certain aspects, the average t_{1/2} of any of the methods is within a range from about 0.15 hr to about 3 hr.

In other aspects, the average t_{1/2} of any of the methods increases non-linearly with increasing dosage.

In some aspects, the average t_{1/2} of any of the methods for dosages greater than 3 mg to 100 mg is up to 2 times, up to 3 times, up to 4 times, up to 5 times, up to 6 times, up to 7 times, up to 8 times, up to 9 times, up to 10 times, up to 15 times, or up to 20 times greater than the average t_{1/2} for dosages of 0.1 mg to 3 mg.

In other aspects, the average t_{1/2} of any of the methods varies based on the rate of administration of the compound.

In some aspects, the average t_{1/2} of any of the methods increases non-linearly as the rate of administration of the compound decreases. In further aspects, the average t_{1/2} of the compound of any of the methods administered at a rate of 0.0007 mg/min to 0.2 mg/min is up to 2 times, up to 3 times, up to 4 times, up to 5 times, up to 6 times, up to 7 times, up to 8 times, up to 9 times, or up to 10 times greater than the average t_{1/2} of the compound administered at a rate of greater than 0.2 mg/min to 120 mg/min.

In some aspects, the pharmacokinetic profile of any of the methods comprises an average clearance (average CL) in the subject within a range from about 2,000 mL/hr to about 100,000 mL/hr. In further aspects, the average CL of the compound of any of the methods administered is greater than or equal to 2,000 mL/hr, 4,000 mL/hr, 6,000 mL/hr, 8,000 mL/hr, 10,000 mL/hour, 15,000 mL/hr, 20,000 mL/hr, 25,000 mL/hr, 30,000 mL/hr, 35,000 mL/hr, 40,000 mL/hr, 45,000 mL/hr, or 50,000 mL/hr. In other aspects, the average CL of the compound of any of the methods administered is less than or equal to 60,000 mL/hr, 70,000 mL/hr, 80,000 mL/hr, 90,000 mL/hr, or 100,000 mL/hr. In further aspects, the average CL of the compound administered of any of the methods is within a range from 4,000 mL/hr to 46,000 mL/hr.

In other aspects, the average CL of any of the methods decreases non-linearly with increasing dosage. In certain aspects, the average CL of the compound of any of the methods administered for dosages of 0.1 mg to 3 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average CL of the compound administered for dosages greater than 3 mg to 100 mg.

In some aspects, the average CL of any of the methods varies based on a rate of administration of the compound.

In other aspects, the average CL of any of the methods decreases non-linearly as the rate of administration of the compound decreases. In further aspects, the average CL of the compound of any of the methods administered at a rate of greater than 0.2 mg/min to 120 mg/min is up to 2 times, up to 3 times, up to 4 times, or up to 5 times greater than the average CL of the compound administered at a rate of 0.0007 mg/min to 0.2 mg/min.

In other aspects, the pharmacokinetic profile of any of the methods comprises an average volume of distribution (average V_{d}) in the subject within a range from about 200 mL to about 20,000 mL. In some aspects, the average V_{d} of the compound administered of any of the methods is greater than or equal to 200 mL, 300 mL, 400 mL, 500 mL, 1,000 mL, 1,500 mL, 2,000 mL, 2,500 mL, 3,000 mL, 4,000 mL, 5,000 mL, 6,000 mL, 7,000 mL, 8,000 mL, 9,000 mL, or 10,000 mL. In other aspects, the average V_{d} of the compound administered of any of the methods is less than or equal to 11,000 mL, 12,000 mL, 13,000 mL, 14,000 mL, 15,000 mL, 16,000 mL, 17,000 mL, 18,000 mL, 19,000 mL, or 20,000 mL.

In other aspects, the average V_{d} of the compound administered of any of the methods is within a range from 3,000 mL to 10,000 mL.

In some aspects, the average V_{d} of any of the methods increases non-linearly with increasing dosage. In some aspects, the average V_{d} of the compound of any of the methods administered for dosages greater than 3 to 100 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average V_{d} of the compound administered for dosages of 0.1 mg to 3 mg.

In some aspects, the average V_{d} of any of the methods varies based on a rate of administration of the compound.

In certain aspects, the average V_{d} of any of the methods decreases non-linearly as the rate of administration of the compound decreases.

In some aspects, the average V_{d} of any of the methods decreases as the rate of administration of the compound decreases. In other aspects, the average V_{d} of the compound of any of the methods administered at a rate of greater than 0.2 mg/min to 120 mg/min is up to 2 times, up to 3 times, up to 4 times, or up to 5 times greater than the average V_{d} of the compound administered at a rate of 0.0007 mg/min to 0.2 mg/min.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a graph of mean Compound 76 concentration versus time profiles following a single 15 minute intravenous infusion.

FIG. 2 shows a graph of mean Compound 76 concentration versus time profiles following a single intravenous slow-bolus injection.

FIG. 3 shows a graph of mean Compound 76 concentration versus time profiles following a single intravenous slow-bolus injection (BB-002) of 3 mg compared to following a single 15 minute intravenous infusion of 3 mg (BB-001).

FIG. 4 shows a graph of mean Compound 76 concentration versus time profiles following a single intravenous slow-bolus injection of 18 mg (BB-002) compared to following a single 15 minute intravenous infusion of 18 mg (BB-001).

FIG. 5 shows a graph of mean Compound 76 concentration versus time profile following a single intravenous infusion of 1 mg (BB-001) compared to predicted human mean Compound 76 concentration versus time profiles determined by data from animal studies.

FIG. 6 shows a graph of mean Compound 76 concentration versus time profiles following a single intravenous infusion of 3 mg (BB-001) compared to predicted human mean Compound 76 concentration versus time profiles determined by data from animal studies and from a single intravenous slow-bolus injection of 3 mg (BB-002).

FIG. 7 shows a graph of mean Compound 76 concentration versus time profiles following a single intravenous infusion of 6 mg (BB-001) compared to predicted human mean Compound 76 concentration versus time profiles determined by data from animal studies.

FIG. 8 shows a graph of mean Compound 76 concentration versus time profiles following a single intravenous infusion of 12 mg (BB-001) compared to predicted human mean Compound 76 concentration versus time profiles determined by data from animal studies and from a single 18 mg intravenous slow-bolus injection clinical trial (BB-002).

FIG. 9A shows an Infrared LED image of a basal cell carcinoma lesion before a single 3 mg 15-minute intravenous infusion of Compound 76.

FIG. 9B shows a FLUOBEAM 800 image of a basal cell carcinoma lesion before a single 3 mg 15-minute intravenous infusion of Compound 76.

FIG. 9C shows a FLUOBEAM 800 image of a basal cell carcinoma lesion 2 hours after a single 3 mg 15-minute intravenous infusion of Compound 76.

FIG. 9D shows a FLUOBEAM 800 image of a basal cell carcinoma lesion 4 hours after a single 3 mg 15-minute intravenous infusion of Compound 76.

FIG. 9E shows a FLUOBEAM 800 image of a basal cell carcinoma lesion 24 hours after a single 3 mg 15-minute intravenous infusion of Compound 76.

FIG. 9F shows a FLUOBEAM 800 image of a basal cell carcinoma lesion 48 hours after a single 3 mg 15-minute intravenous infusion of Compound 76.

FIG. 10A shows an Infrared LED image of a melanoma lesion before a single 6 mg 15-minute intravenous infusion of Compound 76.

FIG. 10B shows a FLUOBEAM 800 image of a melanoma lesion before a single 6 mg 15-minute intravenous infusion of Compound 76.

FIG. 10C shows a FLUOBEAM 800 image of a melanoma lesion 2 hours after a single 6 mg 15-minute intravenous infusion of Compound 76.

FIG. 10D shows a FLUOBEAM 800 image of a melanoma lesion 4 hours after a single 6 mg 15-minute intravenous infusion of Compound 76.

FIG. 10E shows a FLUOBEAM 800 image of a melanoma lesion 24 hours after a single 6 mg 15-minute intravenous infusion of Compound 76.

FIG. 10F shows a FLUOBEAM 800 image of a melanoma lesion 48 hours after a single 12 mg 15-minute intravenous infusion of Compound 76.

FIG. 11A shows an Infrared LED image of a melanoma lesion 24 hours after a single 12 mg 15-minute intravenous infusion of Compound 76.

FIG. 11B shows a FLUOBEAM 800 image of a melanoma lesion before a single 12 mg 15-minute intravenous infusion of Compound 76.

FIG. 11C shows a FLUOBEAM 800 image of a melanoma lesion 2 hours after a single 12 mg 15-minute intravenous infusion of Compound 76.

FIG. 11D shows a FLUOBEAM 800 image of a melanoma lesion 4 hours after a single 12 mg 15-minute intravenous infusion of Compound 76.

FIG. 11E shows a FLUOBEAM 800 image of a melanoma lesion 24 hours after a single 12 mg 15-minute intravenous infusion of Compound 76.

FIG. 11F shows a FLUOBEAM 800 image of a melanoma lesion 48 hours after a single 12 mg 15-minute intravenous infusion of Compound 76.

FIG. 12 shows a FLUOBEAM 800 image of a melanoma lesion 2 hours after a single 12 mg 15-minute intravenous infusion of Compound 76.

FIG. 13A shows a white light in situ image of an exposed glioblastoma multiforme tumor from human subject given 18 mg Compound 76.

FIG. 13B shows a Near Infrared (NIR) light in situ image of an exposed glioblastoma multiforme tumor from human subject given 18 mg Compound 76.

FIG. 13C shows the combined white light and Near Infrared (NIR) light in situ image of an exposed glioblastoma multiforme tumor from human subject given 18 mg Compound 76.

FIG. 14A shows Near Infrared (NIR) light image of ex vivo tissue from a human subject given an 18 mg dose of Compound 76.

FIG. 14B shows Near Infrared (NIR) light image overlaid on a white light image of the same ex vivo tissue from a human subject given an 18 mg dose of Compound 76 as FIG. 14A.

FIG. 14C shows an H&E staining image of a tissue slice from the upper fluorescent region of the ex vivo tissue from a human subject given an 18 mg dose of Compound 76 corresponding to tissue area in FIG. 14A marked by an arrow from FIG. 14A to this Figure. The entire tissue slice shown in this figure contains tumor.

FIG. 14D shows an Odyssey scan of the tissue slice shown in FIG. 14C, in which the tissue is ex vivo tissue from a human subject given an 18 mg dose of Compound 76 and the entire tissue slice is from the fluorescent tumor region in the upper portion of FIG. 14A. The entire tissue slice shown in this figure contains tumor. Fluorescence signal intensity varied in the tissue, but overall the NIR signal intensity was high.

FIG. 14E shows an H&E staining image of the a tissue slice from the lower dark region of the ex vivo tissue from a human subject given an 18 mg dose of Compound 76 corresponding to tissue area in FIG. 14A marked by an arrow from FIG. 14A to this Figure. The entire tissue slice shown in this figure is mostly from necrotic tissue and has less viable tumor than FIG. 14C and FIG. 14D.

FIG. 14F shows an Odyssey scan of the tissue slice shown in FIG. 14E, in which the tissue is ex vivo tissue from a human subject given an 18 mg dose of Compound 76 and the entire tissue slice is from the dark necrotic tissue region in the lower portion of FIG. 14A. The entire tissue slice shown in this figure is mostly from necrotic tissue and has less viable tumor than FIG. 14C and FIG. 14D. Fluorescence signal intensity has significantly less NIR fluorescence signal and is consistent with being sections from the dark region of FIG. 14A. Only a very few regions had bright NIR tumor signal, further indicated that the tissue has significantly less tumor and is largely necrotic tissue.

FIG. 14G shows an Odyssey scan of untreated cerebellum used as a negative control.

FIG. 15A shows an Odyssey scan of an ex vivo low-grade pleomorphic xanthocytoma tumor from a pediatric subject dosed with the equivalent of a 3 mg adult dose of Compound 76.

FIG. 15B shows an H&E staining of an ex vivo low-grade pleomorphic xanthocytoma tumor from a pediatric subject dosed with the equivalent of a 3 mg adult dose of Compound 76, which is from the area of the tumor indicated by the corresponding arrow from FIG. 15A.

FIG. 15C shows an H&E staining of an ex vivo low-grade pleomorphic xanthocytoma tumor from a pediatric subject dosed with the equivalent of a 3 mg adult dose of Compound 76, which is from the area of the tumor indicated by the corresponding arrow from FIG. 15A.

FIG. 16A shows a white light image of ex vivo gross tissue specimens of breast cancer from a human subject dosed with 12 mg Compound 76.

FIG. 16B shows a Near infrared (NIR) light image overlay with the white light image of FIG. 16A, in which the images are of ex vivo gross tissue specimens of breast cancer from a human subject dosed with 12 mg Compound 76.

FIG. 17A shows a graph of predicted Compound 76 concentration versus time profiles after administration of 12 mg Compound 76 at different rates of administration.

FIG. 17B shows a graph of predicted Compound 76 concentration versus time profiles after administration of 24 mg Compound 76 at different rates of administration.

FIG. 18A shows single dose pharmacokinetic rat data at several dose levels including 0.292 mg/kg, 1 mg/kg, 2.90 mg/kg, 22 mg/kg, and 29.8 mg/kg.

FIG. 18B shows a pharmacokinetic comparison between rats receiving single dose administration at doses of 1 mg/kg or 22 mg/kg versus repeat dose administration every day for 7 days at doses of 1 mg/kg or 22 mg/kg.

FIG. 19A illustrates a pharmacokinetic comparison of BB-001 (15-min IV infusion) and BB-005 (IV bolus administration) clinical trials at the 6 mg dose level.

FIG. 19B illustrates a pharmacokinetic comparison of BB-001 (15-min IV infusion) and BB-005 (IV bolus administration) clinical trials at the 12 mg dose level.

### DETAILED DESCRIPTION

The present disclosure provides compositions and methods for the detection and/or treatment of cancers. The compositions described herein comprise peptide conjugates comprising a detectable label, such as a fluorescent or radio label, which are suitable for the detection and treatment of various cancers. In certain aspects, the compositions are provided in combination with a pharmaceutically acceptable carrier, which can be administered to a subject by any parenteral route of administration. The compositions described herein give rise to a pharmacokinetic profile when administered intravenously to a human subject. Following administration of the compositions described herein, the conjugates bind selectively to cancer cells. The cancer cells can then be detected, for example, by imaging or other visualization or method suitable for detecting, visualizing, or observing the labeled peptide conjugate. In further aspects, the presently described compositions can be used to treat cancer by way of a therapeutic agent, which is attached to the peptide and which acts on the cancer cells following binding to the cancer cells. Furthermore, the present disclosure provides compounds that at the same dosage produce pharmacokinetic profiles that vary according to the rate of administration of the compound, and are therefore considered to be “context-sensitive” compounds. These and other aspects are described in detail herein.

The invention will best be understood by reference to the following detailed description of the aspects and embodiments of the invention, taken in conjunction with the accompanying drawings and figures. The discussion below is descriptive, illustrative and exemplary and is not to be taken as limiting the scope defined by any appended claims.

As used in the specification and appended claims, unless specified to the contrary, the following terms have the meaning indicated.

As used herein and in the appended claims, the singular forms “a,” “and,” and “the” include plural referents unless the context clearly dictates otherwise. Thus, for example, reference to “a compound” includes a plurality of such compounds, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, and where each of the above substituents is unsubstituted unless otherwise indicated.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl, fluorenyl, and naphthyl. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which may include fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl may be saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

“Heterocyclyl” refers to a 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e., thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

The compounds, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. When the compounds described herein contain olefinic double bonds or other centers of geometric asymmetry, and unless specified otherwise, it is intended that the compounds include both E (or trans) and Z (cis) geometric isomers. Likewise, all possible isomers, as well as their racemic and optically pure forms, and all tautomeric forms are also intended to be included.

A “stereoisomer” refers to a compound made up of the same atoms bonded by the same bonds but having different three-dimensional structures, which are not interchangeable. It is therefore contemplated that various stereoisomers and mixtures thereof and includes “enantiomers,” which refers to two stereoisomers whose molecules are nonsuperimposable mirror images of one another.

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In solutions where tautomerization is possible, a chemical equilibrium of the tautomers will exist. The exact ratio of the tautomers depends on several factors, including temperature, solvent, and pH. Some examples of tautomeric pairs include:

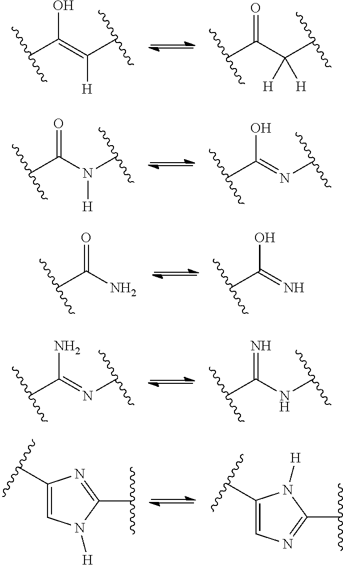

“Optional” or “optionally” means that a subsequently described event or circumstance may or may not occur and that the description includes instances when the event or circumstance occurs and instances in which it does not.

“Pharmaceutically acceptable salt” includes both acid and base addition salts. A pharmaceutically acceptable salt of any one of the alkoxyphenyl-linked amine derivative compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication, reduction, or amelioration of the underlying disorder being treated. Also, a therapeutic benefit is achieved with the eradication, reduction, or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

### Chlorotoxin Conjugates

The present disclosure provides methods for administering compounds that selectively bind to cancerous cells and tissues. In various aspects, these compounds comprise a peptide portion and a detectable agent conjugated together.

In various aspects of the compounds used in the present disclosure, the peptide portions of the compounds described herein have certain features in common with the native chlorotoxin (CTX) peptide. The native chlorotoxin peptide was originally isolated from the scorpion Leiurus quinquestriatus. Chlorotoxin is a 36 amino acid peptide that selectively binds to cancerous cells. The peptide portions of the present compounds have advantageously retained at least some of the cancer-cell binding activity of chlorotoxin. The cancer-cell binding activity of chlorotoxin provides certain advantages for the detection and treatment of cancer because it facilitates the selective localization of imaging agents and therapeutic agents to the cancer cells for the detection and treatment of cancer. In certain aspects, peptides used in the present disclosure are conjugated to moieties, such as detectable labels (e.g., dyes or radiolabels) that are detected (e.g., visualized) in a subject. In some aspects, the chlorotoxin and/or chlorotoxin variants are conjugated to detectable labels to enable tracking of the bio-distribution of a conjugated peptide. The fluorescent moiety can be covalently coupled to the chlorotoxin to allow for the visualization of the conjugate by fluorescence imaging, either directly or through a cleavable or non-cleavable linker as described herein and known to one of ordinary skill in the art.

In some aspects, the fluorescent label used has emission characteristics that are desired for a particular application. For example, the fluorescent label is a fluorescent dye that has an emission wavelength maximum from 500 nm to 1100 nm, from 600 nm to 1000 nm, from 800 nm to 1000 nm, from 600 to 800 nm, from 800 nm to 900 nm, from 650 nm to 850 nm, from 650 nm to 800 nm, from 700 nm to 800 nm, from 800 nm to 880 nm, from 810 nm to 875 nm, from 825 nm to 875 nm, or from 790 nm to 840 nm, or from 800 nm to 830 nm. One of ordinary skill in the art will appreciate the various dyes that are used as detectable labels and that have the emission characteristics herein. In addition, excitation spectra can be used to optimize imaging of visualization of the conjugate. The absorption spectrum of a fluorophore can determine the wavelengths of light energy that excites the molecule to produce its fluorescence. One of ordinary skill in the art will appreciate that the range of illumination wavelengths used to excite a molecule can include light energies over a broad range of wavelengths or over a narrow range of wavelengths within the absorption spectra of the fluorophore molecule. The emission spectrum is the spectrum of light wavelengths that are given off (emitted) from the fluorophore molecule after excitation. With respect to the excitation light, depending on the environment that the fluorophore molecule is in (e.g., surgical bed, tumor tissue, solution, and the like), the fluorophore molecule has an optimal excitation spectrum at around 785 nm (e.g., from 770 nm to 795 nm), for example, from 770 nm to 800 nm, from 775 nm to 795 nm, from 780 nm to 790 nm, from 775 nm to 780 nm, from 780 nm to 785 nm, from 780 nm to 795 nm, from 785 nm to 790 nm, from 790 nm to 795 nm, from 795 nm to 800 nm, from 800 nm to 805 nm, or from 805 nm to 810 nm. In addition the fluorophore is a fluorescent dye that has an optimal excitation spectrum at 750 nm, 755 nm, 760 nm, 765 nm, 770 nm, 775 nm, 780 nm, 785 nm, 790 nm, 795 nm, 800 nm, 805 nm, or 810 nm, or any of the foregoing +/−3 nm, +/−2 nm, or +/−1 nm. In some embodiments, dependeing on the environment that the fluorophore molecule is in (e.g., surgical bed, tumor tissue, solution, and the like), the fluorophore molecule has an optimal excitation spectrum) from 600 nm to 900 nm.

Some other exemplary dyes used in the present disclosure include near-infrared dyes, such as, but not limited to, DyLight-680, DyLight-750, VivoTag-750, DyLight-800, IRDye-800, VivoTag-680, Cy5.5, or indocyanine green (ICG). In some aspects, near infrared dyes often include cyanine dyes. Additional non-limiting examples of fluorescent dyes for use as a conjugating molecule in the present disclosure include acradine orange or yellow, Alexa Fluors and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. In some aspects, conjugates of the present disclosure comprise other dyes, including but not limited to those provided below in TABLE 1. Regarding TABLE 1, the peak absorption and emission values for a given fluorophore can vary depending on the environment (e.g., solution, tissue, etc.) that the fluorophore is present in as well as the concentration of fluorophore or fluorophore conjugate utilized.

TABLE 1
Exemplary Fluorescent Reporter Molecules With Peak Absorbance
(Abs.) and Emission (Em.) Wavelengths Specified (in nanometer)

| | Peak | Peak |
|---|---|---|
| Dye | Abs. | Em. |
| Methoxycoumarin | 360 | 410 |
| Fluospheres Blue | 356 | 412 |
| Cascade Blue | 377 | 420 |
| PBFI | 360 | 420 |
| DyeLight 405 | 400 | 420 |
| Cascade Blue | 400 | 420 |
| Alexa Fluor 405 | 401 | 421 |
| Alexa Fluor 405 | 401 | 421 |
| LysoTracker Blue | 373 | 422 |
| LysoSensor Blue | 374 | 424 |
| AMCA | 345 | 425 |
| True Blue | 365 | 425 |
| 7-amino-4-methylcoumarin (AMC) | 351 | 430 |
| Phorwite AR | 360 | 430 |
| DyLight 350 | 353 | 432 |
| Uvitex SFC | 365 | 435 |
| 4-methylumbelliferone | 360 | 440 |
| CellTrace Calcein Blue | 373 | 440 |
| Calcofluor White | 350 | 440 |
| Fast Blue | 360 | 440 |
| LysoSensor Yellow/Blue (pH 8.0) | 329 | 440 |
| LysoSensor Yellow/Blue (pH 8.0) | 329 | 440 |
| LysoSensor Yellow/Blue (pH 8.0) | 329 | 440 |
| LysoSensor Yellow/Blue (pH 8.0) | 329 | 440 |
| Alexa Fluor 350 | 346 | 442 |
| AMCA-X | 353 | 442 |
| LIVE/DEAD Fixable Blue Dead Cell Stain | 344 | 442 |
| Y66H | 360 | 442 |
| ABQ | 344 | 445 |
| BFP | 382 | 448 |
| BFP | 382 | 448 |
| 7-hydroxy-4-methylcoumarin | 360 | 449 |
| SpectrumBlue | 405 | 449 |
| DiFMU (pH 9.0) | 357 | 450 |
| sgBFP (Super Glow BFP) | 387 | 450 |
| SpectrumBlue | 400 | 450 |
| CellTrace Calcein Violet | 401 | 451 |
| DAPI | 345 | 455 |
| NucBlue Fixed Cell Stain | 345 | 455 |
| Pacific Blue | 405 | 455 |
| Pacific Blue | 410 | 455 |
| PO-PRO-1 | 435 | 455 |
| PO-PRO-1 | 435 | 455 |
| POPO-1 | 434 | 456 |
| POPO-1 | 434 | 456 |
| TagBFP | 402 | 457 |
| Marina Blue | 365 | 460 |
| SITS | 365 | 460 |
| Thioflavin TCN | 350 | 460 |
| Monochlorobimane(mBCI) | 380 | 461 |
| Quinine Sulfate | 349 | 461 |
| Acridine | 362 | 462 |
| CellLights CFP | 434 | 477 |
| ECFP | 434 | 477 |
| CFP | 434 | 477 |
| 1,8-ANS | 372 | 480 |
| SYTOX Blue | 444 | 480 |
| SYTOX Blue | 444 | 480 |
| Hoechst 33342 | 347 | 483 |
| NucBlue Live Cell Stain | 347 | 483 |
| Thiolyte | 378 | 483 |
| SYTO 45 | 452 | 484 |
| SYTO 45 | 452 | 484 |
| SYTO 45 | 452 | 484 |
| SYTO 45 | 452 | 484 |
| SYTO 45 | 452 | 484 |
| Hoechst 33258 | 345 | 487 |
| AmCyan | 548 | 489 |
| Auramine O | 445 | 500 |
| SYTO 9 | 482 | 500 |
| SYTO 9 | 482 | 500 |
| SYTO 9 | 482 | 500 |
| SYTO 9 | 482 | 500 |
| SYTO 9 | 482 | 500 |
| DiO | 484 | 501 |
| DiO | 484 | 501 |
| DiO | 484 | 501 |
| LysoSensor Green | 448 | 503 |
| LysoSensor Green | 448 | 503 |
| LysoSensor Green | 448 | 503 |
| LysoSensor Green | 448 | 503 |
| LysoSensor Green | 448 | 503 |
| SYTO 13 | 487 | 505 |
| LysoSensor Green (pH 5) | 442 | 505 |
| SYTO 13 | 487 | 505 |
| SYTO 13 | 487 | 505 |
| SYTO 13 | 487 | 505 |
| SYTO 13 | 487 | 505 |
| DiO (Vybrant DiO) | 489 | 506 |
| HCS LipidTox Green | 498 | 506 |
| LIVE/DEAD Fixable Green | 498 | 506 |
| LIVE/DEAD Fixable Green | 498 | 506 |
| ATTO 465 | 453 | 507 |
| CellLights GFP | 488 | 507 |
| CellEvent Caspase-3/7 Green | 488 | 507 |
| Diversa Green-FP | 484 | 507 |
| GFP (EGFP) | 488 | 507 |
| S65C | 479 | 507 |
| YO-PRO-1 | 491 | 507 |
| GFP | 488 | 507 |
| YO-PRO-1 | 491 | 507 |
| GFP | 488 | 507 |
| YO-PRO-1 | 491 | 507 |
| GFP | 488 | 507 |
| YO-PRO-1 | 491 | 507 |
| Premo FUCCI Cell Cycle Sensor (S/G2/M phases) | 474 | 509 |
| sgGFP (Super Glow GFP) | 474 | 509 |
| wtGFP (wild type GFP, non-UV excitation) | 475 | 509 |
| YOYO-1 | 491 | 509 |
| YOYO-1 | 491 | 509 |
| YOYO-1 | 491 | 509 |
| YOYO-1 | 491 | 509 |
| YOYO-1 | 491 | 509 |
| HPTS (Solvent Green 7) | 455 | 510 |
| Nitrobenzoxadiazole | 465 | 510 |
| S65L | 484 | 510 |
| LysoTracker Green | 504 | 511 |
| S65T | 488 | 511 |
| LysoTracker Green | 504 | 511 |
| LysoTracker Green | 504 | 511 |
| MitoTracker Green FM | 490 | 512 |
| MitoTracker Green FM | 490 | 512 |
| MitoTracker Green FM | 490 | 512 |
| MitoTracker Green FM | 490 | 512 |
| FluoSpheres Yellow-Green | 501 | 513 |
| Evans Blue | 460 | 515 |
| Evans Blue | 460 | 515 |
| rsGFP (red shifted GFP, S65 T) | 498 | 516 |
| CellTracker Violet BMQC | 415 | 516 |
| HCS CellMask Green | 493 | 516 |
| CellTracker Violet BMQC | 415 | 516 |
| CellTracker Violet BMQC | 415 | 516 |
| CellTracker Violet BMQC | 415 | 516 |
| CellTracker Violet BMQC | 415 | 516 |
| HCS CellMask Green | 493 | 516 |
| 5-carboxyfluorescein(5-FAM) | 492 | 518 |
| ActinGreen (Alexa Fluor 488 phalloidin) | 496 | 518 |
| Alexa Fluor 488 | 496 | 518 |
| Click-iT EdU Alexa Fluor 488 | 496 | 518 |
| DyLight + C110 488 | 493 | 518 |
| Fluoro-Emerald | 494 | 518 |
| Aiexa Fluor 488 | 496 | 518 |
| Carboxyfluorescein (5-FAM) | 492 | 518 |
| Aiexa Fluor 488 | 496 | 518 |
| Carboxyfluorescein (5-FAM) | 492 | 518 |
| CellRox Green | 485 | 520 |
| FITC (Fluorescein) | 492 | 520 |
| Fluor-X | 494 | 520 |
| Rhodamine 110 | 496 | 520 |
| SYTO 16 | 490 | 520 |
| FITC | 492 | 520 |
| Rhodamine 110 | 496 | 520 |
| SYTO 16 | 490 | 520 |
| FITC | 492 | 520 |
| Rhodamine 110 | 496 | 520 |
| SYTO 16 | 490 | 520 |
| SYTO 16 | 490 | 520 |
| FITC | 492 | 520 |
| Rhodamine 110 | 496 | 520 |
| SYTO 16 | 490 | 520 |
| SYBR Green I | 497 | 521 |
| SYBR Green I | 497 | 521 |
| SYBR Green I | 497 | 521 |
| SYBR Green I | 497 | 521 |
| SYBR Green I | 497 | 521 |
| Quant-iT PicoGreen | 502 | 522 |
| Spectru mgreen | 498 | 522 |
| NucGreen Dead Cell Stain | 504 | 523 |
| Rhodamine Green | 497 | 523 |
| Rhodol Green | 496 | 523 |
| SYTOX Green | 504 | 523 |
| Rhodamine Green | 497 | 523 |
| Rhodamine Green | 497 | 523 |
| Rhodamine Green | 497 | 523 |
| Neurotrace 500/525 Green | 497 | 524 |
| Oregon Green 488 | 498 | 524 |
| SYBR Safe | 507 | 524 |
| NeuroTrace 500/525 Nissl stain | 497 | 524 |
| Oregon Green 488 | 498 | 524 |
| NeuroTrace 500/525 Nissl stain | 497 | 524 |
| Oregon Green 488 | 498 | 524 |
| NeuroTrace 500/525 Nissl stain | 497 | 524 |
| NeuroTrace 500/525 Nissl stain | 497 | 524 |
| Oregon Green 488 | 498 | 524 |
| Dansyl | 335 | 525 |
| Fluoro-Jade B | 480 | 525 |
| Qdot 525 | UV | 525 |
| SYTO 11 | 506 | 525 |
| Qdot 525 | UV | 525 |
| Qdot 525 | UV | 525 |
| Acridine Orange + DNA | 500 | 526 |
| LIVE/DEAD Fixable Green | 498 | 526 |
| Surf Green EX | 469 | 526 |
| Acridine Orange + DNA | 500 | 526 |
| Acridine Orange + DNA | 500 | 526 |
| Acridine Orange + DNA | 500 | 526 |
| Acridine Orange (+DNA) | 500 | 526 |
| ThiolTracker Violet | 405 | 526 |
| ThiolTracker Violet | 405 | 526 |
| ThiolTracker Violet | 405 | 526 |
| ThiolTracker Violet | 405 | 526 |
| Acridine Orange (+DNA) | 500 | 526 |
| ThiolTracker Violet | 405 | 526 |
| SYTO RNASelect | 503 | 527 |
| EYFP | 514 | 527 |
| SYTO RNASelect | 503 | 527 |
| SYTO RNASelect | 503 | 527 |
| SYTO RNASelect | 503 | 527 |
| SYTO RNASelect | 503 | 527 |
| Rhodamine 123 | 507 | 529 |
| YFP | 512 | 529 |
| F2N12S | 405 | 530, |
| | | 585 |
| F2N12S | 405 | 530, |
| | | 585 |
| F2N12S | 405 | 530, |
| | | 585 |
| F2N12S | 405 | 530, |
| | | 585 |
| F2N12S | 405 | 530, |
| | | 585 |
| F2N12S | 405 | 530, |
| | | 585 |
| F2N12S | 405 | 530, |
| | | 585 |
| Magnesium Green | 506 | 530 |
| NBD Amine | 450 | 530 |
| TO-PRO-1 | 515 | 530 |
| TOTO-1 | 513 | 531 |
| Oregon Green 514 | 512 | 532 |
| Sodium Green | 506 | 532 |
| Vybrant DyeCycle Green | 505 | 532 |
| pHrodo Green | 509 | 533 |
| NBD-X | 467 | 538 |
| NBD-X | 467 | 538 |
| NBD-X | 467 | 538 |
| NBD-X | 467 | 538 |
| NBD-X | 467 | 538 |
| NBD-X | 467 | 538 |
| NBD-X | 467 | 538 |
| SYBR Gold | 495 | 539 |
| SYBR Gold | 495 | 539 |
| SYBR Gold | 495 | 539 |
| SYBR Gold | 495 | 539 |
| SYBR Gold | 495 | 539 |
| Alexa Fluor 430 | 432 | 540 |
| Auramine | 460 | 540 |
| Aurophosphine | 470 | 540 |
| BCECF | 499 | 540 |
| BODIPY 492/515 | 490 | 540 |
| BODIPY 505/515 | 502 | 540 |
| BODIPY FL | 502 | 540 |
| BTC | 464 | 540 |
| Calcein | 494 | 540 |
| Calcium Green-1 | 506 | 540 |
| Catskill Green 540 | 482 | 540 |
| CellTracker Green | 490 | 540 |
| CFDA | 494 | 540 |
| CFP | 434 | 540 |
| Cy2 | 492 | 540 |
| CyQUANT Direct (CyQUANT GR) | 500 | 540 |
| DAF-FM | 493 | 540 |
| Emerald Green | 490 | 540 |
| Fluo-3 | 506 | 540 |
| Fluo-4 | 494 | 540 |
| H2DCFDA (H2-DCF, DCFR) | 504 | 540 |
| Alexa Fluor 430 | 434 | 540 |
| Alexa Fluor 430 | 432 | 540 |
| BCECF (pH 5.2) | 499 | 540 |
| Calcein | 494 | 540 |
| CellTracker Green CMFDA | 490 | 540 |
| CFP | 434 | 540 |
| Cy2 | 492 | 540 |
| CyQUANT Direct | 500 | 540 |
| DAF-FM | 493 | 540 |
| Fluo-4 | 494 | 540 |
| Alexa Fluor 430 | 432 | 540 |
| BCECF (pH 5.2) | 499 | 540 |
| Calcein | 494 | 540 |
| CellTracker Green CMFDA | 490 | 540 |
| CFP | 434 | 540 |
| Cy2 | 492 | 540 |
| CyQUANT Direct | 500 | 540 |
| Alexa Fluor 430 | 432 | 540 |
| BCECF (pH 5.2) | 499 | 540 |
| CFP | 434 | 540 |
| Cy2 | 492 | 540 |
| Alexa Fluor 430 | 432 | 540 |
| BCECF (pH 5.2) | 499 | 540 |
| Alexa Fluor 430 | 432 | 540 |
| BCECF (pH 5.2) | 499 | 540 |
| Alexa Fluor 430 | 432 | 540 |
| BCECF (pH 5.2) | 499 | 540 |
| Calcein | 494 | 540 |
| CellTracker Green CMFDA | 490 | 540 |
| CFP | 434 | 540 |
| Cy2 | 492 | 540 |
| CyQUANT Direct | 500 | 540 |
| DAF-FM | 493 | 540 |
| Fluo-4 | 494 | 540 |
| TET | 520 | 541 |
| TET | 521 | 542 |
| Lucifer Yellow | 423 | 543 |
| Qdot 545 | UV | 543 |
| Lucifer Yellow | 423 | 543 |
| Lucifer Yellow | 423 | 543 |
| Lucifer Yellow | 423 | 543 |
| Lucifer Yellow | 423 | 543 |
| Lucifer Yellow | 423 | 543 |
| Lucifer Yellow | 423 | 543 |
| Lucifer Yellow | 423 | 543 |
| Lucifer yellow | 428 | 544 |
| Lucifer Yellow | 428 | 544 |
| Lucifer yellow | 428 | 544 |
| Eosin | 524 | 545 |
| JOJO-1 | 529 | 545 |
| Qdot 545 | UV | 545 |
| Qdot 545 | UV | 545 |
| Auramine O | 460 | 550 |
| Pacific Orange | 440 | 551 |
| Pacific Orange | 440 | 551 |
| Pacific Orange | 440 | 551 |
| Pacific Orange | 440 | 551 |
| Pacific Orange | 440 | 551 |
| Pacific Orange | 440 | 551 |
| mBanana | 540 | 553 |
| ER-Tracker Blue-White DPX | 371 | 554 |
| Alexa Fluor 532 | 532 | 554 |
| FocalCheck Double Orange | 540 | 555 |
| HEX | 533 | 558 |
| Fluospheres Orange | 539 | 560 |
| mHoneydew | 478 | 561 |
| Vybrant DyeCycle Orange | 518 | 562 |
| ActinRed 555 (rhodamin pphalloidin) | 540 | 565 |
| Alexa Fluor 555 | 555 | 565 |
| CellRox Orange | 545 | 565 |
| Qdot 565 | UV | 565 |
| Qdot 565 | UV | 565 |
| DiI (CellTracker DiI) | 551 | 568 |
| mOrange | 548 | 568 |
| OFP | 546 | 568 |
| Bodipy TMR | 544 | 569 |
| Cy3 | 552 | 570 |
| PO-PRO-3 | 539 | 570 |
| SYTOX Orange | 567 | 570 |
| CellMask Orange | 556 | 571 |
| Alexa Fluor 546 | 561 | 572 |
| POPO-3 | 532 | 573 |
| TurboRFP | 553 | 574 |
| Calcium Orange | 549 | 575 |
| CellTracker Orange | 547 | 575 |
| LIVE/DEAD Fixable Yellow | 405 | 575 |
| LIVE/DEAD Fixable Yellow | 405 | 575 |
| LIVE/DEAD Fixable Yellow | 405 | 575 |
| LIVE/DEAD Fixable Yellow | 405 | 575 |
| LIVE/DEAD Fixable Yellow | 405 | 575 |
| LIVE/DEAD Fixable Yellow | 405 | 575 |
| DyLight 594 | 562 | 576 |
| MitoTracker Orange CMTMRos(MitoTracker | 551 | 576 |
| Orange CM-H2TMRos) | | |
| Phycoerythrin (PE, R-phycoerythrin) | 567 | 576 |
| Rhod-2 | 551 | 576 |
| Rhodamine Phalloidin | 557 | 576 |
| X-Rhod-1 | 570 | 576 |
| DsRed-Express | 557 | 579 |
| Rhodamine Red | 560 | 580 |
| TAMRA | 565 | 580 |
| Tetramethylrhodamine (TRITC) | 555 | 580 |
| dTomato | 554 | 581 |
| DsRed2 | 563 | 582 |
| Amplex Ultra Red | 567 | 582 |
| Amplex Red | 571 | 583 |
| Amplex UltraRed | 568 | 583 |
| Amplex Red | 570 | 583 |
| Premo FUCCI Cell Cycle Sensor (G1 phase) | 555 | 584 |
| TagRFP | 555 | 584 |
| CellLights RFP | 552 | 585 |
| mTangerine | 568 | 585 |
| Resorufin | 570 | 585 |
| RFP | 552 | 585 |
| Qdot 585 | UV | 585 |
| Qdot 585 | UV | 585 |
| DsRed Monomer | 556 | 586 |
| pHrodo Red | 559 | 586 |
| Carboxy SNARF-1 | 548 | 587 |
| pHrodo Red | 559 | 587 |
| SpectrumOrange | 559 | 588 |
| DsRed2 | 563 | 588 |
| DiA | 456 | 590 |
| DiA | 456 | 590 |
| DiA | 456 | 590 |
| DiA | 456 | 590 |
| DiA | 456 | 590 |
| DiA | 456 | 590 |
| DiA | 456 | 590 |
| DiA | 456 | 590 |
| rhodamine Red-X | 572 | 591 |
| CellTrace calcein red-orange | 575 | 592 |
| LysoTracker Red | 573 | 592 |
| Sulforhodamine 101 | 578 | 593 |
| sulforhodamine 101 | 577 | 593 |
| ROX (6-ROX) | 568 | 595 |
| 2-dodecylresorufin | 582 | 595 |
| Cy3.5 | 579 | 597 |
| Cy 3.5 | 581 | 597 |
| MitoTracker Red CMXRos | 578 | 597 |
| BOBO-3 | 570 | 602 |
| Ethidium Bromide | 521 | 602 |
| X-rhod-1 | 579 | 602 |
| BOBO-1 | 570 | 602 |
| BOBO-1 | 570 | 602 |
| BOBO-1 | 570 | 602 |
| 5-ROX | 577 | 603 |
| Alexa Fluor 568 | 578 | 603 |
| Qdot 605 | UV | 605 |
| Qdot 605 | UV | 605 |
| BOBO-3 | 571 | 606 |
| Calcium Crimson | 589 | 608 |
| Fluospheres Red microspheres | 577 | 608 |
| ReAsH (TC-ReAsH) | 593 | 608 |
| CellTracker Red | 585 | 612 |
| LIVE/DEAD Fixable Red | 593 | 613 |
| CellTracker Red CMTPX | 584 | 613 |
| LIVE/DEAD Fixable Red Dead Cell stain | 595 | 613 |
| DiA (FAST DiA) | 491 | 613 |
| DiA | 491 | 613 |
| HCS CellMask Red stain | 587 | 614 |
| HCS LipidTox Red | 582 | 615 |
| HCS LipidTOX Red | 582 | 615 |
| mCherry | 587 | 615 |
| Texas Red | 592 | 615 |
| Ethidium Homodimer-1 (EthD-1) | 530 | 618 |
| Propidium Iodide (PI) | 530 | 618 |
| Alexa Fluor 594 | 590 | 618 |
| Click-iT Alexa Fluor 594 | 590 | 618 |
| DyLight 594 | 593 | 618 |
| SYPRO Ruby | 450 | 618 |
| SYPRO Ruby | 450 | 618 |
| SYPRO Ruby | 450 | 618 |
| SYPRO Ruby | 450 | 618 |
| SYPRO Ruby | 450 | 618 |
| SYPRO Ruby | 450 | 618 |
| Bodipy TR-X | 588 | 621 |
| CellTrace BODIPY TR methyl esther | 597 | 625 |
| mRaspberry | 598 | 625 |
| Qdot 625 | UV | 625 |
| Qdot 625 | UV | 625 |
| FM 1-43 | 510 | 626 |
| FM 1-43 | 510 | 626 |
| FM 1-43 | 510 | 626 |
| FM 1-43 | 510 | 626 |
| FM 1-43 | 510 | 626 |
| FM 1-43 | 510 | 626 |
| FM 1-43 | 510 | 626 |
| FM 1-43 | 510 | 626 |
| YO-PRO-3 | 612 | 628 |
| Alexa Fluor 610 | 610 | 629 |
| Magic Red | 570 | 630 |
| CTC Formazan | 450 | 630 |
| CTC Formazan | 450 | 630 |
| YOYO-3 | 612 | 631 |
| Katushka (Turbo FP635) | 588 | 635 |
| mKate | 588 | 635 |
| SYTO 17 | 620 | 635 |
| Di-8 ANEPPS | 468 | 635 |
| Di-8 ANEPPS | 468 | 635 |
| Di-8-ANEPPS | 465 | 635 |
| Di-8-ANEPPS | 465 | 635 |
| Di-8-ANEPPS | 465 | 635 |
| Di-8-ANEPPS | 465 | 635 |
| Di-8-ANEPPS | 465 | 635 |
| Di-8-ANEPPS | 465 | 635 |
| Di-8-ANEPPS | 465 | 635 |
| Nile Red | 551 | 636 |
| Nile red (triglyceride) | 552 | 636 |
| Nile red (triglyceride) | 552 | 636 |
| Nile red (triglyceride) | 552 | 636 |
| Fura Red (high Ca2+) | 436 | 637 |
| Nile Red phospholipid | 551 | 638 |
| SYTO 17 | 619 | 638 |
| Bodipy 630/650-X | 625 | 641 |
| BODIPY 630/650X | 626 | 641 |
| 7-AAD | 549 | 644 |
| HCS NuclearMask Red | 624 | 644 |
| HCS NuclearMask Red | 622 | 644 |
| SYTO 59 | 621 | 644 |
| SYTO 59 | 622 | 645 |
| Fluospheres Crimson microspheres | 620 | 646 |
| FluoSpheres crimson microspheres | 621 | 646 |
| SYTOX AADvanced dead cell stain | 546 | 647 |
| Alexa Fluor 635 | 634 | 647 |
| HcRed | 594 | 649 |
| mPlum | 590 | 649 |
| SYTO 61 | 619 | 649 |
| Alexa Fluor 633 | 631 | 650 |
| Acridine Orange + RNA | 460 | 650 |
| Acridine Orange + RNA | 460 | 650 |
| Acridine Orange (+RNA) | 460 | 650 |
| Acridine Orange (+RNA) | 460 | 650 |
| HCS LipidTOX Deep Red | 634 | 652 |
| Fura Red (+Ca2+) | 436 | 655 |
| Fura Red (+Ca2+) | 436 | 655 |
| Fura Red (+Ca2+) | 436 | 655 |
| Fura Red (+Ca2+) | 436 | 655 |
| Qdot 655 | UV | 655 |
| Fura Red (+Ca2+) | 436 | 655 |
| Fura Red (+Ca2+) | 436 | 655 |
| Qdot 655 | UV | 655 |
| FxCycle Far Red | 641 | 657 |
| TO-PRO-3 | 642 | 657 |
| DDAO | 648 | 658 |
| DyLight 633 | 638 | 658 |
| SYTOX Red | 640 | 658 |
| ATTO 635 | 635 | 658 |
| APC (Allophycocyanin) | 651 | 660 |
| MitoTracker Deep Red FM | 641 | 661 |
| NucRed Dead 647 | 642 | 661 |
| TOTO-3 | 642 | 661 |
| BODIPY 650/665 | 647 | 665 |
| CellRox Deep Red | 640 | 665 |
| LIVE/DEAD Fixable Far Red | 650 | 665 |
| Cy5 | 648 | 666 |
| Lysotracker Deep Red | 647 | 668 |
| Alexa Fluor 647 | 650 | 670 |
| Click-iT Alexa Fluor 647 | 650 | 670 |
| DiD (Vybrant DiD) | 645 | 670 |
| HCS CellMask Deep Red stain | 649 | 670 |
| ATTO 647 | 644 | 670 |
| Fura Red (−Ca2+) | 473 | 670 |
| Fura Red (−Ca2+) | 473 | 670 |
| Fura Red (−Ca2+) | 473 | 670 |
| Fura Red (−Ca2+) | 473 | 670 |
| Fura Red (−Ca2+) | 473 | 670 |
| DyLight 649 | 654 | 673 |
| Carboxynaphthofluorescein | 600 | 674 |
| PerCP | 488 | 675 |
| CellMask Deep Red plasma membrane stain | 658 | 676 |
| DRAQ5 | 650 | 680 |
| SYTO 60 | 649 | 681 |
| SYTO 62 | 650 | 681 |
| SYTO 60 | 650 | 681 |
| FluoSpheres dark red microspheres | 657 | 683 |
| ATTO 655 | 663 | 683 |
| FluoSpheres Dark Red fluorescent microspheres | 656 | 683 |
| NucRed Live 647 | 638 | 686 |
| Vybrant DyeCycle Ruby | 638 | 686 |
| HCS NuclearMask Deep Red | 635 | 687 |
| Cy5.5 | 672 | 690 |
| Alexa Fluor 660 | 663 | 691 |
| Alexa Fluor 660 | 663 | 691 |
| Cy5.5 | 678 | 696 |
| DY-675 | 675 | 699 |
| IRDye 700 Phosphoramidite | 691 | 699 |
| ATTO 680 | 680 | 700 |
| Alexa Fluor 680 | 679 | 702 |
| HiLyte Fluor 680 | 688 | 702 |
| Qdot 705 Nanocrystals | 300 | 702 |
| Alexa Fluor 680 | 679 | 704 |
| DyLight 680 | 676 | 705 |
| Qdot 705 | UV | 705 |
| Qdot 705 | UV | 705 |
| Quasa 705 | 688 | 706 |
| IRDye 680 NHS Ester | 683 | 710 |
| RH 795 | 530 | 712 |
| RH 795 | 530 | 712 |
| RH 795 | 530 | 712 |
| RH 795 | 530 | 712 |
| RH 795 | 530 | 712 |
| Alexa Fluor 700 | 696 | 719 |
| ATTO 700 | 699 | 719 |
| FM 4-64 | 558 | 734 |
| FM 4-64 | 558 | 734 |
| FM 4-64 | 558 | 734 |
| FM 4-64 | 558 | 734 |
| Cy7 | 745 | 766 |
| LIVE/DEAD Fixable near-IR | 750 | 775 |
| CellVue NIR780 | 743 | 776 |
| DyLight 750 | 752 | 778 |
| IRDye 800CW | 774 | 789 |
| XenoLight CF770 | 770 | 797 |
| Qdot 800 | UV | 800 |
| Qdot 800 | UV | 800 |
| Indocyanine Green | 768 | 807 |

In some other aspects, the conjugate compounds used include a chemiluminescent compound, colloidal metal, luminescent compound, phosphorescent compound, enzyme, radioisotope, or paramagnetic labels.

In certain aspects, the conjugates used in the present disclosure are conjugated to radioactive isotopes instead of or in addition to other types of detectable agents. Certain isotopes suitable for use in the present compounds can include, but are not limited to, iodine-131, iodine-125, bismuth-212, bismuth-213, lutetium-177, rhenium-186, rhenium-188, yttrium-90, astatine-211, phosphorus-32 and/or samarium-153. In some aspects, the conjugates of the present disclosure contain one or more atoms having an atomic mass or mass number different from the atomic mass or mass number usually found in nature, including but not limited to hydrogen, carbon, fluorine, phosphorous, copper, gallium, yttrium, technetium, indium, iodine, rhenium, thallium, bismuth, astatine, samarium, and lutetium (for example, ³H, ³H, ¹³C, ¹⁴C, ¹⁸F, ³²P, ³⁵S, ⁶⁴Cu, ⁶⁷Ga, ⁹⁰Y, ^{99M}Tc, ¹¹¹In, ¹²⁵I, ¹²³I, ¹³¹I, ¹³⁵I, ¹⁸⁶Re, ¹⁸⁷Re, ²⁰¹Tl, ²¹²Bi, ²¹¹At, ¹⁵³Sm and/or ¹⁷⁷Lu). In other aspects, the conjugates of the present disclosure are labeled with a paramagnetic metal ion that is a good contrast enhancer in Magnetic Resonance Imaging (MRI). Examples of such paramagnetic metal ions include, but are not limited to, gadolinium III (Gd³⁺), chromium 111 (Cr³⁺), dysprosium III (Dy³⁺), iron 111 (Fe³⁺), manganese II (Mn²⁺), and ytterbium III (Yb³⁺). In certain embodiments, the labeling moiety comprises gadolinium III (Gd³⁺).

In some aspects, the conjugates used in the present disclosure are conjugated to biotin. In addition of extension of half-life, biotin can also act as an affinity handle for retrieval of the peptides from tissues or other locations. In one aspect, the conjugates are conjugated, e.g., to a biotinidase resistant biotin with a PEG linker (e.g., NHS-dPEG4-Biotinidase resistant biotin). In some aspects, fluorescent biotin conjugates that can act both as a detectable label and an affinity handle are used. Non-limiting examples of commercially available fluorescent biotin conjugates include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, alexa fluor 488 biocytin, alexa flour 546, alexa fluor 549, lucifer yellow cadaverine biotin-X, Lucifer yellow biocytin, Oregon green 488 biocytin, biotin-rhodamine and tetramethylrhodamine biocytin.

In certain embodiments, the chlorotoxin and chlorotoxin variants can be conjugated to moieties, such as detectable labels (e.g., dyes) that can be detected (e.g., visualized) in a subject. In some embodiments, the chlorotoxin and/or chlorotoxin variants can be conjugated to detectable labels to enable tracking of the bio-distribution of a conjugated peptide. The detectable labels can include fluorescent dyes. Non-limiting examples of fluorescent dyes that could be used as a conjugating molecule in the present disclosure include rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, and thiorhodamine, cyanine, indocarbocyanine, oxacarbocyanine, thiacarbocyanine, merocyanine, a cyanine dye (e.g., cyanine 2, cyanine 3, cyanine 3.5, cyanine 5, cyanine 5.5, cyanine 7), oxadiazole derivatives, pyridyloxazole, nitrobenzoxadiazole, benzoxadiazole, pyrene derivatives, cascade blue, oxazine derivatives, Nile red, Nile blue, cresyl violet, oxazine 170, acridine derivatives, proflavin, acridine orange, acridine yellow, arylmethine derivatives, auramine, xanthene dyes, sulfonated xanthenes dyes, Alexa Fluors (e.g., Alexa Fluor 594, Alexa Fluor 633, Alexa Fluor 647, Alexa Fluor 700), crystal violet, malachite green, tetrapyrrole derivatives, porphyrin, phtalocyanine, and bilirubin. Some other example dyes include near-infrared dyes, such as, but not limited to, Cy5.5, indocyanine green (ICG), DyLight 750 or IRdye 800. In some embodiments, near infrared dyes can include cyanine dyes.

Chemotherapeutics, anti-cancer drugs, and anti-cancer agents can include, but are not limited to: radioisotopes, toxins, enzymes, sensitizing drugs, nucleic acids, including interfering RNAs, antibodies, anti-angiogenic agents, cisplatin, anti-metabolites, mitotic inhibitors, growth factor inhibitors, paclitaxel, temozolomide, topotecan, fluorouracil, vincristine, vinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, and their equivalents, as well as photo-ablation.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

Suitable diagnostic agents can include agents that provide for the detection by fluorescence methods as well as methods other than fluorescence imaging. Other suitable diagnostic agents can include radiolabels (e.g., radio isotopically labeled compounds) such as ¹²⁵ I, ¹⁴C, and ³¹P, among others; and magnetic resonance imaging agents.

Suitable targeting agents can include antibodies, polypeptides, polysaccharides, nucleic acids, fatty acids, lipids, glycolipids, sterols, vitamins, cofactors, hormones, neurotransmitters, and metabolites.

In another aspect of the invention, compositions used include the modified chlorotoxin peptide conjugates as provided. The composition used can include a pharmaceutically acceptable carrier or diluent for delivery of the modified chlorotoxin peptide conjugate. Suitable pharmaceutically acceptable carriers or diluents can include saline or dextrose for injection.

In various aspects, the presently described compounds used further comprise a detectable label, which can be used for the detection of the peptide-label conjugate and the cancerous cells to which they are bound.

In various aspects, compounds used in the present disclosure have the structure of Formula (I), or a pharmaceutically acceptable salt thereof:

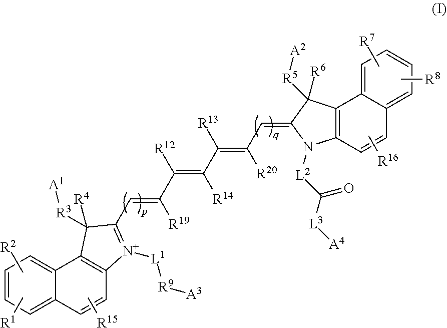

wherein:
R¹, R², R³, R⁴, R⁵, R⁶, R⁷, R⁸, R¹⁵, and R¹⁶ are each independently selected from hydrogen, C₁-C₆ alkyl, C₁-C₆ alkylene-COOH, sulfonate, —COOH, —SO₂—NH₂, C₁-C₆ alkoxy, C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—;
R⁹ is hydrogen, sulfonate, —COOH, C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—;
L¹ is C₃-C₆ alkylene;
L² is C₁-C₁₀ alkylene;
L³ is a bond, —O—, —NR¹⁰—, —NR¹⁰—C₁-C₆ alkylene-, —O—NR¹⁰—, —NR¹⁰—C₁-C₆ alkylene-(O—C₁-C₆ alkylene)ₙ-, —NR¹⁰-L⁴-, —NR¹⁰—C₁-C₆ alkylene-NR¹¹—(C(═O)—C₁-C₆ alkylene-O—)ₘ—, or —NR¹⁰—C₁-C₆ alkylene-NR¹⁰—C₁-C₆ alkylene-NR¹⁰—C₁-C₆ alkylene-;
L⁴ is a bond, -heterocyclyl-, or -heterocyclyl-C₁-C₆ alkylene-;
R¹⁰ is hydrogen or C₁-C₆ alkyl;
R¹¹ is hydrogen or C₁-C₆ alkyl;
R¹² and R¹³ are each independently selected from hydrogen, C₁-C₆ alkyl, or R¹² and
R¹³ are joined together along with the other atoms to which they are attached to form a 5-membered or 6-membered carbocyclic or heterocyclic ring;
R¹⁴ is hydrogen or C₁-C₆ alkylene, -(L⁵)-aryl, -(L⁵)-aryl-A, -(L⁵)-heteroaryl, -(L⁵)-heteroaryl-A⁵, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
L⁵ is a bond, C₁-C₁₀ alkylene, —O—, or —NR¹⁰—;
R¹⁷ and R¹⁸ are each independently hydrogen or aryl;
R¹⁹ and R²⁰ are each independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
n is 0, 1, 2, or 3;
m is 0, 1, 2, or 3;
p is 0, 1, 2, or 3;
q is 0, 1, 2, or 3;
x is 0 or 1; and
one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof and the others of A¹, A², A³, A⁴, or A⁵ are each independently absent, hydrogen, —COOH, or sulfonate.

In various aspects, the presently described compounds used further comprise a detectable label, which can be used for the detection of the peptide-label conjugate and the cancerous cells to which they are bound.

In various aspects, compounds used in the present disclosure have the structure of Formula (II), or a pharmaceutically acceptable salt thereof:

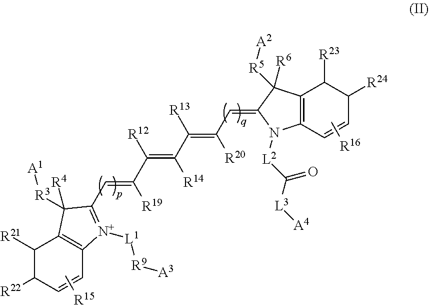

wherein:
R³, R⁴, R⁵, R⁶, R¹⁵, and R¹⁶ are each independently selected from hydrogen, C₁-C₆ alkyl, C₁-C₆ alkylene-COOH, sulfonate, —COOH, —SO₂—NH₂, C₁-C₆ alkoxy, C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—;
R⁹ is hydrogen, sulfonate, —COOH, C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—;
L¹ is C₃-C₆ alkylene;
L² is C₁-C₁₀ alkylene;
L³ is a bond, —O—, —NR¹⁰—, —NR¹⁰—C₁-C₆ alkylene-, —O—NR¹⁰—, —NR¹⁰—C₁-C₆ alkylene-(O—C₁-C₆ alkylene)ₙ-, —NR¹⁰-L⁴-, —NR¹⁰—C₁-C₆ alkylene-NR¹¹—(C(═O)—C₁-C₆ alkylene-O—)ₘ—, or —NR¹⁰—C₁-C₆ alkylene-NR¹⁰—C₁-C₆ alkylene-NR¹⁰—C₁-C₆ alkylene-;
L⁴ is a bond, -heterocyclyl-, or -heterocyclyl-C₁-C₆ alkylene-;
R¹⁰ is hydrogen or C₁-C₆ alkyl;
R¹¹ is hydrogen or C₁-C₆ alkyl;
R¹² and R¹³ are each independently selected from hydrogen, C₁-C₆ alkyl, or R¹² and R¹³ are joined together along with the other atoms to which they are attached to form a 5-membered or 6-membered carbocyclic or heterocyclic ring;
R¹⁴ is hydrogen or C₁-C₆ alkylene, -(L⁵)-aryl, -(L⁵)-aryl-A⁵, -(L⁵)-heteroaryl, -(L⁵)-heteroaryl-A⁵, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
L⁵ is a bond, C₁-C₁₀ alkylene, —O—, or —NR¹⁰—;
R¹⁷ and R¹⁸ are each independently hydrogen or aryl;
R¹⁹ and R²⁰ are each independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
R²¹ and R²² are each independently selected from hydrogen, C₁-C₆ alkyl, sulfonate, or R²¹ and R²² are joined together along with the other atoms to which they are attached to form a 5-membered or 6-membered aryl;
R²³ and R²⁴ are each independently selected from hydrogen, C₁-C₆ alkyl, sulfonate, or R²³ and R²⁴ are joined together along with the other atoms to which they are attached to form a 5-membered or 6-membered aryl;
n is 0, 1, 2, or 3;
m is 0, 1, 2, or 3;
p is 0, 1, 2, or 3;
q is 0, 1, 2, or 3;
x is 0 or 1; and
one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof and the others of A¹, A², A³, A⁴, or A⁵ are each independently absent, hydrogen, —COOH, or sulfonate.

In some aspects, the compounds used in the present disclosure have a structure of Formula (III), or a pharmaceutically acceptable salt thereof:

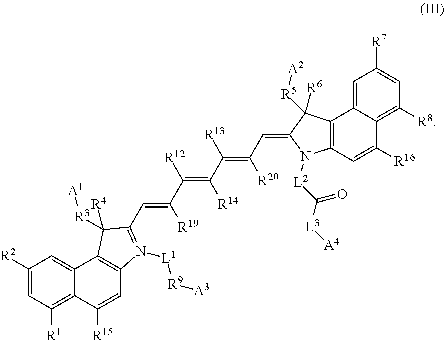

In certain aspects, the present compounds have a structure of Formula (IV), or a pharmaceutically acceptable salt thereof:

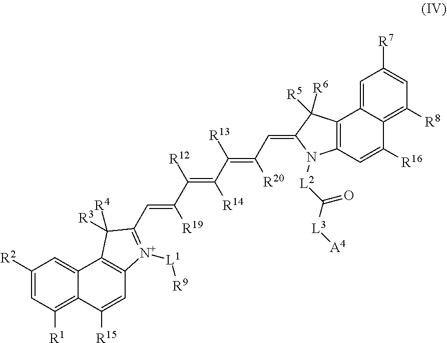

wherein:
R¹, R², R³, R⁴, R⁵, R⁶, R⁷, R⁸, R¹⁵, and R¹⁶ are each independently selected from hydrogen, C₁-C₆ alkyl, C₁-C₆ alkylene-COOH, sulfonate, C₁-C₆ alkylene-sulfonate, —COOH, —SO₂—NH₂, or C₁-C₆ alkoxy;
R⁹ is hydrogen, sulfonate, amine or —COOH;
L¹ is C₃-C₆ alkylene;
L² is C₁-C₁₀ alkylene;
L³ is a bond, —O—, —NR¹⁰—, —NR¹⁰—C₁-C₆ alkylene-, —O—NR¹⁰—, —NR¹⁰—C₁-C₆ alkylene-(O—C₁-C₆ alkylene)ₙ-, —NR¹⁰-L⁴-, —NR¹⁰—C₁-C₆ alkylene-NR¹¹—(C(═O)—C₁-C₆ alkylene-O—)ₘ—, or —NR¹⁰—C₁-C₆ alkylene-NR¹⁰—C₁-C₆ alkylene-NR¹⁰—C₁-C₆ alkylene-;
L⁴ is a bond, -heterocyclyl-, or -heterocyclyl-C₁-C₆ alkylene-;
R¹⁰ is hydrogen or C₁-C₆ alkyl;
R¹ is hydrogen or C₁-C₆ alkyl;
R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;
R¹⁴ is hydrogen or C₁-C₆ alkylene, -(L⁵)-aryl, -(L⁵)-aryl-R²¹, -(L⁵)-heteroaryl, -(L⁵)-heteroaryl-R²¹, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
L⁵ is a bond, C₁-C₁₀ alkylene, —O—, —NR¹⁰—;
R¹⁷ and R¹⁸ are each independently hydrogen or aryl;
R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
R²¹ is hydrogen, sulfonate, or —COOH;
n is 0, 1, 2, or 3;
m is 0, 1, 2, or 3;
p is 0, 1, 2, or 3;
q is 0, 1, 2, or 3; and
A⁴ is a polypeptide having at least 80% sequence identity with MCMPCFTTDHQMARRCDDCCGGRGRGKCYGPQCLCR (SEQ ID NO: 9) or a fragment thereof.

In other aspects, compounds used in the present disclosure have a structure of Formula (V), or a pharmaceutically acceptable salt thereof:

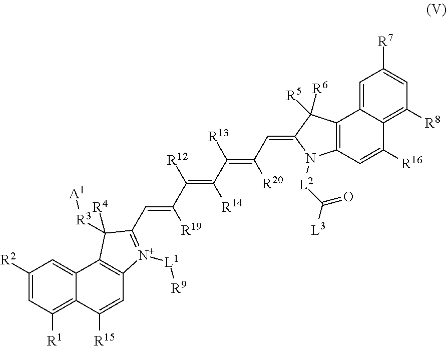

wherein:
R¹, R², R⁴, R⁵, R⁶, R⁷, R¹⁵, and R¹⁶ are each independently selected from hydrogen, C₁-C₆ alkyl, C₁-C₆ alkylene-COOH, sulfonate, —COOH, —SO₂—NH₂, or C₁-C₆ alkoxy;
R³ is selected from C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—;
R⁹ is hydrogen, sulfonate, or —COOH, or C₁-C₁₀ alkyl;
L¹ is C₃-C₆ alkylene;
L² is C₁-C₁₀ alkylene;
L³ is hydrogen, sulfonate, —COOH, C₁-C₁₀ alkyl;
L⁴ is a bond, -heterocyclyl-, or -heterocyclyl-C₁-C₆ alkylene-;
R¹⁰ is hydrogen or C₁-C₆ alkyl;
R¹¹ is hydrogen or C₁-C₆ alkyl;
R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;
R¹⁴ is hydrogen or C₁-C₆ alkylene, -(L⁵)-aryl, -(L⁵)-heteroaryl, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
L⁵ is a bond, C₁-C₁₀ alkylene, —O—, —NR¹⁰—;
R¹⁷ and R¹⁸ are each independently hydrogen or aryl;
R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
n is 0, 1, 2, or 3;
m is 0, 1, 2, or 3;
p is 0, 1, 2, or 3;
q is 0, 1, 2, or 3;
x is 0 or 1; and
A¹ is a polypeptide having at least 85% sequence identity with MCMPCFTTDHQMARRCDDCCGGRGRGKCYGPQCLCR (SEQ ID NO: 9) or a fragment thereof.

In other aspects, compounds used in the present disclosure have a structure of Formula (VI), or a pharmaceutically acceptable salt thereof:

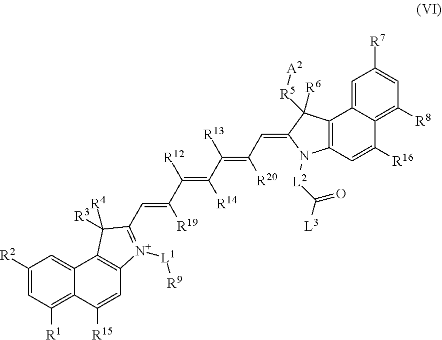

wherein:
R¹, R², R³, R⁴, R⁶, R⁷, R⁸, R¹⁵, and R¹⁶ are each independently selected from hydrogen, C₁-C₆ alkyl, C₁-C₆ alkylene-COOH, sulfonate, —COOH, —SO₂—NH₂, or C₁-C₆ alkoxy;
R⁵ is selected from C₁-C₁₀ alkylene-(C(═O))ₓ—, C₁-C₁₀ alkylene-(C(═O))ₓ—O—, or C₁-C₁₀ alkylene-(C(═O))ₓ—NR¹⁰—;
R⁹ is hydrogen, sulfonate, or —COOH, or C₁-C₁₀ alkyl;
L¹ is C₃-C₆ alkylene;
L² is C₁-C₁₀ alkylene;
L³ is hydrogen, sulfonate, —COOH, or C₁-C₁₀ alkyl;
L⁴ is a bond, -heterocyclyl-, or -heterocyclyl-C₁-C₆ alkylene-;
R¹⁰ is hydrogen or C₁-C₆ alkyl;
R¹¹ is hydrogen or C₁-C₆ alkyl;
R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;
R¹⁴ is hydrogen or C₁-C₆ alkylene, -(L⁵)-aryl, -(L⁵)-heteroaryl, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
L⁵ is a bond, C₁-C₁₀ alkylene, —O—, —NR¹⁰—;
R¹⁷ and R¹⁸ are each independently hydrogen or aryl;
R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
n is 0, 1, 2, or 3;
m is 0, 1, 2, or 3;
p is 0, 1, 2, or 3;
q is 0, 1, 2, or 3;
x is 0 or 1; and
A² is a polypeptide having at least 85% sequence identity with MCMPCFTTDHQMARRCDDCCGGRGRGKCYGPQCLCR (SEQ ID NO: 9) or a fragment thereof.

In some aspects, compounds used in the present disclosure have a structure of Formula (VII), or a pharmaceutically acceptable salt thereof:

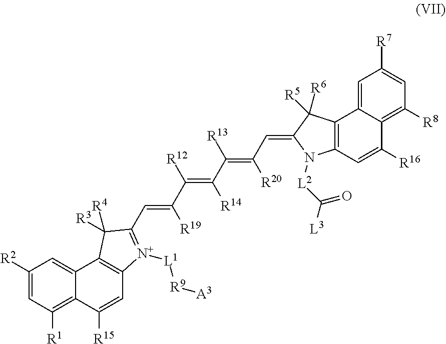

wherein:
R¹, R², R³, R⁴, R⁵, R⁶, R⁷, R⁸, R¹⁵, and R¹⁶ are each independently selected from hydrogen, C₁-C₆ alkyl, C₁-C₆ alkylene-COOH, sulfonate, —COOH, —SO₂—NH₂, or C₁-C₆ alkoxy;
R⁹ is selected from C₁-C₁₀ alkylene-(C(═O))ₓ—, C₁-C₁₀ alkylene-(C(═O))ₓ—O—, or
C₁-C₁₀ alkylene-(C(═O))ₓ—NR¹⁰—;
L¹ is C₃-C₆ alkylene;
L² is C₁-C₁₀ alkylene;
L³ is hydrogen, sulfonate, —COOH, or C₁-C₁₀ alkyl;
L⁴ is a bond, -heterocyclyl-, or -heterocyclyl-C₁-C₆ alkylene-;
R¹⁰ is hydrogen or C₁-C₆ alkyl;
R¹¹ is hydrogen or C₁-C₆ alkyl;
R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;
R¹⁴ is hydrogen or C₁-C₆ alkylene, -(L⁵)-aryl, -(L⁵)-heteroaryl, —NR¹⁷R¹⁸, R¹⁴ and
R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
R¹⁷ and R¹⁸ are each independently hydrogen or aryl;
R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
n is 0, 1, 2, or 3;
m is 0, 1, 2, or 3;
p is 0, 1, 2, or 3;
q is 0, 1, 2, or 3;
x is 0 or 1;
L⁵ is a bond, C₁-C₁₀ alkylene, —O—, —NR¹⁰—;
   A³ is a polypeptide having at least 85% sequence identity with MCMPCFTTDHQMARRCDDCCGGRGRGKCYGPQCLCR (SEQ ID NO: 9) or a fragment thereof.

In additional aspects, compounds used in the present disclosure have a structure Formula (VIII), or a pharmaceutically acceptable salt thereof:

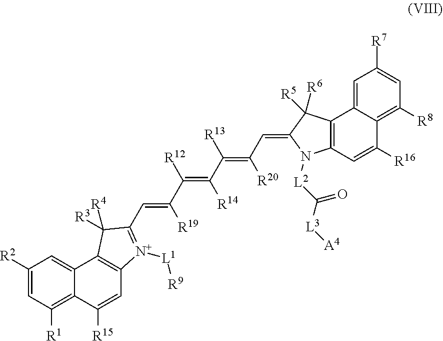

wherein:
R¹, R², R³, R⁴, R⁵, R⁶, R⁷, R⁸, R¹⁵, and R¹⁶ are each independently selected from hydrogen, C₁-C₆ alkyl, C₁-C₆ alkylene-COOH, sulfonate, —COOH, —SO₂—NH₂, or C₁-C₆ alkoxy;
R⁹ is hydrogen, sulfonate, or —COOH;
L¹ is C₃-C₆ alkylene;
L² is C₁-C₁₀ alkylene;
L³ is a bond, —O—, —NR¹⁰—, —NR¹⁰—C₁-C₆ alkylene-, —O—NR¹⁰—, —NR¹⁰—C₁-C₆ alkylene-(O—C₁-C₆ alkylene)ₙ-, —NR¹⁰-L⁴-, —NR¹⁰—C₁-C₆ alkylene-NR¹¹—(C(═O)—C₁-C₆ alkylene-O—)ₘ—, or —NR¹⁰—C₁-C₆ alkylene-NR¹⁰—C₁-C₆ alkylene-NR¹⁰—C₁-C₆ alkylene-;
L⁴ is a bond, -heterocyclyl-, or -heterocyclyl-C₁-C₆ alkylene-;
R¹⁰ is hydrogen or C₁-C₆ alkyl;
R¹¹ is hydrogen or C₁-C₆ alkyl;
R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;
R¹⁴ is -(L⁵)-aryl-A⁵, or -(L⁵)-heteroaryl-A⁵;
L⁵ is a bond, C₁-C₁₀ alkylene, —O—, —NR¹⁰—;
R¹⁷ and R¹⁸ are each independently hydrogen or aryl;
R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
n is 0, 1, 2, or 3;
m is 0, 1, 2, or 3;
p is 0, 1, 2, or 3;
q is 0, 1, 2, or 3;
x is 0 or 1;
A⁴ is hydrogen, —COOH, or sulfonate; and
A⁵ is a polypeptide having at least 85% sequence identity with MCMPCFTTDHQMARRCDDCCGGRGRGKCYGPQCLCR (SEQ ID NO: 9) or a fragment thereof.

In certain aspects, A¹, A², and A³ are absent. In some aspects, A⁵ is hydrogen. In certain aspects, R³, R⁴, R⁵, and R⁶ are each independently C₁-C₆ alkyl. In some aspects, R³, R⁴, R⁵, and R⁶ are each independently methyl. In certain aspects, R¹, R², R⁷, R⁸, R¹⁵, and R¹⁶ are each independently selected from hydrogen or sulfonate. In further aspects, R¹, R², R⁷, R⁸, R¹⁵, and R¹⁶ are each independently hydrogen. In some aspects, R¹², R¹³, R¹⁴, R¹⁹, R² are each independently hydrogen.

In certain aspects, R¹² and R¹³ join together along with the atoms to which they are attached to form a six-membered carbocyclic ring. In other aspects, R¹² and R¹³ join together along with the atoms to which they are attached to form a five-membered carbocyclic ring. In certain aspects, R¹⁴ and R¹⁹ join together along with the atoms to which they are attached to form a six-membered carbocyclic ring. In some aspects, R¹⁴ and R²⁰ join together along with the atoms to which they are attached to form a six-membered carbocyclic ring. In certain aspects, L¹ is C₃-C₆ alkylene. In other aspects, L¹ is C₃-C₅ alkylene. In still other aspects, L¹ is propylene. In still other aspects, L¹ is butylene. In other aspects, L¹ is pentylene. In some aspects, L² is C₃-C₆ alkylene. In other aspects, L² is propylene. In still other aspects, L² is butylene. In other aspects, L² is pentylene. In some aspects, R⁹ is sulfonate. In other aspects, R⁹ is hydrogen. In some aspects, R¹⁴ is hydrogen. In other aspects, R¹⁴ is -(L⁵)-aryl. In still other aspects, R¹⁴ is -(L⁵)-aryl-A⁵.

In some aspects, R¹ is hydrogen. In certain aspects, R² is hydrogen. In some aspects, R³ is methyl. In certain aspects, R⁴ is methyl. In some aspects, R⁵ is methyl. In certain aspects R⁶ is methyl. In some aspects, R⁷ is hydrogen. In certain aspects, R⁸ is hydrogen. In some aspects, R¹² is hydrogen. In certain aspects, R¹³ is hydrogen. In some aspects, R¹⁴ is hydrogen. In certain aspects, R¹⁹ is hydrogen. In some aspects, R²⁰ is hydrogen. In certain aspects, R¹⁰ is hydrogen. In some aspects, R¹¹ is hydrogen.

In some aspects, R¹⁷ and R¹⁸ are independently phenyl. In some aspects, L¹ is buytlene. In some aspects, L² is pentylene. In some aspects, L³ is selected from a bond, —O—, —NR¹⁰—, —NR¹⁰—C₁-C₆ alkylene-, —O—NR¹⁰—, or —NR¹⁰-L⁴-. In further aspects, L³ is a bond.

In some aspects, L⁴ is -heterocyclyl- or -heterocyclyl-C₁-C₆ alkylene-. In further aspects, L⁴ is -piperizinyl-(C₁-C₆ alkylene)-. In still further aspects, L⁴ is

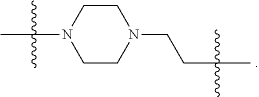 [SMILES: *CCN1CCN(*)CC1]

In some aspects, p is 1. In certain aspects, q is 1.

In some aspects, the compound used has the structure of any one of Formulas (IX), (X), (XI), (XII), (XIII), (XIV), (XV), or (XVI):

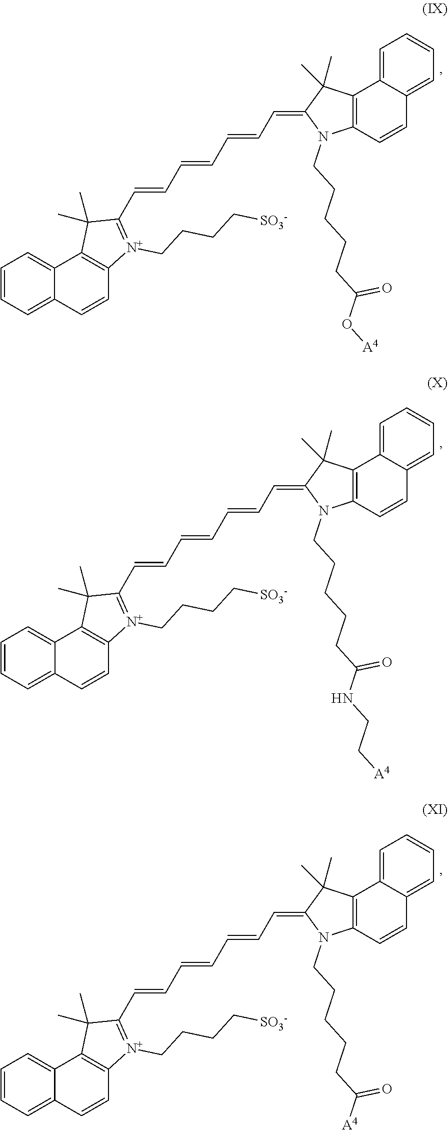

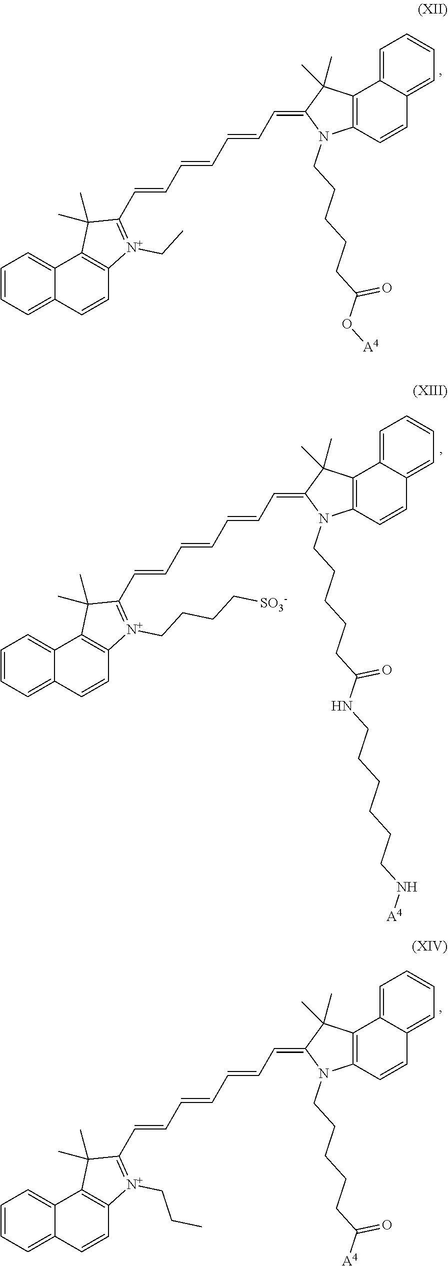

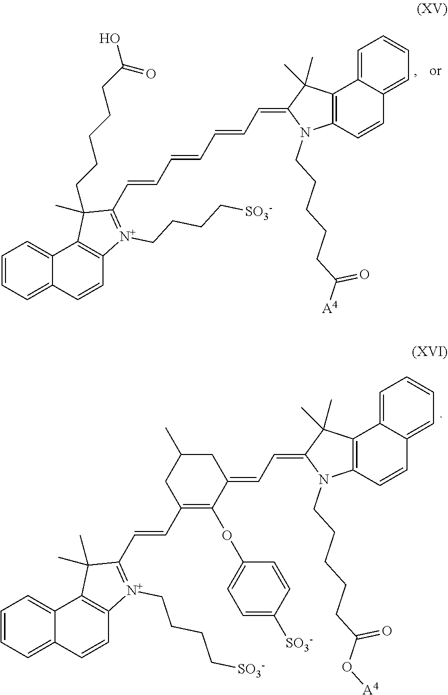

In some aspects, the compound has the structures of any one of Formulas (IX), (X), (XI), (XII), (XIII), (XIV), (XV), or (XVI), wherein A⁴ is a polypeptide.

In some aspects, one of A¹, A², A³, A⁴, or A⁵ is a polypeptide having at least 87% sequence identity with MCMPCFTTDHQMARRCDDCCGGRGRGKCYGPQCLCR (SEQ ID NO: 9) or a fragment thereof. In further aspects, one of A¹, A², A³, A⁴, or A⁵ is a polypeptide having at least 90% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having at least 92% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having at least 95% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having at least 97% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having 100% sequence identity with MCMPCFTTDHQMARRCDDCCGGRGRGKCYGPQCLCR (SEQ ID NO: 9) or a fragment thereof. In still further aspects, one of A¹, A², A³, A⁴, or A⁵ is a polypeptide having the sequence MCMPCFTTDHQMARRCDDCCGGRGRGKCYGPQCLCR (SEQ ID NO: 9) or a fragment thereof.

In some aspects, the fragment of A¹, A², A³, A⁴, or A⁵ has a length of at least 25 amino acid residues. In further aspects, the fragment of A¹, A², A³, A⁴, or A⁵ has a length of at least 27 amino acid residues. In still further aspects, the fragment of A¹, A², A³, A⁴, or A⁵ has a length of at least 29 amino acid residues. In still further aspects, the fragment of A¹, A², A³, A⁴, or A⁵ has a length of at least 31 amino acid residues. In still further aspects, the fragment of A¹, A², A³, A⁴, or A⁵ has a length of at least 33 amino acid residues.

In some aspects, one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof having the tumor cell binding affinity of native chlorotoxin. In certain aspects, one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof having about the same the tumor cell binding affinity of native chlorotoxin. In some aspects, one of A¹, A², A³, A⁴, or A⁵ is a polypeptide having at least 85% sequence identity with MCMPCFTTDHQMARRCDDCCGGRGRGKCYGPQCLCR (SEQ ID NO: 9) or a fragment thereof having the tumor cell binding affinity of native chlorotoxin wherein one of A¹, A², A³, A⁴, or A⁵ has a sequence selected from SEQ ID NO: 1-SEQ ID NO: 485.

In some aspect, the polypeptide contains no lysine residues. In some aspects, the polypeptide used comprises at least one lysine amino acid residue. In certain aspects, the polypeptide comprises a single lysine amino acid residue. In some aspects, the polypeptide comprises one, two, or three lysine amino acid residues. In some aspects, the polypeptide comprises a lysine residue at the position corresponding to K-27 of native chlorotoxin. In some aspects, the polypeptide comprises a lysine residue at the position corresponding to K-23 of native chlorotoxin. In some aspects, the polypeptide comprises a lysine residue at the position corresponding to K-15 of native chlorotoxin.

In some aspects, one or more of the amino acids of the polypeptide used is substituted with a non-naturally occurring amino acid residue. In further aspects the non-naturally occurring amino acid residue is a citrulline amino acid residue. In still further aspects, L³ is attached to A⁴ at a citrulline amino acid residue of the polypeptide.

In some aspects, L³ is attached to A⁴ at a lysine amino acid residue of the polypeptide. In certain aspects, L³ is attached to A⁴ at the N-terminus of the polypeptide. In some aspects, L³ is attached to A⁴ at the C-terminus of the polypeptide. In some aspects, the R³ is attached to A¹ at a lysine amino acid residue of the peptide, a citrulline amino acid residue of the polypeptide, the N-terminus of the polypeptide, or the C-terminus of the polypeptide. In some aspects, the R⁵ is attached to A² at a lysine amino acid residue of the polypeptide, a citrulline amino acid residue of the polypeptide, the N-terminus of the polypeptide, or the C-terminus of the polypeptide. In some aspects, the R⁹ is attached to A³ at a lysine amino acid residue of the polypeptide, a citrulline amino acid residue of the polypeptide, the N-terminus of the polypeptide, or the C-terminus of the polypeptide. In some aspects, the aryl is attached to A⁵ at a lysine amino acid residue of the polypeptide, a citrulline amino acid residue of the polypeptide, the N-terminus of the polypeptide, or the C-terminus of the polypeptide.

In some aspects, the compound used has the structure of any one of compounds 1 to 60 as found in TABLE 2, in which A is a peptide portion and can comprise any of the peptides described herein, such as any one of SEQ ID NO: 1-SEQ ID NO: 485. In other aspects, the compound used has the structure of any one of compounds 1 to 60 as found in TABLE 2, in which A is a peptide fragment and can comprise a fragment of any of the peptides described herein, such as any one of SEQ ID NO: 1-SEQ ID NO: 485. In some embodiments, the fragment of the polypeptide has a length of at least 25 residues.

In some aspects, the compound used is conjugated to polyethylene glycol (PEG), hydroxyethyl starch, polyvinyl alcohol, a water soluble polymer, a zwitterionic water soluble polymer, a water soluble poly(amino acid), an albumin derivative, or a fatty acid.

In some aspects, the polypeptide used has an isoelectric point of from 5.5 to 9.5. In some aspects, the polypeptide has an isoelectric point of from 7.5 to 9.0. In some aspects, the polypeptide has an isoelectric point of from 8.0 to 9.0. In some aspects, the polypeptide has an isoelectric point of from 8.5 to 9.0. In some aspects, the polypeptide is basic and has an isoelectric point of greater than 7.5. In some aspects, the polypeptide has an isoelectric point of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In other aspects, the polypeptide comprises an isoelectric point of at least 5.5, at least 6.0, at least 6.5, at least 7.0, at least 7.5, at least 8.0, at least 8.5, at least 9.0, or at least 9.5.

In some aspects, the polypeptide used comprises at least eight cysteine amino acid residues. In some aspects, the polypeptide comprises eight cysteine amino acid residues. In some aspects, the polypeptide comprises four disulfide bonds. In some aspects, the polypeptide comprises from six to seven cysteine amino acid residues. In some aspects, the polypeptide comprises three disulfide bonds. In some aspects, the polypeptide comprises at least 1 disulfide bond, at least 2 disulfide bonds, at least 3 disulfide bonds, at least 4 disulfide bonds, at least 5 disulfide bonds, or at least 6 disulfide bonds. In some aspects, the spacing between the cysteine amino acid residues in the polypeptide is about the same as in native chlorotoxin. In some aspects, the distribution of charge on the surface of the polypeptide is about the same as in native chlorotoxin.

In some aspects, the N-terminus of the polypeptide is blocked by acetylation or cyclization.

In some aspects, one or more of the methionine amino acid residues used is replaced with an amino acid residue selected from isoleucine, threonine, valine, leucine, serine, glycine, alanine, or a combination thereof. In other aspects, one, two, or three methionine residues of the polypeptide are replaced with other amino acids.

In some aspects, each amino acid of the polypeptide is independently selected as an L- or D-enantiomer.

In some aspects, the compound used is capable of passing across the blood brain barrier. In some aspects, the compound used further comprises a therapeutic agent. In some aspects, the polypeptide is conjugated to the therapeutic agent. In some aspects, the compound used further comprises a therapeutic agent attached to A. In further aspects, the therapeutic agent is a cytotoxic agent. In still other aspects, the therapeutic agent comprises a radioisotype, toxin, enzyme, sensitizing drug, radiosensitizer, nucleic acid, interfering RNA, antibody, antibody fragment, aptamer, anti-angiogenic agent, cisplatin, carboplatin, oxaliplatin, anti-metabolite, mitotic inhibitor, growth factor inhibitor, cytotoxin, microtubule disrupting agent, DNA modifying agent, maytansine derivative, auristatin derivative, dolostatin derivative, monomethyl auristatin E, monomethyl auristatin F, DM1, calicheamicin, duocarmycin derivative, campthotecin, pyrrolobenzodiazepine, paclitaxel, cyclophosphamide, chlorambucil, melphlan, bufulfan, carmustine, ifosfamide, temozolomide, topotecan, fluorouracil, vincristine, vinblastine, procarbazine, dacarbazine, altretamine, methotrexate, pemetrexed, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane, amifostine, lenalidomide, imatinib, abiraterone, erlotinib, enzalutimide, everolimus palbociclib, pomalidomide, sutininib, sorafenib, imatinib, gefitinib, afatinib, axitinib, crizotinib, vismoegib, dabrefenib, vemurafenib, or a combination thereof.

In various aspects, the present disclosure uses a composition comprising a polypeptide having at least 80% sequence identity with MCMPCFTTDHQMARRCDDCCGGRGRGKCYGPQCLCR (SEQ ID NO: 9) or a fragment thereof, wherein when the composition is intravenously administering to a human subject at a dosage within a range of from about 1 mg to 100 mg over a time period within a range from about 1 minute to about 120 minutes, and the composition produces in the human subject an average maximum compound blood plasma concentration (average Cₘₐₓ) within a range from about 15 ng/mL to 600 ng/mL per each 1 mg dosage of the compound administered.

In some aspects, the compound of the composition used is any suitable compound described herein. In other aspects, the compound of the composition further comprises an agent. In some aspects, the compound comprises a detectable agent. In one embodiment, the polypeptide is conjugated to an agent. In another embodiment, the polypeptide is conjugated to a detectable agent. In some embodiments, a detectable agent is a detectable label. In some embodiments, a detectable agent comprises a dye, a fluorophore, a fluorescent biotin compound, a luminescent compound, a chemiluminescent compound, a radioisotope, a paramagnetic metal ion, or a combination thereof. In some embodiments, the polypeptide comprises a single lysine residue and the agent is conjugated to the polypeptide at the single lysine residue. In some embodiments, the polypeptide comprises no lysine residues and the agent is conjugated to the polypeptide at the N-terminus of the polypeptide.

Certain exemplary compounds falling within the scope of these genuses are provided below in TABLE 2 and further described herein, including both the peptide portion (indicated by A) and the detectable label portion.

TABLE 2
Compounds according to the present disclosure.

| No. | Structure |
|---|---|
| 1 | 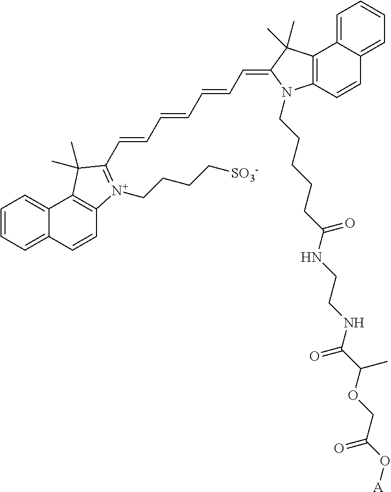 |
| 2 | 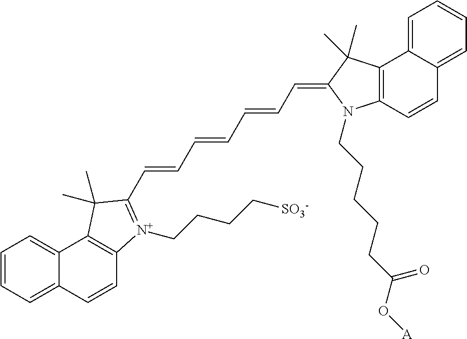 |
| 3 | 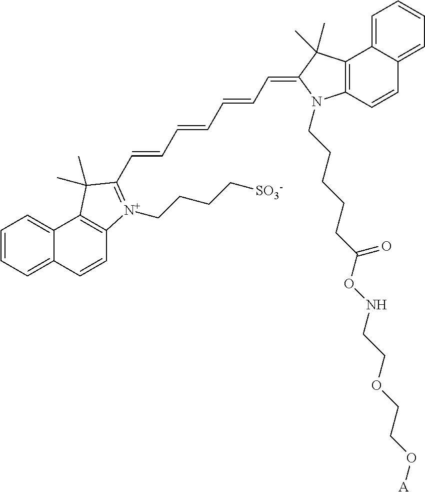 |
| 4 | 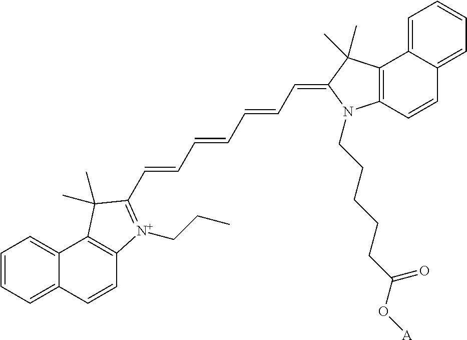 |
| 5 | 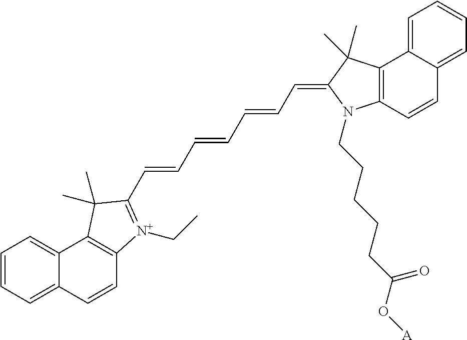 |
| 6 | 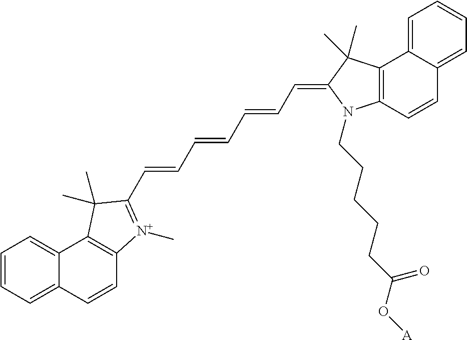 |
| 7 | 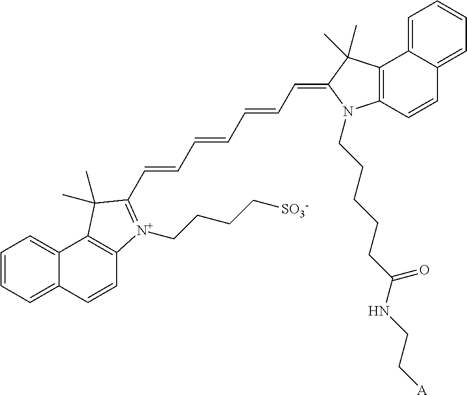 |
| 8 | 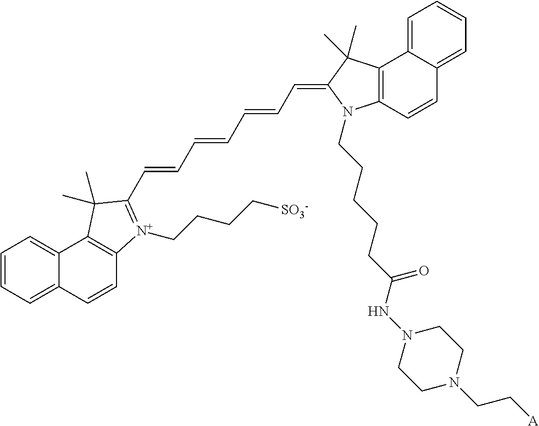 |
| 9 | 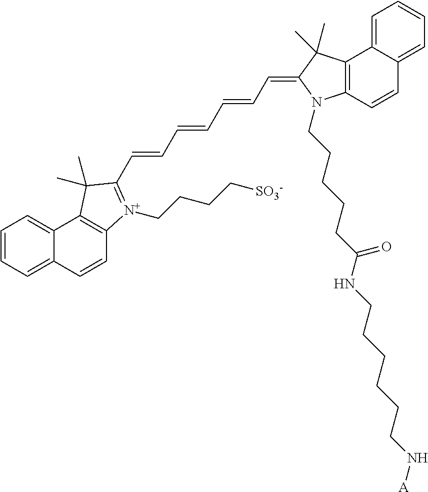 |
| 10 | 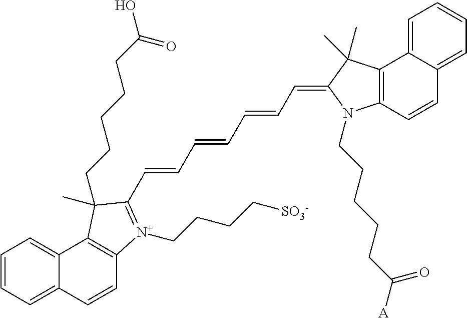 |
| 11 | 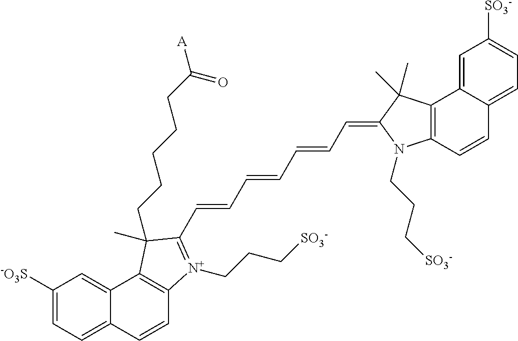 |
| 12 | 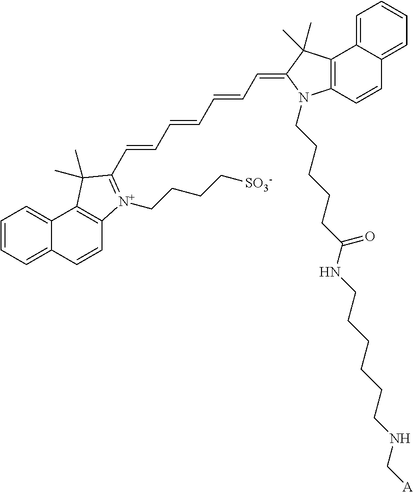 |
| 13 | 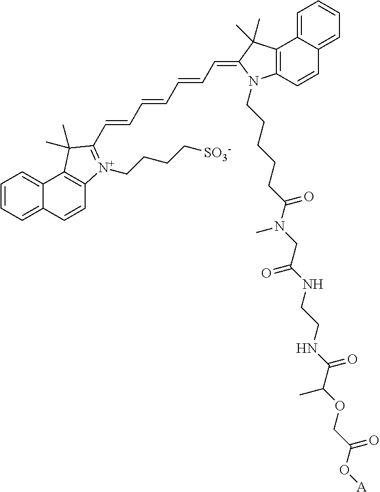 |
| 14 | 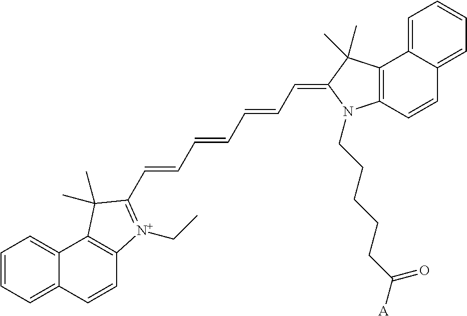 |
| 15 | 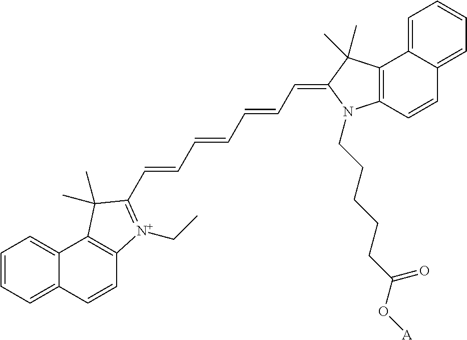 |
| 16 | 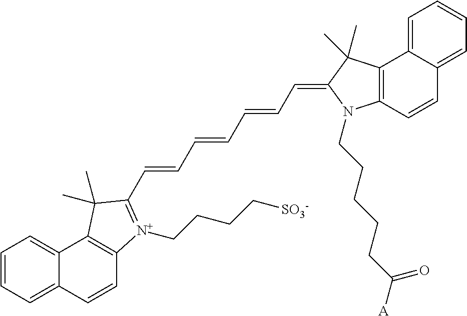 |
| 17 | 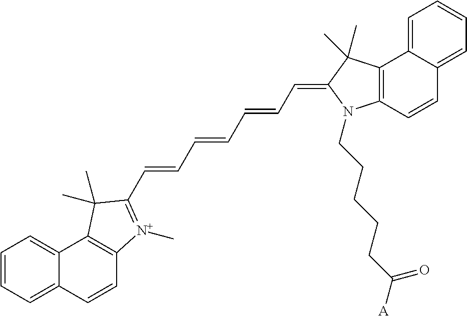 |
| 18 | 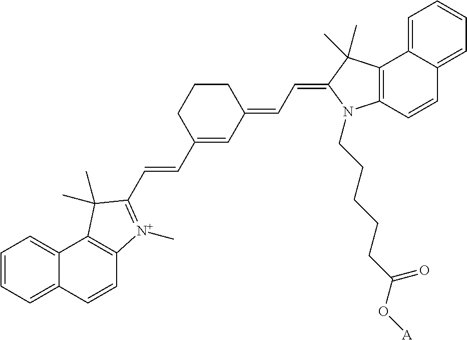 |
| 19 | 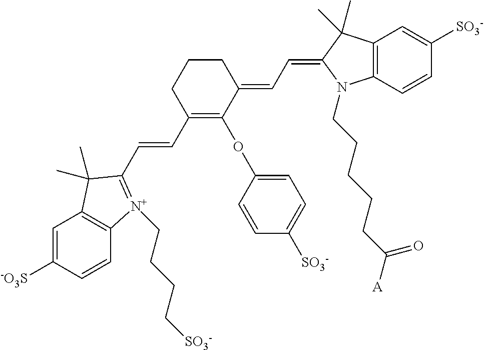 |
| 20 | 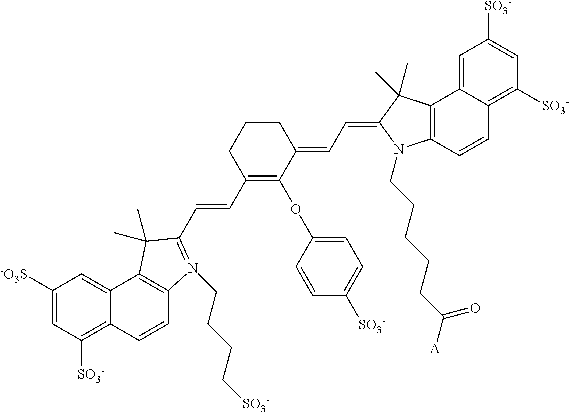 |
| 21 | 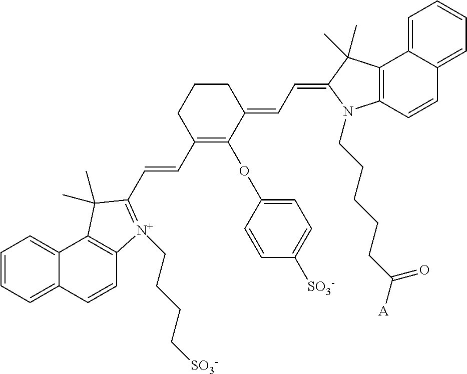 |
| 22 | 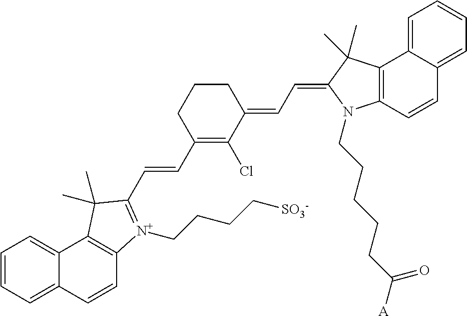 |
| 23 | 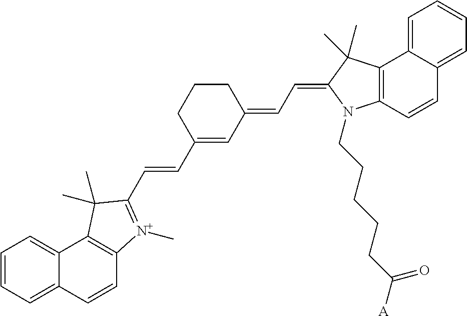 |
| 24 | 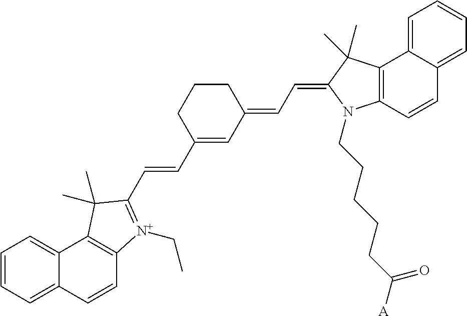 |
| 25 | 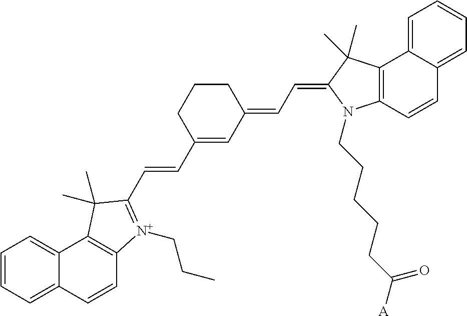 |
| 26 | 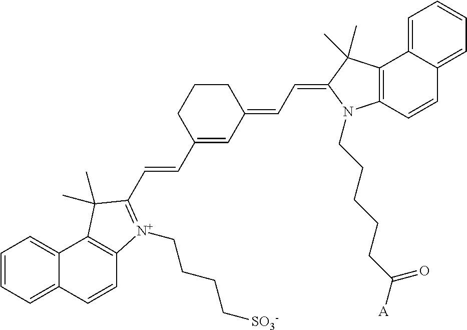 |
| 27 | 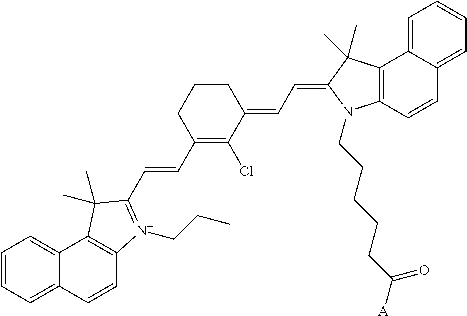 |
| 28 | 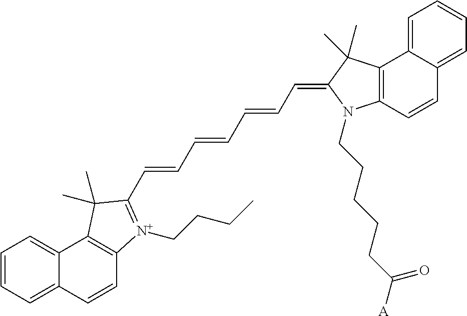 |
| 29 | 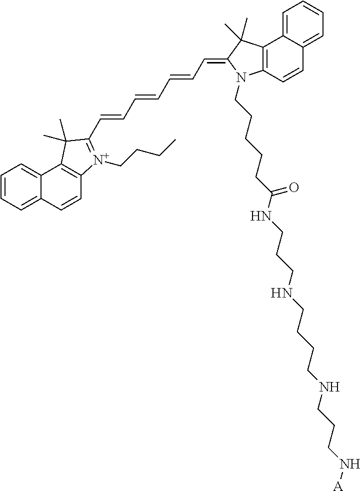 |
| 30 | 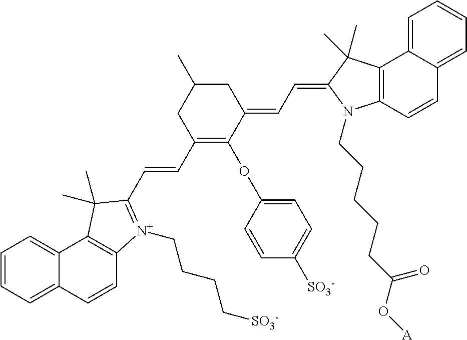 |
| 31 | 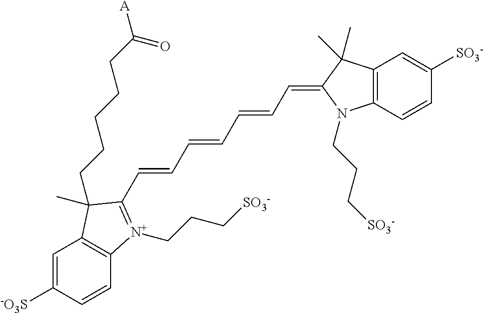 |
| 32 | 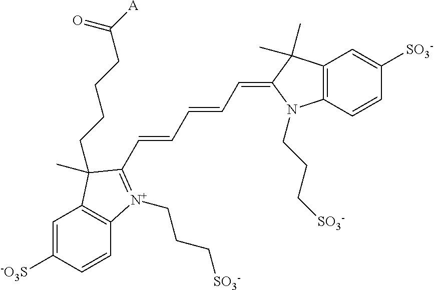 |
| 33 | 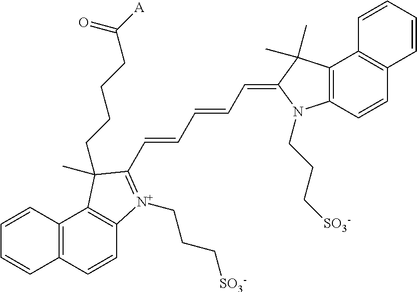 |
| 34 | 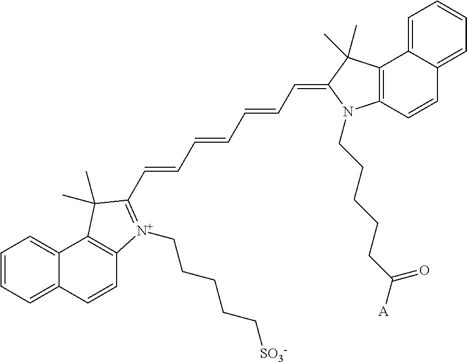 |
| 35 | 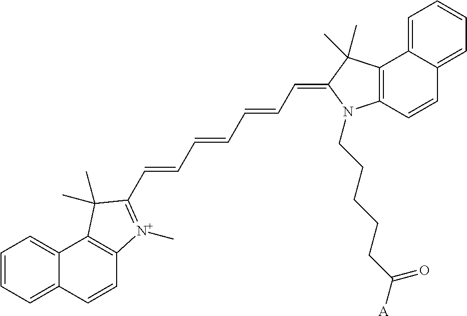 |
| 36 | 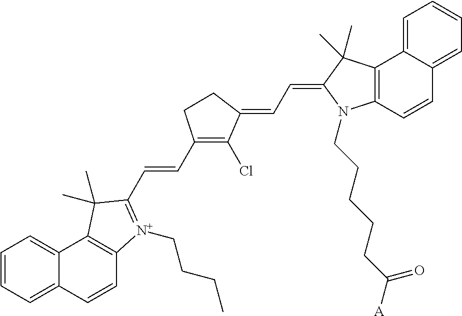 |
| 37 | 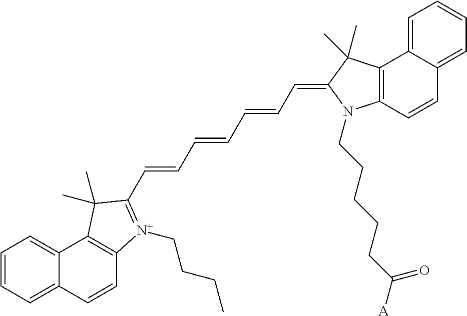 |
| 38 | 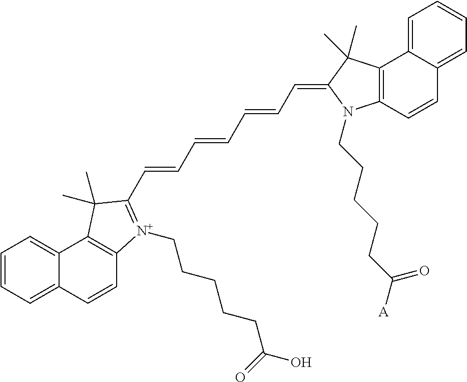 |
| 39 | 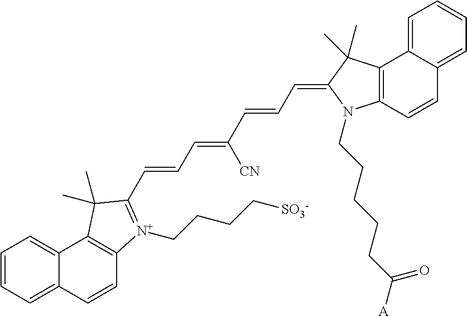 |
| 40 | 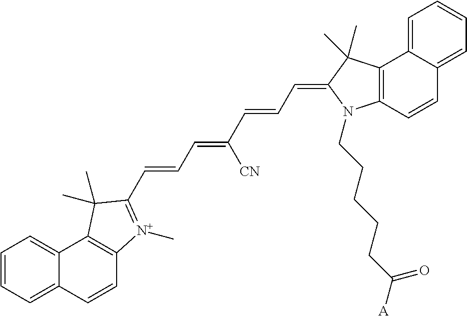 |
| 41 | 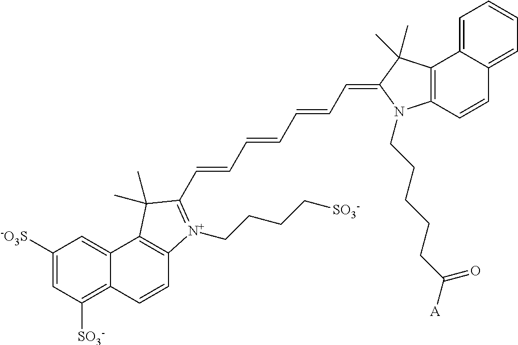 |
| 42 | 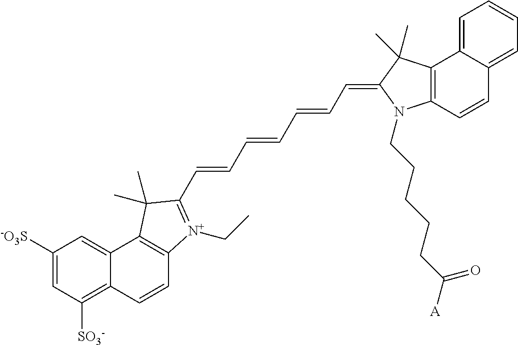 |
| 43 | 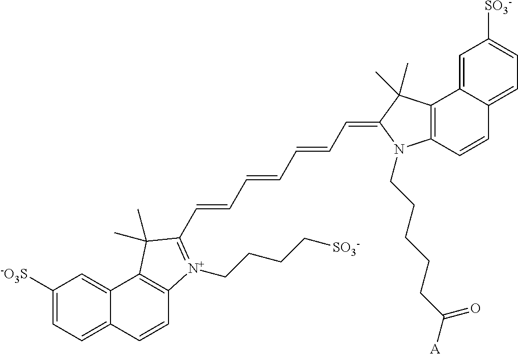 |
| 44 | 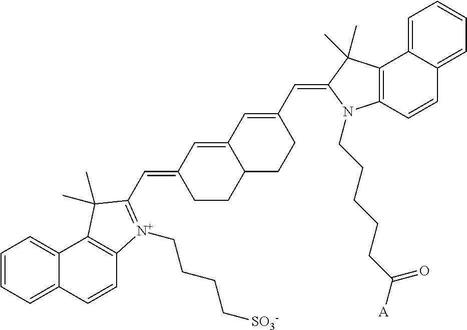 |
| 45 | 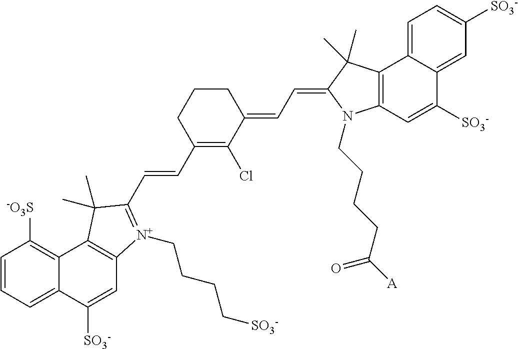 |
| 46 | 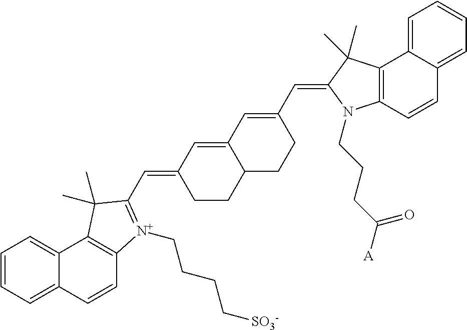 |
| 47 | 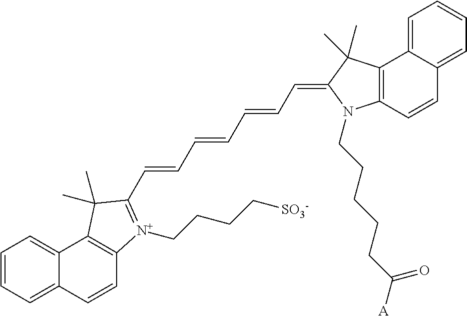 |
| 48 | 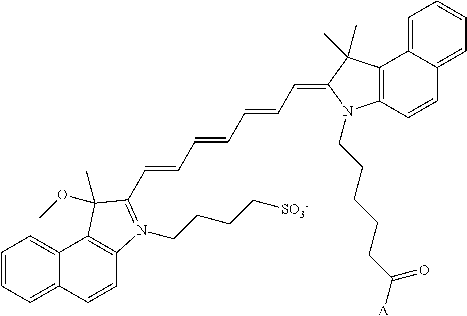 |
| 49 | 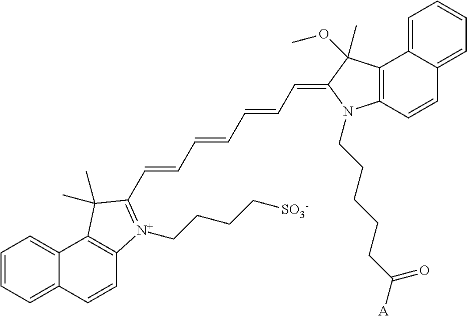 |
| 50 | 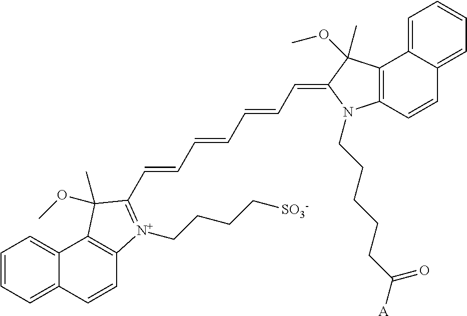 |
| 51 | 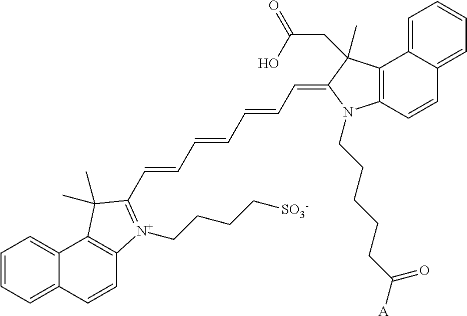 |
| 52 | 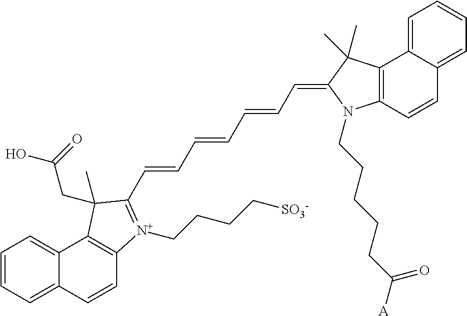 |
| 53 | 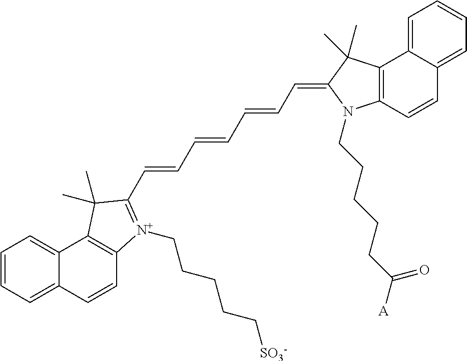 |
| 54 | 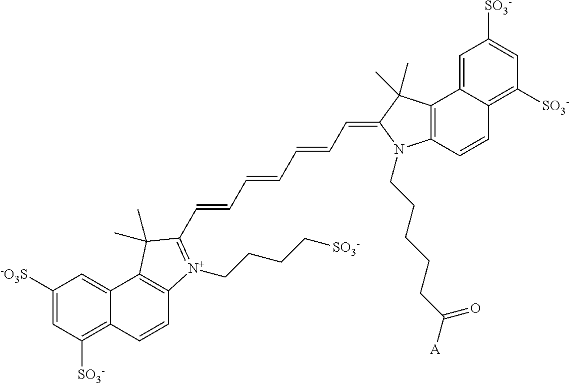 |
| 55 | 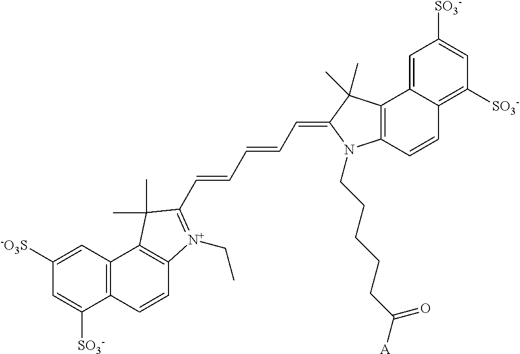 |
| 56 | 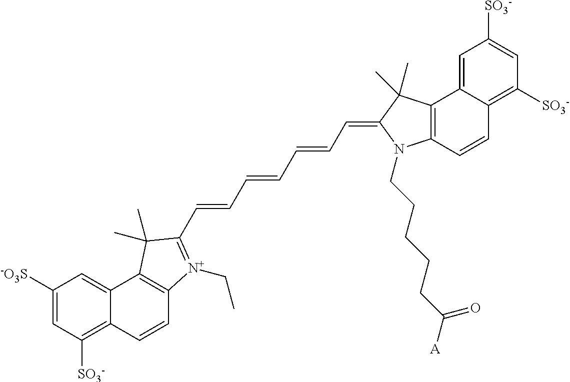 |
| 57 | 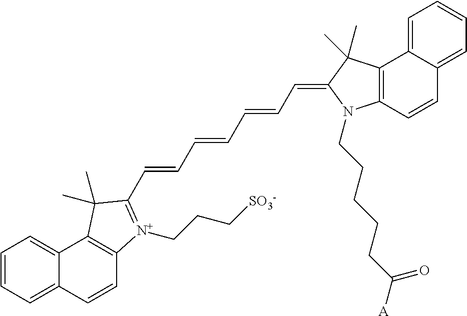 |
| 58 | 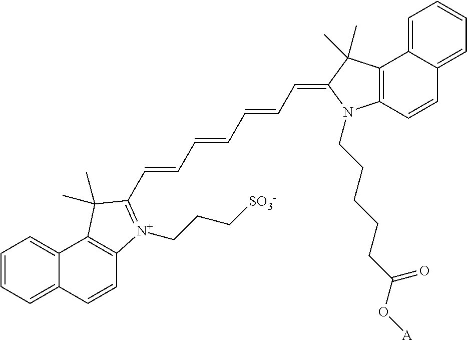 |
| 59 | 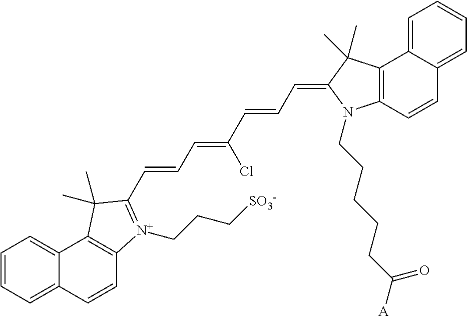 |
| 60 | 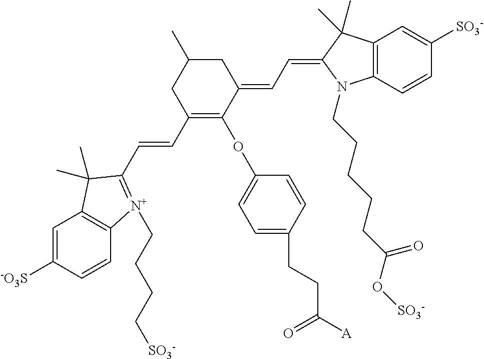 |

The peptide portion A in compounds 1-60 can comprise any of the peptides described herein, such as any one of SEQ ID NO: 1-SEQ ID NO: 485. In some embodiments, the peptide portion A is SEQ ID NO: 5 attached at K-27 to any one of compounds 1-60. In some embodiments, the peptide portion A is SEQ ID NO: 6 attached at K-27 to any one of compounds 1-60. In some embodiments, the peptide portion A is SEQ ID NO: 8 attached at K-27 to any one of compounds 1-60. In some embodiments, the peptide portion A is SEQ ID NO: 9 attached at K-27 to any one of compounds 1-60. In some embodiments, the peptide portion A is SEQ ID NO: 11 attached at K-23 to any one of compounds 1-60. In some embodiments, the peptide portion A is SEQ ID NO: 12 attached at K-23 to any one of compounds 1-60. In some embodiments, the peptide portion A is SEQ ID NO: 13 attached at K-15 to any one of compounds 1-60. In some embodiments, the peptide portion A is SEQ ID NO: 16 attached at K-15 to any one of compounds 1-60. In some embodiments, the peptide portion A is SEQ ID NO: 20 attached at K-23 to any one of compounds 1-60. In some embodiments, the peptide portion A is SEQ ID NO: 21 attached at K-23 to any one of compounds 1-60. In some embodiments, the peptide portion A is SEQ ID NO. 22 attached at K-15 to any one of compounds 1-60. In some embodiments, the peptide portion A is SEQ ID NO: 25 attached at K-15 to any one of compounds 1-60.

TABLE 3 below sets forth certain polypeptide sequences for use with the present disclosure. Citrulline is designated as “Cit” in the sequences.

TABLE 3
Exemplary Peptide Sequences Suitable For Use In The Compounds
of the Present Disclosure. Cit = Citrulline.

| SEQ ID NO | Polypeptide Sequence |
|---|---|
| 1 | MCMPCFTTDHQMARKCDDCCGGKGRGKCYGPQCLCR |
| 2 | MCMPCFTTDHQMARACDDCCGGKGRGKCYGPQCLCR |
| 3 | MCMPCFTTDHQMARRCDDCCGGKGRGKCYGPQCLCR |
| 4 | MCMPCFTTDHQMARKCDDCCGGAGRGKCYGPQCLCR |
| 5 | MCMPCFTTDHQMARACDDCCGGAGRGKCYGPQCLCR |
| 6 | MCMPCFTTDHQMARRCDDCCGGAGRGKCYGPQCLCR |
| 7 | MCMPCFTTDHQMARKCDDCCGGRGRGKCYGPQCLCR |
| 8 | MCMPCFTTDHQMARACDDCCGGRGRGKCYGPQCLCR |
| 9 | MCMPCFTTDHQMARRCDDCCGGRGRGKCYGPQCLCR |
| 10 | MCMPCFTTDHQMARKCDDCCGGKGRGACYGPQCLCR |
| 11 | MCMPCFTTDHQMARACDDCCGGKGRGACYGPQCLCR |
| 12 | MCMPCFTTDHQMARRCDDCCGGKGRGACYGPQCLCR |
| 13 | MCMPCFTTDHQMARKCDDCCGGAGRGACYGPQCLCR |
| 14 | MCMPCFTTDHQMARACDDCCGGAGRGACYGPQCLCR |
| 15 | MCMPCFTTDHQMARRCDDCCGGAGRGACYGPQCLCR |
| 16 | MCMPCFTTDHQMARKCDDCCGGRGRGACYGPQCLCR |
| 17 | MCMPCFTTDHQMARACDDCCGGRGRGACYGPQCLCR |
| 18 | MCMPCFTTDHQMARRCDDCCGGRGRGACYGPQCLCR |
| 19 | MCMPCFTTDHQMARKCDDCCGGKGRGRCYGPQCLCR |
| 20 | MCMPCFTTDHQMARACDDCCGGKGRGRCYGPQCLCR |
| 21 | MCMPCFTTDHQMARRCDDCCGGKGRGRCYGPQCLCR |
| 22 | MCMPCFTTDHQMARKCDDCCGGAGRGRCYGPQCLCR |
| 23 | MCMPCFTTDHQMARACDDCCGGAGRGRCYGPQCLCR |
| 24 | MCMPCFTTDHQMARRCDDCCGGAGRGRCYGPQCLCR |
| 25 | MCMPCFTTDHQMARKCDDCCGGRGRGRCYGPQCLCR |
| 26 | MCMPCFTTDHQMARACDDCCGGRGRGRCYGPQCLCR |
| 27 | MCMPCFTTDHQMARRCDDCCGGRGRGRCYGPQCLCR |
| 28 | MCMPCFTTDHQMARRCDDCCGGRGRGRCYGPQCLCR |
| 29 | KCMPCFTTDHQMARRCDDCCGGRGRGRCYGPQCLCR |
| 30 | ACAPCFTTDHQAARRCDDCCGGRGRGRCYGPQCLCR |
| 31 | KCAPCFTTDHQAARRCDDCCGGRGRGRCYGPQCLCR |
| 32 | MCMPCFTTDHQMAR(Cit)CDDCCGG(Cit)GRGKCYGPQCLCR |
| 33 | MCMPCFTTDHQMAR(Cit)CDDCCGG(Cit)GRG(Cit)CYGPQCLCR |
| 34 | KCMPCFTTDHQMAR(Cit)CDDCCGG(Cit)GRG(Cit)CYGPQCLCR |
| 35 | ACAPCFTTDHQAAR(Cit)CDDCCGG(Cit)GRGKCYGPQCLCR |
| 36 | ACAPCFTTDHQAAR(Cit)CDDCCGG(Cit)GRG(Cit)CYGPQCLCR |
| 37 | KCAPCFTTDHQAAR(Cit)CDDCCGG(Cit)GRG(Cit)CYGPQCLCR |
| 38 | MCMPCFTTDHQMARKCDDCCGGKGRGKCYGPQCLCRGAGAAGG |
| 39 | MCMPCFTTDHQMARACDDCCGGKGRGKCYGPQCLCRGAGAAGG |
| 40 | MCMPCFTTDHQMARRCDDCCGGKGRGKCYGPQCLCRGAGAAGG |
| 41 | MCMPCFTTDHQMARKCDDCCGGAGRGKCYGPQCLCRGAGAAGG |
| 42 | MCMPCFTTDHQMARACDDCCGGAGRGKCYGPQCLCRGAGAAGG |
| 43 | MCMPCFTTDHQMARRCDDCCGGAGRGKCYGPQCLCRGAGAAGG |
| 44 | MCMPCFTTDHQMARKCDDCCGGRGRGKCYGPQCLCRGAGAAGG |
| 45 | MCMPCFTTDHQMARACDDCCGGRGRGKCYGPQCLCRGAGAAGG |
| 46 | MCMPCFTTDHQMARRCDDCCGGRGRGKCYGPQCLCRGAGAAGG |
| 47 | MCMPCFTTDHQMARKCDDCCGGKGRGACYGPQCLCRGAGAAGG |
| 48 | MCMPCFTTDHQMARACDDCCGGKGRGACYGPQCLCRGAGAAGG |
| 49 | MCMPCFTTDHQMARRCDDCCGGKGRGACYGPQCLCRGAGAAGG |
| 50 | MCMPCFTTDHQMARKCDDCCGGAGRGACYGPQCLCRGAGAAGG |
| 51 | MCMPCFTTDHQMARACDDCCGGAGRGACYGPQCLCRGAGAAGG |
| 52 | MCMPCFTTDHQMARRCDDCCGGAGRGACYGPQCLCRGAGAAGG |
| 53 | MCMPCFTTDHQMARKCDDCCGGRGRGACYGPQCLCRGAGAAGG |
| 54 | MCMPCFTTDHQMARACDDCCGGRGRGACYGPQCLCRGAGAAGG |
| 55 | MCMPCFTTDHQMARRCDDCCGGRGRGACYGPQCLCRGAGAAGG |
| 56 | MCMPCFTTDHQMARKCDDCCGGKGRGRCYGPQCLCRGAGAAGG |
| 57 | MCMPCFTTDHQMARACDDCCGGKGRGRCYGPQCLCRGAGAAGG |
| 58 | MCMPCFTTDHQMARRCDDCCGGKGRGRCYGPQCLCRGAGAAGG |
| 59 | MCMPCFTTDHQMARKCDDCCGGAGRGRCYGPQCLCRGAGAAGG |
| 60 | MCMPCFTTDHQMARACDDCCGGAGRGRCYGPQCLCRGAGAAGG |
| 61 | MCMPCFTTDHQMARRCDDCCGGAGRGRCYGPQCLCRGAGAAGG |
| 62 | MCMPCFTTDHQMARKCDDCCGGRGRGRCYGPQCLCRGAGAAGG |
| 63 | MCMPCFTTDHQMARACDDCCGGRGRGRCYGPQCLCRGAGAAGG |
| 64 | MCMPCFTTDHQMARRCDDCCGGRGRGRCYGPQCLCRGAGAAGG |
| 65 | MCMPCFTTDHQMARRCDDCCGGRGRGRCYGPQCLCRGAGAAGG |
| 66 | KCMPCFTTDHQMARRCDDCCGGRGRGRCYGPQCLCRGAGAAGG |
| 67 | ACAPCFTTDHQAARRCDDCCGGRGRGRCYGPQCLCRGAGAAGG |
| 68 | KCAPCFTTDHQAARRCDDCCGGRGRGRCYGPQCLCRGAGAAGG |
| 69 | MCMPCFTTDHQMAR(Cit)CDDCCGG(Cit)GRGKCYGPQCLCRGAGAAGG |
| 70 | MCMPCFTTDHQMAR(Cit)CDDCCGG(Cit)GRG(Cit)CYGPQCLCRGAGAAGG |
| 71 | KCMPCFTTDHQMAR(Cit)CDDCCGG(Cit)GRG(Cit)CYGPQCLCRGAGAAGG |
| 72 | ACAPCFTTDHQAAR(Cit)CDDCCGG(Cit)GRGKCYGPQCLCRGAGAAGG |
| 73 | ACAPCFTTDHQAAR(Cit)CDDCCGG(Cit)GRG(Cit)CYGPQCLCRGAGAAGG |
| 74 | KCAPCFTTDHQAAR(Cit)CDDCCGG(Cit)GRG(Cit)CYGPQCLCRGAGAAGG |
| 75 | MCMPCFTTDHQMVRKCDDCCGGKGRGKCYGPQCLCR |
| 76 | MCMPCFTTDHQMVRVCDDCCGGKGRGKCYGPQCLCR |
| 77 | MCMPCFTTDHQMVRRCDDCCGGKGRGKCYGPQCLCR |
| 78 | MCMPCFTTDHQMVRKCDDCCGGVGRGKCYGPQCLCR |
| 79 | MCMPCFTTDHQMVRVCDDCCGGVGRGKCYGPQCLCR |
| 80 | MCMPCFTTDHQMVRRCDDCCGGVGRGKCYGPQCLCR |
| 81 | MCMPCFTTDHQMVRKCDDCCGGRGRGKCYGPQCLCR |
| 82 | MCMPCFTTDHQMVRVCDDCCGGRGRGKCYGPQCLCR |
| 83 | MCMPCFTTDHQMVRRCDDCCGGRGRGKCYGPQCLCR |
| 84 | MCMPCFTTDHQMVRKCDDCCGGKGRGVCYGPQCLCR |
| 85 | MCMPCFTTDHQMVRVCDDCCGGKGRGVCYGPQCLCR |
| 86 | MCMPCFTTDHQMVRRCDDCCGGKGRGVCYGPQCLCR |
| 87 | MCMPCFTTDHQMVRKCDDCCGGVGRGVCYGPQCLCR |
| 88 | MCMPCFTTDHQMVRVCDDCCGGVGRGVCYGPQCLCR |
| 89 | MCMPCFTTDHQMVRRCDDCCGGVGRGVCYGPQCLCR |
| 90 | MCMPCFTTDHQMVRKCDDCCGGRGRGVCYGPQCLCR |
| 91 | MCMPCFTTDHQMVRVCDDCCGGRGRGVCYGPQCLCR |
| 92 | MCMPCFTTDHQMVRRCDDCCGGRGRGVCYGPQCLCR |
| 93 | MCMPCFTTDHQMVRKCDDCCGGKGRGRCYGPQCLCR |
| 94 | MCMPCFTTDHQMVRVCDDCCGGKGRGRCYGPQCLCR |
| 95 | MCMPCFTTDHQMVRRCDDCCGGKGRGRCYGPQCLCR |
| 96 | MCMPCFTTDHQMVRKCDDCCGGVGRGRCYGPQCLCR |
| 97 | MCMPCFTTDHQMVRVCDDCCGGVGRGRCYGPQCLCR |
| 98 | MCMPCFTTDHQMVRRCDDCCGGVGRGRCYGPQCLCR |
| 99 | MCMPCFTTDHQMVRKCDDCCGGRGRGRCYGPQCLCR |
| 100 | MCMPCFTTDHQMVRVCDDCCGGRGRGRCYGPQCLCR |
| 101 | MCMPCFTTDHQMVRRCDDCCGGRGRGRCYGPQCLCR |
| 102 | MCMPCFTTDHQMVRRCDDCCGGRGRGRCYGPQCLCR |
| 103 | KCMPCFTTDHQMVRRCDDCCGGRGRGRCYGPQCLCR |
| 104 | VCVPCFTTDHQVVRRCDDCCGGRGRGRCYGPQCLCR |
| 105 | KCVPCFTTDHQVVRRCDDCCGGRGRGRCYGPQCLCR |
| 106 | MCMPCFTTDHQMVR(Cit)CDDCCGG(Cit)GRGKCYGPQCLCR |
| 107 | MCMPCFTTDHQMVR(Cit)CDDCCGG(Cit)GRG(Cit)CYGPQCLCR |
| 108 | KCMPCFTTDHQMVR(Cit)CDDCCGG(Cit)GRG(Cit)CYGPQCLCR |
| 109 | VCVPCFTTDHQVVR(Cit)CDDCCGG(Cit)GRGKCYGPQCLCR |
| 110 | VCVPCFTTDHQVVR(Cit)CDDCCGG(Cit)GRG(Cit)CYGPQCLCR |
| 111 | KCVPCFTTDHQVVR(Cit)CDDCCGG(Cit)GRG(Cit)CYGPQCLCR |
| 112 | MCMPCFTTDHQMVRKCDDCCGGKGRGKCYGPQCLCRGAGAAGG |
| 113 | MCMPCFTTDHQMVRVCDDCCGGKGRGKCYGPQCLCRGAGAAGG |
| 114 | MCMPCFTTDHQMVRRCDDCCGGKGRGKCYGPQCLCRGAGAAGG |
| 115 | MCMPCFTTDHQMVRKCDDCCGGVGRGKCYGPQCLCRGAGAAGG |
| 116 | MCMPCFTTDHQMVRVCDDCCGGVGRGKCYGPQCLCRGAGAAGG |
| 117 | MCMPCFTTDHQMVRRCDDCCGGVGRGKCYGPQCLCRGAGAAGG |
| 118 | MCMPCFTTDHQMVRKCDDCCGGRGRGKCYGPQCLCRGAGAAGG |
| 119 | MCMPCFTTDHQMVRVCDDCCGGRGRGKCYGPQCLCRGAGAAGG |
| 120 | MCMPCFTTDHQMVRRCDDCCGGRGRGKCYGPQCLCRGAGAAGG |
| 121 | MCMPCFTTDHQMVRKCDDCCGGKGRGVCYGPQCLCRGAGAAGG |
| 122 | MCMPCFTTDHQMVRVCDDCCGGKGRGVCYGPQCLCRGAGAAGG |
| 123 | MCMPCFTTDHQMVRRCDDCCGGKGRGVCYGPQCLCRGAGAAGG |
| 124 | MCMPCFTTDHQMVRKCDDCCGGVGRGVCYGPQCLCRGAGAAGG |
| 125 | MCMPCFTTDHQMVRVCDDCCGGVGRGVCYGPQCLCRGAGAAGG |
| 126 | MCMPCFTTDHQMVRRCDDCCGGVGRGVCYGPQCLCRGAGAAGG |
| 127 | MCMPCFTTDHQMVRKCDDCCGGRGRGVCYGPQCLCRGAGAAGG |
| 128 | MCMPCFTTDHQMVRVCDDCCGGRGRGVCYGPQCLCRGAGAAGG |
| 129 | MCMPCFTTDHQMVRRCDDCCGGRGRGVCYGPQCLCRGAGAAGG |
| 130 | MCMPCFTTDHQMVRKCDDCCGGKGRGRCYGPQCLCRGAGAAGG |
| 131 | MCMPCFTTDHQMVRVCDDCCGGKGRGRCYGPQCLCRGAGAAGG |
| 132 | MCMPCFTTDHQMVRRCDDCCGGKGRGRCYGPQCLCRGAGAAGG |
| 133 | MCMPCFTTDHQMVRKCDDCCGGVGRGRCYGPQCLCRGAGAAGG |
| 134 | MCMPCFTTDHQMVRVCDDCCGGVGRGRCYGPQCLCRGAGAAGG |
| 135 | MCMPCFTTDHQMVRRCDDCCGGVGRGRCYGPQCLCRGAGAAGG |
| 136 | MCMPCFTTDHQMVRKCDDCCGGRGRGRCYGPQCLCRGAGAAGG |
| 137 | MCMPCFTTDHQMVRVCDDCCGGRGRGRCYGPQCLCRGAGAAGG |
| 138 | MCMPCFTTDHQMVRRCDDCCGGRGRGRCYGPQCLCRGAGAAGG |
| 139 | MCMPCFTTDHQMVRRCDDCCGGRGRGRCYGPQCLCRGAGAAGG |
| 140 | KCMPCFTTDHQMVRRCDDCCGGRGRGRCYGPQCLCRGAGAAGG |
| 141 | VCVPCFTTDHQVVRRCDDCCGGRGRGRCYGPQCLCRGAGAAGG |
| 142 | KCVPCFTTDHQVVRRCDDCCGGRGRGRCYGPQCLCRGAGAAGG |
| 143 | MCMPCFTTDHQMVR(Cit)CDDCCGG(Cit)GRGKCYGPQCLCRGAGAAGG |
| 144 | MCMPCFTTDHQMVR(Cit)CDDCCGG(Cit)GRG(Cit)CYGPQCLCRGAGAAGG |
| 145 | KCMPCFTTDHQMVR(Cit)CDDCCGG(Cit)GRG(Cit)CYGPQCLCRGAGAAGG |
| 146 | VCVPCFTTDHQVVR(Cit)CDDCCGG(Cit)GRGKCYGPQCLCRGAGAAGG |
| 147 | VCVPCFTTDHQVVR(Cit)CDDCCGG(Cit)GRG(Cit)CYGPQCLCRGAGAAGG |
| 148 | KCVPCFTTDHQVVR(Cit)CDDCCGG(Cit)GRG(Cit)CYGPQCLCRGAGAAGG |
| 149 | MCMPCFTTDHQMLRKCDDCCGGKGRGKCYGPQCLCR |
| 150 | MCMPCFTTDHQMLRLCDDCCGGKGRGKCYGPQCLCR |
| 151 | MCMPCFTTDHQMLRRCDDCCGGKGRGKCYGPQCLCR |
| 152 | MCMPCFTTDHQMLRKCDDCCGGLGRGKCYGPQCLCR |
| 153 | MCMPCFTTDHQMLRLCDDCCGGLGRGKCYGPQCLCR |
| 154 | MCMPCFTTDHQMLRRCDDCCGGLGRGKCYGPQCLCR |
| 155 | MCMPCFTTDHQMLRKCDDCCGGRGRGKCYGPQCLCR |
| 156 | MCMPCFTTDHQMLRLCDDCCGGRGRGKCYGPQCLCR |
| 157 | MCMPCFTTDHQMLRRCDDCCGGRGRGKCYGPQCLCR |
| 158 | MCMPCFTTDHQMLRKCDDCCGGKGRGLCYGPQCLCR |
| 159 | MCMPCFTTDHQMLRLCDDCCGGKGRGLCYGPQCLCR |
| 160 | MCMPCFTTDHQMLRRCDDCCGGKGRGLCYGPQCLCR |
| 161 | MCMPCFTTDHQMLRKCDDCCGGLGRGLCYGPQCLCR |
| 162 | MCMPCFTTDHQMLRLCDDCCGGLGRGLCYGPQCLCR |
| 163 | MCMPCFTTDHQMLRRCDDCCGGLGRGLCYGPQCLCR |
| 164 | MCMPCFTTDHQMLRKCDDCCGGRGRGLCYGPQCLCR |
| 165 | MCMPCFTTDHQMLRLCDDCCGGRGRGLCYGPQCLCR |
| 166 | MCMPCFTTDHQMLRRCDDCCGGRGRGLCYGPQCLCR |
| 167 | MCMPCFTTDHQMLRKCDDCCGGKGRGRCYGPQCLCR |
| 168 | MCMPCFTTDHQMLRLCDDCCGGKGRGRCYGPQCLCR |
| 169 | MCMPCFTTDHQMLRRCDDCCGGKGRGRCYGPQCLCR |
| 170 | MCMPCFTTDHQMLRKCDDCCGGLGRGRCYGPQCLCR |
| 171 | MCMPCFTTDHQMLRLCDDCCGGLGRGRCYGPQCLCR |
| 172 | MCMPCFTTDHQMLRRCDDCCGGLGRGRCYGPQCLCR |
| 173 | MCMPCFTTDHQMLRKCDDCCGGRGRGRCYGPQCLCR |
| 174 | MCMPCFTTDHQMLRLCDDCCGGRGRGRCYGPQCLCR |
| 175 | MCMPCFTTDHQMLRRCDDCCGGRGRGRCYGPQCLCR |
| 176 | MCMPCFTTDHQMLRRCDDCCGGRGRGRCYGPQCLCR |
| 177 | KCMPCFTTDHQMLRRCDDCCGGRGRGRCYGPQCLCR |
| 178 | LCLPCFTTDHQLLRRCDDCCGGRGRGRCYGPQCLCR |
| 179 | KCLPCFTTDHQLLRRCDDCCGGRGRGRCYGPQCLCR |
| 180 | MCMPCFTTDHQMLR(Cit)CDDCCGG(Cit)GRGKCYGPQCLCR |
| 181 | MCMPCFTTDHQMLR(Cit)CDDCCGG(Cit)GRG(Cit)CYGPQCLCR |
| 182 | KCMPCFTTDHQMLR(Cit)CDDCCGG(Cit)GRG(Cit)CYGPQCLCR |
| 183 | LCLPCFTTDHQLLR(Cit)CDDCCGG(Cit)GRGKCYGPQCLCR |
| 184 | LCLPCFTTDHQLLR(Cit)CDDCCGG(Cit)GRG(Cit)CYGPQCLCR |
| 185 | KCLPCFTTDHQLLR(Cit)CDDCCGG(Cit)GRG(Cit)CYGPQCLCR |
| 186 | MCMPCFTTDHQMLRKCDDCCGGKGRGKCYGPQCLCRGAGAAGG |
| 187 | MCMPCFTTDHQMLRLCDDCCGGKGRGKCYGPQCLCRGAGAAGG |
| 188 | MCMPCFTTDHQMLRRCDDCCGGKGRGKCYGPQCLCRGAGAAGG |
| 189 | MCMPCFTTDHQMLRKCDDCCGGLGRGKCYGPQCLCRGAGAAGG |
| 190 | MCMPCFTTDHQMLRLCDDCCGGLGRGKCYGPQCLCRGAGAAGG |
| 191 | MCMPCFTTDHQMLRRCDDCCGGLGRGKCYGPQCLCRGAGAAGG |
| 192 | MCMPCFTTDHQMLRKCDDCCGGRGRGKCYGPQCLCRGAGAAGG |
| 193 | MCMPCFTTDHQMLRLCDDCCGGRGRGKCYGPQCLCRGAGAAGG |
| 194 | MCMPCFTTDHQMLRRCDDCCGGRGRGKCYGPQCLCRGAGAAGG |
| 195 | MCMPCFTTDHQMLRKCDDCCGGKGRGLCYGPQCLCRGAGAAGG |
| 196 | MCMPCFTTDHQMLRLCDDCCGGKGRGLCYGPQCLCRGAGAAGG |
| 197 | MCMPCFTTDHQMLRRCDDCCGGKGRGLCYGPQCLCRGAGAAGG |
| 198 | MCMPCFTTDHQMLRKCDDCCGGLGRGLCYGPQCLCRGAGAAGG |
| 199 | MCMPCFTTDHQMLRLCDDCCGGLGRGLCYGPQCLCRGAGAAGG |
| 200 | MCMPCFTTDHQMLRRCDDCCGGLGRGLCYGPQCLCRGAGAAGG |
| 201 | MCMPCFTTDHQMLRKCDDCCGGRGRGLCYGPQCLCRGAGAAGG |
| 202 | MCMPCFTTDHQMLRLCDDCCGGRGRGLCYGPQCLCRGAGAAGG |
| 203 | MCMPCFTTDHQMLRRCDDCCGGRGRGLCYGPQCLCRGAGAAGG |
| 204 | MCMPCFTTDHQMLRKCDDCCGGKGRGRCYGPQCLCRGAGAAGG |
| 205 | MCMPCFTTDHQMLRLCDDCCGGKGRGRCYGPQCLCRGAGAAGG |
| 206 | MCMPCFTTDHQMLRRCDDCCGGKGRGRCYGPQCLCRGAGAAGG |
| 207 | MCMPCFTTDHQMLRKCDDCCGGLGRGRCYGPQCLCRGAGAAGG |
| 208 | MCMPCFTTDHQMLRLCDDCCGGLGRGRCYGPQCLCRGAGAAGG |
| 209 | MCMPCFTTDHQMLRRCDDCCGGLGRGRCYGPQCLCRGAGAAGG |
| 210 | MCMPCFTTDHQMLRKCDDCCGGRGRGRCYGPQCLCRGAGAAGG |
| 211 | MCMPCFTTDHQMLRLCDDCCGGRGRGRCYGPQCLCRGAGAAGG |
| 212 | MCMPCFTTDHQMLRRCDDCCGGRGRGRCYGPQCLCRGAGAAGG |
| 213 | MCMPCFTTDHQMLRRCDDCCGGRGRGRCYGPQCLCRGAGAAGG |
| 214 | KCMPCFTTDHQMLRRCDDCCGGRGRGRCYGPQCLCRGAGAAGG |
| 215 | LCLPCFTTDHQLLRRCDDCCGGRGRGRCYGPQCLCRGAGAAGG |
| 216 | KCLPCFTTDHQLLRRCDDCCGGRGRGRCYGPQCLCRGAGAAGG |
| 217 | MCMPCFTTDHQMLR(Cit)CDDCCGG(Cit)GRGKCYGPQCLCRGAGAAGG |
| 218 | MCMPCFTTDHQMLR(Cit)CDDCCGG(Cit)GRG(Cit)CYGPQCLCRGAGAAGG |
| 219 | KCMPCFTTDHQMLR(Cit)CDDCCGG(Cit)GRG(Cit)CYGPQCLCRGAGAAGG |
| 220 | LCLPCFTTDHQLLR(Cit)CDDCCGG(Cit)GRGKCYGPQCLCRGAGAAGG |
| 221 | LCLPCFTTDHQLLR(Cit)CDDCCGG(Cit)GRG(Cit)CYGPQCLCRGAGAAGG |
| 222 | KCLPCFTTDHQLLR(Cit)CDDCCGG(Cit)GRG(Cit)CYGPQCLCRGAGAAGG |
| 223 | GCGPCFTTDHQGARKCDDCCGGKGRGKCYGPQCLCR |
| 224 | GCGPCFTTDHQGARACDDCCGGKGRGKCYGPQCLCR |
| 225 | GCGPCFTTDHQGARRCDDCCGGKGRGKCYGPQCLCR |
| 226 | GCGPCFTTDHQGARKCDDCCGGAGRGKCYGPQCLCR |
| 227 | GCGPCFTTDHQGARACDDCCGGAGRGKCYGPQCLCR |
| 228 | GCGPCFTTDHQGARRCDDCCGGAGRGKCYGPQCLCR |
| 229 | GCGPCFTTDHQGARKCDDCCGGRGRGKCYGPQCLCR |
| 230 | GCGPCFTTDHQGARACDDCCGGRGRGKCYGPQCLCR |
| 231 | GCGPCFTTDHQGARRCDDCCGGRGRGKCYGPQCLCR |
| 232 | GCGPCFTTDHQGARKCDDCCGGKGRGACYGPQCLCR |
| 233 | GCGPCFTTDHQGARACDDCCGGKGRGACYGPQCLCR |
| 234 | GCGPCFTTDHQGARRCDDCCGGKGRGACYGPQCLCR |
| 235 | GCGPCFTTDHQGARKCDDCCGGAGRGACYGPQCLCR |
| 236 | GCGPCFTTDHQGARACDDCCGGAGRGACYGPQCLCR |
| 237 | GCGPCFTTDHQGARRCDDCCGGAGRGACYGPQCLCR |
| 238 | GCGPCFTTDHQGARKCDDCCGGRGRGACYGPQCLCR |
| 239 | GCGPCFTTDHQGARACDDCCGGRGRGACYGPQCLCR |
| 240 | GCGPCFTTDHQGARRCDDCCGGRGRGACYGPQCLCR |
| 241 | GCGPCFTTDHQGARKCDDCCGGKGRGRCYGPQCLCR |
| 242 | GCGPCFTTDHQGARACDDCCGGKGRGRCYGPQCLCR |
| 243 | GCGPCFTTDHQGARRCDDCCGGKGRGRCYGPQCLCR |
| 244 | GCGPCFTTDHQGARKCDDCCGGAGRGRCYGPQCLCR |
| 245 | GCGPCFTTDHQGARACDDCCGGAGRGRCYGPQCLCR |
| 246 | GCGPCFTTDHQGARRCDDCCGGAGRGRCYGPQCLCR |
| 247 | GCGPCFTTDHQGARKCDDCCGGRGRGRCYGPQCLCR |
| 248 | GCGPCFTTDHQGARACDDCCGGRGRGRCYGPQCLCR |
| 249 | GCGPCFTTDHQGARRCDDCCGGRGRGRCYGPQCLCR |
| 250 | GCGPCFTTDHQGARRCDDCCGGRGRGRCYGPQCLCR |
| 251 | KCGPCFTTDHQGARRCDDCCGGRGRGRCYGPQCLCR |
| 252 | ACAPCFTTDHQAARRCDDCCGGRGRGRCYGPQCLCR |
| 253 | KCAPCFTTDHQAARRCDDCCGGRGRGRCYGPQCLCR |
| 254 | GCGPCFTTDHQGAR(Cit)CDDCCGG(Cit)GRGKCYGPQCLCR |
| 255 | GCGPCFTTDHQGAR(Cit)CDDCCGG(Cit)GRG(Cit)CYGPQCLCR |
| 256 | KCGPCFTTDHQGAR(Cit)CDDCCGG(Cit)GRG(Cit)CYGPQCLCR |
| 257 | ACAPCFTTDHQAAR(Cit)CDDCCGG(Cit)GRGKCYGPQCLCR |
| 258 | ACAPCFTTDHQAAR(Cit)CDDCCGG(Cit)GRG(Cit)CYGPQCLCR |
| 259 | KCAPCFTTDHQAAR(Cit)CDDCCGG(Cit)GRG(Cit)CYGPQCLCR |
| 260 | ACAPCFTTDHQAARKCDDCCGGKGRGKCYGPQCLCR |
| 261 | ACAPCFTTDHQAARACDDCCGGKGRGKCYGPQCLCR |
| 262 | ACAPCFTTDHQAARRCDDCCGGKGRGKCYGPQCLCR |
| 263 | ACAPCFTTDHQAARKCDDCCGGAGRGKCYGPQCLCR |
| 264 | ACAPCFTTDHQAARACDDCCGGAGRGKCYGPQCLCR |
| 265 | ACAPCFTTDHQAARRCDDCCGGAGRGKCYGPQCLCR |
| 266 | ACAPCFTTDHQAARKCDDCCGGRGRGKCYGPQCLCR |
| 267 | ACAPCFTTDHQAARACDDCCGGRGRGKCYGPQCLCR |
| 268 | ACAPCFTTDHQAARRCDDCCGGRGRGKCYGPQCLCR |
| 269 | ACAPCFTTDHQAARKCDDCCGGKGRGACYGPQCLCR |
| 270 | ACAPCFTTDHQAARACDDCCGGKGRGACYGPQCLCR |
| 271 | ACAPCFTTDHQAARRCDDCCGGKGRGACYGPQCLCR |
| 272 | ACAPCFTTDHQAARKCDDCCGGAGRGACYGPQCLCR |
| 273 | ACAPCFTTDHQAARACDDCCGGAGRGACYGPQCLCR |
| 274 | ACAPCFTTDHQAARRCDDCCGGAGRGACYGPQCLCR |
| 275 | ACAPCFTTDHQAARKCDDCCGGRGRGACYGPQCLCR |
| 276 | ACAPCFTTDHQAARACDDCCGGRGRGACYGPQCLCR |
| 277 | ACAPCFTTDHQAARRCDDCCGGRGRGACYGPQCLCR |
| 278 | ACAPCFTTDHQAARKCDDCCGGKGRGRCYGPQCLCR |
| 279 | ACAPCFTTDHQAARACDDCCGGKGRGRCYGPQCLCR |
| 280 | ACAPCFTTDHQAARRCDDCCGGKGRGRCYGPQCLCR |
| 281 | ACAPCFTTDHQAARKCDDCCGGAGRGRCYGPQCLCR |
| 282 | ACAPCFTTDHQAARACDDCCGGAGRGRCYGPQCLCR |
| 283 | ACAPCFTTDHQAARRCDDCCGGAGRGRCYGPQCLCR |
| 284 | ACAPCFTTDHQAARKCDDCCGGRGRGRCYGPQCLCR |
| 285 | ACAPCFTTDHQAARACDDCCGGRGRGRCYGPQCLCR |
| 286 | ACAPCFTTDHQAARRCDDCCGGRGRGRCYGPQCLCR |
| 287 | ACAPCFTTDHQAARRCDDCCGGRGRGRCYGPQCLCR |
| 288 | KCAPCFTTDHQAARRCDDCCGGRGRGRCYGPQCLCR |
| 289 | ACAPCFTTDHQAARRCDDCCGGRGRGRCYGPQCLCR |
| 290 | KCAPCFTTDHQAARRCDDCCGGRGRGRCYGPQCLCR |
| 291 | ACAPCFTTDHQAAR(Cit)CDDCCGG(Cit)GRGKCYGPQCLCR |
| 292 | ACAPCFTTDHQAAR(Cit)CDDCCGG(Cit)GRG(Cit)CYGPQCLCR |
| 293 | KCAPCFTTDHQAAR(Cit)CDDCCGG(Cit)GRG(Cit)CYGPQCLCR |
| 294 | ACAPCFTTDHQAAR(Cit)CDDCCGG(Cit)GRGKCYGPQCLCR |
| 295 | ACAPCFTTDHQAAR(Cit)CDDCCGG(Cit)GRG(Cit)CYGPQCLCR |
| 296 | KCAPCFTTDHQAAR(Cit)CDDCCGG(Cit)GRG(Cit)CYGPQCLCR |
| 297 | ICIPCFTTDHQIARKCDDCCGGKGRGKCYGPQCLCR |
| 298 | ICIPCFTTDHQIARACDDCCGGKGRGKCYGPQCLCR |
| 299 | ICIPCFTTDHQIARRCDDCCGGKGRGKCYGPQCLCR |
| 300 | ICIPCFTTDHQIARKCDDCCGGAGRGKCYGPQCLCR |
| 301 | ICIPCFTTDHQIARACDDCCGGAGRGKCYGPQCLCR |
| 302 | ICIPCFTTDHQIARRCDDCCGGAGRGKCYGPQCLCR |
| 303 | ICIPCFTTDHQIARKCDDCCGGRGRGKCYGPQCLCR |
| 304 | ICIPCFTTDHQIARACDDCCGGRGRGKCYGPQCLCR |
| 305 | ICIPCFTTDHQIARRCDDCCGGRGRGKCYGPQCLCR |
| 306 | ICIPCFTTDHQIARKCDDCCGGKGRGACYGPQCLCR |
| 307 | ICIPCFTTDHQIARACDDCCGGKGRGACYGPQCLCR |
| 308 | ICIPCFTTDHQIARRCDDCCGGKGRGACYGPQCLCR |
| 309 | ICIPCFTTDHQIARKCDDCCGGAGRGACYGPQCLCR |
| 310 | ICIPCFTTDHQIARACDDCCGGAGRGACYGPQCLCR |
| 311 | ICIPCFTTDHQIARRCDDCCGGAGRGACYGPQCLCR |
| 312 | ICIPCFTTDHQIARKCDDCCGGRGRGACYGPQCLCR |
| 313 | ICIPCFTTDHQIARACDDCCGGRGRGACYGPQCLCR |
| 314 | ICIPCFTTDHQIARRCDDCCGGRGRGACYGPQCLCR |
| 315 | ICIPCFTTDHQIARKCDDCCGGKGRGRCYGPQCLCR |
| 316 | ICIPCFTTDHQIARACDDCCGGKGRGRCYGPQCLCR |
| 317 | ICIPCFTTDHQIARRCDDCCGGKGRGRCYGPQCLCR |
| 318 | ICIPCFTTDHQIARKCDDCCGGAGRGRCYGPQCLCR |
| 319 | ICIPCFTTDHQIARACDDCCGGAGRGRCYGPQCLCR |
| 320 | ICIPCFTTDHQIARRCDDCCGGAGRGRCYGPQCLCR |
| 321 | ICIPCFTTDHQIARKCDDCCGGRGRGRCYGPQCLCR |
| 322 | ICIPCFTTDHQIARACDDCCGGRGRGRCYGPQCLCR |
| 323 | ICIPCFTTDHQIARRCDDCCGGRGRGRCYGPQCLCR |
| 324 | ICIPCFTTDHQIARRCDDCCGGRGRGRCYGPQCLCR |
| 325 | KCIPCFTTDHQIARRCDDCCGGRGRGRCYGPQCLCR |
| 326 | ACAPCFTTDHQAARRCDDCCGGRGRGRCYGPQCLCR |
| 327 | KCAPCFTTDHQAARRCDDCCGGRGRGRCYGPQCLCR |
| 328 | ICIPCFTTDHQIAR(Cit)CDDCCGG(Cit)GRGKCYGPQCLCR |
| 329 | ICIPCFTTDHQIAR(Cit)CDDCCGG(Cit)GRG(Cit)CYGPQCLCR |
| 330 | KCIPCFTTDHQIAR(Cit)CDDCCGG(Cit)GRG(Cit)CYGPQCLCR |
| 331 | ACAPCFTTDHQAAR(Cit)CDDCCGG(Cit)GRGKCYGPQCLCR |
| 332 | ACAPCFTTDHQAAR(Cit)CDDCCGG(Cit)GRG(Cit)CYGPQCLCR |
| 333 | KCAPCFTTDHQAAR(Cit)CDDCCGG(Cit)GRG(Cit)CYGPQCLCR |
| 334 | TCTPCFTTDHQTARKCDDCCGGKGRGKCYGPQCLCR |
| 335 | TCTPCFTTDHQTARACDDCCGGKGRGKCYGPQCLCR |
| 336 | TCTPCFTTDHQTARRCDDCCGGKGRGKCYGPQCLCR |
| 337 | TCTPCFTTDHQTARKCDDCCGGAGRGKCYGPQCLCR |
| 338 | TCTPCFTTDHQTARACDDCCGGAGRGKCYGPQCLCR |
| 339 | TCTPCFTTDHQTARRCDDCCGGAGRGKCYGPQCLCR |
| 340 | TCTPCFTTDHQTARKCDDCCGGRGRGKCYGPQCLCR |
| 341 | TCTPCFTTDHQTARACDDCCGGRGRGKCYGPQCLCR |
| 342 | TCTPCFTTDHQTARRCDDCCGGRGRGKCYGPQCLCR |
| 343 | TCTPCFTTDHQTARKCDDCCGGKGRGACYGPQCLCR |
| 344 | TCTPCFTTDHQTARACDDCCGGKGRGACYGPQCLCR |
| 345 | TCTPCFTTDHQTARRCDDCCGGKGRGACYGPQCLCR |
| 346 | TCTPCFTTDHQTARKCDDCCGGAGRGACYGPQCLCR |
| 347 | TCTPCFTTDHQTARACDDCCGGAGRGACYGPQCLCR |
| 348 | TCTPCFTTDHQTARRCDDCCGGAGRGACYGPQCLCR |
| 349 | TCTPCFTTDHQTARKCDDCCGGRGRGACYGPQCLCR |
| 350 | TCTPCFTTDHQTARACDDCCGGRGRGACYGPQCLCR |
| 351 | TCTPCFTTDHQTARRCDDCCGGRGRGACYGPQCLCR |
| 352 | TCTPCFTTDHQTARKCDDCCGGKGRGRCYGPQCLCR |
| 353 | TCTPCFTTDHQTARACDDCCGGKGRGRCYGPQCLCR |
| 354 | TCTPCFTTDHQTARRCDDCCGGKGRGRCYGPQCLCR |
| 355 | TCTPCFTTDHQTARKCDDCCGGAGRGRCYGPQCLCR |
| 356 | TCTPCFTTDHQTARACDDCCGGAGRGRCYGPQCLCR |
| 357 | TCTPCFTTDHQTARRCDDCCGGAGRGRCYGPQCLCR |
| 358 | TCTPCFTTDHQTARKCDDCCGGRGRGRCYGPQCLCR |
| 359 | TCTPCFTTDHQTARACDDCCGGRGRGRCYGPQCLCR |
| 360 | TCTPCFTTDHQTARRCDDCCGGRGRGRCYGPQCLCR |
| 361 | TCTPCFTTDHQTARRCDDCCGGRGRGRCYGPQCLCR |
| 362 | KCTPCFTTDHQTARRCDDCCGGRGRGRCYGPQCLCR |
| 363 | ACAPCFTTDHQAARRCDDCCGGRGRGRCYGPQCLCR |
| 364 | KCAPCFTTDHQAARRCDDCCGGRGRGRCYGPQCLCR |
| 365 | TCTPCFTTDHQTAR(Cit)CDDCCGG(Cit)GRGKCYGPQCLCR |
| 366 | TCTPCFTTDHQTAR(Cit)CDDCCGG(Cit)GRG(Cit)CYGPQCLCR |
| 367 | KCTPCFTTDHQTAR(Cit)CDDCCGG(Cit)GRG(Cit)CYGPQCLCR |
| 368 | ACAPCFTTDHQAAR(Cit)CDDCCGG(Cit)GRGKCYGPQCLCR |
| 369 | ACAPCFTTDHQAAR(Cit)CDDCCGG(Cit)GRG(Cit)CYGPQCLCR |
| 370 | KCAPCFTTDHQAAR(Cit)CDDCCGG(Cit)GRG(Cit)CYGPQCLCR |
| 371 | VCVPCFTTDHQVARKCDDCCGGKGRGKCYGPQCLCR |
| 372 | VCVPCFTTDHQVARACDDCCGGKGRGKCYGPQCLCR |
| 373 | VCVPCFTTDHQVARRCDDCCGGKGRGKCYGPQCLCR |
| 374 | VCVPCFTTDHQVARKCDDCCGGAGRGKCYGPQCLCR |
| 375 | VCVPCFTTDHQVARACDDCCGGAGRGKCYGPQCLCR |
| 376 | VCVPCFTTDHQVARRCDDCCGGAGRGKCYGPQCLCR |
| 377 | VCVPCFTTDHQVARKCDDCCGGRGRGKCYGPQCLCR |
| 378 | VCVPCFTTDHQVARACDDCCGGRGRGKCYGPQCLCR |
| 379 | VCVPCFTTDHQVARRCDDCCGGRGRGKCYGPQCLCR |
| 380 | VCVPCFTTDHQVARKCDDCCGGKGRGACYGPQCLCR |
| 381 | VCVPCFTTDHQVARACDDCCGGKGRGACYGPQCLCR |
| 382 | VCVPCFTTDHQVARRCDDCCGGKGRGACYGPQCLCR |
| 383 | VCVPCFTTDHQVARKCDDCCGGAGRGACYGPQCLCR |
| 384 | VCVPCFTTDHQVARACDDCCGGAGRGACYGPQCLCR |
| 385 | VCVPCFTTDHQVARRCDDCCGGAGRGACYGPQCLCR |
| 386 | VCVPCFTTDHQVARKCDDCCGGRGRGACYGPQCLCR |
| 387 | VCVPCFTTDHQVARACDDCCGGRGRGACYGPQCLCR |
| 388 | VCVPCFTTDHQVARRCDDCCGGRGRGACYGPQCLCR |
| 389 | VCVPCFTTDHQVARKCDDCCGGKGRGRCYGPQCLCR |
| 390 | VCVPCFTTDHQVARACDDCCGGKGRGRCYGPQCLCR |
| 391 | VCVPCFTTDHQVARRCDDCCGGKGRGRCYGPQCLCR |
| 392 | VCVPCFTTDHQVARKCDDCCGGAGRGRCYGPQCLCR |
| 393 | VCVPCFTTDHQVARACDDCCGGAGRGRCYGPQCLCR |
| 394 | VCVPCFTTDHQVARRCDDCCGGAGRGRCYGPQCLCR |
| 395 | VCVPCFTTDHQVARKCDDCCGGRGRGRCYGPQCLCR |
| 396 | VCVPCFTTDHQVARACDDCCGGRGRGRCYGPQCLCR |
| 397 | VCVPCFTTDHQVARRCDDCCGGRGRGRCYGPQCLCR |
| 398 | VCVPCFTTDHQVARRCDDCCGGRGRGRCYGPQCLCR |
| 399 | KCVPCFTTDHQVARRCDDCCGGRGRGRCYGPQCLCR |
| 400 | ACAPCFTTDHQAARRCDDCCGGRGRGRCYGPQCLCR |
| 401 | KCAPCFTTDHQAARRCDDCCGGRGRGRCYGPQCLCR |
| 402 | VCVPCFTTDHQVAR(Cit)CDDCCGG(Cit)GRGKCYGPQCLCR |
| 403 | VCVPCFTTDHQVAR(Cit)CDDCCGG(Cit)GRG(Cit)CYGPQCLCR |
| 404 | KCVPCFTTDHQVAR(Cit)CDDCCGG(Cit)GRG(Cit)CYGPQCLCR |
| 405 | ACAPCFTTDHQAAR(Cit)CDDCCGG(Cit)GRGKCYGPQCLCR |
| 406 | ACAPCFTTDHQAAR(Cit)CDDCCGG(Cit)GRG(Cit)CYGPQCLCR |
| 407 | KCAPCFTTDHQAAR(Cit)CDDCCGG(Cit)GRG(Cit)CYGPQCLCR |
| 408 | LCLPCFTTDHQLARKCDDCCGGKGRGKCYGPQCLCR |
| 409 | LCLPCFTTDHQLARACDDCCGGKGRGKCYGPQCLCR |
| 410 | LCLPCFTTDHQLARRCDDCCGGKGRGKCYGPQCLCR |
| 411 | LCLPCFTTDHQLARKCDDCCGGAGRGKCYGPQCLCR |
| 412 | LCLPCFTTDHQLARACDDCCGGAGRGKCYGPQCLCR |
| 413 | LCLPCFTTDHQLARRCDDCCGGAGRGKCYGPQCLCR |
| 414 | LCLPCFTTDHQLARKCDDCCGGRGRGKCYGPQCLCR |
| 415 | LCLPCFTTDHQLARACDDCCGGRGRGKCYGPQCLCR |
| 416 | LCLPCFTTDHQLARRCDDCCGGRGRGKCYGPQCLCR |
| 417 | LCLPCFTTDHQLARKCDDCCGGKGRGACYGPQCLCR |
| 418 | LCLPCFTTDHQLARACDDCCGGKGRGACYGPQCLCR |
| 419 | LCLPCFTTDHQLARRCDDCCGGKGRGACYGPQCLCR |
| 420 | LCLPCFTTDHQLARKCDDCCGGAGRGACYGPQCLCR |
| 421 | LCLPCFTTDHQLARACDDCCGGAGRGACYGPQCLCR |
| 422 | LCLPCFTTDHQLARRCDDCCGGAGRGACYGPQCLCR |
| 423 | LCLPCFTTDHQLARKCDDCCGGRGRGACYGPQCLCR |
| 424 | LCLPCFTTDHQLARACDDCCGGRGRGACYGPQCLCR |
| 425 | LCLPCFTTDHQLARRCDDCCGGRGRGACYGPQCLCR |
| 426 | LCLPCFTTDHQLARKCDDCCGGKGRGRCYGPQCLCR |
| 427 | LCLPCFTTDHQLARACDDCCGGKGRGRCYGPQCLCR |
| 428 | LCLPCFTTDHQLARRCDDCCGGKGRGRCYGPQCLCR |
| 429 | LCLPCFTTDHQLARKCDDCCGGAGRGRCYGPQCLCR |
| 430 | LCLPCFTTDHQLARACDDCCGGAGRGRCYGPQCLCR |
| 431 | LCLPCFTTDHQLARRCDDCCGGAGRGRCYGPQCLCR |
| 432 | LCLPCFTTDHQLARKCDDCCGGRGRGRCYGPQCLCR |
| 433 | LCLPCFTTDHQLARACDDCCGGRGRGRCYGPQCLCR |
| 434 | LCLPCFTTDHQLARRCDDCCGGRGRGRCYGPQCLCR |
| 435 | LCLPCFTTDHQLARRCDDCCGGRGRGRCYGPQCLCR |
| 436 | KCLPCFTTDHQLARRCDDCCGGRGRGRCYGPQCLCR |
| 437 | ACAPCFTTDHQAARRCDDCCGGRGRGRCYGPQCLCR |
| 438 | KCAPCFTTDHQAARRCDDCCGGRGRGRCYGPQCLCR |
| 439 | LCLPCFTTDHQLAR(Cit)CDDCCGG(Cit)GRGKCYGPQCLCR |
| 440 | LCLPCFTTDHQLAR(Cit)CDDCCGG(Cit)GRG(Cit)CYGPQCLCR |
| 441 | KCLPCFTTDHQLAR(Cit)CDDCCGG(Cit)GRG(Cit)CYGPQCLCR |
| 442 | ACAPCFTTDHQAAR(Cit)CDDCCGG(Cit)GRGKCYGPQCLCR |
| 443 | ACAPCFTTDHQAAR(Cit)CDDCCGG(Cit)GRG(Cit)CYGPQCLCR |
| 444 | KCAPCFTTDHQAAR(Cit)CDDCCGG(Cit)GRG(Cit)CYGPQCLCR |
| 445 | SCSPCFTTDHQSARKCDDCCGGKGRGKCYGPQCLCR |
| 446 | SCSPCFTTDHQSARACDDCCGGKGRGKCYGPQCLCR |
| 447 | SCSPCFTTDHQSARRCDDCCGGKGRGKCYGPQCLCR |
| 448 | SCSPCFTTDHQSARKCDDCCGGAGRGKCYGPQCLCR |
| 449 | SCSPCFTTDHQSARACDDCCGGAGRGKCYGPQCLCR |
| 450 | SCSPCFTTDHQSARRCDDCCGGAGRGKCYGPQCLCR |
| 451 | SCSPCFTTDHQSARKCDDCCGGRGRGKCYGPQCLCR |
| 452 | SCSPCFTTDHQSARACDDCCGGRGRGKCYGPQCLCR |
| 453 | SCSPCFTTDHQSARRCDDCCGGRGRGKCYGPQCLCR |
| 454 | SCSPCFTTDHQSARKCDDCCGGKGRGACYGPQCLCR |
| 455 | SCSPCFTTDHQSARACDDCCGGKGRGACYGPQCLCR |
| 456 | SCSPCFTTDHQSARRCDDCCGGKGRGACYGPQCLCR |
| 457 | SCSPCFTTDHQSARKCDDCCGGAGRGACYGPQCLCR |
| 458 | SCSPCFTTDHQSARACDDCCGGAGRGACYGPQCLCR |
| 459 | SCSPCFTTDHQSARRCDDCCGGAGRGACYGPQCLCR |
| 460 | SCSPCFTTDHQSARKCDDCCGGRGRGACYGPQCLCR |
| 461 | SCSPCFTTDHQSARACDDCCGGRGRGACYGPQCLCR |
| 462 | SCSPCFTTDHQSARRCDDCCGGRGRGACYGPQCLCR |
| 463 | SCSPCFTTDHQSARKCDDCCGGKGRGRCYGPQCLCR |
| 464 | SCSPCFTTDHQSARACDDCCGGKGRGRCYGPQCLCR |
| 465 | SCSPCFTTDHQSARRCDDCCGGKGRGRCYGPQCLCR |
| 466 | SCSPCFTTDHQSARKCDDCCGGAGRGRCYGPQCLCR |
| 467 | SCSPCFTTDHQSARACDDCCGGAGRGRCYGPQCLCR |
| 468 | SCSPCFTTDHQSARRCDDCCGGAGRGRCYGPQCLCR |
| 469 | SCSPCFTTDHQSARKCDDCCGGRGRGRCYGPQCLCR |
| 470 | SCSPCFTTDHQSARACDDCCGGRGRGRCYGPQCLCR |
| 471 | SCSPCFTTDHQSARRCDDCCGGRGRGRCYGPQCLCR |
| 472 | SCSPCFTTDHQSARRCDDCCGGRGRGRCYGPQCLCR |
| 473 | KCSPCFTTDHQSARRCDDCCGGRGRGRCYGPQCLCR |
| 474 | ACAPCFTTDHQAARRCDDCCGGRGRGRCYGPQCLCR |
| 475 | KCAPCFTTDHQAARRCDDCCGGRGRGRCYGPQCLCR |
| 476 | SCSPCFTTDHQSAR(Cit)CDDCCGG(Cit)GRGKCYGPQCLCR |
| 477 | SCSPCFTTDHQSAR(Cit)CDDCCGG(Cit)GRG(Cit)CYGPQCLCR |
| 478 | KCSPCFTTDHQSAR(Cit)CDDCCGG(Cit)GRG(Cit)CYGPQCLCR |
| 479 | ACAPCFTTDHQAAR(Cit)CDDCCGG(Cit)GRGKCYGPQCLCR |
| 480 | ACAPCFTTDHQAAR(Cit)CDDCCGG(Cit)GRG(Cit)CYGPQCLCR |
| 481 | KCAPCFTTDHQAAR(Cit)CDDCCGG(Cit)GRG(Cit)CYGPQCLCR |

Chlorotoxin conjugates used in this disclosure comprise a chlorotoxin and a labeling agent or detectable label. In an embodiment, chlorotoxin is a variant comprising at least 60%, 65%, 70%, 75%, 80%, 83%, 85%, 86%, 89%, 90%, 92%, 93%, 94%, 95%, 96%, 97%, 98%, or 99% identical to the sequence of the natural peptide of chlorotoxin or a fragment thereof.

In another embodiment, the compound comprises a polypeptide having at least at least 60%, 65%, 70%, 75%, 80%, 83%, 85%, 86%, 89%, 90%, 92%, 93%, 94%, 95%, 96%, 97%, 98%, or 99% sequence identity with any one of SEQ ID NO: 1-SEQ ID NO: 481, or any fragment thereof.

In another embodiment, the present disclosure provides a chlorotoxin having the following amino acid sequence: MCMPCFTTDHQMARKCDDCCGGKGRGKCYGPQCLCR (SEQ ID NO: 1) or a fragment thereof. In a further embodiment, the present disclosure provides chlorotoxin variants comprising at least 60%, 65%, 70%, 75%, 80%, 83%, 85%, 86%, 89%, 90%, 92%, 93%, 94%, 95%, 96%, 97%, 98%, or 99% identical to the following amino acid sequence: MCMPCFTTDHQMARKCDDCCGGKGRGKCYGPQCLCR (SEQ ID NO: 1) or a fragment thereof.

In another embodiment, the present disclosure provides a chlorotoxin having the following amino acid sequence: MCMPCFTTDHQMARRCDDCCGGRGRGKCYGPQCLCR (SEQ ID NO: 9) or a fragment thereof. In a further embodiment, the present disclosure provides chlorotoxin variants comprising at least 60%, 65%, 70%, 75%, 80%, 83%, 85%, 86%, 89%, 90%, 92%, 93%, 94%, 95%, 96%, 97%, 98%, or 99% identical to the following amino acid sequence: MCMPCFTTDHQMARRCDDCCGGRGRGKCYGPQCLCR (SEQ ID NO: 9) or a fragment thereof.

In a further embodiment, the present disclosure provides chlorotoxin variants comprising at least 80%, identical to the following amino acid sequence: MCMPCFTTDHQMARRCDDCCGGRGRGKCYGPQCLCR (SEQ ID NO: 9) or a fragment thereof.

In a further embodiment, the present disclosure provides chlorotoxin variants comprising at least 83% identical to the following amino acid sequence: MCMPCFTTDHQMARRCDDCCGGRGRGKCYGPQCLCR (SEQ ID NO: 9) or a fragment thereof.

In a still further embodiment, the present disclosure provides chlorotoxin variants comprising at least 86% identical to the following amino acid sequence: MCMPCFTTDHQMARRCDDCCGGRGRGKCYGPQCLCR (SEQ ID NO: 9) or a fragment thereof.

In another embodiment, the present disclosure provides chlorotoxin variants comprising at least 88% identical to the following amino acid sequence: MCMPCFTTDHQMARRCDDCCGGRGRGKCYGPQCLCR (SEQ ID NO: 9) or a fragment thereof.

In a further embodiment, the present disclosure provides chlorotoxin variants comprising at least 90% identical to the following amino acid sequence: MCMPCFTTDHQMARRCDDCCGGRGRGKCYGPQCLCR (SEQ ID NO: 9) or a fragment thereof.

In a still further embodiment, the present disclosure provides chlorotoxin variants comprising at least 91% identical to the following amino acid sequence: MCMPCFTTDHQMARRCDDCCGGRGRGKCYGPQCLCR (SEQ ID NO: 9) or a fragment thereof.

In a still further embodiment, the present disclosure provides chlorotoxin variants comprising at least 94% identical to the following amino acid sequence: MCMPCFTTDHQMARRCDDCCGGRGRGKCYGPQCLCR (SEQ ID NO: 9) or a fragment thereof.

In yet another embodiment, the present disclosure provides chlorotoxin variants comprising at least 97% identical to the following amino acid sequence: MCMPCFTTDHQMARRCDDCCGGRGRGKCYGPQCLCR (SEQ ID NO: 9) or a fragment thereof.

In another embodiment, the present disclosure provides a chlorotoxin having the following amino acid sequence: MCMPCFTTDHQMARXCDDCCGGXGRGXCYGPQCLCR (SEQ ID NO: 482) or a fragment thereof, wherein each X can each independently be any amino acid.

In another embodiment, the present disclosure provides a chlorotoxin having the following amino acid sequence: MCMPCFTTDHQMARXCDDCCGGXGRGXCYGPQCLCR (SEQ ID NO: 483) or a fragment thereof, wherein X is selected from K, A and R.

In another embodiment, the cholorotoxin is a chlorotoxin or variant thereof having the following amino acid sequence: MCMPCFTTDHQMARXCDDCCGGXGRGXCYGPQCLCR (SEQ ID NO: 484) or a fragment thereof, wherein each X can independently be R or A.

In another embodiment, the cholorotoxin is a chlorotoxin or variant thereof having the following amino acid sequence: MCMPCFTTDHQMARXCDDCCGGXGRGKCYGPQCLCR (SEQ ID NO: 485) or a fragment thereof, wherein each X can independently be R or A.

In still other instances, the variant nucleic acid molecules of a peptide of any one of SEQ ID NO: 1-SEQ ID NO: 485 can be identified by either a determination of the sequence identity of the encoded peptide amino acid sequence with the amino acid sequence of any one of SEQ ID NO: 1-SEQ ID NO: 481, or by a nucleic acid hybridization assay. Such peptide variants can include nucleic acid molecules (1) that remain hybridized with a nucleic acid molecule having the nucleotide sequence of any one of SEQ ID NO: 1-SEQ ID NO: 481 (or its complement) under stringent washing conditions, in which the wash stringency is equivalent to 0.5×-2×SSC with 0.1% SDS at 55-65° C., and (2) that encode a peptide having at least 70%, at least 80%, at least 90%, at least 95% or greater than 95% sequence identity to the amino acid sequence of any one of SEQ ID NO: 1-SEQ ID NO: 481. Alternatively, peptide variants of any one of SEQ ID NO: 1-SEQ ID NO: 481 can be characterized as nucleic acid molecules (1) that remain hybridized with a nucleic acid molecule having the nucleotide sequence of any one of SEQ ID NO: 1-SEQ ID NO: 481 (or its complement) under highly stringent washing conditions, in which the wash stringency is equivalent to 0.1×-0.2×SSC with 0.1% SDS at 50-65° C., and (2) that encode a peptide having at least 70%, at least 80%, at least 90%, at least 95% or greater than 95% sequence identity to the amino acid sequence of any one of SEQ ID NO: 1-SEQ ID NO: 481.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id.). The sequence identity is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 9) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments determination of structure can typically be accompanied by evaluating activity of modified molecules.

In another embodiment, the chlorotoxin is Compound 76, which is a chlorotoxin variant comprising the sequence of MCMPCFTTDHQMARRCDDCCGGRGRGKCYGPQCLCR (SEQ ID NO: 9), wherein the lysine residue is conjugated to a cyanine fluorescent label. The peptide can be further cross-linked by four disulfide bonds formed among the cysteine residues present in the sequence.

TABLE 4
Exemplary Compounds According to the Present Disclosure.

| No. | Strucure |
|---|---|
| 61 | 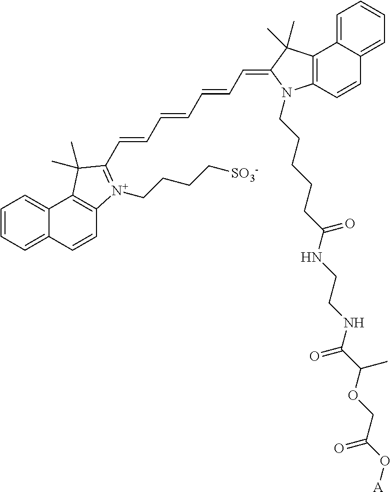 |
| 62 | 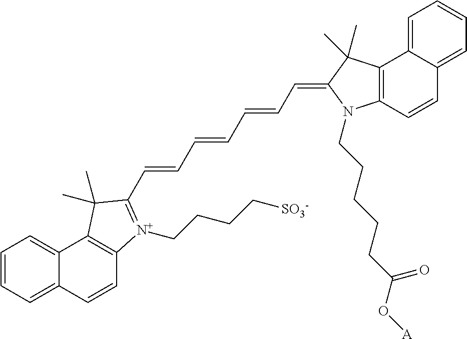 |
| 63 | 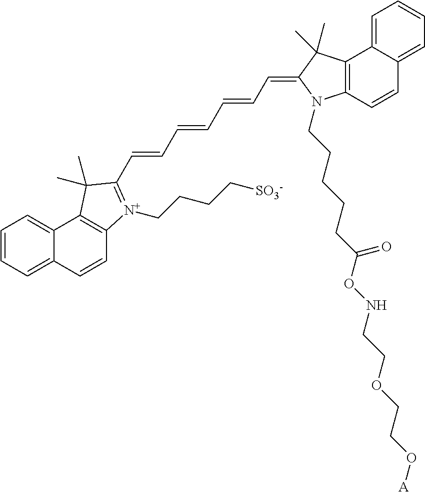 |
| 64 | 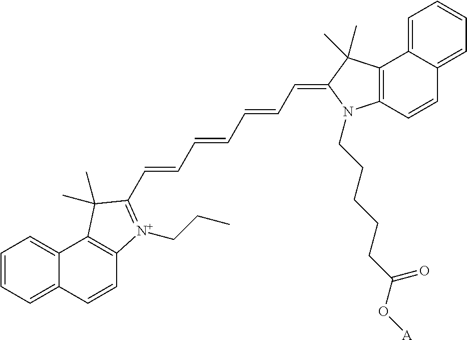 |
| 65 | 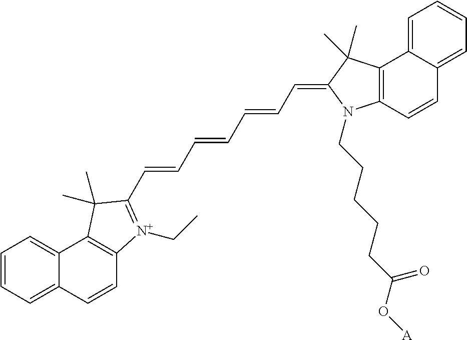 |
| 66 | 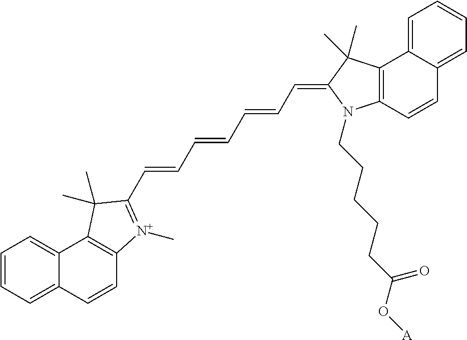 |
| 67 | 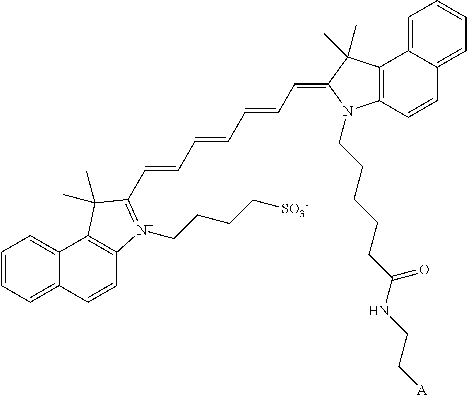 |
| 68 | 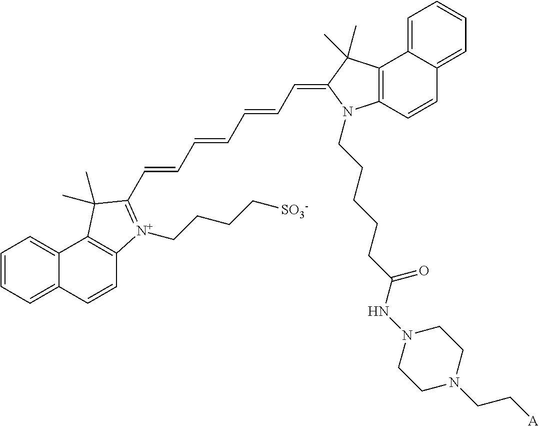 |
| 69 | 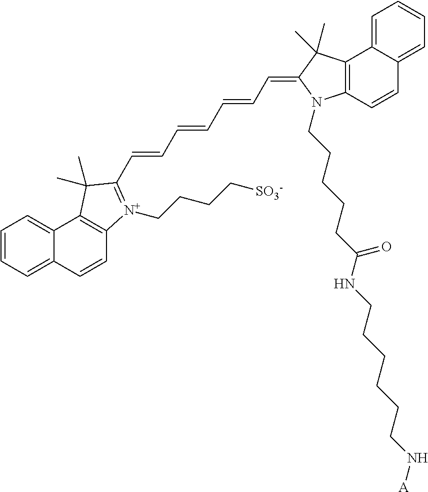 |
| 70 | 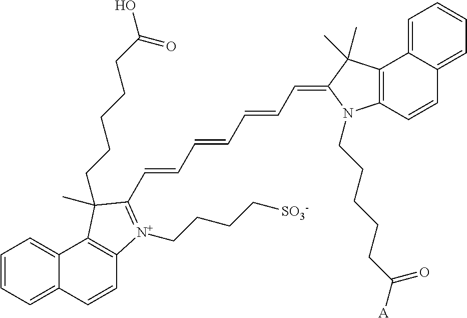 |
| 71 | 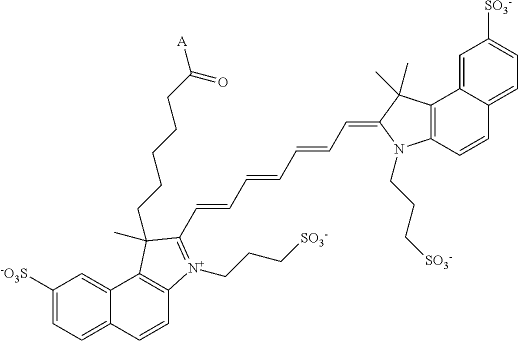 |
| 72 | 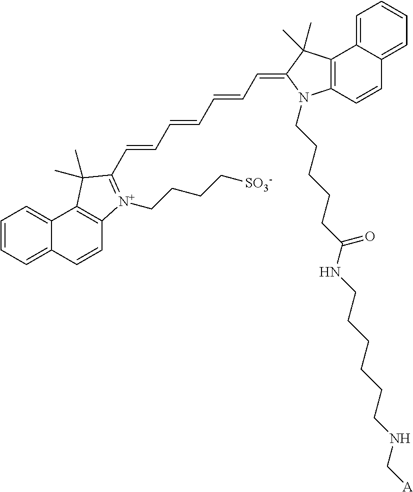 |
| 73 | 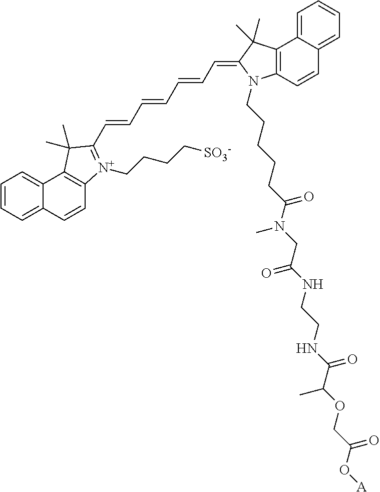 |
| 74 | 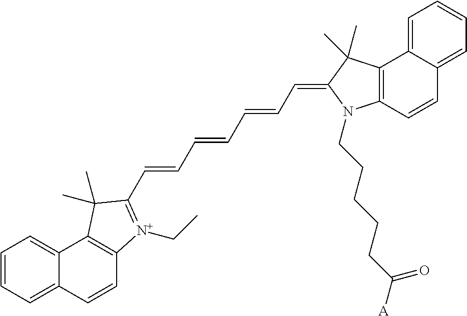 |
| 75 | 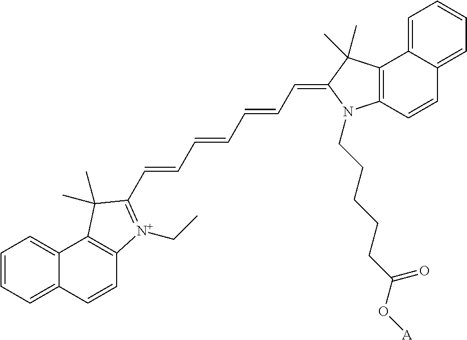 |
| 76 | 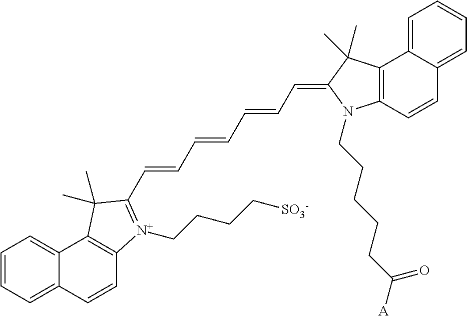 |
| 77 | 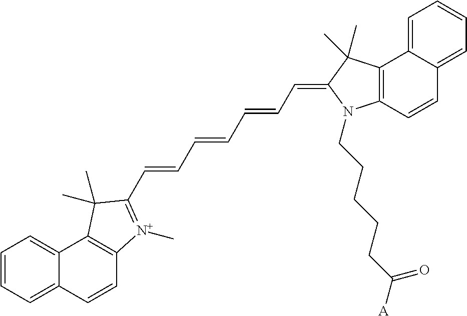 |
| 78 | 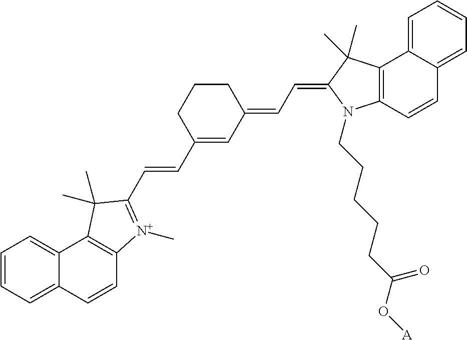 |
| 79 | 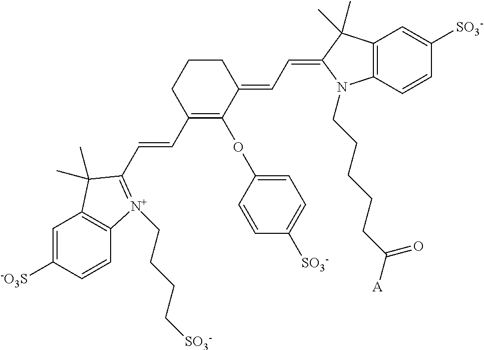 |
| 80 | 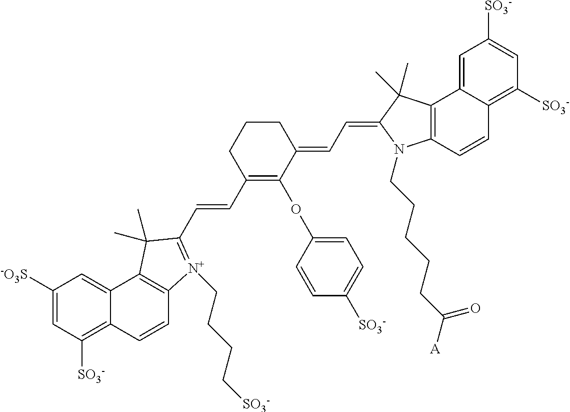 |
| 81 | 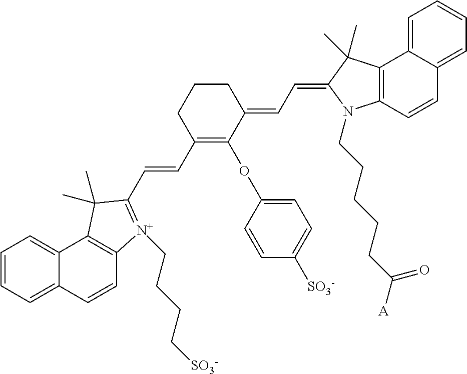 |
| 82 | 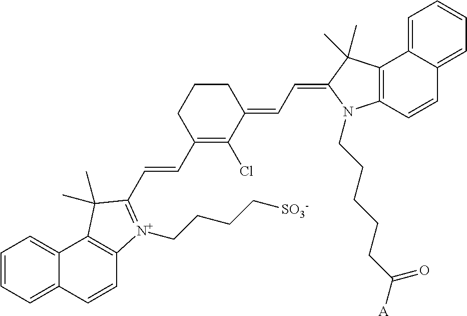 |
| 83 | 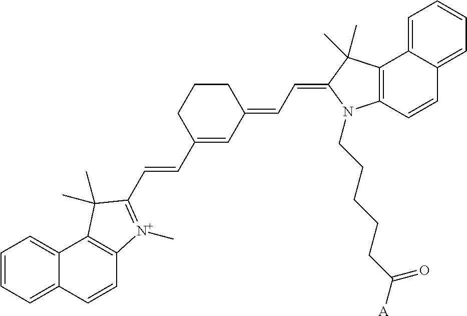 |
| 84 | 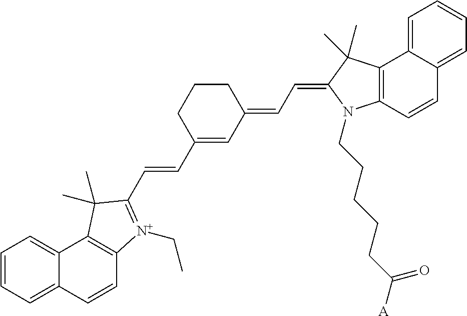 |
| 85 | 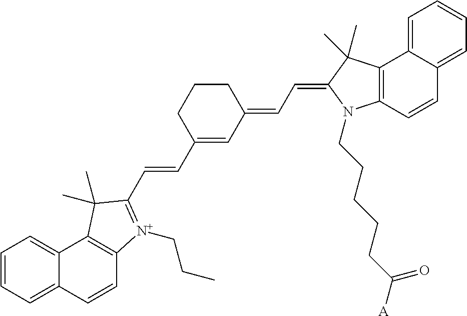 |
| 86 | 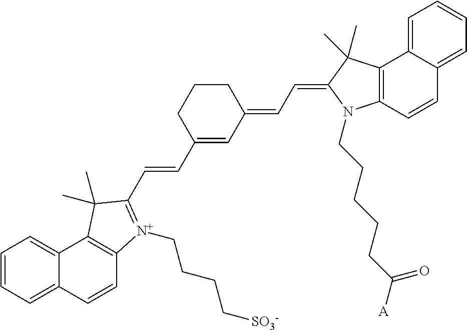 |
| 87 | 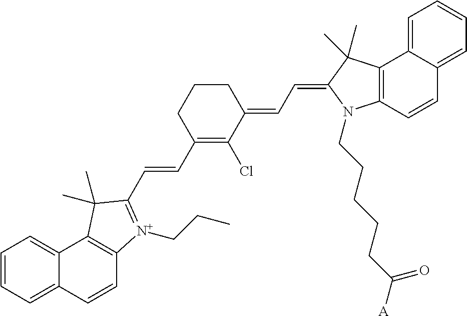 |
| 88 | 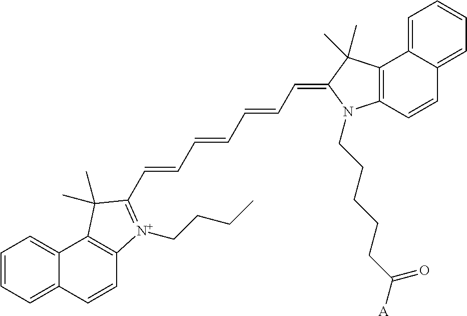 |
| 89 | 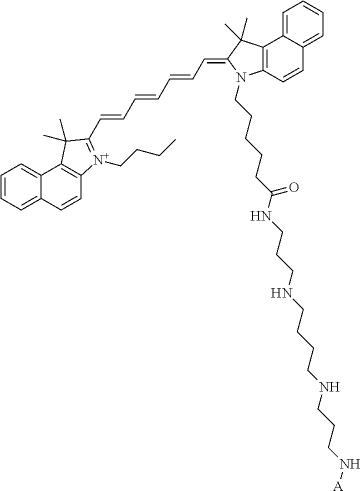 |
| 90 | 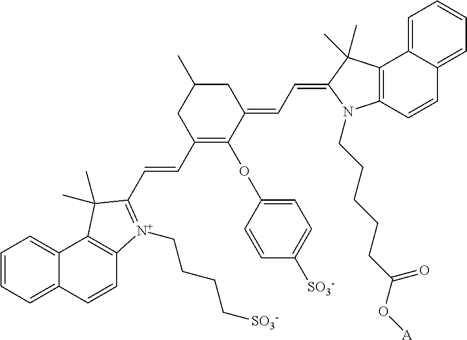 |
| 91 | 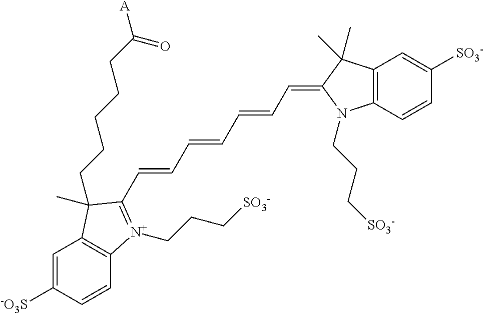 |
| 92 | 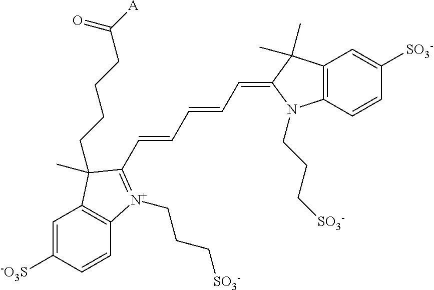 |
| 93 | 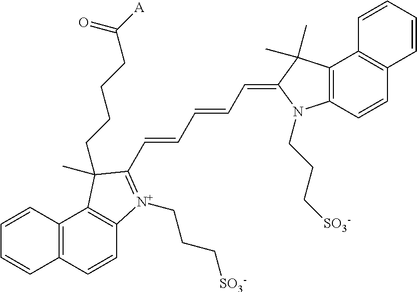 |
| 94 | 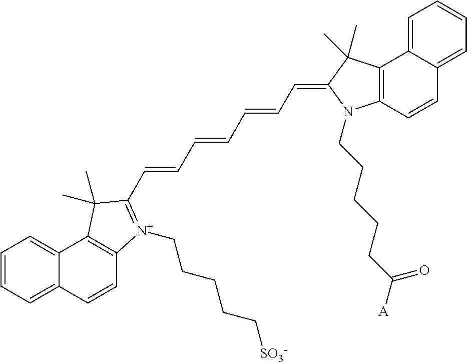 |
| 95 | 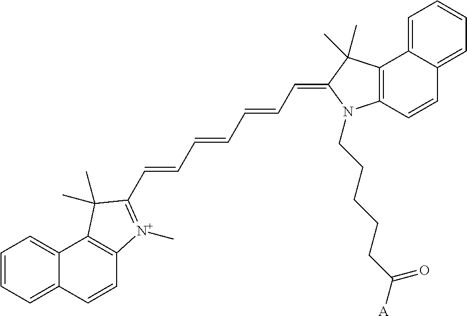 |
| 96 | 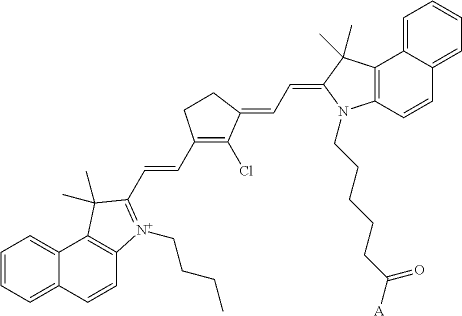 |
| 97 | 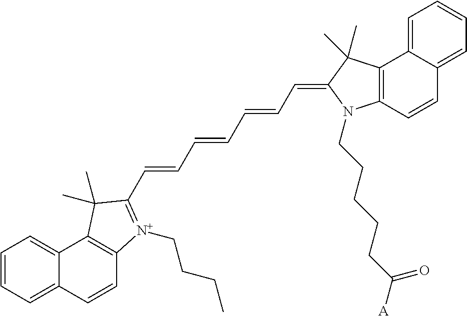 |
| 98 | 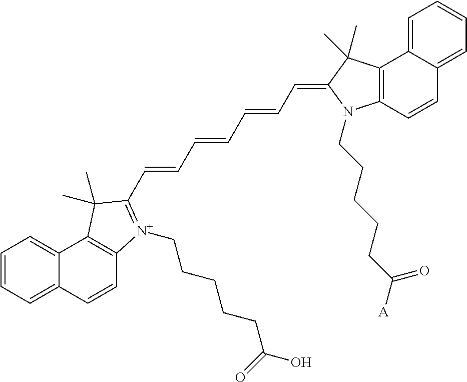 |
| 99 | 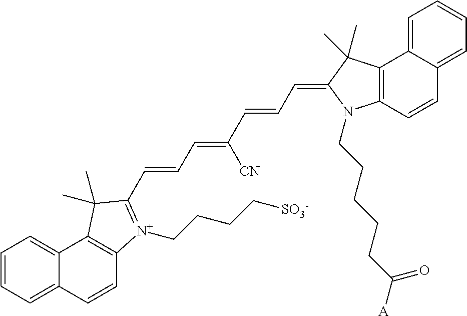 |
| 100 | 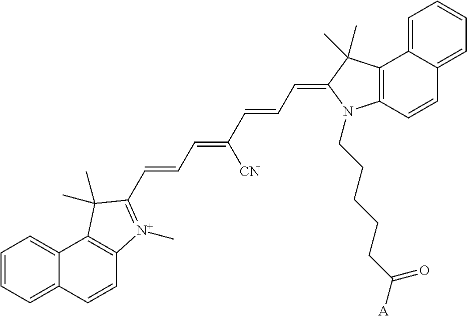 |
| 101 | 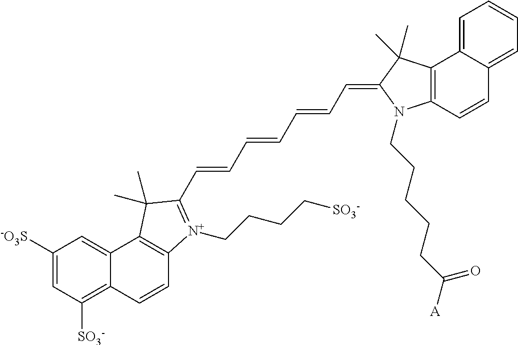 |
| 102 | 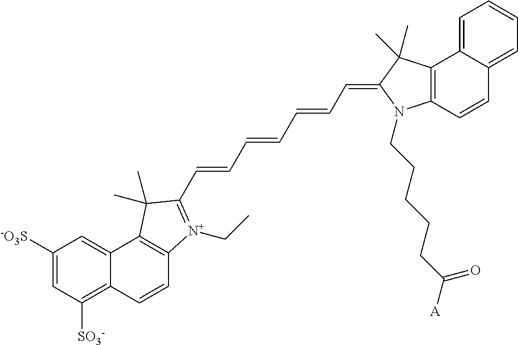 |
| 103 | 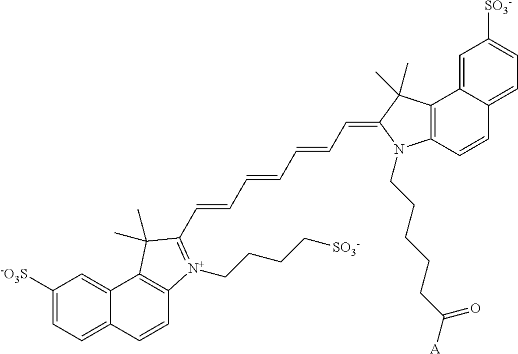 |
| 104 | 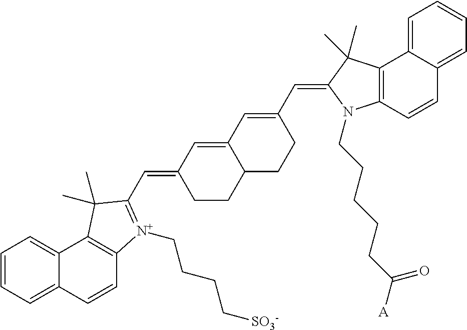 |
| 105 | 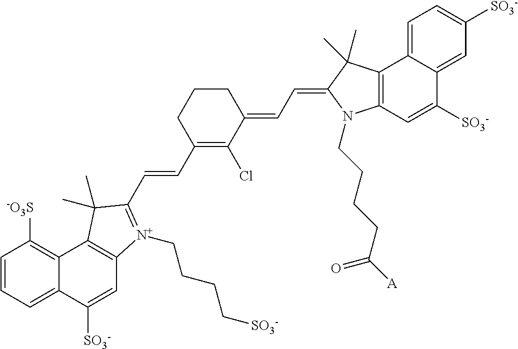 |
| 106 | 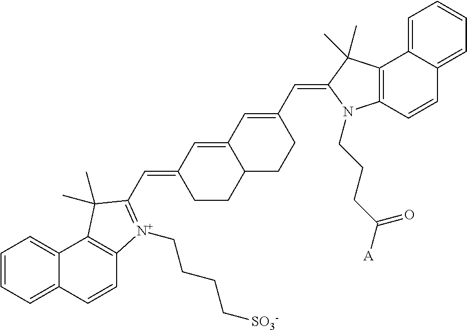 |
| 107 | 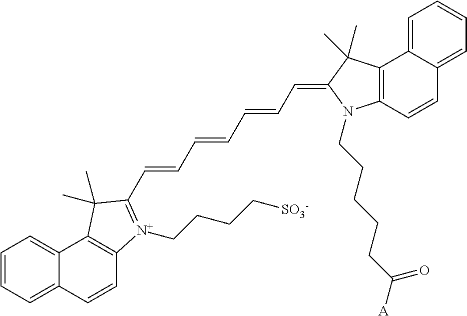 |
| 108 | 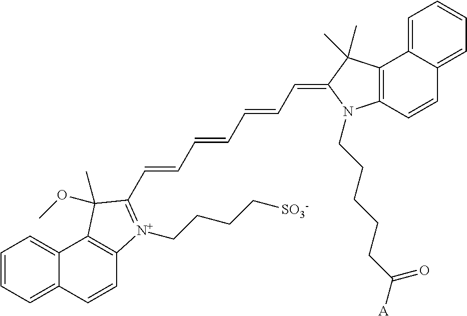 |
| 109 | 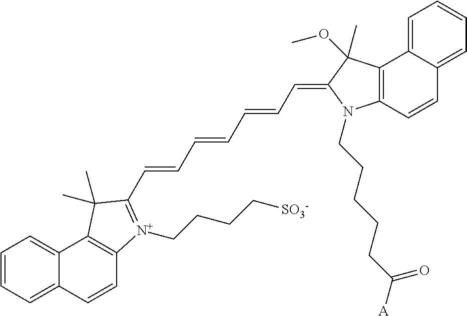 |
| 110 | 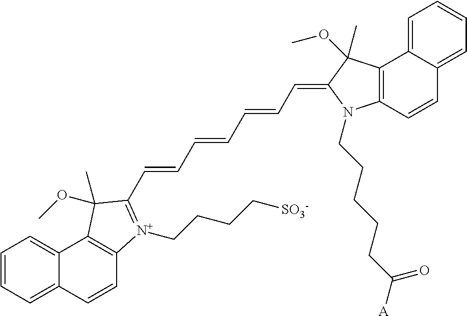 |
| 111 | 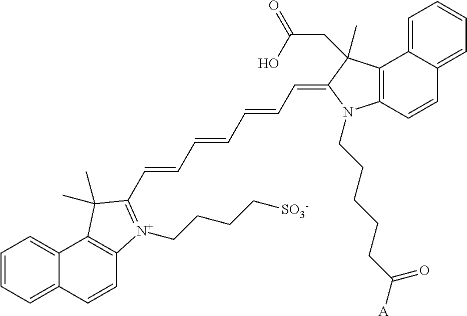 |
| 112 | 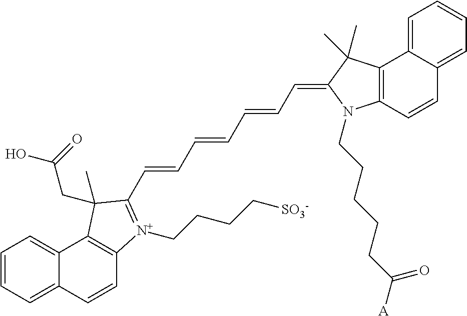 |
| 113 | 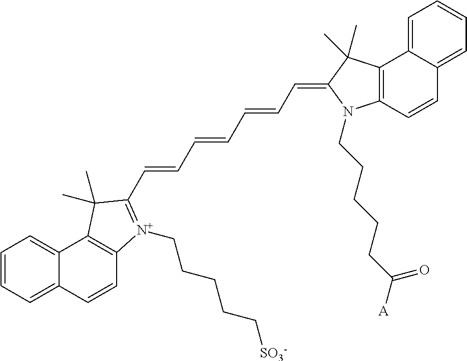 |
| 114 | 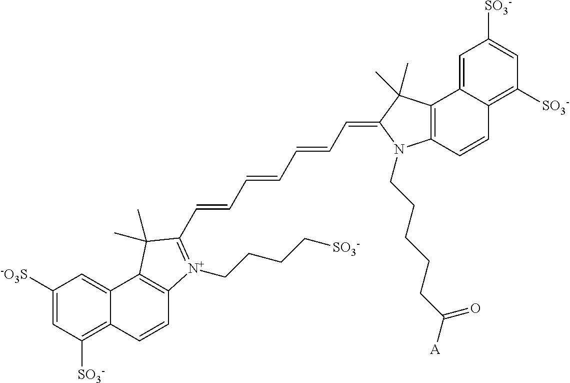 |
| 115 | 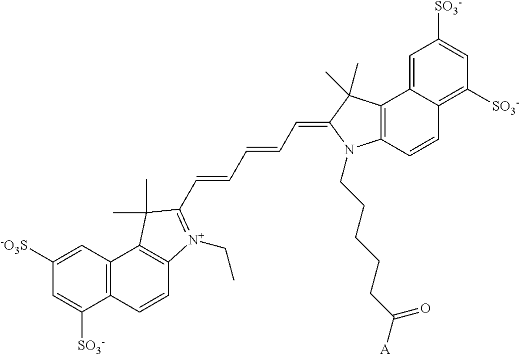 |
| 116 | 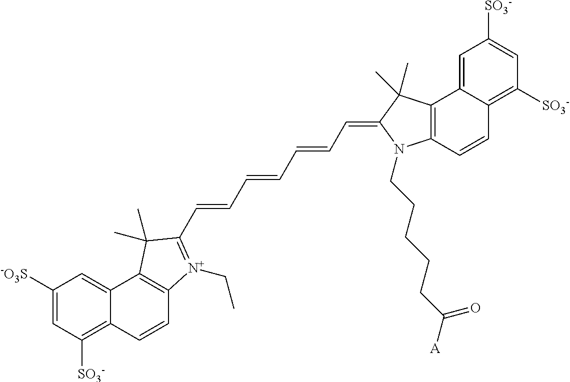 |
| 117 | 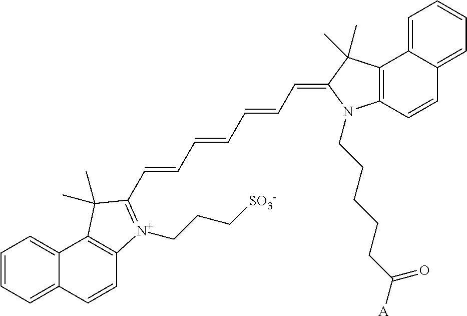 |
| 118 | 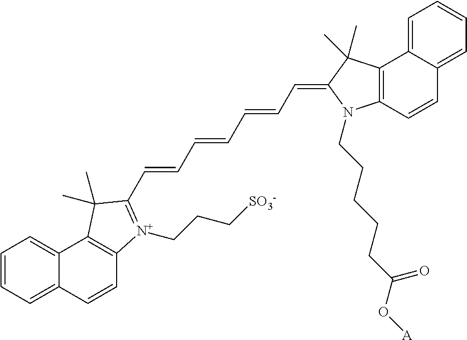 |
| 119 | 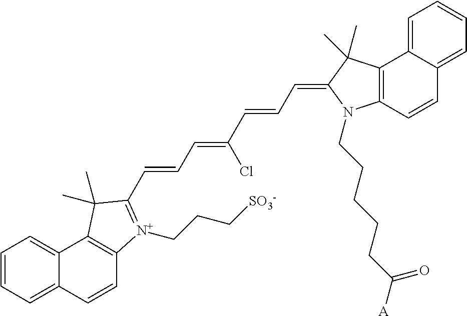 |
| 120 | 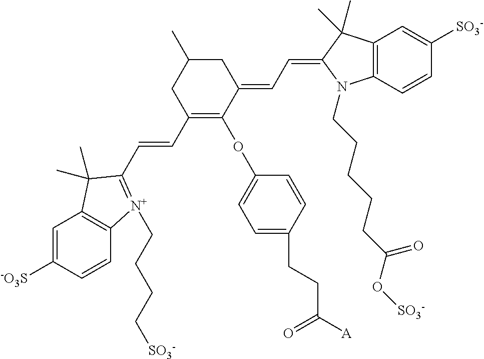 |

A = MCMPCFTTDHQMARRCDDCCGGRGRGKCYGPQCLCR (SEQ ID NO: 9)

In some aspects, the peptide is a variant of the natural peptide of chlorotoxin but retains all eight cysteine residues of the natural peptide, enabling cross-linking by up to four disulfide bonds. Conservation of cysteine residues helps to preserve the secondary structure, charge distribution, isoelectric point (pI) and other features of the natural chlorotoxin peptide because of the disulfide bonds that form between the cysteine residues.

In some aspects, the chlorotoxin peptide variant retains all eight cysteine residues of the natural peptide and has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, the chlorotoxin peptide variant has eight cysteine residues positioned so that the distances between pairs of cysteines is the same as the distances between pairs of cysteines found in the natural peptide, and the chlorotoxin peptide variant has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, the chlorotoxin peptide variant has eight cysteine residues positioned so that the distances between pairs of cysteines is functionally equivalent or functionally similar to the distances between pairs of cysteines found in the natural peptide, and the chlorotoxin peptide variant has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, the chlorotoxin peptide variant has eight cysteine residues positioned so that the distances between pairs of cysteines allows for secondary structure and isoelectric point of the native chlorotoxin peptide to be preserved, and the chlorotoxin peptide variant has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, the chlorotoxin peptide variant has eight cysteine residues positioned so that the distances between pairs of cysteines is sufficient to allow disulfide bonds to form, and the chlorotoxin peptide variant has at least 40%, 45%, 50%, 55%, 60%, 65%, 70%, 75%, 80%, 83%, 85%, 86%, 89%, 90%, 92%, 93%, 94%, 95%, 96%, 97%, 98%, or 99% sequence identity with the native chlorotoxin peptide.

In some aspects, one or more methionines of the chlorotoxin peptide variant are replaced with other amino acids. In some aspects, one or more methionines of the chlorotoxin peptide variant are replaced with other amino acids selected from glycine, alanine, isoleucine, threonine, valine, leucine, serine or a combination thereof.

In some embodiments, the chlorotoxin can be a chlorotoxin variant. Chlorotoxin and chlorotoxin variants have are further described in PCT Patent Application Publication Numbers WO2006115633 and WO2011142858, which are incorporated in their entirety herein by reference.

In one embodiment, the peptide can have the following formula: H-Met-Cys-Met-Pro-Cys-Phe-Thr-Thr-Asp-His-Gln-Met-Ala-Arg-X₁-Cys-Asp-Asp-Cys-Cys-Gly-Gly-X₂-Gly-Arg-Gly-X₃-Cys-Tyr-Gly-Pro-Gln-Cys-Leu-Cys-Arg-OH (SEQ ID NO: 482) acetate salt (disulfide bonds, air oxidized), wherein X₁, X₂, and X₃ can each independently be any amino acid.

In one embodiment, the peptide can have the following formula: H-Met-Cys-Met-Pro-Cys-Phe-Thr-Thr-Asp-His-Gln-Met-Ala-Arg-X₁-Cys-Asp-Asp-Cys-Cys-Gly-Gly-X₂-Gly-Arg-Gly-X₃-Cys-Tyr-Gly-Pro-Gln-Cys-Leu-Cys-Arg-OH (SEQ ID NO: 483) acetate salt (disulfide bonds, air oxidized), wherein X₁, X₂, and X₃ can each independently be Arg, Ala, or Lys.

In another embodiment, the all peptide can have the following formula: H-Met-Cys-Met-Pro-Cys-Phe-Thr-Thr-Asp-His-Gln-Met-Ala-Arg-X₁-Cys-Asp-Asp-Cys-Cys-Gly-Gly-X₂-Gly-Arg-Gly-X₃-Cys-Tyr-Gly-Pro-Gln-Cys-Leu-Cys-Arg-OH (SEQ ID NO: 484) acetate salt (disulfide bonds, air oxidized), wherein X₁, X₂, and X₃ can each independently be Arg or Ala.

In another embodiment, the all peptide can have the following formula: H-Met-Cys-Met-Pro-Cys-Phe-Thr-Thr-Asp-His-Gln-Met-Ala-Arg-X₁-Cys-Asp-Asp-Cys-Cys-Gly-Gly-X₂-Gly-Arg-Gly-Lys-Cys-Tyr-Gly-Pro-Gln-Cys-Leu-Cys-Arg-OH (SEQ ID NO: 485) acetate salt (disulfide bonds, air oxidized), wherein X₁ and X₂ can each independently be Arg or Ala.

In another embodiment, the peptide can have the following formula: H-Met-Cys-Met-Pro-Cys-Phe-Thr-Thr-Asp-His-Gln-Met-Ala-Arg-Arg-Cys-Asp-Asp-Cys-Cys-Gly-Gly-Arg-Gly-Arg-Gly-Lys-Cys-Tyr-Gly-Pro-Gln-Cys-Leu-Cys-Arg-OH (SEQ ID NO: 9) acetate salt (disulfide bonds, air oxidized).

In another embodiment, the peptide can have the following formula: H-Met-Cys-Met-Pro-Cys-Phe-Thr-Thr-Asp-His-Gln-Met-Ala-Arg-Arg-Cys-Asp-Asp-Cys-Cys-Gly-Gly-Ala-Gly-Arg-Gly-Lys-Cys-Tyr-Gly-Pro-Gln-Cys-Leu-Cys-Arg-OH (SEQ ID NO: 6) acetate salt (disulfide bonds, air oxidized).

In another embodiment, the peptide can have the following formula: H-Met-Cys-Met-Pro-Cys-Phe-Thr-Thr-Asp-His-Gln-Met-Ala-Arg-Ala-Cys-Asp-Asp-Cys-Cys-Gly-Gly-Arg-Gly-Arg-Gly-Lys-Cys-Tyr-Gly-Pro-Gln-Cys-Leu-Cys-Arg-OH (SEQ ID NO: 8) acetate salt (disulfide bonds, air oxidized).

In another embodiment, the peptide can have the following formula: H-Met-Cys-Met-Pro-Cys-Phe-Thr-Thr-Asp-His-Gln-Met-Ala-Arg-Ala-Cys-Asp-Asp-Cys-Cys-Gly-Gly-Ala-Gly-Arg-Gly-Lys-Cys-Tyr-Gly-Pro-Gln-Cys-Leu-Cys-Arg-OH (SEQ ID NO: 5) acetate salt (disulfide bonds, air oxidized).

### Linkers

In some aspects, the peptides of the present disclosure are directly conjugated to a detectable label, such as a dye, fluorescent moiety or the like such that no additional amino acids, carbohydrates, nucleic acids, polymers, organic chains, or the like are added to the chlorotoxin or chlorotoxin variant and/or the dye, fluorescent moiety or the like to comprise the chlorotoxin conjugates described herein. In some other aspects, a linker is used to conjugate the chlorotoxin or chlorotoxin variant is not directly conjugated to a dye, fluorescent moiety or the like such that additional amino acids, carbohydrates, nucleic acids or the like are added to the chlorotoxin or chlorotoxin variant and/or the dye, fluorescent moiety or the like to comprise the chlorotoxin conjugates described herein. A “linker” as used herein refers to at least one compound comprising two functional groups that are capable of reacting specifically with other moieties to form covalent or non-covalent linkages. Such moieties include, but are not limited to, the side groups on natural or non-natural amino acids or peptides which contain such natural or non-natural amino acids. By way of example, a linker has a functional group reactive with a group on a first peptide, and another functional group which is reactive with a group on a second peptide, whereby forming a conjugate that includes the first peptide, the linker and the second peptide. Many procedures and linker molecules for attachment of various compounds to peptides are known. See, e.g., European Patent Application No. 188,256; U.S. Pat. Nos. 4,671,958; 4,659,839; 4,414,148; 4,699,784; 4,680,338; and 4,569,789, which are incorporated by reference herein in their entirety.

The term “linkage,” as used herein refers to a bond or a chemical moiety formed from a chemical reaction between the functional group of a linker and another molecule. Such bonds include, but are not limited to, covalent linkages and non-covalent bonds, while such chemical moieties include, but are not limited to, esters, carbonates, imines phosphate esters, hydrazones, acetals, orthoesters, peptide linkages, and oligonucleotide linkages. Hydrolytically stable linkages means that the linkages are substantially stable in water and do not react with water at neutral pH values, including but not limited to, under physiological conditions for an extended period of time, perhaps even indefinitely. Hydrolytically unstable or degradable linkages mean that the linkages are degradable in water or in aqueous solutions, including for example, blood. Enzymatically unstable or degradable linkages mean that the linkage is often degraded by one or more enzymes. By way of example, PEG and related polymers include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

The conjugates for use in the method described herein can be conjugated by using any art-recognized method forming a complex including covalent, ionic, or hydrogen bonding of the ligand to the imaging agent, either directly or indirectly via a linking group such as a linker. The conjugate can be typically formed by covalent bonding of the ligand to the imaging agent through the formation of amide, ester or imino bonds between acid, aldehyde, hydroxy, amino, or hydrazo groups on the respective components of the complex or, for example, by the formation of disulfide bonds.

In addition, structural modifications of a linker portion of the conjugates are contemplated herein. For example, a number of amino acid substitutions are often made to the linker portion of the conjugate, including but not limited to naturally occurring amino acids, as well as those available from conventional synthetic methods. In one aspect, beta, gamma, and longer chain amino acids are used in place of one or more alpha amino acids. In another aspect, the stereochemistry of the chiral centers found in such molecules is selected to form various mixture of optical purity of the entire molecule, or only of a subset of the chiral centers present. In another aspect, the length of the peptide chain included in the linker is shortened or lengthened, either by changing the number of amino acids included therein, or by including more or fewer beta, gamma, or longer chain amino acids. In another aspect, the selection of amino acid side chains in the peptide portion is made to increase or decrease the relative hydrophilicity of the linker portion specifically or of the overall molecule generally.

Similarly, the length and shape of other chemical fragments of the linkers described herein is often modified. In some aspects, the linker includes an alkylene chain. The alkylene chain often varies in length, or includes branched groups, or includes a cyclic portion, which are in line or spiro relative to the allylene chain. In another aspect, where the linker includes a beta thiol releasable fragment, it is appreciated that other intervening groups connecting the thiol end to the hydroxy or carbonate end are used in place of the ethylene bridge, such as but not limited to optionally substituted benzyl groups, where the hydroxy end is connected at the benzyl carbon and the thiol end is connected through the ortho or para phenyl position, and vice versa

Direct attachment can occur via covalent attachment of a peptide to another molecule. For example, the peptide is attached to a terminus of the amino acid sequence of a larger polypeptide or peptide molecule, or could be attached to a side chain, such as the side chain of a lysine, serine, threonine, cysteine, tyrosine, aspartic acid, a non-natural amino acid residue, or glutamic acid residue. The attachment can be via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond, or linker as described herein) may be used to link other molecules.

Attachment via a linker can involve incorporation of a linker moiety between the other molecule and the peptide. The peptide and the other molecule can both be covalently attached to the linker. The linker can be cleavable, non-cleavable, self-immolating, hydrophilic, or hydrophobic. The linker can have at least two functional groups, one bonded to the other molecule, one bonded to the peptide, and a linking portion between the two functional groups. The use of a cleavable linker can permit release of the conjugated moiety (e.g., a detectable agent or a therapeutic agent) from the peptide, e.g., after targeting to a tissue of interest. The cleavable linker can comprise a cleavage site for matrix metalloproteinases, thrombin, cathepsins, or beta-glucuronidase. In other aspects, the linker can be a hydrolytically labile linker. A hydrolytically labile linker, (amongst other cleavable linkers described herein) can be advantageous in terms of releasing a fluorophore molecule or other detectable or therapeutic agents from the peptide. For example, an agent (e.g., a detectable agent or a therapeutic agent) in a conjugate form with the peptide may not be active, but upon release from the conjugate after targeting to the cartilage, the agent can be active. In some cases the linker can be enzyme cleavable, e.g., a valine-citrulline linker. Alternatively or in combination, the linker can be cleavable by other mechanisms, such as via pH, reduction, or hydrolysis. Other cleavable linkers can include an ester bond using standard 1-ethyl-3-(3-dimethylaminopropyl)carbodiimide (EDC)-, dicylcohexylcarbodiimide (DCC)-, thionyl chloride-, or phosphorous chloride-based bioconjugation chemistries. These linkers can be cleaved by esterases, MMP, cathepsin B, a protease, or thrombin. In still other aspects, the peptide can be linked to the detectable agent via a noncleavable linker.

Non-limiting examples of the functional groups for attachment can include functional groups capable of forming, for example, an amide bond, an ester bond, an ether bond, a carbonate bond, a carbamate bond, or a thioether bond. Non-limiting examples of functional groups capable of forming such bonds include amino groups; carboxyl groups; hydroxyl groups; aldehyde groups; azide groups; alkyne and alkene groups; ketones; hydrazides; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion can include alkylene, alkenylene, alkynylene, polyether, such as polyethylene glycol (PEG), hydroxy carboxylic acids, polyester, polyamide, polyamino acids, polypeptides, cleavable peptides, valine-citrulline, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, and ester groups.

Non-limiting examples of linkers can include:

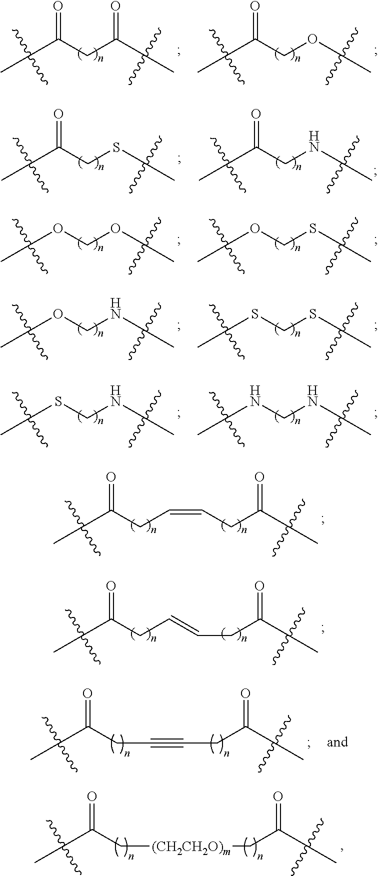

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50.

### Formulations of Chlorotoxin Conjugates

In various aspects, the present disclosure provides compositions comprising the above-described compounds and a pharmaceutically acceptable carrier. In some aspects, the composition is formulated for parenteral administration. In further aspects, the composition is formulated for intravenous administration, intramuscular administration, subcutaneous administration, or a combination thereof.

Certain methods described herein can comprise administering to the subject an intravenous pharmaceutical composition comprising a chlorotoxin conjugate, for example, as described herein. Intravenous pharmaceutical compositions of chlorotoxin conjugates can include any formulation suitable for administration to a subject via any intravenous method, including a bolus, a slow-bolus, an infusion which occurs over time, or any other intravenous method known in the art, as discussed further herein. “Product” or “dosage form” as used herein refers to any solid, semi-solid, lyophilized, aqueous, liquid or frozen formulation or preparation used for administration. Upon administration, the rate of release of an active moiety from a product can often be greatly influenced by the excipients and/or product characteristics which make up the product itself. For example, an enteric coat on a tablet is designed to separate that tablet's contents from the stomach contents to prevent, for example, degradation of the stomach which often induces gastrointestinal discomfort or injury. According to the currently accepted conventional understanding, systemic exposure of the active moiety will be relatively insensitive to the small formulation changes.

As used herein “pharmaceutically acceptable” or “pharmacologically acceptable” includes molecular entities and compositions that do not produce an adverse, allergic or other untoward reaction when administered to a subject, as appropriate. “Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients are often also incorporated into the compositions.

In various aspects, the present compositions comprise a concentration of the compound as an active pharmaceutical ingredient having a concentration from 0.1 mg/mL to 100 mg/mL. In some aspects, the concentration of the compound is from 0.1 mg/mL to 5 mg/mL, from 0.1 mg/mL to 10 mg/mL, from 0.1 mg/mL to 15 mg/mL, from 0.1 mg/mL to 20 mg/mL, from 0.1 mg/mL to 30 mg/mL, from 0.1 mg/mL to 40 mg/mL, from 0.1 mg/mL to 50 mg/mL, from 0.1 mg/mL to 60 mg/mL, from 0.1 mg/mL to 70 mg/mL, from 0.1 mg/mL to 80 mg/mL, or from 0.1 mg/mL to 90 mg/mL. In further aspects, the concentration of the compound is from 1 mg/mL to 20 mg/mL. In still other aspects, the concentration of the compound is from 4 mg/mL to 10 mg/mL. In additional aspects, the concentration of the compound is from 5 mg/mL to 8 mg/mL. In yet further aspects, the concentration of the compound is from 5 mg/mL to 6 mg/mL. In other aspects, the concentration of the compound is from 15 mg/mL to 35 mg/mL. In still other aspects, the concentration of the compound is from 15 mg/mL to 25 mg/mL. In yet other aspects, the concentration of the compound is from 15 mg/mL to 50 mg/mL, from 15 mg/mL to 60 mg/mL, 15 mg/mL to 70 mg/mL, 15 mg/mL to 80 mg/mL, or 15 mg/mL to 90 mg/mL.

In some embodiments, the pharmaceutically acceptable carrier has a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In still other embodiments, the pharmaceutically acceptable carrier has a pH within a range from about 6.0 to about 7.5. In other embodiments, the pharmaceutically acceptable carrier has a pH within a range from about 5.0 to about 9.0.

In some embodiments, the composition has a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In still other embodiments, the composition has a pH within a range from about 6.0 to about 7.5. In other embodiments, the composition has a pH within a range from about 5.0 to about 9.0.

In some aspects, a pharmaceutically acceptable carrier comprises tris, D-mannitol, L-histidine, L-methionine, polysorbate 20, or a combination thereof. For example, in some aspects, a pharmaceutically acceptable carrier comprises tris and D-mannitol. In some aspects, a pharmaceutically acceptable carrier comprises L-histidine and D-mannitol. In some aspects, the pharmaceutically acceptable carrier comprises L-histidine and D-mannitol with polysorbate 20. In some aspects, the pharmaceutically acceptable carrier comprises L-histidine, D-mannitol, and L-methionine.

In some aspects, the pharmaceutically acceptable carrier comprises L-histidine, D-mannitol, polysorbate 20, and a pH of about 6.8. In some aspects, the pharmaceutically acceptable carrier comprises L-histidine, D-mannitol, polysorbate 20, and a pH within a range of about 6 to about 7.5. In some aspects, the pharmaceutically acceptable carrier comprises L-histidine, D-mannitol, polysorbate 20, and a pH within a range of about 5 to about 9. In some aspects, the pharmaceutically acceptable carrier comprises L-histidine, D-mannitol, and a pH of about 6.8. In some aspects, the pharmaceutically acceptable carrier comprises L-histidine, D-mannitol, and a pH within a range of about 6 to about 7.5. In some aspects, the pharmaceutically acceptable carrier comprises L-histidine, D-mannitol, and a pH within a range of about 5 to about 9. In some aspects, the pharmaceutically acceptable carrier comprises L-histidine, D-mannitol, polysorbate 20, trehalose, and a pH of about 6.8. In some aspects, the pharmaceutically acceptable carrier comprises L-histidine, D-mannitol, polysorbate 20, trehalose, and a pH within a range of about 6 to about 7.5. In some aspects, the pharmaceutically acceptable carrier comprises L-histidine, D-mannitol, polysorbate 20, trehalose, and a pH within a range of about 5 to about 9.

A pharmaceutical composition comprising a chlorotoxin conjugate can be formulated according to known methods to prepare pharmaceutically useful compositions, for example, as found in “Excipient Selection in Parenteral Formulation Development” Pramanick et al., Pharma Times, Vol. 45., No. 3, March 2013, incorporated in its entirety herein by reference. In some aspects, the chlorotoxin conjugate is combined with a pharmaceutically acceptable carrier. A composition is said to be a pharmaceutically acceptable carrier if its administration is tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

Formulations for administration of chlorotoxin conjugates can typically be provided but are not limited to as liquid, solid or semi-solid products or dosage forms, exemplified by tablets, capsules, pellets, a powder or a lyophilized product. In some aspects, the chlorotoxin conjugate is formulated to comprise no additional materials except for a pharmaceutical carrier. In some other aspects, the chlorotoxin conjugate is formulated such that it comprises a core “matrix material” which encapsulates, binds to, coats or is adjacent to the chlorotoxin conjugate. In some other aspects, the chlorotoxin conjugate and matrix material further comprises a protective coatings. Various formulations are well-known to those in the art. See, for example, Gennaro (ed.), Remington's Pharmaceutical Sciences, 19th Edition (Mack Publishing Company 1995).

Suitable excipients for use with chlorotoxin conjugates can often be included in formulations for intravenous use, for example, an injection. Injections are sterile, pyrogen-free solutions or dispersions (emulsions or suspensions) of one or more active ingredients in a suitable vehicle or carrier. Injections that are dispersions can remain sufficiently stable so that, after shaking, a homogeneous dose is withdrawn. More specifically, formulations which can include chlorotoxin conjugates and one or more, but not limited to suitable excipients, exemplified by matrix materials, binders, lubricants, glidants or disintegrants which aid in modulating the PK profile of administered chlorotoxin conjugates are preferred. In some aspects, compositions comprise chlorotoxin conjugates in combination with one or more suitable excipients and one or more specific product characteristics (such as dissolution or water content) which result in improved pharmacokinetic profiles of chlorotoxin conjugates in vivo. Thus, the in vivo performance of chlorotoxin conjugates dosage forms/products included herein can be based upon the composition of the excipients added during manufacturing and/or the final product characteristics generated through specific processing parameters and methods. Other excipients are well-known to those in the art. See, for example, Gennaro (ed.), Remington's Pharmaceutical Sciences, 19th Edition (Mack Publishing Company 1995).

Suitable carriers for intravenous administration can include, for example, but are not limited to, physiological saline or phosphate buffered saline (PBS), Tris, and solutions containing solubilizing agents, such as glucose, polyethylene glycol, polypropylene glycol, additional agents such as histidine, dextrose, mannitol and mixtures thereof. In some aspects, carriers for intravenous administration include a mixture of histidine and dextrose, Tris and dextrose or Tris and mannitol. Other carriers are well-known to those in the art. See, for example, Gennaro (ed.), Remington's Pharmaceutical Sciences, 19th Edition (Mack Publishing Company 1995).

The formulation often can include an aqueous vehicle. Aqueous vehicles can include, by way of example and without limitation, sodium chloride solution, Ringers solution, isotonic dextrose solution, sterile water solution, dextrose and lactated Ringers solution. Nonaqueous vehicles can include, by way of example and without limitation, fixed oils of vegetable origin, cottonseed oil, corn oil, sesame oil and peanut oil, benzyl benzoate, castor oil, N,N-dimethylacetamide, ethanol, dehydrated ethanol, glycerin, glycerol, N-methyl-2-pyrrolidone, polyethylene glycol and any derivative thereof, propylene glycol, safflower oil and soybean oil. Other vehicles are well-known to those in the art. See, for example, Gennaro (ed.), Remington's Pharmaceutical Sciences, 19th Edition (Mack Publishing Company 1995).

In some aspects, the composition the pharmaceutically acceptable carrier comprises an osmolyte. In some aspects, the osmolyte comprises a sugar, a sugar alcohol, or a combination thereof.

In certain aspects, the composition comprises a sugar alcohol. In certain aspects, the composition comprises a sugar alcohol selected from sorbitol, inositol, mannitol, xylitol, glycerol, or a combination thereof. In further aspects, the sugar alcohol comprises mannitol. In certain aspects, the composition comprises from about 2% to about 20% (wt/vol %) sugar alcohol. In some aspects, the composition comprises from about 2% to about 10% (wt/vol %) sugar alcohol. In some aspects, the composition comprises from about 3% to about 10% (wt/vol %) sugar alcohol. In further aspects, the composition comprises about 5% (wt/vol %) sugar alcohol. In certain aspects, the composition comprises from about 2% to about 20% (wt/vol %) mannitol. In some aspects, the composition comprises from about 2% to about 10% (wt/vol %) mannitol. In further aspects, the composition comprises about 5% (wt/vol %) mannitol.

In other aspects, the composition comprises a sugar. In certain aspects, the sugar is selected from trehalose, lactose, sucrose, glucose, galactose, maltose, mannose, fructose, dextrose, or a combination thereof. In additional aspects, the sugar is selected from trehalose, sucrose, or a combination thereof. In some aspects, the composition comprises from about 1% to about 40% (wt/vol %) of sugar. In other aspects, the composition comprises from about 1% to about 20% (wt/vol %) of sugar. In additional aspects, the composition comprises about 2% (wt/vol %) of sugar. In some aspects, the composition comprises from about 1% to about 40% (wt/vol %) of trehalose, sucrose, or a combination of trehalose and sucrose. In other aspects, the composition comprises from about 1% to about 20% (wt/vol %) of trehalose, sucrose, or a combination of trehalose and sucrose. In additional aspects, the composition comprises about 2% (wt/vol %) of trehalose, sucrose, or a combination of trehalose and sucrose.

In certain aspects, the composition further comprises an osmolyte selected from glycine, carnitine, ethanolamine, their phosphates, mono sugars, or a combination thereof.

In some aspects, the present compositions are isotonic. In other aspects, the compositions are about isotonic.

In certain aspects, the ionic strength of the composition is less than or equal to 60 mM. In certain aspects, the composition comprises an ionic strength less than or equal to 50 mM. In certain aspects, the ionic strength of the composition is less than or equal to 40 mM. In certain aspects, the ionic strength of the composition is less than or equal to 30 mM. In certain aspects, the ionic strength of the composition is less than or equal to 20 mM. In other aspects, the ionic strength of the composition is less than or equal to 10 mM.

Antimicrobial agents in bacteriostatic or fungistatic concentrations can be typically added to preparations packaged in multiple dose containers which can include by way of example and without limitation, phenols or cresols, mercurials, benzyl alcohol, chlorobutanol, methyl and propyl p-hydroxybenzoic acid esters, thimerosal, benzalkonium chloride and benzethonium chloride. Other antimicrobial agents are well-known to those in the art. See, for example, Gennaro (ed.), Remington's Pharmaceutical Sciences, 19th Edition (Mack Publishing Company 1995).

Buffers can include by way of example and without limitation, acetate, ammonium sulfate, ammonium hydroxide, arginine, aspartic acid, benzene sulfonic acid, benzoate sodium, benzoate acid, carbonate, sodium carbonate, carbon dioxide, citrate, diethanolamine, glucono delta lactone, glycine, glycine HCl, histidine, histidine HCl, hydrochloric acid, hydrobromic acid, lysine maleic acid, meglumine, methanesulfonic acid, monoethanolamine, phosphate, sodium phosphate, citrate, succinate sodium, sulfuric acid, tartarate sodium, trimethamine, sodium citrate, hydroxide, sodium hydroxide, Tris base, Tris base −65, Tris acetate, Tris HCl, and Tris HCl-65.

In various aspects, the pharmaceutically acceptable carrier comprises a buffer. In some aspects, the buffer is selected from tris, HEPES, histidine, ethylene diamine, or a combination thereof. In other aspects, the buffer is selected from tris, histidine, or a combination thereof. In further aspects, the buffer comprises histidine, which is optionally L-histidine. In another aspect, the composition comprises a buffer comprising histidine, tris, HEPES, ethylene diamine, or a combination thereof. In additional aspects, the composition comprises at least 100 mM histidine. In further aspects, the composition comprises at least or equal to 50 mM histidine. In some aspects, the composition comprises at least or equal to 20 mM histidine. In additional aspects, the composition comprises 10 to 100 mM histidine. In other aspects, the composition comprises 10 to 20 mM histidine. In other aspects, the composition comprises 0 to 50 mM histidine. In further aspects, the composition comprises at least 100 mM tris. In some aspects, the composition comprises at least or equal to 50 mM tris. In additional aspects, the composition comprises at least or equal to 20 mM tris. In other aspects, the composition comprises 10 to 20 mM tris. In other aspects, the composition comprises 0 to 20 mM tris. In some aspects, the composition comprises from about 0 mM to about 50 mM histidine, from about 0 mM to about 20 mM tris, about 20 mM methionine, from about 3% to about 10% (wt/vol %) sugar alcohol, and a pH within a range from about 6 to about 7.5.

Antioxidants can include by way of example and without limitation, sodium bisulfate, acetone sodium bisulfate, argon, ascorbyl palmitate, ascorbate sodium, ascorbate acid, butylated hydroxy anisole, butylated hydroxy toluene, cysteine, cystenate HCl, dithionite sodium, gentistic acid, gentistic acid ethanoloamine, glutamate monosodium, glutathione, formaldehyde solfoxylate sodium, metabisulfite potassium, metabisulfite sodium, methionine, monothioglycerol, nitrogen, propyl gallate, sulfite sodium, tocopherol alpha, alpha tocopherol hydrogen succinate and thioglycolyate sodium.

In some aspects, the compositions comprise an antioxidant, a free radical scavenger, a quencher, an antioxidant synergist or a combination thereof. In some aspects, the antioxidant is selected from methionine, butylated hydroxytoluene, butylated hydroxyanisole, propyl gallate, or a combination thereof. In other aspects, the antioxidant comprises methionine. In further aspects, the antioxidant is L-methionine. In certain aspects, the compositions comprise at least or equal to 20 mM methionine. In other aspects, the compositions comprise at least or equal to 5 mM methionine. In still other aspects, the compositions comprise at least or equal to 10 mM methionine. In further aspects, the compositions comprise at least or equal to 50 mM methionine. In other aspects, the compositions comprise 10 to 20 mM methionine. In other aspects, the compositions comprise 0 to 50 mM methionine.

Suspending, emulsifying and/or dispersing agents can include by way of example and without limitation, sodium carboxymethylcellulose, hydroxypropyl methylcellulose, Polysorbate 80 (TWEEN® 80), and polyvinylpyrrolidone.

In various aspects, the compositions comprise a surfactant. In certain aspects, the surfactant is selected from polysorbate 20, polysorbate 80, a pluronic, polyoxyethylene sorbitan mono-oleate, polyethylene mono-laureate, N-actylglucoside, or a combination thereof. In certain aspects, the surfactant is polysorbate 20. In further aspects, the compositions comprise from 0.0001% to 0.1% (wt/vol %) polysorbate 20. In additional aspects, the compositions comprise cyclodextrin. In further aspects, the cyclodextrin comprises (2-hydroxypropyl)-β-cyclodextrin.

A sequestering or chelating agent of metal ions can, include by way of example and without limitation, calcium disodium EDTA, disodium EDTA, sodium EDTA, calcium versetaminde sodium, calteridol, and DPTA. In some aspects, the present compositions comprise a metal chelator. In certain aspects, the metal chelator is selected from EDTA, deferoxamine mesylate, EGTA, fumaric acid, and malic acid, salts thereof, or combinations thereof. In further aspects, the metal chelator comprises EDTA or salts thereof. In certain aspects, the compositions have an EDTA concentration of about 0.1 mg/ml to about 1.0 mg/ml.

Other isotonic agents, buffers, antioxidants, anesthetics, suspending and dispersing agents, emulsifying agents and chelating agents are well-known to those in the art. See, for example, Gennaro (ed.), Remington's Pharmaceutical Sciences, 19th Edition (Mack Publishing Company 1995).

Pharmaceutical carriers also can include, by way of example and without limitation, ethyl alcohol, polyethylene glycol and propylene glycol for water miscible vehicles and sodium hydroxide, hydrochloric acid, citric acid, or lactic acid. Other pharmaceutical carriers are well-known to those in the art. See, for example, Gennaro (ed.), Remington's Pharmaceutical Sciences, 19th Edition (Mack Publishing Company 1995).

The chlorotoxin conjugates described herein can often be formulated using a variety of parameters including by way of example and without limitation, pH, molarity, % weight/volume, % volume/volume and the like. Other factors considered in the formulation of, stability of, storage of, shipping of chlorotoxin conjugates include by way of example and without limitation, the gas environment, container material, container color, cap material, cap color, presence of additional aspects, such as antioxidants, stabilizers, photoprotective compounds, protectants, sugars, ion chelators, ion donors or the like. Any factor which serves as any one of the above factors known to one of ordinary skill in the art can often be used with the chlorotoxin conjugates described herein but not limited as such.

The preparation of pharmaceutical or pharmacological compositions are known to those of skill in the art in light of the present disclosure. General techniques for formulation and administration can be found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants, and aerosols are examples of such formulations.

The chlorotoxin conjugates can often be stored at various temperatures, including by way of example and without limitation, freezing, for example at about −20° C., about −70° C., about −80° C., about −100° C., about −120° C., about −150° C., about −200° C. or more than about −200° C.; cold storage, for example at about 10° C., about 5° C., about 4° C., about 2° C., about 0° C., about −2° C. or more than about −5° C.; or any other suitable temperature such that the composition remains stable.

In some aspects, compositions comprising the compounds described herein are stored as lyophilized solids. In some aspects, the present disclosure provides methods for producing the lyophilized composition, the method comprising providing the composition, and lyophilizing the composition, thereby producing the lyophilized composition.

Using lyophilization, it can be possible to store the compounds in a manner that maintains physiological or otherwise optimal pH, isotonicity and stability. Such materials can include pH buffers, preservatives, tonicity adjusting agents, anti-oxidants, other polymers (e.g., viscosity adjusting agents or extenders), and excipients to stabilize the labile protein against the stresses of drying and storage of the dried product. Specific illustrative examples of such additives can include phosphate, citrate, or borate buffers; thimerosal; sorbic acid; methyl or propyl paraben, and chlorobutanol preservatives; sodium chloride: polyvinyl alcohol, polyvinyl pyrrolidone; mannitol, dextrose, dextran, lactose, sucrose, ethylene diamine tetra-acetic acid, and the like. Suitable formulations, known in the art, (Remington's Pharmaceutical Sciences (latest edition), Mack Publishing Company, Easton, Pa.; Arakawa et al. (1990), supra; Carpenter et al. (1991), supra; and Pikal (1990), supra).

In certain aspects, the pharmaceutically acceptable carrier comprises a reconstitution stabilizer. In other aspects, the reconstitution stabilizer comprises a water-soluble polymer. In additional aspects, the water-soluble polymer is selected from a polaxamer, a polyol, a polyethylene glycol, a polyvinylalcohol, a hydroxyethyl starch, dextran, polyvinylpyrrolidene poly(acrylic acid), or a combination thereof.

The term “reconstitution stabilizer” means any excipient which is capable of preventing aggregation of a reconstituted protein in an aqueous medium. Excipients possessing the necessary characteristics for the present invention are well-known in the art and generally function by the mechanisms of charge replusion, steric hindrance, hydrophobic binding or specific high-affinity binding to the dried protein. Exemplary excipients can include various osmolytes, various salts, water soluble synthetic and natural polymers, surfactants, sulfated polysaccharides, carrier proteins, buffers, and the like (Manning et al. (1989), Pharmaceutical Research, 6:903-918; and Paborji, et al. (1994), Pharmaceutical Research, 11:764-771).

The present compounds and an effective amount of the reconstitution stabilizer can be admixed under conditions effective to reduce aggregation of present compounds upon reconstitution with the reconstitution medium (e.g., a solvent and optionally other components such as antibacterials). The reconstitution stabilizer may be admixed with the compounds at a suitable time before, during, or after reconstitution. In one aspect, the reconstitution stabilizer is pre-dissolved in the reconstitution medium. The compound can be reconstituted at a temperature which is above the freezing point of the reconstitution medium, but which will not degrade the compound and which will not be deleterious to the reconstitution stabilizer. In one aspect, the temperature can be between about 2° C. to 50° C.

The time taken to mix the reconstitution stabilizer and the dried compound can be for a sufficient period to prepare a suitable admixture. In one aspect, the mixing can be from 1 to 30 minutes. Generally, the reconstituted formulation can be used soon after reconstitution.

In certain aspects, the present compositions are reconstituted from a lyophilized form. In other aspects, the present disclosure provides methods for producing the reconstituted composition, the method comprising providing a lyophilized composition; and reconstituting the composition with a solution to produce a reconstituted composition. In various aspects, the reconstituting solution comprises water. In some aspects, the reconstituting solution is selected from sterile water, physiological saline solution, glucose solution or other aqueous solvents (e.g., alcohols such as ethyl, n-propyl or isopropyl, butyl alcohol), or a combination thereof, which are capable of dissolving the dried composition and compatible with the selected administration route and which does not negatively interfere with the compound and the reconstitution stabilizers employed.

### Dosages and Methods of Administration of Compounds

The product or dosage form characteristics can result from processing methods and/or parameters for generating formulations, such as powders, lyophilized compositions, and the like include, but are not limited to, density, water content, friability, disintegration, dissolution profile(s), shape, size, weight, uniformity and composition of the particles. These product characteristics can often be modulated in a number of ways and can affect the final in vitro and/or in vivo performance of the formulations. Product or dosage form characteristics can often be a consequence of excipient selection, excipient composition, manufacturing methods applied, or a combination of any of these. The combination of excipients as well as product characteristics (including processing methods or processing parameters) of the final dosage form can ultimately determine the pharmacokinetic profile of the active ingredient in vivo. The administered chlorotoxin conjugate formulations described herein can often be processed or manufactured under specific conditions such as, for example, mixing methods (including sieve size, rpm, and milling), drying time, conditions, environmental parameters (e.g., temperature, humidity and combinations thereof) which themselves can modulate the pharmacokinetic profile of chlorotoxin compositions in vivo (i.e., increase the average Cₘₐₓ or AUC). In order to quantitatively compare one formulation to another, it is customary to measure several of these product or dosage form characteristics. This is also necessary when attempting to duplicate multiple batches.

Dissolution and drug release from formulations can depend on many factors including the solubility and concentration of the active ingredient, the nature and composition of the excipients, content uniformity, water content, product shape and size, porosity, disintegration time, and other factors. The release of a drug or active ingredient from a final dosage form in vitro can typically be characterized by its dissolution profile under standardized conditions (using United States Pharmacopeia (USP) or similar accepted methods for reference) and at the appropriate pH, often a neutral pH. The dissolution profile can show the amount of drug released over time into the test media under specified conditions. Standard conditions can make use of buffers at an appropriate pH in order to best mimic the pH of a subject's blood.

Typically a therapeutically effective dosage can be formulated to contain a dose of at least about 0.1 mg up to about 100 mg or more, such as more than 100 mg of chlorotoxin conjugate. In some aspects, the effective dosage is formulated to contain a dose of at least about 0.01 mg, about 0.02 mg, about 0.03 mg, about 0.05 mg, about 0.07 mg, about 0.1 mg, about 0.2 mg, about 0.3 mg, about 0.35 mg, about 0.375 mg, about 0.4 mg, about 0.5 mg, about 0.6 mg, about 0.7 mg, about 0.75 mg, about 0.8 mg, about 0.9 mg, about 1 mg, about 1.3 mg, about 1.4 mg, about 1.5 mg, about 1.8 mg, about 1.9 mg, about 2 mg, about 2.4 mg, about 3 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 40 mg, about 45 mg, about 50 mg, about 55 mg, about 60 mg, about 70 mg, about 80 mg, about 90 mg, about 100 mg, about 150 mg or about 200 mg or more of chlorotoxin conjugate.

In some exemplary aspects, a dose is 0.1 mg to 1 mg for a mouse. In other aspects, the effective dosage is formulated to contain a dose of 0.1 mg to 0.2 mg, of 0.1 mg to 0.3 mg, of 0.1 mg to 0.4 mg, of 0.1 mg to 0.5 mg, of 0.1 mg to 0.6 mg, of 0.1 mg to 0.7 mg, of 0.1 mg to 0.8 mg, of 0.1 mg to 0.9 mg, of 0.3 mg to 0.6 mg, of 0.3 mg to 0.8 mg, of 0.3 mg to 1 mg, of 0.5 mg to 0.8 mg, of 0.5 mg to 1 mg, of 0.8 mg to 0.9 mg, or of 0.8 mg to 1 mg.

In some exemplary aspects, a therapeutically effective dosage is formulated to contain a dose of 1 mg to 10 mg for a dog. In other aspects, the effective dosage is formulated to contain a dose of 1 mg to 2 mg, of 1 mg to 3 mg, of 1 mg to 4 mg, of 1 mg to 5 mg, of 1 mg to 6 mg, of 1 mg to 7 mg, of 1 mg to 8 mg, of 1 mg to 9 mg, of 3 mg to 6 mg, of 3 mg to 8 mg, of 3 mg to 10 mg, of 5 mg to 8 mg, of 5 mg to 10 mg, of 8 mg to 9 mg, or of 8 mg to 10 mg.

In some exemplary aspects, a therapeutically effective dosage is formulated to contain a dose of 0.3 mg to 3 mg for a rat. In other aspects, the effective dosage is formulated to contain a dose of 0.3 mg to 0.8 mg, of 0.3 mg to 1 mg, of 0.3 mg to 1.5 mg, of 0.3 mg to 2 mg, of 0.3 mg to 2.5 mg, of 1 mg to 1.5 mg, of 1 mg to 2 mg, of 1 mg to 2.5 mg, of 1 mg to 3 mg, of 1.5 mg to 2 mg, of 1.5 mg to 2.5 mg, of 1.5 mg to 3 mg, of 2 mg to 2.5 mg, or of 2.5 mg to 3 mg.

In some exemplary aspects, a therapeutically effective dosage is formulated to contain a dose of 0.6 mg to 60 mg for a monkey. In other aspects, the effective dosage is formulated to contain a dose of 0.6 mg to 2 mg, of 0.6 mg to 10 mg, of 0.6 mg to 20 mg, of 0.6 mg to 30 mg, of 0.6 mg to 40 mg, of 0.6 mg to 50 mg, of 5 mg to 10 mg, of 5 mg to 20 mg, of 5 mg to 30 mg, of 5 mg to 40 mg, of 5 mg to 50 mg, of 5 mg to 60 mg, of 10 mg to 20 mg, of 10 mg to 30 mg, of 10 mg to 40 mg, of 10 mg to 50 mg, of 10 mg to 60 mg, of 25 mg to 40 mg, of 25 mg to 50 mg, of 25 mg to 60 mg, of 40 mg to 50 mg, of 40 mg to 60 mg, of 50 mg to 60 mg, or of 55 mg to 60 mg.

In some exemplary aspects, a therapeutically effective dosage is formulated to contain a dose of 1 mg to 100 mg or more for a human. In other aspects, the effective dosage is formulated to contain a dose of 1 mg to 5 mg, of 1 mg to 10 mg, of 1 mg to 20 mg, of 1 mg to 30 mg, of 1 mg to 40 mg, of 1 mg to 50 mg, of 1 mg to 60 mg, of 1 mg to 70 mg, of 1 mg to 80 mg, of 1 mg to 90 mg, 3 mg to 5 mg, of 3 mg to 10 mg, of 3 mg to 20 mg, of 3 mg to 30 mg, of 3 mg to 40 mg, of 3 mg to 50 mg, of 3 mg to 60 mg, of 3 mg to 70 mg, of 3 mg to 80 mg, of 3 mg to 90 mg, of 3 mg to 100 mg, of 10 mg to 20 mg, of 10 mg to 30 mg, of 10 mg to 40 mg, of 10 mg to 50 mg, of 10 mg to 60 mg, of 10 mg to 70 mg, of 10 mg to 80 mg, of 10 mg to 90 mg, of 10 mg to 100 mg, of 20 mg to 50 mg, of 20 mg to 75 mg, of 20 mg to 100 mg, of 30 mg to 50 mg, of 30 mg to 75 mg, of 30 mg to 100 mg, of 50 mg to 60 mg, of 50 mg to 75 mg, of 50 mg to 100 mg, of 75 mg to 80 mg, of 75 mg to 90 mg, of 75 mg to 100 mg, of 90 mg to 95 mg, or of 95 mg to 100 mg. The amount of chlorotoxin conjugate administered to a subject can often be the total about amount listed herein. In some aspects, the amount of chlorotoxin conjugate administered to a subject is often the about per milligram, gram or kilogram of subject weight for each amount listed herein. In other aspects, the amount of chlorotoxin conjugate administered to a subject is often the about per milliliter or liter of fluid volume for each amount listed herein. In yet other aspects, the amount of chlorotoxin conjugate administered to a subject is often the about per square millimeter, square centimeter, or square meter of subject surface body area or subject body area for each amount listed herein.

As used herein a “dosage regimen” refers to the protocol used to administer an intravenous pharmaceutical formulation comprising chlorotoxin conjugate to a subject. In some aspects, the dosage regimen comprises a dose amount and dosing interval. In some aspects, the dosage regimen further comprises a dosing duration. As used herein “dosing duration” refers to the period of time over which a dose is administered. Furthermore, the dosage regimen can comprise a method of administration. In some aspects, a method of administration comprises a bolus, a slow bolus, or an infusion.

As used herein, a “bolus” can refer to an intravenous injection administered over a short period of time. In one aspect, a bolus is manually administered over a short period of time. In other aspects, a bolus is administered via a pump or other automated mechanism over a short period of time. In some aspects, a bolus is administered over a period of time less than or equal to 5 seconds, less than or equal to 10 seconds, less than or equal to 15 seconds, less than or equal to 20 seconds, less than or equal to 25 seconds, less than or equal to 30 seconds, less than or equal to 35 seconds, less than or equal to 40 seconds, less than or equal to 45 seconds, less than or equal to 50 seconds, less than or equal to 55 seconds, less than or equal to 60 seconds, less than or equal to 65 seconds, less than or equal to 70 seconds, less than or equal to 75 seconds, less than or equal to 80 seconds, less than or equal to 85 seconds, less than or equal to 90 seconds, less than or equal to 95 seconds, less than or equal 100 seconds, less than or equal to 105 seconds, less than or equal to 110 seconds, less than or equal to 115 seconds, or less than or equal to 120 seconds.

As used herein, a “slow bolus” can refer to an intravenous injection administered over longer period of time than a bolus, but a shorter period of time than an infusion. In one aspect, a slow bolus is manually administered over longer period of time than a bolus, but a shorter period of time than an infusion. In other aspects, a slow bolus is administered via a pump or other automated mechanism over longer period of time than a bolus, but a shorter period of time than an infusion. In one aspect, a slow bolus is administered over a period of time within a range from about 2 minutes to about 5 minutes. In other aspects, a slow bolus is administered over a period of time within a range from about 2 minutes to about 4.9 minutes, about 2 minutes to about 4.8 minutes, about 2 minutes to about 4.8 minutes, about 2 minutes to about 4.7 minutes, about 2 minutes to about 4.6 minutes, about 2 minutes to about 4.5 minutes, about 2 minutes to about 4.4 minutes, about 2 minutes to about 4.3 minutes, about 2 minutes to about 4.4 minutes, about 2 minutes to about 4.3 minutes, about 2 minutes to about 4.2 minutes, about 2 minutes to about 4.1 minutes, about 2 minutes to about 4 minutes, about 2 minutes to about 3.9 minutes, about 2 minutes to about 3.8 minutes, about 2 minutes to about 3.7 minutes, about 2 minutes to about 3.6 minutes, about 2 minutes to about 3.5 minutes, about 2 minutes to about 3.4 minutes, about 2 minutes to about 3.3 minutes, about 2 minutes to about 3.2 minutes, about 2 minutes to about 3.1 minutes, about 2 minutes to about 3 minutes, about 2 minutes to about 2.9 minutes, about 2 minutes to about 2.8 minutes, about 2 minutes to about 2.7 minutes, about 2 minutes to about 2.6 minutes, about 2 minutes to about 2.5 minutes, about 2 minutes to about 2.4 minutes, about 2 minutes to about 2.3 minutes, about 2 minutes to about 2.2 minutes, or about 2 minutes to about 2.1 minutes. In other aspects, a slow bolus is administered over a period of time within the range of about 2.5 minutes to about 3 minutes, about 2.5 minutes to about 3.5 minutes, about 2.5 minutes to about 4 minutes, about 2.5 minutes to about 4.5 minutes, about 2.5 minutes to about 5 minutes, about 3 minutes to about 3.5 minutes, about 3 minutes to about 4 minutes, about 3 minutes to about 4.5 minutes, about 3 minutes about 5 minutes, about 3.5 minutes to about 4 minutes, about 3.5 minutes to about 4.5 minutes, about 3.5 minutes to about 5 minutes, about 4 minutes to about 4.5 minutes, about 4 minutes about 5 minutes, or about 4.5 minutes to about 5 minutes.

As used herein, an “infusion” can refer to an intravenous injection administered over longer period of time than a bolus or a slow bolus. In one aspect, an infusion is administered via a pump or other automated mechanism over longer period of time than a bolus or a slow bolus. In other aspects, an infusion is manually administered over longer period of time than a bolus or a slow bolus. In other aspects, the infusion is administered over a period of time that is greater than or equal to 5 minutes, greater than or equal to 5.5 minutes, greater than or equal to 6 minutes, greater than or equal to 6.5 minutes, greater than or equal to 7 minutes, greater than or equal to 7.5 minutes, greater than or equal to 8 minutes, greater than or equal to 8.5 minutes, greater than or equal to 9 minutes, greater than or equal to 9.5 minutes, greater than or equal to 10 minutes, greater than or equal to 10.5 minutes, greater than or equal to 11 minutes, greater than or equal to 11.5 minutes, greater than or equal to 12 minutes, greater than or equal to 12.5 minutes, greater than or equal to 13 minutes, greater than or equal to 13.5 minutes, greater than or equal to 14 minutes, greater than or equal to 14.5 minutes, greater than or equal to 15 minutes, greater than or equal to 15.5 minutes greater than or equal to 16 minutes, greater than or equal to 16.5 minutes, greater than or equal to 17 minutes, greater than or equal to 17.5 minutes, greater than or equal to 18 minutes, greater than or equal to 18.5 minutes, greater than or equal to 19 minutes, greater than or equal to 19.5 minutes, greater than or equal to 20 minutes, greater than or equal to 30 minutes, greater than or equal to 45 minutes, greater than or equal to 60 minutes, greater than or equal to 75 minutes, greater than or equal to 90 minutes, greater than or equal to 105 minutes, greater than or equal to 120 minutes, greater than or equal to 150 minutes, greater than or equal to 180 minutes, greater than or equal to 210 minutes, greater than or equal to 240 minutes, greater than or equal to 270 minutes, greater than or equal to 300 minutes. In still other aspects, the infusion is administered over a period of time that is within a range of about 5 minutes to about 20 minutes, about 5 minutes to about 19 minutes, about 5 minutes to about 18 minutes, about 5 minutes to about 17 minutes, about 5 minutes to about 16 minutes, about 5 minutes to about 15 minutes, about 5 minutes to about 14 minutes, about 5 minutes to about 13 minutes, about 5 minutes to about 12 minutes, about 5 minutes to about 10 minutes, about 5 minutes to about 9 minutes, about 5 minutes to about 8 minutes, about 5 minutes to about 7 minutes, or about 5 minutes to about 6 minutes. In yet still further aspects, the infusion is administered over a period of time that is within the range of about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 45 minutes, about 5 minutes to about 60 minutes, about 5 minutes to about 90 minutes, about 5 minutes to about 120 minutes, about 5 minutes to about 150 minutes, about 5 minutes to about 180 minutes, about 5 minutes to about 210 minutes, about 240 minutes to about 270 minutes, about 5 minutes to about 300 minutes, about 30 minutes to about 75 minutes, about 30 minutes to about 90 minutes, about 30 minutes to about 120 minutes, about 30 minutes to about 150 minutes, about 30 minutes to about 180 minutes, about 30 minutes to about 210 minutes, about 30 minutes to about 240 minutes, about 30 minutes to about 270 minutes, about 30 minutes to about 300 minutes, about 60 minutes to about 90 minutes, about 60 minutes to about 120 minutes, about 60 minutes to about 150 minutes, about 60 minutes to about 180 minutes, about 60 minutes to about 210 minutes, about 60 minutes to about 240 minutes, about 60 minutes to about 270 minutes, about 60 minutes to about 300 minutes, about 90 minutes to about 120 minutes, about 90 minutes to about 180 minutes, about 90 minutes to about 240 minutes, about 60 minutes to about 300 minutes, about 120 minutes to about 180 minutes, about 120 minutes to about 240 minutes, about 120 minutes to about 300 minutes, about 180 minutes to about 240 minutes, about 180 minutes to about 300 minutes, or about 240 minutes to about 300 minutes.

In some aspects, the dose of chlorotoxin conjugate is administrated to a subject using a single dose administration regimen or a repeat dose administration regimen. For example, a single dose administration regimen can include a single, one-time administration of a bolus, a slow bolus, or an infusion of a chlorotoxin conjugate to a subject via an intravenous administration route at any desired dose set forth in this application. Alternatively, a repeat dose administration regimen can include a number of administrations greater than a single, one-time administration of a bolus, a slow bolus, or an infusion of a chlorotoxin conjugate to a subject via an intravenous administration route at any desired dose set forth in this application. In repeat dose administration regimens, a dose can be delivered once daily for 2 days, 3 days, 4 days, 5 days, 6 days, 7 days, 8 days, 9 days, 10 days, or more. In some repeat dose administration regimens, a dose can be delivered once a week, once every two weeks, once every three weeks, once every month, once every two months, once every three months, once every four months, once every five months, once every six months, or more. In some repeat dose administrations, a dose can be delivered once to a subject prior to surgery and a second dose is administered to the subject prior to a second surgery. The second surgery can be hours, days, months, or years after the first surgery. In some repeat dose administration regimens, a dose can also be delivered more than once per day. For example, a dose can be administered once every 4 hours, 6 hours, 8 hours, 12 hours, or more. In repeat dose administration, if a subject is administered doses that are less than 4 hours apart, the dose can be given by infusion. For example, in a repeat dose administration regimen, a dose can be delivered by infusion every 15 minutes, once every one hour, once every two hours, or once every three hours.

In some aspects, the dose of chlorotoxin conjugate is administered to a subject using either a fixed or a scaling dosing scheme. For example, a fixed dosing scheme includes administration of a bolus, a slow bolus or an infusion of chlorotoxin conjugate to a subject via an intravenous administration route wherein the fixed dose is, for example and without limitation, 0.1 mg to 100 mg and does not account or adjust for a subject's age, weight, height, body mass index, metabolism, or the like, or 1 mg to 30 mg and does not account or adjust for a subject's age, weight, height, body mass index, metabolism, or the like. For example, a scaling dosing scheme includes administration of a bolus, a slow bolus or an infusion of chlorotoxin conjugate to a subject via an intravenous administration route wherein the scaled dose is, for example and without limitation, 0.1 mg to 100 mg and accounts or adjusts for a subject's age, weight, height, body mass index, metabolism, or the like, or 1 mg to 30 mg and accounts or adjusts for a subject's age, weight, height, body mass index, metabolism, or the like. In some aspects, the fixed dose and/or the scaled dose are determined for one subject based upon the dose administered to a different subject wherein the subjects are or are not the same species, for example a mouse and a human, a rat and a human, a dog and a human, a monkey and a human, or a non-human primate and a human. Often in a fixed dose, the same dose or about the same dose can be administered to all subjects, for example a mouse and a human or a rat and a human, a dog and a human, a monkey and a human, or a non-human primate and a human. In some aspects, the scaled dose to be administered to a subject is determined from the dose administered to a different subject wherein the subjects are or are not the same species, for example a mouse and a human, a rat and a human, a dog and a human, a monkey and a human, or a non-human primate and a human. The scaled dose can therefore be increased from the dose administered to the mouse, rat, dog, monkey, or non-human primate to the dose administered to the human based upon the difference between the mouse, rat, dog, monkey, or non-human primate and the human on factors such as subject age, weight, height, body surface area, metabolism, size, physiological influences on pharmacokinetics, or the like. In one aspect, the dose is scaled from a rat to a human.

In some aspects, the compounds and compositions described herein, are used for detecting the presence or absence of the compound in a tissue or cell, wherein the presence of the compound in the tissue or cell indicates the presence of a cancerous tissue or cancer cell. In some embodiments, the compound binds to the cancerous tissue or cancer cell. In some aspects, the detecting of the cancerous tissue or cancer cell is performed using fluorescence imaging. In some aspects, the cancerous tissue or cancer cell is associated with one or more of: brain cancer, glioma, astrocytoma, medulloblastoma, oligiodendroglioma, choroids plexus carcinoma, ependymoma, pituitary cancer, neuroblastoma, basal cell carcinoma, cutaneous squamous cell carcinoma, melanoma, head and neck cancer, lung cancer, small cell lung cancer, non-small cell lung cancer, breast cancer, ductal carcinoma in situ, intestinal cancer, pancreatic cancer, liver cancer, kidney cancer, bladder cancer, carcinoma of unknown primary, sarcoma, osteosarcoma, rhabdomyosarcoma, Ewing's sarcoma, gastrointestinal stromal tumors, melanoma, ovarian cancer, cervical cancer, lymphoma, Hodgkin's lymphoma, non-Hodgkin's lymphoma, thyroid cancer, anal cancer, colo-rectal cancer, laryngeal cancer, multiple myeloma, prostate cancer, retinoblastoma, gastric cancer, esophageal cancer, testicular cancer, or Wilm's tumor.

In further aspects, the compounds and compositions described herein, are used for detecting the presence or absence of the compound in a tissue or cell, wherein the presence of the compound in the tissue or cell indicates the presence of a cancerous tissue or cancer cell, and wherein the detecting allows for surgically removing the cancerous tissue or cancer cell from the human subject. In some aspects, the compound is administered at a dosage sufficient to treat cancer in the human subject. In some aspects, the compound binds to a cancerous tissue or cancer cell. In some aspects, the cancer being treated comprises one or more of: brain cancer, glioma, astrocytoma, medulloblastoma, oligiodendroglioma, choroids plexus carcinoma, ependymoma, pituitary cancer, neuroblastoma, basal cell carcinoma, cutaneous squamous cell carcinoma, melanoma, head and neck cancer, lung cancer, small cell lung cancer, non-small cell lung cancer, breast cancer, ductal carcinoma in situ, intestinal cancer, pancreatic cancer, liver cancer, kidney cancer, bladder cancer, carcinoma of unknown primary, sarcoma, osteosarcoma, rhabdomyosarcoma, Ewing's sarcoma, gastrointestinal stromal tumors, melanoma, ovarian cancer, cervical cancer, lymphoma, Hodgkin's lymphoma, non-Hodgkin's lymphoma, thyroid cancer, anal cancer, colo-rectal cancer, laryngeal cancer, multiple myeloma, prostate cancer, retinoblastoma, gastric cancer, esophageal cancer, testicular cancer, or Wilm's tumor. Furthermore, the compounds and compositions described herein are administered to a subject before surgery and/or during surgery, in which the excised tissue from the subject is contacted with compositions of the chlorotoxin conjugates. In some aspects, the compositions of the chlorotoxin conjugates are administered during surgery. In certain aspects, compositions of chlorotoxin conjugates are intravenously administered to a subject about 0.25 hours, about 0.5 hours, about 0.75 hours, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, or about 72 hours prior to performing surgery on a human subject. In some aspects, compositions of chlorotoxin conjugates are intravenously administered to a subject between 0 and 1 hours, between 1 and 2 hours, between 2 and 3 hours, between 3 and 4 hours, between 4 and 5 hours, between 5 and 6 hours, between 6 and 9 hours, between 9 and 12 hours, between 12 and 24 hours, between 24 and 36 hours, between 36 and 48 hours or between 48 and 72 hours (inclusive) before surgery.

Tissue or fluid samples, such as blood, normal tissue, and tumor tissue, can often be isolated from a subject prior to administration of a chlorotoxin conjugate, or sometimes as a baseline reference. Samplescan also be isolated from a subject after administration of the compounds of the present disclosure, often less than about 1 minute after, less than about 2 minutes after, less than about 3 minutes after, less than about 4 minutes after, less than about 5 minutes after, less than about 6 minutes after, less than about 7 minutes after, less than about 8 minutes after, less than about 9 minutes after, less than about 10 minutes after, less than about 11 minutes after, less than about 12 minutes after, less than about 13 minutes after, less than about 14 minutes after, less than about 15 minutes after, less than about 20 minutes after, less than about 30 minutes after, less than about 40 minutes after, less than about 50 minutes after, less than about 60 minutes after, less than about 1 hour after, less than about 2 hours after, less than about 3 hours after, less than about 4 hours after, less than about 5 hours after, less than about 6 hours after, less than about 12 hours after, less than about 18 hours after, less than about 24 hours after, less than about 36 hours after, less than about 48 hours after, less than about 72 hours after, less than about 96 hours after, less than about 5 days after, less than about 7 days after, less than about 10 days after, less than about 14 days after, less than about 21 days after, less than about 4 weeks after, less than about 6 weeks after, less than about 8 weeks after, less than about 12 weeks after, less than about 16 weeks after, less than about 20 weeks after or more than 20 weeks after.

### Pharmacokinetics

The methods and compositions described herein relate to pharmacokinetics of intravenous administration of chlorotoxin conjugates to a subject. Pharmacokinetics can often be described using methods and models, for example, compartmental models or noncompartmental methods. Compartmental models can include but are not limited to, a monocompartmental model, the two compartmental model, the multicompartmental model, or the like. Models can often be divided into different compartments and described by the corresponding scheme. For example, one scheme is the absorption, distribution, metabolism and excretion (ADME) scheme. For another example, another scheme is the liberation, absorption, distribution, metabolism and excretion (LADME) scheme. In some aspects, metabolism and excretion are grouped into one compartment referred to as the elimination compartment. For example, liberation includes liberation of the active portion of the composition from the delivery system, absorption includes absorption of the active portion of the composition by the subject, distribution includes distribution of the composition through the blood plasma and to different tissues, metabolism, which includes metabolism or inactivation of the composition and finally excretion, which includes excretion or elimination of the composition or the products of metabolism of the composition. Often, compositions administered intravenously to a subject can be subject to multiphasic pharmacokinetic profiles, which can include, but are not limited to, aspects of tissue distribution and metabolism/excretion. As such, the decrease in plasma or serum concentration of the composition can often be biphasic, including, for example, an alpha phase and a beta phase, or occasionally a gamma, delta or other phase can be observed

Pharmacokinetics can include determining at least one parameter associated with intravenous administration of chlorotoxin conjugates to a subject. In some aspects, parameters include at least the dose (D), dosing interval (τ), area under curve (AUC) (which can be calculated by the linear/linear trapezoidal rule or by the linear up/log down trapezoidal rule), maximum concentration (Cₘₐₓ), minimum concentration reached before a subsequent dose is administered (Cₘᵢₙ), minimum time (Tₘᵢₙ), maximum time to reach Cₘₐₓ (Tₘₐₓ), volume of distribution (V_{d}), steady-state volume of distribution (Vₛₛ), back-extrapolated concentration at time 0 (C₀), steady state concentration (Cₛₛ), elimination rate constant (kₑ), infusion rate (kᵢₙ), clearance (CL), bioavailability (f), fluctuation (% PTF), and elimination half-life (t_{1/2}).

The compounds described herein can have values for at least one of the pharmacokinetic parameters listed herein and can be known to those of ordinary skill in the art. Often, the values for the pharmacokinetic parameters can be recorded, observed, measured, processed, analyzed, or the like, as data. The pharmacokinetics parameters can be any parameters suitable for describing the plasma or serum profiles of chlorotoxin conjugates described herein. In some aspects, the pharmacokinetic samples are used to produce a pharmacokinetic profile in a human subject. For example, the pharmacokinetic samples are often obtained at a time after dosing of, for example, about zero minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about zero hours, about 0.5 hours, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 36 hours, about 48 hours, about 60 hours, about 72 hours, about 84 hours, about 96 hours, about 108 hours, about 120 hours, about 132 hours, about 146 hours, or about 168 hours.

The pharmacokinetics parameters can be any parameters suitable for describing the plasma or serum profiles of chlorotoxin conjugates described herein. In some aspects, the dose (D) includes, by way of example, but is not limited to, about 0.01 mg, about 0.02 mg, about 0.03 mg, about 0.5 mg, about 0.07 mg, about 0.1 mg, about 0.2 mg, about 0.3 mg, about 0.35 mg, about 0.375 mg, about 0.4 mg, about 0.5 mg, about 0.6 mg, about 0.7 mg, about 0.75 mg, about 0.8 mg, about 0.9 mg, about 1 mg, about 1.3 mg, about 1.4 mg, about 1.5 mg, about 1.8 mg, about 1.9 mg, about 2 mg, about 2.4 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 22 mg, about 24 mg, about 26 mg, about 28 mg, about 30 mg, about 35 mg, about 40 mg, about 45 mg, about 50 mg, about 60 mg, about 70 mg, about 80 mg, about 90 mg, about 100 mg or about 110 mg or more of chlorotoxin conjugate. In some aspects, the dosing interval (τ) includes by way of example but is not limited to, about 0.25 hours, about 0.5 hours, about 1 hours, about 6 hours, about 12 hours, about 24 hours, about 36 hours, about 48 hours, or about 72 hours before surgery.

The pharmacokinetics parameters can be any parameters suitable for describing the plasma or serum profiles of chlorotoxin conjugates described herein. In some aspects, the area under curve (AUC) per each 1 mg dosage of the compound administered includes by way of example but is not limited to, is greater than or equal to about 10 hr*ng/mL, greater than or equal to about 20 hr*ng/mL, greater than or equal to about 30 hr*ng/mL, greater than or equal to about 40 hr*ng/mL, greater than or equal to about 50 hr*ng/mL, greater than or equal to about 60 hr*ng/mL, greater than or equal to about 70 hr*ng/mL, greater than or equal to about 80 hr*ng/mL, greater than or equal to about 90 hr*ng/mL, greater than or equal to about 100 hr*ng/mL, greater than or equal to about 150 hr*ng/mL, greater than or equal to about 200 hr*ng/mL, greater than or equal to about 250 hr*ng/mL, greater than or equal to about 300 hr*ng/mL, greater than or equal to about 350 hr*ng/mL, greater than or equal to about 400 hr*ng/mL, greater than or equal to about 450 hr*ng/mL, greater than or equal to about 500 hr*ng/mL, greater than or equal to about 550 hr*ng/mL, greater than or equal to about 600 hr*ng/mL, greater than or equal to about 650 hr*ng/mL, greater than or equal to about 700 hr*ng/mL, greater than or equal to about 750 hr*ng/mL, or greater than or equal to about 800 hr*ng/mL. In some aspects, the AUC per each 1 mg dosage of the compound administered is less than or equal to about 10 hr*ng/mL, less than or equal to about 20 hr*ng/mL, less than or equal to about 30 hr*ng/mL, less than or equal to about 40 hr*ng/mL, less than or equal to about 50 hr*ng/mL, less than or equal to about 60 hr*ng/mL, less than or equal to about 70 hr*ng/mL, less than or equal to about 80 hr*ng/mL, less than or equal to about 90 hr*ng/mL, less than or equal to about 100 hr*ng/mL, less than or equal to about 150 hr*ng/mL, less than or equal to about 200 hr*ng/mL, less than or equal to about 250 hr*ng/mL, less than or equal to about 300 hr*ng/mL, less than or equal to about 350 hr*ng/mL, less than or equal to about 400 hr*ng/mL, less than or equal to about 450 hr*ng/mL, less than or equal to about 500 hr*ng/mL, less than or equal to about 550 hr*ng/mL, less than or equal to about 600 hr*ng/mL, less than or equal to about 650 hr*ng/mL, less than or equal to about 700 hr*ng/mL, less than or equal to about 750 hr*ng/mL, or less than or equal to about 800 hr*ng/mL. In some aspects, the average AUC per each 1 mg dosage of the compound administered is within a range from about 10 hr*ng/mL to about 800 hr*ng/mL, about 10 hr*ng/mL to about 700 hr*ng/mL, about 10 hr*ng/mL to about 600 hr*ng/mL, about 10 hr*ng/mL to about 500 hr*ng/mL, about 10 hr*ng/mL to about 400 hr*ng/mL, about 10 hr*ng/mL to about 300 hr*ng/mL, about 10 hr*ng/mL to about 200 hr*ng/mL, about 10 hr*ng/mL to about 100 hr*ng/mL, about 15 hr*ng/mL to about 800 hr*ng/mL, about 15 hr*ng/mL to about 700 hr*ng/mL, about 15 hr*ng/mL to about 600 hr*ng/mL, about 15 hr*ng/mL to about 500 hr*ng/mL, about 15 hr*ng/mL to about 400 hr*ng/mL, about 15 hr*ng/mL to about 300 hr*ng/mL, about 15 hr*ng/mL to about 200 hr*ng/mL, or about 15 hr*ng/mL to about 100 hr*ng/mL.

In some aspects, the AUC is at least 30 hr*ng/mL, at least 40 hr*ng/mL, at least 50 hr*ng/mL, at least 75 hr*ng/mL, at least 100 hr*ng/mL, at least 125 hr*ng/mL, at least 150 hr*ng/mL, at least 175 hr*ng/mL, at least 200 hr*ng/mL, at least 250 hr*ng/mL, at least 300 hr*ng/mL, at least 350 hr*ng/mL, at least 400 hr*ng/mL, at least 500 hr*ng/mL, at least 600 hr*ng/mL, at least 700 hr*ng/mL, at least 800 hr*ng/mL, at least 900 hr*ng/mL, at least 1,000 hr*ng/mL, at least 2,000 hr*ng/mL, at least 3,000 hr*ng/mL, at least 4,000 hr*ng/mL, at least 5,000 hr*ng/mL, at least 6,000 hr*ng/mL, at least 7,000 hr*ng/mL, at least 8,000 hr*ng/mL, at least 9,000 hr*ng/mL, at least 10,000 hr*ng/mL, at least 11,000 hr*ng/mL, at least 12,000 hr*ng/mL, at least 13,000 hr*ng/mL, at least 14,000 hr*ng/mL, at least 15,000 hr*ng/mL, at least 16,000 hr*ng/mL, at least 17,000 hr*ng/mL, at least 18,000 hr*ng/mL, at least 19,000 hr*ng/mL, at least 20,000 hr*ng/mL, at least 21,000 hr*ng/mL, at least 22,000 hr*ng/mL, at least 23,000 hr*ng/mL, at least 24,000 hr*ng/mL, at least 25,000 hr*ng/mL, at least 26,000 hr*ng/mL, at least 27,000 hr*ng/mL, at least 28,000 hr*ng/mL, at least 29,000 hr*ng/mL, at least 30,000 hr*ng/mL, at least 31,000 hr*ng/mL, at least 32,000 hr*ng/mL, at least 33,000 hr*ng/mL, at least 34,000 hr*ng/mL, at least 35,000 hr*ng/mL, at least 40,000 hr*ng/mL at least 45,000 hr*ng/mL at least 50,000 hr*ng/mL at least 55,000 hr*ng/mL at least 60,000 hr*ng/mL at least 65,000 hr*ng/mL at least 70,000 hr*ng/mL at least 75,000 hr*ng/mL at least 80,000 hr*ng/mL at least 85,000 hr*ng/mL at least 90,000 hr*ng/mL at least 95,000 hr*ng/mL at least 100,000 hr*ng/mL at least 125,000 hr*ng/mL at least 150,000 hr*ng/mL at least 175,000 hr*ng/mL at least 200,000 hr*ng/mL at least 250,000 hr*ng/mL at least 300,000 hr*ng/mL at least 350,000 hr*ng/mL at least 400,000 hr*ng/mL at least 450,000 hr*ng/mL at least 500,000 hr*ng/mL at least 550,000 hr*ng/mL at least 600,000 hr*ng/mL at least 650,000 hr*ng/mL at least 700,000 hr*ng/mL at least 750,000 hr*ng/mL at least 800,000 hr*ng/mL at least 850,000 hr*ng/mL at least 900,000 hr*ng/mL at least 950,000 hr*ng/mL at least 1,000,000 hr*ng/mL at least 1,100,000 hr*ng/mL at least 1,200,000 hr*ng/mL at least 1,300,000 hr*ng/mL at least 1,400,000 hr*ng/mL at least 1,500,000 hr*ng/mL at least 1,600,000 hr*ng/mL at least 1,700,000 hr*ng/mL at least 1,800,000 hr*ng/mL at least 1,900,000 hr*ng/mL at least 2,000,000 hr*ng/mL or any other AUC appropriate for describing a pharmacokinetic profile of a chlorotoxin conjugate described herein.

The AUC of a chlorotoxin described herein by way of example can be, but is not limited to, about 30 hr*ng/mL to about 75 hr*ng/mL, about 75 hr*ng/mL to about 200 hr*ng/mL, about 200 hr*ng/mL to about 600 hr*ng/mL to about 1,000 hr*ng/mL, about 1,000 hr*ng/mL to about 1,250 hr*ng/mL; about 1,250 hr*ng/mL to about 1,500 hr*ng/mL; about 1,500 hr*ng/mL to about 1,750 hr*ng/mL; about 1,750 hr*ng/mL to about 2,000 hr*ng/mL; about 2,000 hr*ng/mL to about 2,500 hr*ng/mL; about 2,500 hr*ng/mL to about 3,000 hr*ng/mL; about 3,000 hr*ng/mL to about 3,500 hr*ng/mL; about 3,500 hr*ng/mL to about 4,000 hr*ng/mL; about 4,000 hr*ng/mL to about 4,500 hr*ng/mL; about 4,500 hr*ng/mL to about 5,000 hr*ng/mL; about 5,000 hr*ng/mL to about 5,500 hr*ng/mL; about 5,500 hr*ng/mL to about 6,000 hr*ng/mL; about 6,000 hr*ng/mL to about 6,500 hr*ng/mL; about 6,500 hr*ng/mL to about 7,000 hr*ng/mL; about 7,000 hr*ng/mL to about 7,500 hr*ng/mL; about 7,500 hr*ng/mL to about 8,000 hr*ng/mL; about 8,000 hr*ng/mL to about 8,500 hr*ng/mL; about 8,500 hr*ng/mL to about 9,000 hr*ng/mL; about 9,000 hr*ng/mL to about 9,500 hr*ng/mL; about 9,500 hr*ng/mL to about 10,000 hr*ng/mL; about 10,000 hr*ng/mL to about 20,000 hr*ng/mL; about 20,000 hr*ng/mL to about 30,000 hr*ng/mL; about 30,000 hr*ng/mL to about 40,000 hr*ng/mL; about 40,000 hr*ng/mL to about 50,000 hr*ng/mL; about 50,000 hr*ng/mL to about 60,000 hr*ng/mL; about 60,000 hr*ng/mL to about 70,000 hr*ng/mL; about 70,000 hr*ng/mL to about 80,000 hr*ng/mL; about 80,000 hr*ng/mL to about 90,000 hr*ng/mL; about 90,000 hr*ng/mL to about 100,000 hr*ng/mL; about 100,000 hr*ng/mL to about 150,000 hr*ng/mL; about 150,000 hr*ng/mL to about 200,000 hr*ng/mL; about 200,000 hr*ng/mL to about 250,000 hr*ng/mL; about 250,000 hr*ng/mL to about 300,000 hr*ng/mL; about 300,000 hr*ng/mL to about 350,000 hr*ng/mL; about 350,000 hr*ng/mL to about 400,000 hr*ng/mL; about 400,000 hr*ng/mL to about 450,000 hr*ng/mL; about 450,000 hr*ng/mL to about 500,000 hr*ng/mL; about 500,000 hr*ng/mL to about 550,000 hr*ng/mL; about 550,000 hr*ng/mL to about 600,000 hr*ng/mL; about 600,000 hr*ng/mL to about 650,000 hr*ng/mL; about 650,000 hr*ng/mL to about 700,000 hr*ng/mL; about 700,000 hr*ng/mL to about 750,000 hr*ng/mL; about 750,000 hr*ng/mL to about 800,000 hr*ng/mL; about 800,000 hr*ng/mL to about 850,000 hr*ng/mL; about 850,000 hr*ng/mL to about 900,000 hr*ng/mL; about 900,000 hr*ng/mL to about 950,000 hr*ng/mL; about 950,000 hr*ng/mL to about 1,000,000 hr*ng/mL; about 1,000,000 hr*ng/mL to about 1,100,000 hr*ng/mL; about 1,100,000 hr*ng/mL to about 1,200,000 hr*ng/mL; about 1,200,000 hr*ng/mL to about 1,300,000 hr*ng/mL; about 1,300,000 hr*ng/mL to about 1,400,000 hr*ng/mL; about 1,40,000 hr*ng/mL to about 1,500,000 hr*ng/mL; or about 1,50,000 hr*ng/mL to about 2,000,000 hr*ng/mL.

The pharmacokinetic parameters can be any parameters suitable for describing a chlorotoxin conjugate described herein. The maximum blood concentration (Cₘₐₓ) per each 1 mg dosage of the compound administered can include, by way of example, but is not limited to, within a range of about 10 ng/mL to about 1000 ng/mL, 10 ng/mL to about 900 ng/mL, about 10 ng/mL to about 800 ng/mL, about 10 ng/mL to about 700 ng/mL, about 10 ng/mL to about 600 ng/mL, 15 ng/mL to about 1000 ng/mL, 15 ng/mL to about 900 ng/mL, about 15 ng/mL to about 800 ng/mL, about 15 ng/mL to about 700 ng/mL, about 15 ng/mL to about 600 ng/mL, about 20 ng/mL to about 600 ng/mL, about 30 ng/mL to about 600 ng/mL, about 30 ng/mL to about 500 ng/mL, about 30 ng/mL to about 400 ng/mL, about 30 ng/mL to about 300 ng/mL. In some aspects, the Cₘₐₓ per each 1 mg dosage of the compound administered is greater than or equal to about 20 ng/mL, greater than or equal to about 30 ng/mL, greater than or equal to about 40 ng/mL, greater than or equal to about 50 ng/mL, greater than or equal to about 60 ng/mL, greater than or equal to about 70 ng/mL, greater than or equal to about 80 ng/mL, greater than or equal to about 90 ng/mL, greater than or equal to about 100 ng/mL, greater than or equal to about 150 ng/mL, greater than or equal to about 200 ng/mL, greater than or equal to about 250 ng/mL, greater than or equal to about 300 ng/mL, greater than or equal to about 350 ng/mL, greater than or equal to about 400 ng/mL, greater than or equal to about 450 ng/mL, greater than or equal to about 500 ng/mL, or greater than or equal to about 550 ng/mL. In some aspects, the Cₘₐₓ per each 1 mg dosage of the compound administered is less than or equal to about 20 ng/mL, less than or equal to about 30 ng/mL, less than or equal to about 40 ng/mL, less than or equal to about 50 ng/mL, less than or equal to about 60 ng/mL, less than or equal to about 70 ng/mL, less than or equal to about 80 ng/mL, less than or equal to about 90 ng/mL, less than or equal to about 100 ng/mL, less than or equal to about 150 ng/mL, less than or equal to about 200 ng/mL, less than or equal to about 250 ng/mL, less than or equal to about 300 ng/mL, less than or equal to about 350 ng/mL, less than or equal to about 400 ng/mL, less than or equal to about 450 ng/mL, less than or equal to about 500 ng/mL, or less than or equal to about 550 ng/mL.

In other aspects, the Cₘₐₓ is at least 1 ng/mL; at least 5 ng/mL; at least 10 ng/mL; at least 15 ng/mL; at least 20 ng/mL; at least 25 ng/mL; at least 50 ng/mL; at least 75 ng/mL; at least 100 ng/mL; at least 200 ng/mL; at least 300 ng/mL; at least 400 ng/mL; at least 500 ng/mL; at least 600 ng/mL; at least 700 ng/mL; at least 800 ng/mL; at least 900 ng/mL; at least 1000 ng/mL; at least 1250 ng/mL; at least 1500 ng/mL; at least 1750 ng/mL; at least 2000 ng/mL; at least 2100 ng/mL; at least 2200 ng/mL; at least 2300 ng/mL; at least 2400 ng/mL; at least 2500 ng/mL; at least 2600 ng/mL; at least 2700 ng/mL; at least 2800 ng/mL; at least 2900 ng/mL; at least 3000 ng/mL; at least 3100 ng/mL; at least 32000 ng/mL; at least 3300 ng/mL; at least 3400 ng/mL; at least 3500 ng/mL; at least 3600 ng/mL; at least 3700 ng/mL; at least 3800 ng/mL; at least 3900 ng/mL; at least 4000 ng/mL; at least 4500 ng/mL; at least 5000 ng/mL; at least 5500 ng/mL; at least 6000 ng/mL; at least 6500 ng/mL; at least 2700 ng/mL; at least 7500 ng/mL; at least 8000 ng/mL; at least 8500 ng/mL; at least 9000 ng/mL; at least 9500 ng/mL; at least 10000 ng/mL; at least 11000 ng/mL; at least 12000 ng/mL; at least 13000 ng/mL; at least 14000 ng/mL; at least 15000 ng/mL; at least 16000 ng/mL; at least 17000 ng/mL; at least 18000 ng/mL; at least 19000 ng/mL; at least 20000 ng/mL; at least 25000 ng/mL; at least 30000 ng/mL; at least 35000 ng/mL; at least 40000 ng/mL; at least 45000 ng/mL; at least 50000 ng/mL; at least 55000 ng/mL; at least 60000 ng/mL; at least 65000 ng/mL; at least 70000 ng/mL; at least 750000 ng/mL; at least 80000 ng/mL; at least 85000 ng/mL; at least 90000 ng/mL; at least 95000 ng/mL; at least 100000 ng/mL; or any other Cₘₐₓ appropriate for describing a pharmacokinetic profile of a chlorotoxin conjugate described herein. The Cₘₐₓ is, for example, about 1 ng/mL to about 100,000 ng/mL; about 1 ng/mL to about 95,000 ng/mL; about 1 ng/mL to about 90,000 ng/mL; about 1 ng/mL to about 85,000 ng/mL; about 1 ng/mL to about 80,000 ng/mL; about 1 ng/mL to about 75,000 ng/mL; about 1 ng/mL to about 70,000 ng/mL; about 1 ng/mL to about 65,000 ng/mL; about 1 ng/mL to about 60,000 ng/mL; about 1 ng/mL to about 55,000 ng/mL; about 1 ng/mL to about 50,000 ng/mL; about 1 ng/mL to about 40,000 ng/mL; about 1 ng/mL to about 30,000 ng/mL; about 1 ng/mL to about 20,000 ng/mL; about 1 ng/mL to about 10,000 ng/mL; about 1 ng/mL to about 5,000 ng/mL; about 1 ng/mL to about 1,000 ng/mL; about 1 ng/mL to about 750 ng/mL; about 1 ng/mL to about 500 ng/mL; about 1 ng/mL to about 100 ng/mL; about 1 ng/mL to about 50 ng/mL; about 10 ng/mL to about 5,000 ng/mL; about 10 ng/mL to about 7,000 ng/mL; about 10 ng/mL to about 10,000 ng/mL; about 10 ng/mL to about 10,500 ng/mL; about 10 ng/mL to about 100,000 ng/mL; about 10 ng/mL to about 90,000 ng/mL; about 10 ng/mL to about 80,000 ng/mL; about 10 ng/mL to about 70,000 ng/mL; about 10 ng/mL to about 60,000 ng/mL; about 10 ng/mL to about 50,000 ng/mL; about 10 ng/mL to about 40,000 ng/mL; about 10 ng/mL to about 30,000 ng/mL; about 10 ng/mL to about 20,000 ng/mL; about 25,000 ng/mL to about 50,000 ng/mL; about 250 ng/mL to about 10,000 ng/mL; about 500 ng/mL to about 50,000 ng/mL; about 50 ng/mL to about 10,000 ng/mL; about 100 ng/mL to about 50,000 ng/mL; about 100 ng/mL to about 40,000 ng/mL; about 100 ng/mL to about 30,000 ng/mL; or about 100 ng/mL to about 20,000 ng/mL.

The plasma concentration of a chlorotoxin conjugate described herein can include, by way of example but is not limited to, at least 1 ng/mL, at least 2 ng/mL, at least 3 ng/mL, at least 4 ng/mL, at least 5 ng/mL, at least 6 ng/mL, at least 7 ng/mL, at least 8 ng/mL, at least 9 ng/mL, at least 10 ng/mL, at least 11 ng/mL, at least 12 ng/mL, at least 13 ng/mL, at least 14 ng/mL, at least 15 ng/mL, at least 16 ng/mL, at least 17 ng/mL, at least 18 ng/mL, at least 19 ng/mL, at least 20 ng/mL, at least 21 ng/mL, at least 22 ng/mL, at least 23 ng/mL, at least 24 ng/mL, at least 25 ng/mL, at least 26 ng/mL, at least 27 ng/mL, at least 28 ng/mL, at least 29 ng/mL, at least 30 ng/mL, at least 31 ng/mL, at least 32 ng/mL, at least 33 ng/mL, at least 34 ng/mL, at least 35 ng/mL, at least 36 ng/mL, at least 37 ng/mL, at least 38 ng/mL, at least 39 ng/mL, at least 40 ng/mL, at least 41 ng/mL, at least 42 ng/mL, at least 43 ng/mL, at least 44 ng/mL, at least 45 ng/mL, at least 46 ng/mL, at least 47 ng/mL, at least 48 ng/mL, at least 49 ng/mL, at least 50 ng/mL, at least 51 ng/mL, at least 52 ng/mL, at least 53 ng/mL, at least 54 ng/mL, at least 55 ng/mL, at least 56 ng/mL, at least 57 ng/mL, at least 58 ng/mL, at least 59 ng/mL, at least 60 ng/mL, at least 61 ng/mL, at least 62 ng/mL, at least 63 ng/mL, at least 64 ng/mL, at least 65 ng/mL, at least 66 ng/mL, at least 67 ng/mL, at least 68 ng/mL, at least 69 ng/mL, at least 70 ng/mL, at least 71 ng/mL, at least 72 ng/mL, at least 73 ng/mL, at least 74 ng/mL, at least 75 ng/mL, at least 76 ng/mL, at least 77 ng/mL, at least 78 ng/mL, at least 79 ng/mL, at least 80 ng/mL, at least 81 ng/mL, at least 82 ng/mL, at least 83 ng/mL, at least 84 ng/mL, at least 85 ng/mL, at least 86 ng/mL, at least 87 ng/mL, at least 88 ng/mL, at least 89 ng/mL, at least 90 ng/mL, at least 91 ng/mL, at least 92 ng/mL, at least 93 ng/mL, at least 94 ng/mL, at least 95 ng/mL, at least 96 ng/mL, at least 97 ng/mL, at least 98 ng/mL, at least 99 ng/mL, at least 100 ng/mL, at least 105 ng/mL, at least 110 ng/mL, at least 115 ng/mL, at least 120 ng/mL, at least 125 ng/mL, at least 130 ng/mL, at least 135 ng/mL, at least 140 ng/mL, at least 145 ng/mL, at least 150 ng/mL, at least 155 ng/mL, at least 160 ng/mL, at least 165 ng/mL, at least 170 ng/mL, at least 175 ng/mL, at least 180 ng/mL, at least 185 ng/mL, at least 190 ng/mL, at least 195 ng/mL, at least 200 ng/mL, at least 205 ng/mL, at least 210 ng/mL, at least 215 ng/mL, at least 220 ng/mL, at least 225 ng/mL, at least 230 ng/mL, at least 235 ng/mL, at least 240 ng/mL, at least 245 ng/mL, at least 250 ng/mL, at least 500 ng/mL, at least 750 ng/mL, at least 1,000 ng/mL, at least 2,000 ng/mL, at least 3,000 ng/mL, at least 4,000 ng/mL, at least 5,000 ng/mL, at least 10,000 ng/mL, at least 15,000 ng/mL, at least 20,000 ng/mL, at least 25,000 ng/mL, at least 30,000 ng/mL, at least 40,000 ng/mL, at least 50,000 ng/mL, or any other plasma concentration of a chlorotoxin conjugate described herein.

The plasma concentration can include, by way of example, but is not limited to, about 1 ng/mL to about 2 ng/mL; about 1 ng/mL to about 5 ng/mL; about 5 ng/mL to about 10 ng/mL; about 10 ng/mL to about 25 ng/mL; about 25 ng/mL to about 50 ng/mL; about 50 ng/mL to about 75 ng/mL; about 75 ng/mL to about 100 ng/mL; about 100 ng/mL to about 150 ng/mL; about 100 ng/mL to about 200 ng/mL about 150 ng/mL to about 200 ng/mL; about 200 ng/mL to about 250 ng/mL; about 250 ng/mL to about 300 ng/mL; about 300 ng/mL to about 350 ng/mL; about 350 ng/mL to about 400 ng/mL; about 400 ng/mL to about 450 ng/mL; about 450 ng/mL to about 500 ng/mL; about 500 ng/mL to about 600 ng/mL; about 600 ng/mL to about 700 ng/mL; about 700 ng/mL to about 800 ng/mL; about 800 ng/mL to about 900 ng/mL; about 900 ng/mL to about 1,000 ng/mL; about 1,000 ng/mL to about 1,100 ng/mL; about 1,100 ng/mL to about 1,200 ng/mL; about 1,200 ng/mL to about 1,300 ng/mL; about 1,300 ng/mL to about 1,400 ng/mL; about 1,400 ng/mL to about 1,500 ng/mL; about 1,500 ng/mL to about 1,600 ng/mL; about 1,600 ng/mL to about 1,700 ng/mL; about 1,700 ng/mL to about 1,800 ng/mL; about 1,800 ng/mL to about 1,900 ng/mL; about 1,900 ng/mL to about 2,000 ng/mL; about 2,000 ng/mL to about 3,000 ng/mL; about 3,000 ng/mL to about 4,000 ng/mL; about 4,000 ng/mL to about 5,000 ng/mL; about 5,000 ng/mL to about 6,000 ng/mL; about 6,000 ng/mL to about 7,000 ng/mL; about 7,000 ng/mL to about 8,000 ng/mL; about 8,000 ng/mL to about 9,000 ng/mL; or about 9,000 ng/mL to about 10,000 ng/mL.

In one aspect, the time (Tₘₐₓ) at which the Cₘₐₓ is reached is within a range from about 0.5 min to about 120 min following administration of the compound. In some aspects, the Tₘₐₓ of a chlorotoxin conjugate described herein includes by way of example but is not limited to, less than 0.5 minutes, less than 1 minute, less than 1.5 minutes, less than 2 minutes, less than 2.5 minutes, less than 3 minutes, less than 3.5 minutes, less than 4 minutes, less than 4.5 minutes, less than 5 minutes, less than 6 minutes, less than 7 minutes, less than 8 minutes, less than 9 minutes, less than 10 minutes, less than 15 minutes, less than 20 minutes, less than 25 minutes, less than 30 minutes, less than 40 minutes, less than 50 minutes, less than 60 minutes, or any other Tₘₐₓ appropriate for describing a pharmacokinetic profile of a chlorotoxin conjugate described herein. The Tₘₐₓ further can include, by way of example but is not limited to, about 0.1 minutes to about 24 minutes; about 0.1 minutes to about 0.5 minutes; about 0.5 minutes to about 1 minute; about 1 minute to about 1.5 minutes; about 1.5 minutes to about 2 minute; about 2 minutes to about 2.5 minutes; about 2.5 minutes to about 3 minutes; about 3 minutes to about 3.5 minutes; about 3.5 minutes to about 4 minutes; about 4 minutes to about 4.5 minutes; about 4.5 minutes to about 5 minutes; about 5 minutes to about 5.5 minutes; about 5.5 minutes to about 6 minutes; about 6 minutes to about 6.5 minutes; about 6.5 minutes to about 7 minutes; about 7 minutes to about 7.5 minutes; about 7.5 minutes to about 8 minutes; about 8 minutes to about 8.5 minutes; about 8.5 minutes to about 9 minutes; about 9 minutes to about 9.5 minutes; about 9.5 minutes to about 10 minutes; about 10 minutes to about 10.5 minutes; about 10.5 minutes to about 11 minutes; about 11 minutes to about 11.5 minutes; about 11.5 minutes to about 12 minutes; about 12 minutes to about 12.5 minutes; about 12.5 minutes to about 13 minutes; about 13 minutes to about 13.5 minutes; about 13.5 minutes to about 14 minutes; about 14 minutes to about 14.5 minutes; about 14.5 minutes to about 15 minutes; about 15 minutes to about 15.5 minutes; about 15.5 minutes to about 16 minutes; about 16 minutes to about 16.5 minutes; about 16.5 minutes to about 17 minutes; about 17 minutes to about 17.5 minutes; about 17.5 minutes to about 18 minutes; about 18 minutes to about 18.5 minutes; about 18.5 minutes to about 19 minutes; about 19 minutes to about 19.5 minutes; about 19.5 minutes to about 20 minutes; about 20 minutes to about 20.5 minutes; about 20.5 minutes to about 21 minutes; about 21 minutes to about 21.5 minutes; about 21.5 minutes to about 22 minutes; about 22 minutes to about 22.5 minutes; about 22.5 minutes to about 23 minutes; about 23 minutes to about 23.5 minutes; about 23.5 minutes to about 24 minutes; about 24 minutes to about 25 minutes; about 25 minutes to about 25.5 minutes; about 25.5 minutes to about 26 minutes; about 26 minutes to about 26.5 minutes; about 26.5 minutes to about 27 minutes; about 27 minutes to about 28 minutes; about 28 minutes to about 28.5 minutes; about 28.5 minutes to about 29 minutes; about 29 minutes to about 29.5 minutes; about 29.5 minutes to about 30 minutes; about 30 minutes to about 31 minutes; about 31 minutes to about 31.5 minutes; about 31.5 minutes to about 32 minutes; about 32 minutes to about 32.5 minutes; about 32.5 minutes to about 33 minutes; about 33 minutes to about 34 minutes; about 34 minutes to about 35 minutes; about 35 minutes to about 36 minutes; about 36 minutes to about 37 minutes; about 37 minutes to about 38 minutes; about 38 minutes to about 39 minutes; about 39 minutes to about 40 minutes; about 40 minutes to about 41 minutes; about 41 minutes to about 42 minutes; about 42 minutes to about 43 minutes; about 43 minutes to about 44 minutes; about 45 minutes to about 46 minutes; about 46 minutes to about 47 minutes; about 47 minutes to about 48 minutes; about 48 minutes to about 49 minutes; about 49 minutes to about 50 minutes; about 50 minutes to about 51 minutes; about 51 minutes to about 52 minutes; about 52 minutes to about 53 minutes; about 53 minutes to about 55 minutes; about 55 minutes to about 56 minutes; about 56 minutes to about 57 minutes; about 57 minutes to about 58 minutes; about 58 minutes to about 59 minutes; about 59 minutes to about 60 minutes; or any other Tₘₐₓ of a chlorotoxin conjugate described herein of a chlorotoxin conjugate described herein.

The Tₘₐₓ of a chlorotoxin conjugate described herein can include, by way of example, but is not limited to, less than 0.5 hours, less than 1 hours, less than 1.5 hours, less than 2 hours, less than 2.5 hours, less than 3 hours, less than 3.5 hours, less than 4 hours, less than 4.5 hours, less than 5 hours, or any other Tₘₐₓ appropriate for describing a pharmacokinetic profile of a chlorotoxin conjugate described herein. The Tₘₐₓ can further include, by way of example, but is not limited to, about 0.1 hours to about 24 hours; about 0.1 hours to about 0.5 hours; about 0.5 hours to about 1 hour; about 1 hour to about 1.5 hours; about 1.5 hours to about 2 hour; about 2 hours to about 2.5 hours; about 2.5 hours to about 3 hours; about 3 hours to about 3.5 hours; about 3.5 hours to about 4 hours; about 4 hours to about 4.5 hours; about 4.5 hours to about 5 hours; about 5 hours to about 5.5 hours; about 5.5 hours to about 6 hours; about 6 hours to about 6.5 hours; about 6.5 hours to about 7 hours; about 7 hours to about 7.5 hours; about 7.5 hours to about 8 hours; about 8 hours to about 8.5 hours; about 8.5 hours to about 9 hours; about 9 hours to about 9.5 hours; about 9.5 hours to about 10 hours; about 10 hours to about 10.5 hours; about 10.5 hours to about 11 hours; about 11 hours to about 11.5 hours; about 11.5 hours to about 12 hours; about 12 hours to about 12.5 hours; about 12.5 hours to about 13 hours; about 13 hours to about 13.5 hours; about 13.5 hours to about 14 hours; about 14 hours to about 14.5 hours; about 14.5 hours to about 15 hours; about 15 hours to about 15.5 hours; about 15.5 hours to about 16 hours; about 16 hours to about 16.5 hours; about 16.5 hours to about 17 hours; about 17 hours to about 17.5 hours; about 17.5 hours to about 18 hours; about 18 hours to about 18.5 hours; about 18.5 hours to about 19 hours; about 19 hours to about 19.5 hours; about 19.5 hours to about 20 hours; about 20 hours to about 20.5 hours; about 20.5 hours to about 21 hours; about 21 hours to about 21.5 hours; about 21.5 hours to about 22 hours; about 22 hours to about 22.5 hours; about 22.5 hours to about 23 hours; about 23 hours to about 23.5 hours; about 23.5 hours to about 24 hours; about 24 hours to about 25 hours; about 25 hours to about 25.5 hours; about 25.5 hours to about 26 hours; about 26 hours to about 26.5 hours; about 26.5 hours to about 27 hours; about 27 hours to about 28 hours; about 28 hours to about 28.5 hours; about 28.5 hours to about 29 hours; about 29 hours to about 29.5 hours; about 29.5 hours to about 30 hours; about 30 hours to about 31 hours; about 31 hours to about 31.5 hours; about 31.5 hours to about 32 hours; about 32 hours to about 32.5 hours; about 32.5 hours to about 33 hours; about 33 hours to about 34 hours; about 34 hours to about 35 hours; about 35 hours to about 36 hours; about 36 hours to about 37 hours; about 37 hours to about 38 hours; about 38 hours to about 39 hours; about 39 hours to about 40 hours; about 40 hours to about 41 hours; about 41 hours to about 42 hours; about 42 hours to about 43 hours; about 43 hours to about 44 hours; about 45 hours to about 46 hours; about 46 hours to about 47 hours; about 47 hours to about 48 hours; about 48 hours to about 49 hours; about 49 hours to about 50 hours; about 50 hours to about 51 hours; about 51 hours to about 52 hours; about 52 hours to about 53 hours; about 53 hours to about 55 hours; about 55 hours to about 56 hours; about 56 hours to about 57 hours; about 57 hours to about 58 hours; about 58 hours to about 59 hours; about 59 hours to about 60 hours; about 60 hours to about 61 hours; about 61 hours to about 62 hours; about 62 hours to about 63 hours; about 63 hours to about 64 hours; about 64 hours to about 66 hours; about 66 hours to about 67 hours; about 67 hours to about 68 hours; about 68 hours to about 69 hours; about 69 hours to about 70 hours; about 70 hours to about 71 hours; about 71 hours to about 72 hours; about 72 hours to about 73 hours; about 73 hours to about 74 hours; about 74 hours to about 75 hours; about 75 hours to about 77 hours; about 77 hours to about 78 hours; about 78 hours to about 79 hours; about 79 hours to about 80 hours; about 80 hours to about 81 hours; about 81 hours to about 82 hours; about 82 hours to about 83 hours; about 83 hours to about 84 hours; about 84 hours to about 85 hours; about 85 hours to about 87 hours; about 87 hours to about 88 hours; about 88 hours to about 89 hours; about 89 hours to about 90 hours; about 90 hours to about 91 hours; about 91 hours to about 92 hours; about 92 hours to about 93 hours; about 93 hours to about 94 hours; about 94 hours to about 95 hours; about 95 hours to about 97 hours; about 97 hours to about 99 hours; about 99 hours to about 100 hours; or any other Tₘₐₓ of a chlorotoxin conjugate described herein of a chlorotoxin conjugate described herein.

The elimination half-life (t_{1/2}) of a chlorotoxin conjugate described herein can include, by way of example, but is not limited to, greater than or equal to about 0.08 hr, greater than or equal to about 0.09 hr, greater than or equal to about 0.1 hr, greater than or equal to about 0.15 hr, greater than or equal to about 0.2 hr, greater than or equal to about 0.25 hr, greater than or equal to about 0.3 hr, greater than or equal to about 0.4 hr, greater than or equal to about 0.5 hr, greater than or equal to about 0.6 hr, greater than or equal to about 0.7 hr, greater than or equal to about 0.8 hr, greater than or equal to about 0.9 hr, greater than or equal to about 1 hr, greater than or equal to about 1.5 hr, greater than or equal to about 2 hr, or greater than or equal to about 2.5 hr. In some aspects, the t_{1/2} is less than or equal to about 0.08 hr, less than or equal to about 0.09 hr, less than or equal to about 0.1 hr, less than or equal to about 0.15 hr, less than or equal to about 0.2 hr, less than or equal to about 0.3 hr, less than or equal to about 0.4 hr, less than or equal to about 0.5 hr, less than or equal to about 0.6 hr, less than or equal to about 0.7 hr, less than or equal to about 0.8 hr, less than or equal to about 0.9 hr, less than or equal to about 1 hr, less than or equal to about 1.5 hr, less than or equal to about 2 hr, or less than or equal to about 2.5 hr. In some aspects, the t_{1/2} is less than 0.08 minutes, less than 0.1 minutes, less than 0.2 minutes, less than 0.4 minutes, less than 0.5 minutes, less than 1 minute, less than 1.5 minutes, less than 2 minutes, less than 2.5 minutes, less than 3 minutes, less than 3.5 minutes, less than 4 minutes, less than 4.5 minutes, less than 5 minutes, less than 6 minutes, less than 7 minutes, less than 8 minutes, less than 9 minutes, less than 10 minutes, less than 15 minutes, less than 20 minutes, less than 25 minutes, less than 30 minutes, less than 40 minutes, less than 50 minutes, less than 60 minutes, or any other t_{1/2} appropriate for describing a pharmacokinetic profile of a chlorotoxin conjugate described herein. The t_{1/2} further can include, by way of example, but is not limited to, about 0.08 minutes about 0.1 minutes to about 24 minutes; about 0.1 minutes to about 0.5 minutes; about 0.5 minutes to about 1 minute; about 1 minute to about 1.5 minutes; about 1.5 minutes to about 2 minute; about 2 minutes to about 2.5 minutes; about 2.5 minutes to about 3 minutes; about 3 minutes to about 3.5 minutes; about 3.5 minutes to about 4 minutes; about 4 minutes to about 4.5 minutes; about 4.5 minutes to about 5 minutes; about 5 minutes to about 5.5 minutes; about 5.5 minutes to about 6 minutes; about 6 minutes to about 6.5 minutes; about 6.5 minutes to about 7 minutes; about 7 minutes to about 7.5 minutes; about 7.5 minutes to about 8 minutes; about 8 minutes to about 8.5 minutes; about 8.5 minutes to about 9 minutes; about 9 minutes to about 9.5 minutes; about 9.5 minutes to about 10 minutes; about 10 minutes to about 10.5 minutes; about 10.5 minutes to about 11 minutes; about 11 minutes to about 11.5 minutes; about 11.5 minutes to about 12 minutes; about 12 minutes to about 12.5 minutes; about 12.5 minutes to about 13 minutes; about 13 minutes to about 13.5 minutes; about 13.5 minutes to about 14 minutes; about 14 minutes to about 14.5 minutes; about 14.5 minutes to about 15 minutes; about 15 minutes to about 15.5 minutes; about 15.5 minutes to about 16 minutes; about 16 minutes to about 16.5 minutes; about 16.5 minutes to about 17 minutes; about 17 minutes to about 17.5 minutes; about 17.5 minutes to about 18 minutes; about 18 minutes to about 18.5 minutes; about 18.5 minutes to about 19 minutes; about 19 minutes to about 19.5 minutes; about 19.5 minutes to about 20 minutes; about 20 minutes to about 20.5 minutes; about 20.5 minutes to about 21 minutes; about 21 minutes to about 21.5 minutes; about 21.5 minutes to about 22 minutes; about 22 minutes to about 22.5 minutes; about 22.5 minutes to about 23 minutes; about 23 minutes to about 23.5 minutes; about 23.5 minutes to about 24 minutes; about 24 minutes to about 25 minutes; about 25 minutes to about 25.5 minutes; about 25.5 minutes to about 26 minutes; about 26 minutes to about 26.5 minutes; about 26.5 minutes to about 27 minutes; about 27 minutes to about 28 minutes; about 28 minutes to about 28.5 minutes; about 28.5 minutes to about 29 minutes; about 29 minutes to about 29.5 minutes; about 29.5 minutes to about 30 minutes; about 30 minutes to about 31 minutes; about 31 minutes to about 31.5 minutes; about 31.5 minutes to about 32 minutes; about 32 minutes to about 32.5 minutes; about 32.5 minutes to about 33 minutes; about 33 minutes to about 34 minutes; about 34 minutes to about 35 minutes; about 35 minutes to about 36 minutes; about 36 minutes to about 37 minutes; about 37 minutes to about 38 minutes; about 38 minutes to about 39 minutes; about 39 minutes to about 40 minutes; about 40 minutes to about 41 minutes; about 41 minutes to about 42 minutes; about 42 minutes to about 43 minutes; about 43 minutes to about 44 minutes; about 45 minutes to about 46 minutes; about 46 minutes to about 47 minutes; about 47 minutes to about 48 minutes; about 48 minutes to about 49 minutes; about 49 minutes to about 50 minutes; about 50 minutes to about 51 minutes; about 51 minutes to about 52 minutes; about 52 minutes to about 53 minutes; about 53 minutes to about 55 minutes; about 55 minutes to about 56 minutes; about 56 minutes to about 57 minutes; about 57 minutes to about 58 minutes; about 58 minutes to about 59 minutes; about 59 minutes to about 60 minutes; or any other t_{1/2} of a chlorotoxin conjugate described herein of a chlorotoxin conjugate described herein.

The t_{1/2} of a chlorotoxin conjugate described herein can include, by way of example, but is not limited to, less than 0.5 hours, less than 1 hours, less than 1.5 hours, less than 2 hours, less than 2.5 hours, less than 3 hours, less than 3.5 hours, less than 4 hours, less than 4.5 hours, less than 5 hours, or any other t_{1/2} appropriate for describing a pharmacokinetic profile of a chlorotoxin conjugate described herein. The t_{1/2} can further include, by way of example, but is not limited to, about 0.1 hours to about 10 hours; about 0.1 hours to about 0.5 hours; about 0.15 hours to about 10 hours; about 0.15 hours to about 5 hours; about 0.15 hours to about 4 hours; about 0.15 hours to about 3 hours; about 0.15 hours to about 2 hours; about 0.15 hours to about 1 hour; about 0.5 hours to about 1 hour; about 1 hour to about 1.5 hours; about 1.5 hours to about 2 hour; about 2 hours to about 2.5 hours; about 2.5 hours to about 3 hours; about 3 hours to about 3.5 hours; about 3.5 hours to about 4 hours; about 4 hours to about 4.5 hours; about 4.5 hours to about 5 hours; about 5 hours to about 5.5 hours; about 5.5 hours to about 6 hours; about 6 hours to about 6.5 hours; about 6.5 hours to about 7 hours; about 7 hours to about 7.5 hours; about 7.5 hours to about 8 hours; about 8 hours to about 8.5 hours; about 8.5 hours to about 9 hours; about 9 hours to about 9.5 hours; about 9.5 hours to about 10 hours; about 10 hours to about 10.5 hours; about 10.5 hours to about 11 hours; about 11 hours to about 11.5 hours; about 11.5 hours to about 12 hours; about 12 hours to about 12.5 hours; about 12.5 hours to about 13 hours; about 13 hours to about 13.5 hours; about 13.5 hours to about 14 hours; about 14 hours to about 14.5 hours; about 14.5 hours to about 15 hours; about 15 hours to about 15.5 hours; about 15.5 hours to about 16 hours; about 16 hours to about 16.5 hours; about 16.5 hours to about 17 hours; about 17 hours to about 17.5 hours; about 17.5 hours to about 18 hours; about 18 hours to about 18.5 hours; about 18.5 hours to about 19 hours; about 19 hours to about 19.5 hours; about 19.5 hours to about 20 hours; about 20 hours to about 20.5 hours; about 20.5 hours to about 21 hours; about 21 hours to about 21.5 hours; about 21.5 hours to about 22 hours; about 22 hours to about 22.5 hours; about 22.5 hours to about 23 hours; about 23 hours to about 23.5 hours; about 23.5 hours to about 24 hours; about 24 hours to about 25 hours; about 25 hours to about 25.5 hours; about 25.5 hours to about 26 hours; about 26 hours to about 26.5 hours; about 26.5 hours to about 27 hours; about 27 hours to about 28 hours; about 28 hours to about 28.5 hours; about 28.5 hours to about 29 hours; about 29 hours to about 29.5 hours; about 29.5 hours to about 30 hours; about 30 hours to about 31 hours; about 31 hours to about 31.5 hours; about 31.5 hours to about 32 hours; about 32 hours to about 32.5 hours; about 32.5 hours to about 33 hours; about 33 hours to about 34 hours; about 34 hours to about 35 hours; about 35 hours to about 36 hours; about 36 hours to about 37 hours; about 37 hours to about 38 hours; about 38 hours to about 39 hours; about 39 hours to about 40 hours; about 40 hours to about 41 hours; about 41 hours to about 42 hours; about 42 hours to about 43 hours; about 43 hours to about 44 hours; about 45 hours to about 46 hours; about 46 hours to about 47 hours; about 47 hours to about 48 hours; about 48 hours to about 49 hours; about 49 hours to about 50 hours; about 50 hours to about 51 hours; about 51 hours to about 52 hours; about 52 hours to about 53 hours; about 53 hours to about 55 hours; about 55 hours to about 56 hours; about 56 hours to about 57 hours; about 57 hours to about 58 hours; about 58 hours to about 59 hours; about 59 hours to about 60 hours; about 60 hours to about 61 hours; about 61 hours to about 62 hours; about 62 hours to about 63 hours; about 63 hours to about 64 hours; about 64 hours to about 66 hours; about 66 hours to about 67 hours; about 67 hours to about 68 hours; about 68 hours to about 69 hours; about 69 hours to about 70 hours; about 70 hours to about 71 hours; about 71 hours to about 72 hours; about 72 hours to about 73 hours; about 73 hours to about 74 hours; about 774 hours to about 75 hours; about 75 hours to about 77 hours; about 77 hours to about 78 hours; about 78 hours to about 79 hours; about 79 hours to about 80 hours; about 80 hours to about 81 hours; about 81 hours to about 82 hours; about 82 hours to about 83 hours; about 83 hours to about 84 hours; about 84 hours to about 85 hours; about 85 hours to about 87 hours; about 87 hours to about 88 hours; about 88 hours to about 89 hours; about 89 hours to about 90 hours; about 90 hours to about 91 hours; about 91 hours to about 92 hours; about 92 hours to about 93 hours; about 93 hours to about 94 hours; about 94 hours to about 95 hours; about 95 hours to about 97 hours; about 97 hours to about 99 hours; about 99 hours to about 100 hours; or any other t_{1/2} of a chlorotoxin conjugate described herein of a chlorotoxin conjugate described herein.

In some aspects, the chlorotoxin conjugates distribute into the subject tissues. For example, distribution into the tissues is often rapid compared to the elimination phase. In some aspects, the chlorotoxin conjugates are eliminated from the subject tissues. For example, elimination from the subject tissues is often slow compared to the distribution phase. Often the kidney can be important in the clearance and elimination of the chlorotoxin conjugates, often contributing to the elimination phase.

The clearance (CL) per each 1 mg dosage of the compound administered can include, by way of example, but is not limited to, is 2,000 mL/hr, 4,000 mL/hr, 6,000 mL/hr, 8,000 mL/hr, 10,000 mL/hour, 15,000 mL/hr, 20,000 mL/hr, 25,000 mL/hr, 30,000 mL/hr, 35,000 mL/hr, 40,000 mL/hr, 45,000 mL/hr, or 50,000 mL/hr. In some aspects, the CL per each 1 mg dosage of compound administered is between the range of 2,000 mL/hr to 100,000 mL/hr. In other aspects, the CL per 1 mg dosage of compound administered is 60,000 mL/hr, 70,000 mL/hr, 80,000 mL/hr, 90,000 mL/hr, or 100,000 mL/hr.

The volume of distribution (V_{d}) per each 1 mg dosage of the compound administered can include, by way of example, but is not limited to, 200 mL, 300 mL, 400 mL, 500 mL, 1,000 mL, 1,500 mL, 2,000 mL, 2,500 mL, 3,000 mL, 4,000 mL, 5,000 mL, 6,000 mL, 7,000 mL, 8,000 mL, 9,000 mL, or 10,000 mL. In some aspects, the V_{d} per each 1 mg dosage of the compound administered is between the range of 200 mL to 20,000 mL. In certain aspects, the V_{d} per each 1 mg dosage of the compound administered is 11,000 mL, 12,000 mL, 13,000 mL, 14,000 mL, 15,000 mL, 16,000 mL, 17,000 mL, 18,000 mL, 19,000 mL, or 20,000 mL.

The pharmacokinetics parameters can be any parameters suitable for describing the plasma profiles of chlorotoxin conjugates described herein and can often be associated with a curve. As described elsewhere herein, dose can be either scaled or fixed, said scaled dose useful for scaling the dose from one subject to another wherein the subjects are the same species, different species, same sex or different sex. The phases of the curve can often be representative of data obtained from at least one subject, sometimes more than one subject, and the phases of the curve and/or data of the curve can often be scaled in a manner similar to the manner in which doses are scaled.

In some aspects, the curve is plotted on a graph, often a graph with an x-axis and a y-axis referred to for example as an x-y plot, a scatter plot or the like. In one embodiment, each axis of the graph has units, the x-axis often having units of time, for example in minutes or hours, and y-axis often having units of concentration in a log scale, for example as ng/Ml or micromolar, of a chlorotoxin conjugate described herein present in a subject sample as described herein and are representative of a single measurement, a mean, an average, or any other suitable mathematical calculation performed on a set of data. When a suitable mathematical calculation is performed, a statistic can also be calculated, for example, a standard error, standard error of the mean, standard deviation, standard deviation of the mean, or any other suitable statistic useful for the described disclosure.

In some aspects, the curve has phases, for example, distribution phase, metabolism phase, and elimination phase. In some aspects, the distribution phase begins at time of about 0 hours and extends until a time of about 0.01 hours, about 0.02 hours, about 0.03 hours, about 0.04 hours, about 0.05 hours, about 0.06 hours, about 0.07 hours, about 0.08 hours, about 0.09 hours, about 0.11 hours, about 0.12 hours, about 0.13 hours, about 0.14 hours, about 0.15 hours, about 0.16 hours, about 0.17 hours, about 0.18 hours, about 0.19 hours, about 0.20 hours, 0.21 hours, about 0.22 hours, about 0.23 hours, about 0.24 hours, about 0.25 hours, about 0.26 hours, about 0.27 hours, about 0.28 hours, about 0.29 hours, about 0.30 hours, about 0.31 hours, about 0.32 hours, about 0.33 hours, about 0.34 hours, about 0.35 hours, about 0.36 hours, about 0.37 hours, about 0.38 hours, about 0.39 hours, about 0.40 hours, about 0.41 hours, about 0.42 hours, about 0.43 hours, about 0.44 hours, about 0.45 hours, about 0.46 hours, about 0.47 hours, about 0.48 hours, about 0.49 hours, about 0.50 hours, about 0.51 hours, about 0.52 hours, about 0.53 hours, about 0.54 hours, about 0.55 hours, about 0.56 hours, about 0.57 hours, about 0.58 hours, about 0.59 hours, about 0.60 hours, about 0.61 hours, about 0.62 hours, about 0.63 hours, about 0.64 hours, about 0.65 hours, about 0.66 hours, about 0.67 hours, about 0.68 hours, about 0.69 hours, about 0.70 hours, about 0.71 hours, about 0.72 hours, about 0.73 hours, about 0.74 hours, about 0.75 hours, about 0.76 hours, about 0.77 hours, about 0.78 hours, about 0.79 hours, about 0.80 hours, about 0.81 hours, about 0.82 hours, about 0.83 hours, about 0.84 hours, about 0.85 hours, about 0.86 hours, about 0.87 hours, about 0.88 hours, about 0.89 hours, about 0.90 hours, about 0.91 hours, about 0.92 hours, about 0.93 hours, about 0.94 hours, about 0.95 hours, about 0.96 hours, about 0.97 hours, about 0.98 hours, about 0.99 hours, about 1.00 hours, about 1.01 hours, about 1.02 hours, about 1.03 hours, about 1.04 hours, about 1.05 hours, about 1.06 hours, about 1.07 hours, about 1.08 hours, about 1.09 hours, about 1.11 hours, about 1.12 hours, about 1.13 hours, about 1.14 hours, about 1.15 hours, about 1.16 hours, about 1.17 hours, about 1.18 hours, about 1.19 hours, about 1.20 hours, 1.21 hours, about 1.22 hours, about 1.23 hours, about 1.24 hours, about 1.25 hours, about 1.26 hours, about 1.27 hours, about 1.28 hours, about 1.29 hours, about 1.30 hours, about 1.31 hours, about 1.32 hours, about 1.33 hours, about 1.34 hours, about 1.35 hours, about 1.36 hours, about 1.37 hours, about 1.38 hours, about 1.39 hours, about 1.40 hours, about 1.41 hours, about 1.42 hours, about 1.43 hours, about 1.44 hours, about 1.45 hours, about 1.46 hours, about 1.47 hours, about 1.48 hours, about 1.49 hours, about 1.50 hours, about 1.51 hours, about 1.52 hours, about 1.53 hours, about 1.54 hours, about 1.55 hours, about 1.56 hours, about 1.57 hours, about 1.58 hours, about 1.59 hours, about 1.60 hours, about 1.61 hours, about 1.62 hours, about 1.63 hours, about 1.64 hours, about 1.65 hours, about 1.66 hours, about 1.67 hours, about 1.68 hours, about 1.69 hours, about 1.70 hours, about 1.71 hours, about 1.72 hours, about 1.73 hours, about 1.74 hours, about 1.75 hours, about 1.76 hours, about 1.77 hours, about 1.78 hours, about 1.79 hours, about 1.80 hours, about 1.81 hours, about 1.82 hours, about 1.83 hours, about 1.84 hours, about 1.85 hours, about 1.86 hours, about 1.87 hours, about 1.88 hours, about 1.89 hours, about 1.90 hours, about 1.91 hours, about 1.92 hours, about 1.93 hours, about 1.94 hours, about 1.95 hours, about 1.96 hours, about 1.97 hours, about 1.98 hours, about 1.99 hours, about 2.00 hours, about 2.20 hours, about 2.40 hours, about 2.60 hours, about 2.80 hours, about 3.00 hours, about 4.20 hours, about 4.40 hours, about 4.60 hours, about 4.80 hours, about 5.00 hours, about 5.20 hours, about 5.40 hours, about 5.60 hours, about 5.80 hours, about 6.00 hours, about 6.20 hours, about 6.40 hours, about 6.60 hours, about 6.80 hours, about 7.00 hours, about 7.20 hours, about 7.40 hours, about 7.60 hours, about 7.80 hours, about 8.00 hours, about 8.20 hours, about 8.40 hours, about 8.60 hours, about 8.80 hours, about 9.00 hours, about 9.20 hours, about 9.40 hours, about 9.60 hours, about 9.80 hours, about 10.00 hours or more than about 10.00 hours.

In some aspects, the metabolism phase begins at time of about 0.5 hours and extends until a time of about 0.50 hours, about 0.51 hours, about 0.52 hours, about 0.53 hours, about 0.54 hours, about 0.55 hours, about 0.56 hours, about 0.57 hours, about 0.58 hours, about 0.59 hours, about 0.60 hours, about 0.61 hours, about 0.62 hours, about 0.63 hours, about 0.64 hours, about 0.65 hours, about 0.66 hours, about 0.67 hours, about 0.68 hours, about 0.69 hours, about 0.70 hours, about 0.71 hours, about 0.72 hours, about 0.73 hours, about 0.74 hours, about 0.75 hours, about 0.76 hours, about 0.77 hours, about 0.78 hours, about 0.79 hours, about 0.80 hours, about 0.81 hours, about 0.82 hours, about 0.83 hours, about 0.84 hours, about 0.85 hours, about 0.86 hours, about 0.87 hours, about 0.88 hours, about 0.89 hours, about 0.90 hours, about 0.91 hours, about 0.92 hours, about 0.93 hours, about 0.94 hours, about 0.95 hours, about 0.96 hours, about 0.97 hours, about 0.98 hours, about 0.99 hours, about 1.00 hours, about 1.01 hours, about 1.02 hours, about 1.03 hours, about 1.04 hours, about 1.05 hours, about 1.06 hours, about 1.07 hours, about 1.08 hours, about 1.09 hours, about 1.11 hours, about 1.12 hours, about 1.13 hours, about 1.14 hours, about 1.15 hours, about 1.16 hours, about 1.17 hours, about 1.18 hours, about 1.19 hours, about 1.20 hours, 1.21 hours, about 1.22 hours, about 1.23 hours, about 1.24 hours, about 1.25 hours, about 1.26 hours, about 1.27 hours, about 1.28 hours, about 1.29 hours, about 1.30 hours, about 1.31 hours, about 1.32 hours, about 1.33 hours, about 1.34 hours, about 1.35 hours, about 1.36 hours, about 1.37 hours, about 1.38 hours, about 1.39 hours, about 1.40 hours, about 1.41 hours, about 1.42 hours, about 1.43 hours, about 1.44 hours, about 1.45 hours, about 1.46 hours, about 1.47 hours, about 1.48 hours, about 1.49 hours, about 1.50 hours, about 1.51 hours, about 1.52 hours, about 1.53 hours, about 1.54 hours, about 1.55 hours, about 1.56 hours, about 1.57 hours, about 1.58 hours, about 1.59 hours, about 1.60 hours, about 1.61 hours, about 1.62 hours, about 1.63 hours, about 1.64 hours, about 1.65 hours, about 1.66 hours, about 1.67 hours, about 1.68 hours, about 1.69 hours, about 1.70 hours, about 1.71 hours, about 1.72 hours, about 1.73 hours, about 1.74 hours, about 1.75 hours, about 1.76 hours, about 1.77 hours, about 1.78 hours, about 1.79 hours, about 1.80 hours, about 1.81 hours, about 1.82 hours, about 1.83 hours, about 1.84 hours, about 1.85 hours, about 1.86 hours, about 1.87 hours, about 1.88 hours, about 1.89 hours, about 1.90 hours, about 1.91 hours, about 1.92 hours, about 1.93 hours, about 1.94 hours, about 1.95 hours, about 1.96 hours, about 1.97 hours, about 1.98 hours, about 1.99 hours, about 2.00 hours, about 2.20 hours, about 2.40 hours, about 2.60 hours, about 2.80 hours, about 3.00 hours, about 4.20 hours, about 4.40 hours, about 4.60 hours, about 4.80 hours, about 5.00 hours, about 5.20 hours, about 5.40 hours, about 5.60 hours, about 5.80 hours, about 6.00 hours, about 6.20 hours, about 6.40 hours, about 6.60 hours, about 6.80 hours, about 7.00 hours, about 7.20 hours, about 7.40 hours, about 7.60 hours, about 7.80 hours, about 8.00 hours, about 8.20 hours, about 8.40 hours, about 8.60 hours, about 8.80 hours, about 9.00 hours, about 9.20 hours, about 9.40 hours, about 9.60 hours, about 9.80 hours, about 10.00 hours, about 10.20 hours, about 10.40 hours, about 10.60 hours, about 10.80 hours, about 12.00 hours, about 12.20 hours, about 12.40 hours, about 12.60 hours, about 12.80 hours, about 14.00 hours, about 14.20 hours, about 14.40 hours, about 14.60 hours, about 14.80 hours, about 16.00 hours, about 16.20 hours, about 16.40 hours, about 16.60 hours, about 16.80 hours, about 18.00 hours, about 18.20 hours, about 18.40 hours, about 18.60 hours, about 18.80 hours, about 20.00 hours, about 20.20 hours, about 20.40 hours, about 20.60 hours, about 20.80 hours, about 22.00 hours, about 22.20 hours, about 22.40 hours, about 22.60 hours, about 22.80 hours, about 24.00 hours, about 24.20 hours, about 24.40 hours, about 24.60 hours, about 24.80 hours, about 26.00 hours, about 26.20 hours, about 26.40 hours, about 26.60 hours, about 26.80 hours, about 28.00 hours, about 28.20 hours, about 28.40 hours, about 28.60 hours, about 28.80 hours, about 30 hours or more than about 30.00 hours.

In some aspects, the elimination phase begins at time of about 2 hours and extends until a time of about 2.00 hours, about 2.20 hours, about 2.40 hours, about 2.60 hours, about 2.80 hours, about 3.00 hours, about 4.20 hours, about 4.40 hours, about 4.60 hours, about 4.80 hours, about 5.00 hours, about 5.20 hours, about 5.40 hours, about 5.60 hours, about 5.80 hours, about 6.00 hours, about 6.20 hours, about 6.40 hours, about 6.60 hours, about 6.80 hours, about 7.00 hours, about 7.20 hours, about 7.40 hours, about 7.60 hours, about 7.80 hours, about 8.00 hours, about 8.20 hours, about 8.40 hours, about 8.60 hours, about 8.80 hours, about 9.00 hours, about 9.20 hours, about 9.40 hours, about 9.60 hours, about 9.80 hours, about 10.00 hours, about 10.20 hours, about 10.40 hours, about 10.60 hours, about 10.80 hours, about 12.00 hours, about 12.20 hours, about 12.40 hours, about 12.60 hours, about 12.80 hours, about 14.00 hours, about 14.20 hours, about 14.40 hours, about 14.60 hours, about 14.80 hours, about 16.00 hours, about 16.20 hours, about 16.40 hours, about 16.60 hours, about 16.80 hours, about 18.00 hours, about 18.20 hours, about 18.40 hours, about 18.60 hours, about 18.80 hours, about 20.00 hours, about 20.20 hours, about 20.40 hours, about 20.60 hours, about 20.80 hours, about 22.00 hours, about 22.20 hours, about 22.40 hours, about 22.60 hours, about 22.80 hours, about 24.00 hours, about 24.20 hours, about 24.40 hours, about 24.60 hours, about 24.80 hours, about 26.00 hours, about 26.20 hours, about 26.40 hours, about 26.60 hours, about 26.80 hours, about 28.00 hours, about 28.20 hours, about 28.40 hours, about 28.60 hours, about 28.80 hours, about 30.00 hours, about 30.20 hours, about 30.40 hours, about 30.60 hours, about 30.80 hours, about 32.00 hours, about 32.20 hours, about 32.40 hours, about 32.60 hours, about 32.80 hours, about 34.00 hours, about 34.20 hours, about 34.40 hours, about 34.60 hours, about 34.80 hours, about 36.00 hours, about 36.20 hours, about 36.40 hours, about 36.60 hours, about 36.80 hours, about 38.00 hours, about 38.20 hours, about 38.40 hours, about 38.60 hours, about 38.80 hours, about 40.00 hours, about 40.20 hours, about 40.40 hours, about 40.60 hours, about 40.80 hours, about 42.00 hours, about 42.20 hours, about 42.40 hours, about 42.60 hours, about 42.80 hours, about 44.00 hours, about 44.20 hours, about 44.40 hours, about 44.60 hours, about 44.80 hours, about 46.00 hours, about 46.20 hours, about 46.40 hours, about 46.60 hours, about 46.80 hours, about 48.00 hours, about 48.20 hours, about 48.40 hours, about 48.60 hours, about 48.80 hours, about 50.00 hours, about 50.20 hours, about 50.40 hours, about 50.60 hours, about 50.80 hours, about 52.00 hours, about 52.20 hours, about 52.40 hours, about 52.60 hours, about 52.80 hours, about 54.00 hours, about 54.20 hours, about 54.40 hours, about 54.60 hours, about 54.80 hours, about 56.00 hours, about 56.20 hours, about 56.40 hours, about 56.60 hours, about 56.80 hours, about 58.00 hours, about 58.20 hours, about 58.40 hours, about 58.60 hours, about 58.80 hours, about 60.00 hours, about 60.20 hours, about 60.40 hours, about 60.60 hours, about 60.80 hours, about 62.00 hours, about 62.20 hours, about 62.40 hours, about 62.60 hours, about 62.80 hours, about 64.00 hours, about 64.20 hours, about 64.40 hours, about 64.60 hours, about 64.80 hours, about 66.00 hours, about 66.20 hours, about 66.40 hours, about 66.60 hours, about 66.80 hours, about 68.00 hours, about 68.20 hours, about 68.40 hours, about 68.60 hours, about 68.80 hours, about 70.00 hours, about 70.20 hours, about 70.40 hours, about 70.60 hours, about 70.80 hours, about 72.00 hours, about 72.20 hours, about 72.40 hours, about 72.60 hours, about 72.80 hours, about 74.00 hours, about 74.20 hours, about 74.40 hours, about 74.60 hours, about 74.80 hours, about 76.00 hours, about 76.20 hours, about 76.40 hours, about 76.60 hours, about 76.80 hours, about 78.00 hours, about 78.20 hours, about 78.40 hours, about 78.60 hours, about 78.80 hours, about 80.00 hours, about 80.20 hours, about 80.40 hours, about 80.60 hours, about 80.80 hours, about 82.00 hours, about 82.20 hours, about 82.40 hours, about 82.60 hours, about 82.80 hours, about 84.00 hours, about 84.20 hours, about 84.40 hours, about 84.60 hours, about 84.80 hours, about 86.00 hours, about 86.20 hours, about 86.40 hours, about 86.60 hours, about 86.80 hours, about 88.00 hours, about 88.20 hours, about 88.40 hours, about 88.60 hours, about 88.80 hours, about 90.00 hours or about more than 90.00 hours.

In some aspects, a single fixed bolus dose intravenous chlorotoxin conjugate often results in mean serum concentrations measurable up to about 1-2 hours post-dose, about 24 hours post-dose, up to about 36 hours post-dose, up to about 48 hours post-dose or more than about 48 hours post-dose. In certain aspects, for subjects such as rats, Cₘₐₓ and C₀ parameters increase in about a dose-proportional manner. In some aspects, the AUC₀₋ₜ parameter, for subjects such as rats, is about dose-proportional at less than about a 1 mg dose levels, and increases in a greater than dose-proportional manner at greater than about 1 mg dose levels. Often there can be no effect of gender on any PK parameters for subjects such as rats. In some aspects, PK parameters are predictive in rats of a human subject.

In some aspects, a single fixed bolus dose intravenous chlorotoxin conjugate often results in mean serum concentrations measurable up to about 12 hours post-dose, about 24 hours post-dose, up to about 36 hours post-dose, up to about 48 hours post-dose or more than about 48 hours post-dose. In certain aspects, for subjects such as rats, Cₘₐₓ and C₀ parameters increase in about a dose-proportional manner. In some aspects, the AUC₀₋ₜ parameter, for subjects such as monkeys, is greater than dose-proportional manner at greater than about 1 mg dose levels for example such that chlorotoxin conjugates exhibit reduced clearance at higher doses in monkeys. In certain aspects, there is an effect of gender on PK parameters for subjects such as monkeys, for example, the C₀ and AUC are about 5 to about 30% higher in females relative to males.

In some aspects, a single fixed intravenous bolus dose of chlorotoxin conjugate often results in mean serum concentrations measurable up to about 0.5 hours post-dose, up to about 1 hour post-dose, up to about 2 hours post-dose, up to about 12 hours post-dose, up to about 24 hours post-dose, up to about 36 hours post-dose, up to about 48 hours post-dose, or more than about 48 hours post-dose. In certain aspects, for subjects such as humans, AUC₀₋ₜ and Cₘₐₓ parameters increase in about a dose-proportional manner, and t_{1/2} parameter is consistent between dose levels. In other aspects, for subjects such as humans, AUC₀₋ₜ, Cₘₐₓ, and t_{1/2} parameters increase in a greater than a dose-proportional manner with increasing dosage.

In some aspects, a single fixed intravenous slow-bolus dose of chlorotoxin conjugate often results in mean serum concentrations measurable up to about 12 hours post-dose, up to about 24 hours post-dose, up to about 36 hours post-dose, up to about 48 hours post-dose, or more than about 48 hours post-dose. In other aspects, for subjects such as humans, AUC₀₋ₜand Cₘₐₓ parameters increase in about a dose-proportional manner, and t_{1/2} parameter is consistent between dose levels. In other aspects, for subjects such as humans, AUC₀₋ₜ, Cₘₐₓ, and t_{1/2} parameters increase in a greater than dose-proportional manner with increasing dosage.

In some aspects, a single fixed intravenous infusion dose of chlorotoxin conjugate often results in mean serum concentrations measurable up to about 12 hours post-dose, about 24 hours post-dose, up to about 36 hours post-dose, up to about 48 hours post-dose or more than about 48 hours post-dose. In certain aspects, for subjects such as humans, AUC₀₋ₜ and Cₘₐₓ parameters increase in about a dose-proportional manner, and t_{1/2} parameter is consistent between dose levels. In other aspects, for subjects such as humans, AUC₀₋ₜ, Cₘₐₓ and t_{1/2} parameters increase in a greater than dose-proportional manner with increasing dosage.

As used herein, two pharmacokinetic profiles are “about equivalent” if they are defined by at least one parameter that is about equivalent between the two profiles. Non-limiting examples of such parameters can include the area under plasma concentration over time curve (AUC) and the maximal plasma concentration reached following administration of a dose (Cₘₐₓ).

In some aspects two pharmacokinetic parameters are about equivalent if the lower value is greater than 70%, greater than 75%, greater than 80%, greater than 85%, greater than 90%, greater than 95%, greater than 96%, greater than 97%, greater than 98%, or greater than 99% of the higher value.

The pharmacokinetic profiles of two dosage regimens can be compared by determining the average pharmacokinetic profile in a population of subjects receiving the first dosage regimen, determining the average pharmacokinetic profile in a population of subjects receiving the second dosage regimen, and then comparing those two population dosage regimens. In some aspects, a population of subjects is one subject. In other aspects, a population of subjects is more than one subject, for example, two subjects, three subjects, four subjects, five subjects, six subjects, seven subjects, eight subjects, nine subjects, ten subjects, 11 subjects, 12 subjects, 13 subjects, 14 subjects, 15 subjects, 20 subjects, 25 subjects, 30 subjects, 35 subjects, 40 subjects, 45 subjects, 50 subjects, or more than 50 subjects.

In some aspects, the compound comprises any suitable compound of the present disclosure.

In various aspects, the present disclosure provides a method for detecting a cancer cell in a subject, the method comprising: administering any suitable compound of the present disclosure; and detecting the presence or absence of the compound in the subject, wherein the presence of the compound indicates the presence of a cancer cell.

In some aspects, the method further comprises administering the compound as a part of a composition.

In some aspects, the cancer cell is from a cancer, in which the cancer is selected from glioma, astrocytoma, medulloblastoma, choroids plexus carcinoma, ependymoma, brain tumor, neuroblastoma, adenocarcinoma, basal cell carcinoma, squamous cell carcinoma, head and neck cancer, lung cancer, breast cancer, intestinal cancer, pancreatic cancer, liver cancer, kidney cancer, sarcoma, osteosarcoma, rhabdomyosarcoma, Ewing's sarcoma, carcinoma, melanoma, ovarian cancer, cervical cancer, lymphoma, thyroid cancer, anal cancer, colo-rectal cancer, endometrial cancer, germ cell tumor, laryngeal cancer, multiple myeloma, prostate cancer, retinoblastoma, gastric cancer, testicular cancer, or Wilm's tumor. In some aspects, the cancer is selected from glioma, medulloblastoma, sarcoma, breast cancer, lung cancer, prostate cancer, or intestinal cancer. In some aspects, the cancer cell expresses a site to which native chlorotoxin binds.

In some aspects, the method comprises detecting the compound by fluorescence imaging.

In some aspects, the method further comprises differentiating a focus of a cancer that expresses a site to which native chlorotoxin binds from non-neoplastic tissue.

In some aspects, the method further comprises surgically removing from the subject a cancer cell that is detected.

In some aspects, the method further comprises determining the location of a cancer cell in the subject before surgically removing the cancer cell from the subject, during surgical removal of the cancer cell from the subject, after removing the cancer cell from the subject, or a combination thereof.

In some aspects, the compound binds to the cancer cell. In some aspects, the subject is a human subject. In some aspects, the detection is performed in vivo or ex vivo.

In various aspects, the present disclosure provides a method of administering any suitable compound of the present disclosure to a subject, the method comprising administering a therapeutically effective amount of the compound to the subject.

In some aspects, the subject is in need thereof.

In some aspects, a therapeutically effective amount is a dosage sufficient for the detection of a cancer cell in the subject. In some aspects, the dosage is from 0.1 mg to 100 mg. In some aspects, dosage is from 1 mg to 30 mg. In some aspects, the dosage is from 3 mg to 30 mg.

In various aspects, the present disclosure provides a method of treating a subject in need thereof, the method comprising administering to the subject any suitable compound of the present disclosure further comprising a therapeutic agent in an amount sufficient to treat cancer in the subject. In certain aspects, the therapeutic agent is a cytotoxic agent.

In some aspects, the cancer is selected from glioma, astrocytoma, medulloblastoma, choroids plexus carcinoma, ependymoma, brain tumor, neuroblastoma, head and neck cancer, lung cancer, breast cancer, intestinal cancer, pancreatic cancer, liver cancer, kidney cancer, sarcoma, osteosarcoma, rhabdomyosarcoma, Ewing's sarcoma, carcinoma, melanoma, ovarian cancer, cervical cancer, lymphoma, thyroid cancer, anal cancer, colo-rectal cancer, endometrial cancer, germ cell tumor, laryngeal cancer, multiple myeloma, prostate cancer, retinoblastoma, gastric cancer, testicular cancer, or Wilm's tumor. In some aspects, the cancer cell is selected from glioma, medulloblastoma, sarcoma, prostate cancer, or intestinal cancer. In certain aspects, the cancer cell expresses a site to which native chlorotoxin binds. In further aspects, the binding is selective.

In some aspects, the compound is administered parenterally. In other aspects, the compound is administered intravenously. In still other aspects, the compound is administered intravenously by a bolus, a slow bolus, or an infusion. In yet other aspects, the compound is administered subcutaneously.

### Pharmacokinetic Profiles and Rate of Administration

In some aspects, the present disclosure provides compounds that, upon administration to a subject, produce pharmacokinetic profiles that vary according to the rate of administration of the compound. Such compounds may be referred to herein as “context-sensitive” compounds. Examples of parameters that may vary according to the rate of administration of the compound can include at least the area under curve (AUC), maximum concentration (Cₘₐₓ), minimum concentration reached before a subsequent dose is administered (Cₘᵢₙ), minimum time (Tₘᵢₙ), maximum time to reach Cₘₐₓ (Tₘₐₓ), volume of distribution (V_{d}), back-extrapolated concentration at time 0 (C₀), steady state concentration (Cₛₛ), elimination rate constant (kₑ), clearance (CL), bioavailability (f), fluctuation (% PTF), and elimination half-life (t_{1/2}). For example, in one aspect, the Cₘₐₓ, t_{1/2}, AUC, CL, or V_{d}, or a combination thereof, vary based on a rate of administration of the compound to the subject.

In some aspects, a dosage of a compound is administered to a subject at a constant or approximately constant rate of administration (e.g., using a pump or other mechanism) over the duration of the dosage. In other aspects, a dosage of a compound is administered to a subject at a variable rate of administration over the duration of the dosage. Variations in administration rate may be intentional, or may be unintentional (e.g., due to fluctuations associated with manual injections). In some aspects, the values for administration rates provided herein are averaged values (e.g., averaged over the duration of the dosage).

In some aspects, a compound of the present disclosure is administered to a subject at a rate of administration within the range of about 0.5 mg/hr to about 400,000 mg/hr. In other aspects, the rate of administration is within the range of about 0.5 mg/hr to about 1 mg/hr, about 0.5 mg/hr to about 2 mg/hr, 0.5 mg/hr to about 3 mg/hr, 0.5 mg/hr to about 4 mg/hr, 0.5 mg/hr to about 5 mg/hr, about 0.5 mg/hr to about 6 mg/hr, about 0.5 mg/hr to about 7 mg/hr, about 0.5 mg/hr to about 8 mg/hr, about 0.5 mg/hr to about 9 mg/hr, about 0.5 mg/hr to about 10 mg/hr, about 0.5 mg/hr to about 11 mg/hr, about 0.5 mg/hr to about 12 mg/hr, about 0.5 mg/hr to about 13 mg/hr, about 0.5 mg/hr to about 14 mg/hr, about 0.5 mg/hr to about 15 mg/hr, about 0.5 mg/hr to about 16 mg/hr, about 0.5 mg/hr to about 17 mg/hr, about 0.5 mg/hr to about 18 mg/hr, about 0.5 mg/hr to about 19 mg/hr, about 0.5 mg/hr to about 20 mg/hr, about 0.5 mg/hr to about 25 mg/hr, about 0.5 mg/hr to about 30 mg/hr, about 0.5 mg/hr to about 35 mg/hr, about 0.5 mg/hr to about 40 mg/hr, about 0.5 mg/hr to about 50 mg/hr, about 0.5 mg/hr to about 60 mg/hr, about 0.5 mg/hr to about 70 mg/hr, about 0.5 mg/hr to about 80 mg/hr, about 0.5 mg/hr to about 90 mg/hr, about 0.5 mg/hr to about 100 mg/hr, about 0.5 mg/hr to about 150 mg/hr, about 0.5 mg/hr to about 200 mg/hr, about 0.5 mg/hr to about 250 mg/hr, about 0.5 mg/hr to about 300 mg/hr, about 0.5 mg/hr to about 400 mg/hr, about 0.5 mg/hr to about 500 mg/hr, about 0.5 mg/hr to about 600 mg/hr, about 0.5 mg/hr to about 700 mg/hr, about 0.5 mg/hr to about 800 mg/hr, about 0.5 mg/hr to about 900 mg/hr, about 0.5 mg/hr to about 1,000 mg/hr, about 0.5 mg/hr to about 5,000 mg/hr, about 0.5 mg/hr to about 10,000 mg/hr, about 0.5 mg/hr to about 20,000 mg/hr, about 0.5 mg/hr to about 30,000 mg/hr, about 0.5 mg/hr to about 40,000 mg/hr, about 0.5 mg/hr to about 50,000 mg/hr, about 0.5 mg/hr to about 100,000 mg/hr, about 0.5 mg/hr to about 200,000 mg/hr, about 0.5 mg/hr to about 300,000 mg/hr, or about 0.5 mg/hr to about 400,000 mg/hr. In other aspects, the rate of administration is within a range of about 1 mg/hr to about 5 mg/hr, about 1 mg/hr to about 10 mg/hr, about 1 mg/hr to about 20 mg/hr, about 1 mg/hr to about 30 mg/hr, about 1 mg/hr to about 40 mg/hr, about 1 mg/hr to about 50 mg/hr, about 1 mg/hr to about 100 mg/hr, about 1 mg/hr to about 1,000 mg/hr, about 1 mg/hr to about 100,000 mg/hr, about 1 mg/hr to about 400,000 mg/hr, about 4 mg/hr to about 5 mg/hr, about 4 mg/hr to about 10 mg/hr, about 4 mg/hr to about 20 mg/hr, about 4 mg/hr to about 30 mg/hr, about 4 mg/hr to about 40 mg/hr, about 4 mg/hr to about 50 mg/hr, about 4 mg/hr to about 100 mg/hr, about 4 mg/hr to about 1,000 mg/hr, about 4 mg/hr to about 100,000 mg/hr, about 4 mg/hr to about 400,000 mg/hr, about 10 mg/hr to about 20 mg/hr, about 10 mg/hr to about 30 mg/hr, about 10 mg/hr to about 40 mg/hr, about 10 mg/hr to about 50 mg/hr, about 10 mg/hr to about 100 mg/hr, about 10 mg/hr to about 1,000 mg/hr, about 10 mg/hr to about 100,000 mg/hr, about 10 mg/hr to about 400,000 mg/hr, about 50 mg/hr about 100 mg/hr, about 50 mg/hr to about 1,000 mg/hr, about 50 mg/hr to about 100,000 mg/hr, about 50 mg/hr to about 4,000 mg/hr, about 100 mg/hr to about 1,000 mg/hr, about 100 mg/hr to about 10,000 mg/hr, about 100 mg/hr to about 50,000 mg/hr, about 100 mg/hr to about 100,000 mg/hr, about 100 mg/hr to about 400,000 mg/hr, about 1,000 mg/hr to about 10,000 mg/hr, about 1,000 mg/hr to about 50,000 mg/hr, about 1,000 mg/hr to about 100,000 mg/hr, about 1,000 mg/hr to about 400,000 mg/hr, about 10,000 mg/hr to about 100,000 mg/hr, about 10,000 mg/hr to about 400,000 mg/hr, about 50,000 mg/hr to about 100,000 mg/hr, about 100,000 mg/hr to about 400,000 mg/hr, about 200,000 mg/hr to about 400,000 mg/hr, about 300,000 mg/hr to about 360,000 mg/hr, or about 300,000 mg/hr to about 400,000 mg/hr.

It shall be appreciated that different rates of administration can be achieved by delivering the same dosage over different dosing durations. For example, in some aspects, a dosage of a compound is administered to a subject over a time period less than or equal to about 0.5 seconds, less than or equal to about 1 second, less than or equal to about 5 seconds, less than or equal to about 10 seconds, less than or equal to about 15 seconds, less than or equal to about 20 seconds, less than or equal to about 25 seconds, less than or equal to about 30 seconds, less than or equal to about 35 seconds, less than or equal to about 40 seconds, less than or equal to about 45 seconds, less than or equal to about 50 seconds, less than or equal to about 55 seconds, less than or equal to about 60 seconds, less than or equal to about 65 seconds, less than or equal to about 70 seconds, less than or equal to about 75 seconds, less than or equal to about 80 seconds, less than or equal to about 85 seconds, less than or equal to about 90 seconds, less than or equal to about 95 seconds, less than or equal about 100 seconds, less than or equal to about 105 seconds, less than or equal to about 110 seconds, less than or equal to about 115 seconds, or less than or equal to about 120 seconds. In other aspects, a dosage of a compound is administered to a subject over a time period less than or equal to about 5 minutes, less than or equal to about 10 minutes, less than or equal to about 15 minutes, less than or equal to about 20 minutes, less than or equal to about 25 minutes, less than or equal to about 30 minutes, less than or equal to about 40 minutes, less than or equal to about 50 minutes, less than or equal to about 60 minutes, less than or equal to about 70 minutes, less than or equal to about 80 minutes, less than or equal to about 90 minutes, less than or equal to about 100 minutes, less than or equal to about 110 minutes, less than or equal to about 120 minutes, less than or equal to about 130 minutes, less than or equal to about 140 minutes, less than or equal to about 150 minutes, less than or equal to about 180 minutes, less than or equal to about 210 minutes, less than or equal to about 240 minutes, less than or equal to about 300 minutes, less than or equal to about 360 minutes, less than or equal to about 420 minutes, less than or equal to about 480 minutes, less than or equal to about 540 minutes, less than or equal to about 600 minutes, less than or equal to about 660 minutes, less than or equal to about 720 minutes, less than or equal to about 780 minutes, less than or equal to about 840 minutes, less than or equal to about 900 minutes, less than or equal to about 960 minutes, less than or equal to about 1,020 minutes, less than or equal to about 1,080 minutes, less than or equal to about 1,140 minutes, less than or equal to about 1,200 minutes, less than or equal to about 1,260 minutes, less than or equal to about 1,320 minutes, less than or equal to about 1,380 minutes, or less than or equal to about 1,440 minutes. In other aspects, a dosage of a compound is administered to a subject over a time period within a range from about 2 minutes to about 5 minutes, about 2 minutes to about 4.9 minutes, about 2 minutes to about 4.8 minutes, about 2 minutes to about 4.8 minutes, about 2 minutes to about 4.7 minutes, about 2 minutes to about 4.6 minutes, about 2 minutes to about 4.5 minutes, about 2 minutes to about 4.4 minutes, about 2 minutes to about 4.3 minutes, about 2 minutes to about 4.4 minutes, about 2 minutes to about 4.3 minutes, about 2 minutes to about 4.2 minutes, about 2 minutes to about 4.1 minutes, about 2 minutes to about 4 minutes, about 2 minutes to about 3.9 minutes, about 2 minutes to about 3.8 minutes, about 2 minutes to about 3.7 minutes, about 2 minutes to about 3.6 minutes, about 2 minutes to about 3.5 minutes, about 2 minutes to about 3.4 minutes, about 2 minutes to about 3.3 minutes, about 2 minutes to about 3.2 minutes, about 2 minutes to about 3.1 minutes, about 2 minutes to about 3 minutes, about 2 minutes to about 2.9 minutes, about 2 minutes to about 2.8 minutes, about 2 minutes to about 2.7 minutes, about 2 minutes to about 2.6 minutes, about 2 minutes to about 2.5 minutes, about 2 minutes to about 2.4 minutes, about 2 minutes to about 2.3 minutes, about 2 minutes to about 2.2 minutes, or about 2 minutes to about 2.1 minutes, about 2.5 minutes to about 3 minutes, about 2.5 minutes to about 3.5 minutes, about 2.5 minutes to about 4 minutes, about 2.5 minutes to about 4.5 minutes, about 2.5 minutes to about 5 minutes, about 3 minutes to about 3.5 minutes, about 3 minutes to about 4 minutes, about 3 minutes to about 4.5 minutes, about 3 minutes about 5 minutes, about 3.5 minutes to about 4 minutes, about 3.5 minutes to about 4.5 minutes, about 3.5 minutes to about 5 minutes, about 4 minutes to about 4.5 minutes, about 4 minutes about 5 minutes, or about 4.5 minutes to about 5 minutes. In yet still other aspects, a dosage of a compound is administered to a subject over a period of time that is greater than or equal to about 5 minutes, greater than or equal to about 5.5 minutes, greater than or equal to about 6 minutes, greater than or equal to about 6.5 minutes, greater than or equal to about 7 minutes, greater than or equal to about 7.5 minutes, greater than or equal to about 8 minutes, greater than or equal to about 8.5 minutes, greater than or equal to about 9 minutes, greater than or equal to about 9.5 minutes, greater than or equal to about 10 minutes, greater than or equal to about 10.5 minutes, greater than or equal to about 11 minutes, greater than or equal to about 11.5 minutes, greater than or equal to about 12 minutes, greater than or equal to about 12.5 minutes, greater than or equal to about 13 minutes, greater than or equal to about 13.5 minutes, greater than or equal to about 14 minutes, greater than or equal to about 14.5 minutes, greater than or equal to about 15 minutes, greater than or equal to about 15.5 minutes greater than or equal to about 16 minutes, greater than or equal to about 16.5 minutes, greater than or equal to about 17 minutes, greater than or equal to about 17.5 minutes, greater than or equal to about 18 minutes, greater than or equal to about 18.5 minutes, greater than or equal to about 19 minutes, greater than or equal to about 19.5 minutes, greater than or equal to about 20 minutes, greater than or equal to about 25 minutes, greater than or equal to about 30 minutes, greater than or equal to about 40 minutes, greater than or equal to about 50 minutes, greater than or equal to about 60 minutes, greater than or equal to about 70 minutes, greater than or equal to about 80 minutes, greater than or equal to about 90 minutes, greater than or equal to about 100 minutes, greater than or equal to about 110 minutes, greater than or equal to about 120 minutes, greater than or equal to about 130 minutes, greater than or equal to about 140 minutes, greater than or equal to about 150 minutes, greater than or equal to about 180 minutes, greater than or equal to about 210 minutes, greater than or equal to about 240 minutes, greater than or equal to about 300 minutes, greater than or equal to about 360 minutes greater than or equal to about 420 minutes, greater than or equal to about 480 minutes, greater than or equal to about 540 minutes, greater than or equal to about 600 minutes, greater than or equal to about 660 minutes, greater than or equal to about 720 minutes, greater than or equal to about 780 minutes, greater than or equal to about 840 minutes, greater than or equal to about 900 minutes, greater than or equal to about 960 minutes, greater than or equal to about 1,020 minutes, greater than or equal to about 1,080 minutes, greater than or equal to about 1,140 minutes, greater than or equal to about 1,200 minutes, greater than or equal to about 1,260 minutes, greater than or equal to about 1,320 minutes, greater than or equal to about 1,380 minutes, and greater than or equal to about 1,440 minutes. In still other aspects, a dosage of a compound is administered to a subject over a period of time within a range of about 5 minutes to about 20 minutes, about 5 minutes to about 19 minutes, about 5 minutes to about 18 minutes, about 5 minutes to about 17 minutes, about 5 minutes to about 16 minutes, about 5 minutes to about 15 minutes, about 5 minutes to about 14 minutes, about 5 minutes to about 13 minutes, about 5 minutes to about 12 minutes, about 5 minutes to about 10 minutes, about 5 minutes to about 9 minutes, about 5 minutes to about 8 minutes, about 5 minutes to about 7 minutes, or about 5 minutes to about 6 minutes. In yet still further aspects, a dosage of a compound is administered to a subject over a period of time that is within the range of about 0 minutes to about 2 minutes, about 1 minute to about 2 minutes, about 2 minutes to about 5 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 45 minutes, about 5 minutes to about 60 minutes, about 5 minutes to about 90 minutes, about 5 minutes to about 120 minutes about 5 minutes to about 130 minutes, about 5 minutes to about 140 minutes, about 5 minutes to about 150 minutes, about 5 minutes to about 180 minutes, about 5 minutes to about 210 minutes, about 5 minutes to about 240 minutes, about 5 minutes to about 300 minutes, about 5 minutes to about 360 minutes, about 5 minutes to about 420 minutes, about 5 minutes to about 480 minutes, about 5 minutes to about 540 minutes, about 5 minutes to about 600 minutes, about 5 minutes to about 660 minutes, about 5 minutes to about 720 minutes, about 5 minutes to about 780 minutes, about 5 minutes to about 840 minutes, about 5 minutes to about 900 minutes, about 5 minutes to about 960 minutes, about 5 minutes to about 1,020 minutes, about 5 minutes to about 1,080 minutes, about 5 minutes to about 1,140 minutes, about 5 minutes to about 1,200 minutes, about 5 minutes to about 1,260 minutes, about 5 minutes to about 1,320 minutes, about 5 minutes to about 1,380 minutes, and about 5 minutes to about 1,440 minutes.

In some aspects, the rate of administration is related to the method of administration. For example, in certain aspects, for the same dose, a bolus has faster rate of administration than slow bolus, and both a bolus and slow bolus have a faster rate of administration of than an infusion. As discussed above and herein, in various aspects, a bolus is delivered over a shorter dosing duration than a slow bolus, and a slow bolus is delivered over a shorter dosing duration than an infusion. In some aspects, a bolus is delivered to a subject at a rate of administration that is about 2,000 mg/hr, or within a range from about 20 mg/hr to about 200,000 mg/hr. In some aspects, a slow bolus is delivered to a subject a rate of administration that is about 400 mg/hr, or within a range from about 4 mg/hr to about 40,000 mg/hr. In some aspects, an infusion is delivered to a subject at a rate of administration that is about 15 mg/hr, or within a range from about 0.2 mg/hr to about 100 mg/hr.

In some aspects, the average Cₘₐₓ produced in a subject varies based on the compound's dosage or rate of administration to the subject. In certain aspects, the average Cₘₐₓ increases non-linearly with increasing dosage. For example, the average Cₘₐₓ/mg of the compound administered for dosages greater than 3 mg to 100 mg is up to 2 times, up to 3 times, up to 4 times, up to 5 times, up to 6 times, up to 7 times, up to 8 times, up to 9 times, or up to 10 times greater than the average Cₘₐₓ/mg of the compound administered for dosages of 0.1 mg to 3 mg. In some aspects, the average Cₘₐₓ decreases non-linearly as the rate of administration of the compound decreases. For example, the average Cₘₐₓ per each 1 mg dosage of the compound administered at a rate of greater than 0.2 mg/min to 120 mg/min is up to 1.5 times, up to 2 times, up to 2.5 times, or up to 3 times greater than the average Cₘₐₓ per each 1 mg dosage of the compound administered at a rate of 0.0007 mg/min to 0.2 mg/min.

In some aspects, the average AUC produced in a subject varies based on the compound's dosage or rate of administration to the subject. In certain aspects, the average AUC increases non-linearly with increasing dosage. For example, the average AUC/mg of the compound administered for dosages greater than 3 mg to 100 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average AUC/mg of the compound administered for dosages of 0.1 mg to 3 mg. In some aspects, the average AUC increases non-linearly as the rate of administration of the compound decreases. For example, the average AUC per each 1 mg dosage of the compound administered at a rate of 0.0007 mg/min to 0.2 mg/min is up to 2 times, up to 3 times, up to 4 times, or up to 5 times greater than the average AUC per each 1 mg dosage of the compound administered at a rate of greater than 0.2 mg/min to 120 mg/min.

In some aspects, the average t_{1/2} produced in a subject varies based on the compound's dosage or rate of administration to the subject. In certain aspects, the average t_{1/2} increases non-linearly with increasing dosage. For example, the average t_{1/2} for dosages greater than 3 mg to 100 mg is up to 2 times, up to 3 times, up to 4 times, up to 5 times, up to 6 times, up to 7 times, up to 8 times, up to 9 times, up to 10 times, up to 15 times, or up to 20 times greater than the average t_{1/2} for dosages of 0.1 mg to 3 mg. In some aspects, the average t_{1/2} increases non-linearly as the rate of administration of the compound decreases. For example, the average t_{1/2} of the compound administered at a rate of 0.0007 mg/min to 0.2 mg/min is up to 2 times, up to 3 times, up to 4 times, up to 5 times, up to 6 times, up to 7 times, up to 8 times, up to 9 times, or up to 10 times greater than the average t_{1/2} of the compound administered at a rate of greater than 0.2 mg/min to 120 mg/min.

In some aspects, the average CL produced in a subject varies based on the compound's dosage or rate of administration to the subject. In certain aspects, the average CL decreases non-linearly with increasing dosage. For example, the average CL of the compound administered for dosages of 0.1 mg to 3 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average CL of the compound administered for dosages greater than 3 mg to 100 mg. In some aspects, the average CL decreases as the rate of administration of the compound decreases. For example, the average CL of the compound administered at a rate of greater than 0.2 mg/min to 120 mg/min is up to 2 times, up to 3 times, up to 4 times, or up to 5 times greater than the average CL of the compound administered at a rate of 0.0007 mg/min to 0.2 mg/min.

In some aspects, the average V_{d} produced in a subject varies based on the compound's dosage or rate of administration to the subject. In certain aspects, the average V_{d} increases non-linearly with increasing dosage. For example, the average V_{d} of the compound administered for dosages greater than 3 to 100 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average V_{d} of the compound administered for dosages of 0.1 mg to 3 mg. In some aspects, the average V_{d} decreases as the rate of administration of the compound decreases. For example, the average V_{d} of the compound administered at a rate of greater than 0.2 mg/min to 120 mg/min is up to 2 times, up to 3 times, up to 4 times, or up to 5 times greater than the average V_{d} of the compound administered at a rate of 0.0007 mg/min to 0.2 mg/min.

In some aspects, the present disclosure provides methods in which the rate of administration of a compound is selected or determined in order to produce a desired pharmacokinetic profile in a subject. Certain pharmacokinetic profiles may be advantageous compared to others in certain situations. For example, in some aspects, a pharmacokinetic profile in which the compound is cleared more slowly from the subject (e.g., a longer t_{1/2} or slower clearance rate) is advantageous if the compound binds and/or is internalized by a target cell or tissue relatively slowly, or if a subsequent medical procedure involving the compound is scheduled for a later time. Alternatively, in some aspects, a pharmacokinetic profile in which the compound is cleared more quickly from the subject (e.g., a shorter t_{1/2}) is advantageous if compound binds and/or is internalized by a target cell or tissue relatively quickly, or if a subsequent medical procedure involving the compound is scheduled for an earlier time. Additionally, in certain aspects, a pharmacokinetic profile is advantageously selected to reduce overall exposure to the compound if the compound results in toxicity or other unwanted side effects in the subject or for imaging agents to keep background signal from the drug in the blood to a minimum. The context-sensitive compounds described herein can permit a user (e.g., a medical professional) to produce differing pharmacokinetic profiles in the subject by keeping the dose level constant while varying the rate of administration of the compound (e.g., bolus, slow bolus, or infusion), thus providing a simple and convenient method for adjusting treatment.

In some aspects, the present disclosure provides methods for producing a pharmacokinetic profile in a subject, e.g., a human subject. In one aspect, the present disclosure provides a method of administering a composition to a human subject, the method comprising: determining a rate of administration of a compound to a human subject, wherein a pharmacokinetic profile of the compound in the human subject varies according to the rate of administration of the compound; and intravenously administering the compound to the human subject at the determined rate. In some aspects, the compound comprises a polypeptide having at least 80% sequence identity with any one of SEQ ID NO: 1-SEQ ID NO: 481, or a fragment thereof. For example, in certain aspects, the compound comprises a polypeptide having at least 80% sequence identity with MCMPCFTTDHQMARRCDDCCGGRGRGKCYGPQCLCR (SEQ ID NO: 9) or fragment thereof.

In some aspects, determining the rate of administration comprises determining the time period over which a predetermined dosage is to be intravenously administered to a human subject. In some aspects, the predetermined dosage is within a range from about 0.1 mg to about 30 mg. In some aspects, the time period is selected from less than or equal to about 2 minutes (bolus), within a range from about 2 minutes to about 5 minutes (slow bolus), or greater than or equal to about 5 minutes (infusion).

In another aspect, the method comprises producing a pharmacokinetic profile wherein the pharmacokinetic profile comprises an average maximum blood concentration (average Cₘₐₓ) in the human subject within a range from about 1 ng/mL to about 600 ng/mL per each 1 mg dosage of the compound administered. In yet further aspects, the average Cₘₐₓ per each 1 mg dosage of the compound administered is within a range from about 50 ng/mL to about 300 ng/mL.

In another aspect, the method comprises producing a pharmacokinetic profile wherein the pharmacokinetic profile comprises the average time (average Tₘₐₓ) at which the average Cₘₐₓ is reached is within a range from about 0.5 min to about 120 min following administration of the compound.

In another aspect, the method comprises producing a pharmacokinetic profile wherein the pharmacokinetic profile comprises an average area under the curve (average AUC) in the subject within a range from about 10 hr*ng/mL to about 750 hr*ng/mL per each 1 mg dosage of the compound administered.

In another aspect, the method comprises a pharmacokinetic profile wherein the pharmacokinetic profile comprises an average elimination half-life (average t_{1/2}) in the human subject within a range from about 0.1 hours to about 10 hours. In other aspects, the average Cₘₐₓ, average AUC and/or average t_{1/2} each vary based on a rate of administration of the compound. In yet other aspects, the average Cₘₐₓ, average AUC, and/or average t_{1/2} each increase as the rate of administration of the compound decreases.

In some aspects, the pharmacokinetic profile is determined by the rate of administration of the compound. In one aspect, determining the rate of administration comprises determining a time period over which a predetermined dosage is to be intravenously administered to the human subject, wherein the predetermined dosage is within a range from about 1 mg to about 100 mg and the time period is selected from: less than or equal to about 5 minutes, within a range from about 5 minutes to about 15 minutes, or greater than or equal to about 15 minutes. In other aspects, the predetermined dosage is within a range from about 1 mg to about 30 mg. In other aspects, the rate of administration is determined by the method of administration, such as administration by bolus, slow bolus or infusion. In some aspect, when the administration is an infusion, the average Cₘₐₓ, average AUC and/or average t_{1/2} each increase non-linearly with increasing dose.

In one aspect, the rate of administration is determined based on one or more characteristics of a cancer in a human subject. In some aspects, the one or more characteristics of a cancer comprise a type of cancer. For example, the cancer comprises one or more of: brain cancer, glioma, astrocytoma, medulloblastoma, oligiodendroglioma, choroids plexus carcinoma, ependymoma, pituitary cancer, neuroblastoma, basal cell carcinoma, cutaneous squamous cell carcinoma, melanoma, head and neck cancer, lung cancer, small cell lung cancer, non-small cell lung cancer, breast cancer, ductal carcinoma in situ, intestinal cancer, pancreatic cancer, liver cancer, kidney cancer, bladder cancer, carcinoma of unknown primary, sarcoma, osteosarcoma, rhabdomyosarcoma, Ewing's sarcoma, gastrointestinal stromal tumors, melanoma, ovarian cancer, cervical cancer, lymphoma, Hodgkin's lymphoma, non-Hodgkin's lymphoma, thyroid cancer, anal cancer, colo-rectal cancer, laryngeal cancer, multiple myeloma, prostate cancer, retinoblastoma, gastric cancer, esophageal cancer, testicular cancer, or Wilm's tumor. In another aspect, the one or more characteristics of a cancer comprise an aggressiveness of the cancer. For example, the determined rate of administration is higher when the cancer is more aggressive and lower when the cancer is less aggressive. In certain aspects, more aggressive cancers uptake the compound faster so that prolonged exposure is not needed compared to less aggressive cancers, which uptake compounds slower so that a prolonged exposure is needed.

An aggressive tumor can also be described in terms of tumor pathology grade. A tumor pathology grade can be a numerical value (e.g., 1, 2, 3, or 4) and based on a pathologist's determination of the amount of abnormality of the tumor cells, and. For example, grade 1 tumors contain tumor cells that have similar organization to cells in normal tissue and are considered well-differentiated as compared to grade 4 tumors, which tend to lack normal tissue structure and are considered undifferentiated or poorly differentiated. In general, a low grade tumor can be less aggressive than a high grade tumor. In yet another aspect, the one or more characteristics of a cancer comprise a location of the cancer. For example, the determined rate of administration is lower when the cancer is located in the brain and higher when the cancer is not located in the brain. In certain aspects, the compound takes longer to cross the blood brain barrier so that a lower rate of administration is needed compared with more accessible locations in the body. In still another aspect, the one or more characteristics of a cancer comprise a rate of uptake of the compound by cancerous tissue or cancer cells. For example, the determined rate of administration is higher when the rate of uptake is higher and lower when the rate of uptake is lower.

In another aspect, the rate of administration is determined based on an amount of time between the administration of the compound and performing of a surgical procedure on the human subject. In one aspect, the determined rate is higher when the amount of time is shorter between the administration of the compound and performing of a surgical procedure and lower when the amount of time is longer between the administration of the compound and performing of a surgical procedure. In certain aspects, the rate of administration of the compound is determined based on a therapeutic usage of the compound. In some aspects, the rate of administration is determined by the clearance of the excess compound from the patient's body so that the excess compound does not interfere with imaging during surgery. In another aspect, the rate of administration is determined by the tumor's characteristics as well as the time until surgery to optimize for imaging during surgery. In another aspect, the rate of administration is based on a type of surgical procedure to be performed on the human subject following administration of the compound. In yet another aspect, the rate of administration further comprises performing the surgical procedure on the human subject, wherein the determined rate of administration results in an average blood plasma concentration of the compound greater than about 10 ng/mL when the surgical procedure is performed. In still another aspect, the rate of administration is based on the surgical procedure, wherein the surgical procedure to be performed is to remove cancerous tissue or cancer cells from the human subject.

In a clinical setting, the non-linear changes observed in Cₘₐₓ, AUC, and t_{1/2} when the rate of administration is varied can have important implications for treating patients. The rate of administration can impact determination of an optimal dose given to a patient for a particular treatment. Alternatively, a pre-determined optimal dose can impact the rate at which the dose is administered for a particular treatment. For example, a patient can receive two independent dosages of the compound, wherein one dosage is given for a therapeutic purpose and another dosage is given for an imaging purpose. In one aspect, the dosage given for the therapeutic purpose is calibrated for longer tumor exposure. In another aspect, the rate of administration for the dosage given for the therapeutic purpose is calibrated for longer tumor exposure. In certain aspects, the dosage given for the therapeutic purposes is administered by an infusion. In one aspect, the dosage for imaging purposes is calibrated for imaging during surgery. In another aspect, the rate of administration for a dosage for imaging purposes is calibrated for imaging during surgery. In certain aspects, the dosage for imaging purposes is administered by a bolus or slow bolus. In yet another aspect, an optimal dosage for a patient is determined, and then based upon the treatment, the rate of administration is optimized for the treatment. For example, the rate of administration can be optimized for different types of surgeries.

### Methods for Analysis to Generate Pharmacokinetic Profiles

In some aspects, samples are analyzed to obtain parameters useful to determine a pharmacokinetic profile. Often the samples can be diluted, for example, using a buffer or pharmaceutically acceptable carrier as defined herein.

Pharmacokinetic standard curves can often be generated using a chlorotoxin conjugate, serum, and a pharmaceutical carrier as described herein. The proportion of each chlorotoxin conjugate, concentrated source of sample (for example serum, urine, etc.), and pharmaceutical carrier can often differ, for example, the concentration of compound of the present disclosure can often be between about 10 μg/mL and about 1 ng/mL. Often the standard curve can be used to calculate the concentration of the compound in the sample.

In some aspects pharmacokinetic data are analyzed using standard pharmacokinetic data analysis methods, including evaluating the concentration of chlorotoxin conjugates versus time. For example, a software program, such as Phoenix WinNonlin v6.3 or another comparable software programs, is used to analyze pharmacokinetic data to obtain pharmacokinetic parameters. In some aspects, the pharmacokinetic data analysis uses standard noncompartmental methods of intravenous bolus, intravenous infusion, or extravascular input as appropriate. Often, the data can be analyzed by the mean serum concentration versus time. The data can also be analyzed by individual subject and the resulting parameters can then be summarized by group descriptive statistics.

Pharmacokinetic profiles of the compositions described herein can often be obtained using at least one, sometimes more than one bioanalytical method. In some aspects, bioanalytical methods include the addition of chemicals to a sample containing a composition of which the pharmacokinetic profile is desired. Addition of the chemical to the sample often can comprise performing a chemical technique to measure the concentration of a composition or a metabolite thereof in a sample or, sometimes, in a biological matrix. For example, microscale thermophoresis, mass spectrometry often including liquid chromatograph and a triple quadropole mass spectrometer, tandem mass spectrometry, fluorescence-based detection methods, ligand binding assays, detection of radioactive substances, MRI signals, light absorbance, radio active decay, high sensitivity mass spectrometry for microdosing studies and the like are often performed.

### EXAMPLES

The invention is further illustrated by the following non-limiting examples.

### Example 1

### Pharmacokinetics of a Chlorotoxin Conjugate Following a Single 15-Minute Intravenous Infusion

This example demonstrates the pharmacokinetic (PK) profile of Compound 76 after a single intravenous (IV) infusion in human subjects before surgical excision of skin cancer tumor(s) from the subjects.

Each subject was given a single 15-minute IV infusion of Compound 76 at fixed dose level of one of: 1, 3, 6, 12 or 18 mg. Three subjects were given 1 mg. Three subjects were given 3 mg. Six subjects were given doses of 6 mg. Six subjects were given 12 mg. Three subjects were given 18 mg. Following the single 15-minute intravenous infusion, blood samples were collected at 1, 5, 15, 30, and 60 minutes, and 2, 4, 8, 12, 24, 48, 96, and 168 hours post-start of infusion. A blood sample was also collected from each subject before Compound 76 administration (To). Samples were analyzed for Compound 76 serum concentration using a validated liquid chromatography/mass spectrometry (LC/MS) method. Fluorescent signal from skin tumors was observed at doses of 3 mg and above as early as 2 hours post-dose.

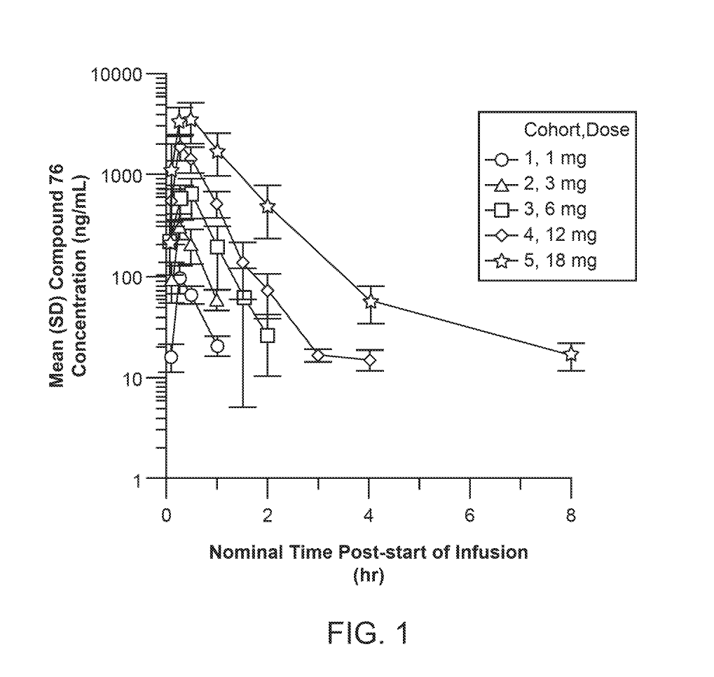

The mean serum concentrations versus time profiles of Compound 76 after each single 15-minute IV infusion are shown in FIG. 1. These profiles show that at higher doses of Compound 76, the mean Compound 76 serum concentrations decrease in a non-linear fashion over time.

As shown in TABLE 5, the pharmacokinetic parameters: Tₘₐₓ, Cₘₐₓ, AUC₀₋ₜ, AUC_{0-∞}, t_{1/2}, CL and Vₛₛ were measured. The Tₘₐₓ parameter is a measure of the time to maximum serum concentration. The Cₘₐₓ parameter is a measure of the maximum observed serum concentration. The AUC₀₋ₜ parameter is a measure of the area under the serum concentration-time curve from time zero (to) to the last measurable serum concentration, calculated by the linear up/log down trapezoidal rule. The AUC_{0-∞} parameter is a measure of the area under the serum concentration-time curve extrapolated from the timepoint with the last measured concentration to a time of infinity. The t_{1/2} parameter is a measure of apparent terminal elimination half-life of Compound 76. The CL parameter is a measure of the apparent clearance of Compound 76. The Vₛₛ parameter is a measure of the apparent volume of distribution at steady state. This data indicates that the Cₘₐₓ increased in a greater than dose-proportional manner over the tested dose range. This data also indicates that the AUC₀₋ₜ increased in a greater than dose-proportional manner over the tested dose range. Furthermore, this data shows that the t_{1/2} values increased with increasing doses. Additionally, this data shows that mean CL values decreased with increasing dose. In contrast, mean Vₛₛ was essentially constant between dose groups.

TABLE 5
Summary of Compound 76 PK Parameters Following a Single
IV 15-minute Infusion (BB-001 Clinical Trial)

| | | Parameter | | | | | | |
|---|---|---|---|---|---|---|---|---|
| | | | | AUC₀₋ₜ | AUC_{0-∞} | | CL | |
| | | Tₘₐₓ | Cₘₐₓ | (hr*ng/ | (hr*ng/ | t_{1/2} | (mL/ | V_{SS} |
| Dose | | (hr) | (ng/mL) | mL) | mL) | (hr) | hr) | (mL) |
| 1 mg | N | 3 | 3 | 3 | 0 | 0 | 0 | 0 |
| | Mean | 0.250 | 95.5 | 52.4 | NE | NE | NE | NE |
| | SD | 0.00 | 29.7 | 11.8 | N/A | N/A | N/A | N/A |
| 3 mg | N | 3 | 3 | 3 | 1 | 1 | 1 | 1 |
| | Mean | 0.250 | 315 | 184 | 278 | 0.327 | 10800 | 4770 |
| | SD | 0.00 | 89.5 | 77.5 | N/A | N/A | N/A | N/A |
| 6 mg | N | 6 | 6 | 6 | 6 | 6 | 6 | 6 |
| | Mean | 0.294 | 783 | 548 | 558 | 0.364 | 12100 | 5710 |
| | SD | 0.101 | 208 | 197 | 200 | 0.0697 | 4460 | 1530 |
| 12 mg | N | 6 | 6 | 6 | 6 | 6 | 6 | 6 |
| | Mean | 0.253 | 1900 | 1470 | 1480 | 0.498 | 8830 | 4740 |
| | SD | 0.00680 | 529 | 450 | 451 | 0.115 | 3060 | 1100 |
| 18 mg | N | 3 | 3 | 3 | 2 | 2 | 2 | 2 |
| | Mean | 0.417 | 3830 | 4430 | 3450 | 1.75 | 5260 | 6110 |
| | SD | 0.144 | 1230 | 1790 | N/A | N/A | N/A | N/A |

NE not estimated due to insufficient characterization of the terminal phase of the concentration versus time profile in all subjects.
N/A not applicable,
N ≤ 2;
SD = standard deviation

### Example 2

### Pharmacokinetics of a Chlorotoxin Conjugate Following a Single Intravenous Slow-Bolus Injection

This example demonstrates the pharmacokinetic (PK) profile of Compound 76 after a single intravenous (IV) slow-bolus injection in human subjects before surgical excision of glioma tumor(s) from the subjects.

Each subject was given a single slow-bolus IV injection of Compound 76. Each single slow-bolus IV injection was given over the course of 3-4 minutes. Three subjects were given a 3 mg dose. Four subjects were given a 9 mg dose. Four subjects were given an 18 mg dose. Following the single slow-bolus IV injection, blood samples were collected at 1, 5, 15, 30, 60 and 120 minutes post-injection from each subject. A blood sample was also collected from each subject before Compound 76 administration (To). Samples were analyzed for Compound 76 serum concentration using a validated liquid chromatography/mass spectrometry (LC/MS) method. Fluorescent signal from portions of resected tumor were observed using ex vivo techniques and was concordant with pathology confirmed tumor.

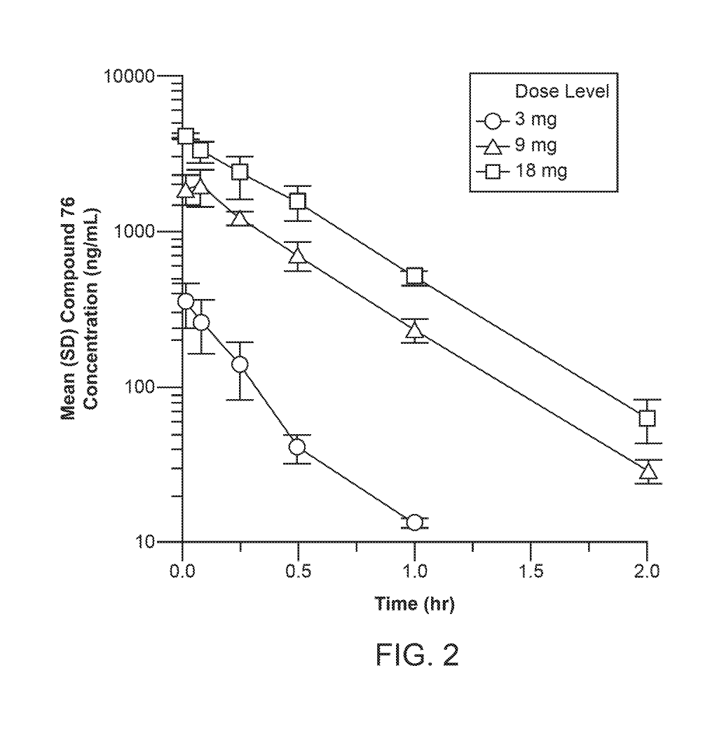

The mean serum concentration versus time profiles of Compound 76 after a single slow-bolus injection are shown in FIG. 2. Following a single slow-bolus administration, Compound 76 levels were detectable out to 1 hr post-dose at the 3 mg dose level and out to 2 hrs post-dose at the 9 mg and 18 mg dose levels. This figure shows that the mean Compound 76 serum concentration decrease is monophasic over time for each dose tested.

As shown in TABLE 6, the pharmacokinetic parameters t_{1/2}, Cₘₐₓ, and AUC₀₋ₜ were measured. This data shows that the t_{1/2} values remained constant at the higher doses. As shown in TABLE 6, the Cₘₐₓ values increased in a dose-dependent manner and the AUC₀₋ₜ values increased in a dose-dependent manner.

TABLE 6
Summary of Compound 76 PK Parameters Following a Single
IV Slow Bolus Injection (BB-002 Clinical Trial)

Parameter

| | t_{1/2} | Cₘₐₓ | AUC₀₋ₜ |
|---|---|---|---|
| Dose | (hr) | (ng/mL) | (hr*ng/mL) |

| | | | | | |
|---|---|---|---|---|---|
| 3 | mg | N | 3 | 3 | 3 |
| | | Mean | 0.242 | 350 | 134 |
| | | SD | 0.0466 | 115 | 36.9 |
| 9 | mg | N | 4 | 4 | 4 |
| | | Mean | 0.326 | 2260 | 1150 |
| | | SD | 0.0131 | 321 | 125 |
| 18 | mg | N | 3 | 4 | 4 |
| | | Mean | 0.392 | 3980 | 2370 |
| | | SD | 0.0838 | 212 | 363 |

SD = standard deviation

A comparison between the PK parameters set forth in TABLES 5 and 6 above suggests that the total level of Compound 76 exposure (as measured AUC₀₋ₜ) is lower following an IV slow-bolus dose (see TABLE 6) than for a 15-minute IV infusion (see TABLE 5).

### Example 3

### Pharmacokinetic Comparison Between Intravenous Slow-Bolus and 15-Minute Intravenous Infusion Injections

This example compares the pharmacokinetic (PK) profiles of Compound 76 after a single intravenous (IV) slow-bolus injection in human subjects with glioma undergoing surgical excision of their tumor(s) compared to after a single IV administration in human subjects with skin cancer undergoing surgical excision of their tumor(s).

Three subjects were given a single 3 mg dose by slow-bolus IV injection of Compound 76. Three subjects were given a single 18 mg dose by slow-bolus IV injection of Compound 76. The single slow-bolus IV injection was given over the course of 3-4 minutes. Three subjects were given a single 3 mg dose by 15-minute IV infusion of Compound 76. Three subjects were given a single 18 mg dose by 15-minute IV infusion of Compound 76. Following the single slow-bolus IV injection, blood samples were collected at 1, 5, 15, 30, 60 and 120 minutes post-injection. A blood sample was also collected from each subject before Compound 76 administration (To). Following each single 15-minute intravenous infusion, blood samples were collected from each subject at 1, 5, 15, 30, and 60 minutes, and 2, 4, 8, 12, 24, 48, 96, and 168 hours post-start of infusion. A blood sample was also collected from each subject before Compound 76 administration (To). All samples were analyzed using a validated liquid chromatography/mass spectrometry (LC/MS) method.

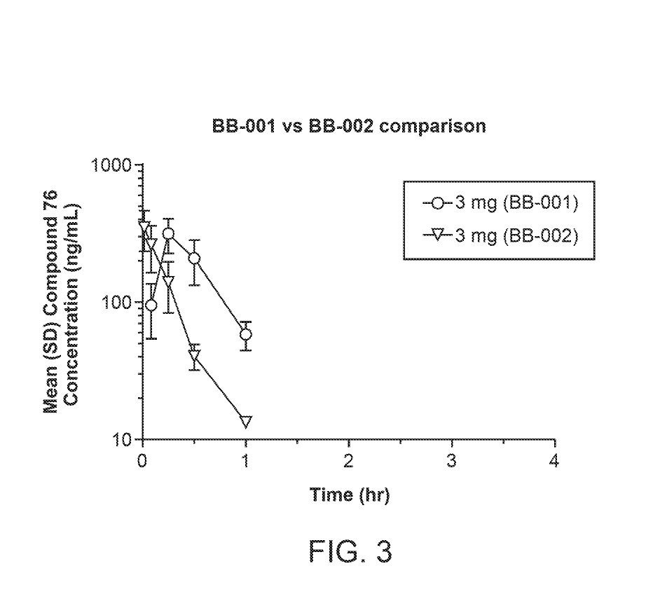

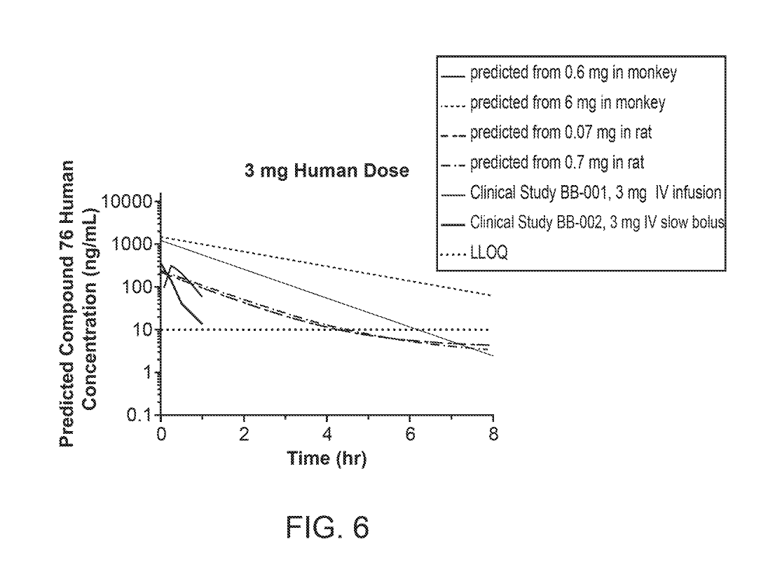

The mean serum concentration versus time profiles of Compound 76 after a single slow-bolus injection of 3 mg compared to after a single 15-minute IV infusion of 3 mg are shown in FIG. 3. The mean serum concentration versus time profiles of Compound 76 after a single slow-bolus injection of 18 mg compared to after a single 15-minute IV infusion of 18 mg are shown in FIG. 6. Both figures show that the mean Compound 76 serum concentration decreases more rapidly after administration by a single slow-bolus as compared to the mean Compound 76 serum concentration after administration by a single 15-minute IV infusion.

### Example 4

### Pharmacokinetic Comparison Between Observed and Predicted Dosing Models

This example compares the observed and predicted PK profiles of Compound 76.

In the first comparison, human subjects were each administered a single 1 mg, a single 3 mg, or a single 6 mg IV infusion of Compound 76 before surgical excision of skin cancer tumor(s). Those results were then compared to the predicted human PK profiles based on scaling of a single IV infusion in monkeys and to the predicted PK profiles based on scaling of a single IV infusion in rats.

In the second comparison, human subjects were administered a single 12 mg IV infusion of Compound 76 before surgical excision of skin cancer tumor(s) and the results compared to the predicted human PK profiles determined by data from a single IV infusion in monkeys or from a single IV infusion in rats.

For the IV infusion data from subjects, samples were collected as described in Example 1. For the slow-bolus IV infusion data from subjects, samples were collected as previously described in Example 2.

Compound 76 serum concentrations from animal studies were used to predict human serum concentration values following administration of single 1 mg, 3 mg, 6 mg, or 12 mg doses at 1, 5, 15, 30, and 60 minutes, and 2, 4, 8, 12, 24, 48, 96, and 168 hours after Compound 76 administration. Compound 76 serum concentration values were predicted using data from monkeys given an IV bolus administration of 0.6 mg or 6 mg Compound 76. Compound 76 serum concentration data were predicted using data from rats given an IV bolus administration of 0.07 mg or 0.7 mg Compound 76. More specifically, the human serum concentration values were predicted by using a scaling method utilizing the fixed exponent approach (CLₕᵤₘₐₙ=CLₐₙᵢₘₐₗ×[BWₕᵤₘₐₙ/BWₐₙᵢₘₐₗ]^{0.8} and Vₕᵤₘₐₙ=Vₐₙᵢₘₐₗ×[BWₕᵤₘₐₙ/BWₐₙᵢₘₐₗ]^{1.0}; Wang et. al, Biopharm. Drug Dispos. 31: 253-263, 2010). Four simulations were run based on PK parameters from: 1) 0.07 mg dose in rat, 2) 0.7 mg dose in rat, 3) 0.6 mg dose in monkey, and 4) 6 mg dose in monkey.

Compound 76 serum concentration data from a single 18 mg slow-bolus IV injection Compound 76 as described in Example 2 was used to predict human Compound 76 serum concentration values following administration of a 12 mg dose.

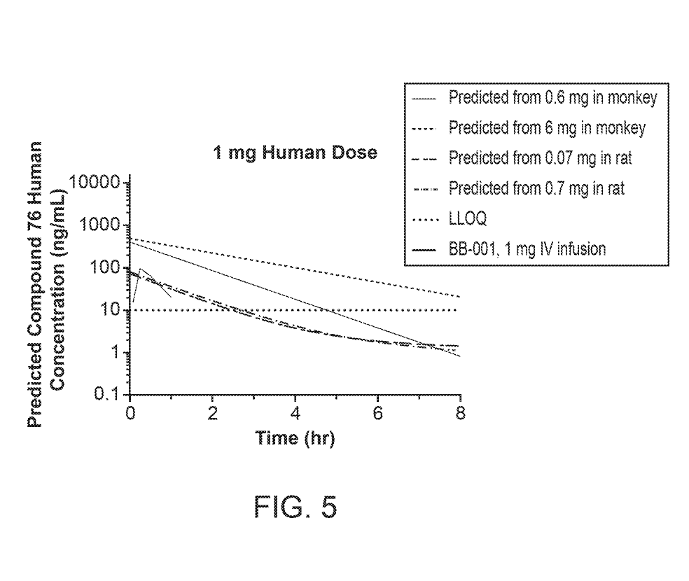

The mean serum concentration versus time profiles of Compound 76 after a single 15-minute IV infusion of a 1 mg dose are shown in FIG. 5. LLOQ indicates the lower limit of quantitation of the assay. These profiles show that the mean Compound 76 serum concentration decreases more rapidly over time in human subjects as compared with the predicted linear decrease in mean Compound 76 serum concentration from monkey data and rat data. In addition, scaling to human from monkey data overpredicts the actual human concentrations.

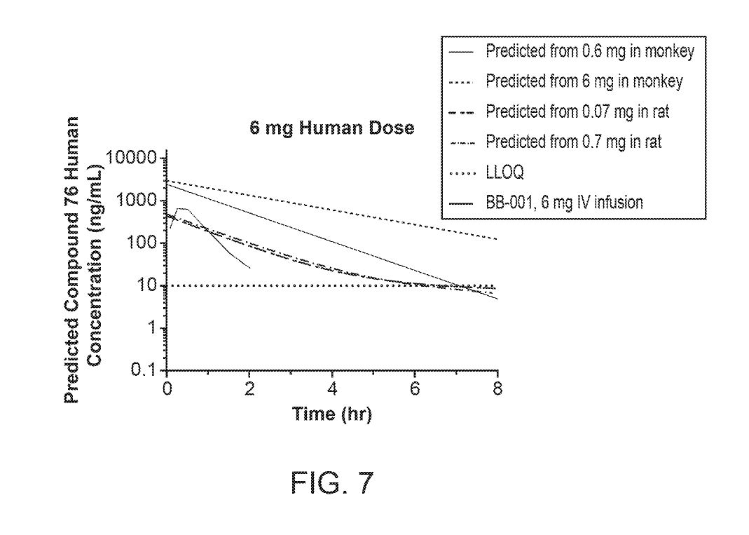

The mean serum concentration versus time profiles of Compound 76 after a single 15-minute IV infusion or a slow bolus of a 3 mg dose are shown in FIG. 6. These profiles show that the mean Compound 76 serum concentration decreases more rapidly over time in human subjects receiving the 15-minute IV infusion or IV bolus as compared with predicted decrease in mean Compound 76 serum concentration from monkey data and rat data. In addition, scaling to human from monkey data overpredicts the actual human concentrations and this discrepancy is more pronounced in the actual IV bolus data. The mean serum concentration versus time profiles of Compound 76 after a single 15-minute IV infusion of a 6 mg dose are shown in FIG. 7. These profiles show that the mean Compound 76 serum concentration decrease more rapidly over time as compared with the predicted decrease in mean Compound 76 serum concentration from monkey data and rat data.

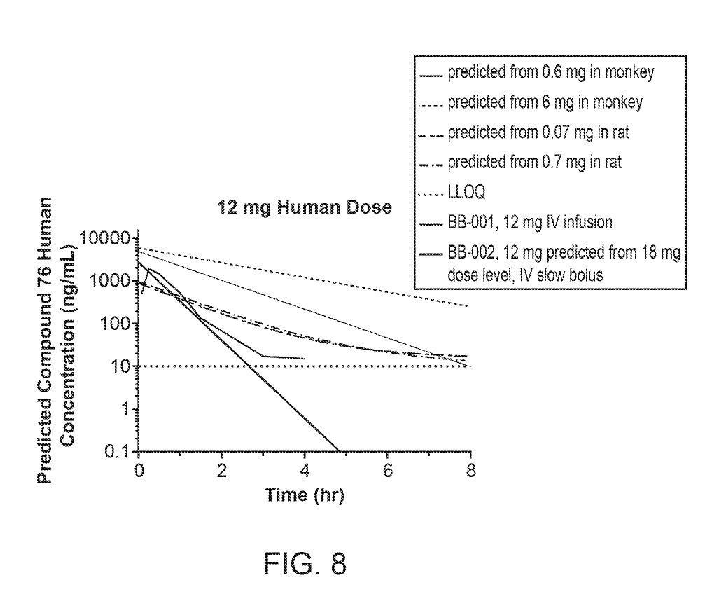

The mean serum concentration versus time profiles of Compound 76 after a single 15-minute IV infusion are shown in FIG. 8. These profiles show that the mean Compound 76 serum concentration decreases more rapidly over time as compared with the predicted decrease in mean Compound 76 serum concentration from human slow-bolus IV inject data, monkey data and rat data.

### Example 5

### Tissue Imaging Following Conjugate Administration

This example describes the fluorescent signal of Compound 76 after a single IV infusion in human subjects with skin cancer undergoing surgical excision of their tumor(s).

Each subject was administered a single 15-minute IV infusion of Compound 76 at fixed dose levels of 1, 3, 6, 12 or 18 mg. Three subjects were given a 1 mg dose. Three subjects were given a 3 mg dose. Six human subjects were given a 6 mg dose. Six human subjects were given a 12 mg dose. Three human subjects were given an 18 mg dose. Following each of the single 15-minute intravenous infusions, images of skin lesions from each subject were taken at 2, 4, 24, and 48 hours post-start of transfusion as well as before Compound 76 administration (To). Images were taken using the FLUOBEAM 800 clinical imaging system.

Compound 76 signal was measurable as early as 2 hours following Compound 76 dosing. Imaging data above the 1 mg dose level and below the 18 mg dose level generally resulted in signal intensities with reliable lesional versus non-lesional assessments and having sufficient contrast between lesional and non-lesional skin. The fluorescence signals observed in the 3, 6, and 12 mg dose cohorts were confirmed to be skin tumors after excision. Several target lesions were not obviously more fluorescent than adjacent skin, and these lesions were generally not tumor by histopathology.

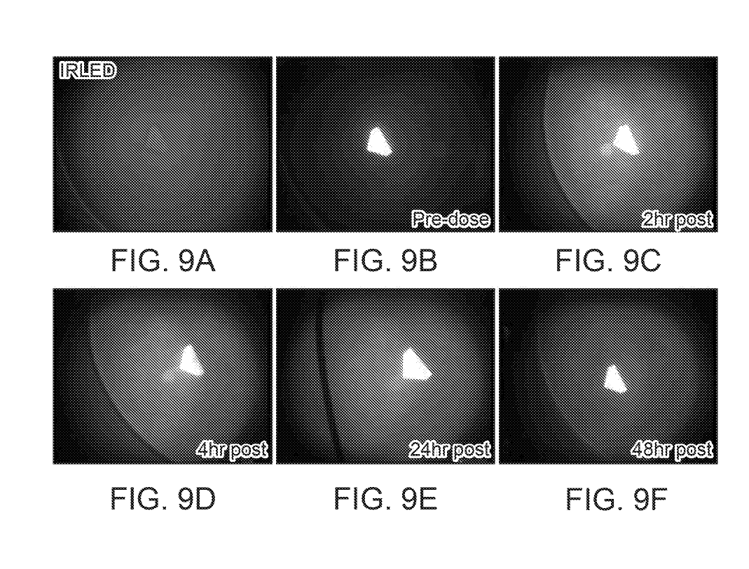

FIGS. 9A, 9B, 9C, 9D, 9E, and 9F depict images of skin lesions before and after administration of a 3 mg Compound 76 dose to a subject. Skin lesions are identified in each image by arrow. For these figures, FLUOBEAM 800 images were collected at 500 msec exposure on the upper back of the patient. FIGS. 9A and 9B were obtained as controls, and did not emit fluorescence. FIG. 9A was obtained as an Infrared LED (IRLED) image. FIG. 9B is a pre-dose image.

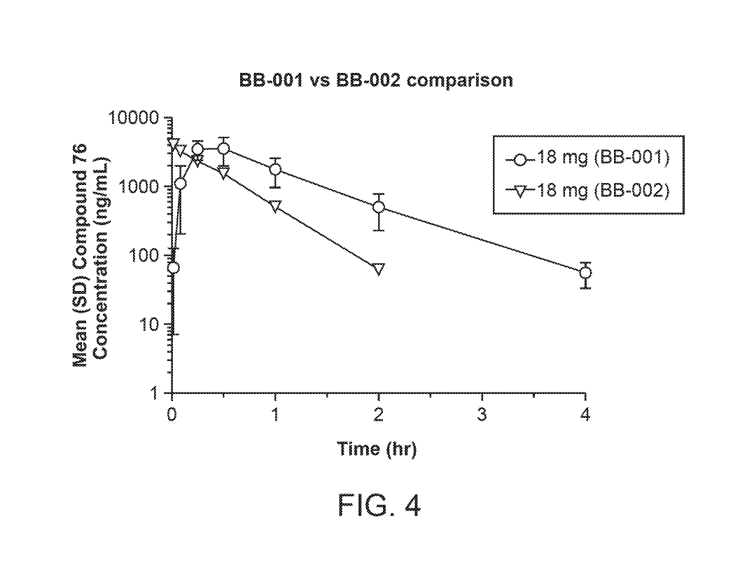

Fluorescence was clearly visible in the pre-specified target area at 2 hours post-dose as shown in FIG. 9C and 4 hours post-dose as shown in FIG. 9D with less intense signal present at 24 hours post-dose as shown in FIG. 9E. No visibly discernable signal was observed 48 hours post-dose as shown in FIG. 9F. The excised lesion was confirmed by histopathology to be basal cell carcinoma.

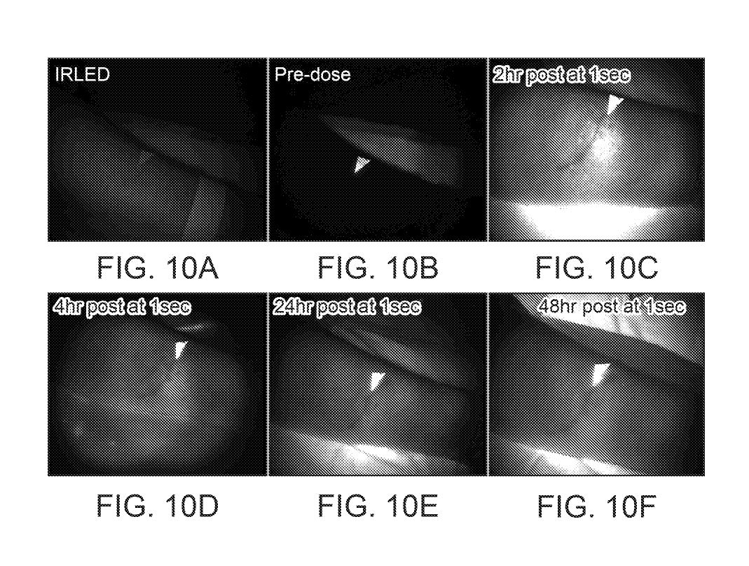

FIGS. 10A, 10B, 10C, 10D, 10E, and 10F show examples of skin lesion images before and after administration of a 6 mg dose of Compound 76 to a subject. Skin lesions are identified in each image by arrow. In these figures, FLUOBEAM 800 images were collected at 1 sec exposure on the arm of the subject. The IRLED image shown in FIG. 10A and pre-dose image shown in FIG. 10B are control images that show no fluorescence. Fluorescence was clearly visible in the pre-specified target area at 2 hours post-dose as shown in FIG. 10C and 4 hours post-dose as shown in FIG. 10D with less intense signal present at 24 hours post-dose as shown in FIG. 10E. No visibly discernable signal was observed 48 hours post-dose as shown in FIG. 10F. The excised lesion was confirmed by histopathology to be melanoma.

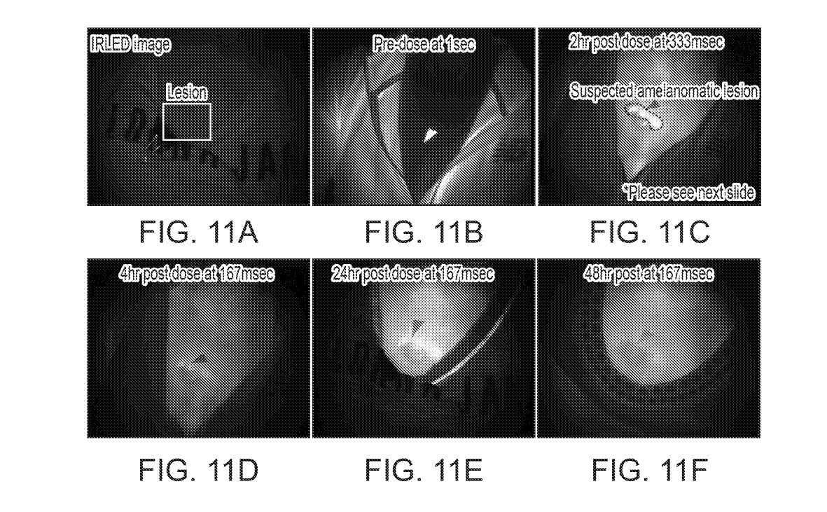

FIGS. 11A, 11B, 11C, 11D, 11E, and 11F show examples of skin lesion images before and after administration of a 12 mg dose to a human subject. Skin lesions are identified in each image by arrow in FIGS. 11B, 11C, 11D, 11E, and 11F, and by a box in FIG. 11A. The IRLED image as shown in FIG. 11A and the pre-dose image as shown in FIG. 11B are control images that show no fluorescence. FLUOBEAM 800 images were collected on the chest of the patient at 333 msec exposure in FIG. 11C and at 167 msec exposure in FIGS. 11D, 11E, and 11F. Fluorescence was clearly visible in the pre-specified target area at 2 hours post-dose as shown in FIG. 11C and 4 hours post-dose as shown in FIG. 11D with less intense signal present at 24 hours post-dose as shown in FIG. 11E. No visibly discernable signal was observed 48 hours post-dose as shown in FIG. 11F.

FIG. 11C depicts a dashed line encircling a suspected amelanomic lesion. The excised lesion was confirmed by histopathology to be melanoma.

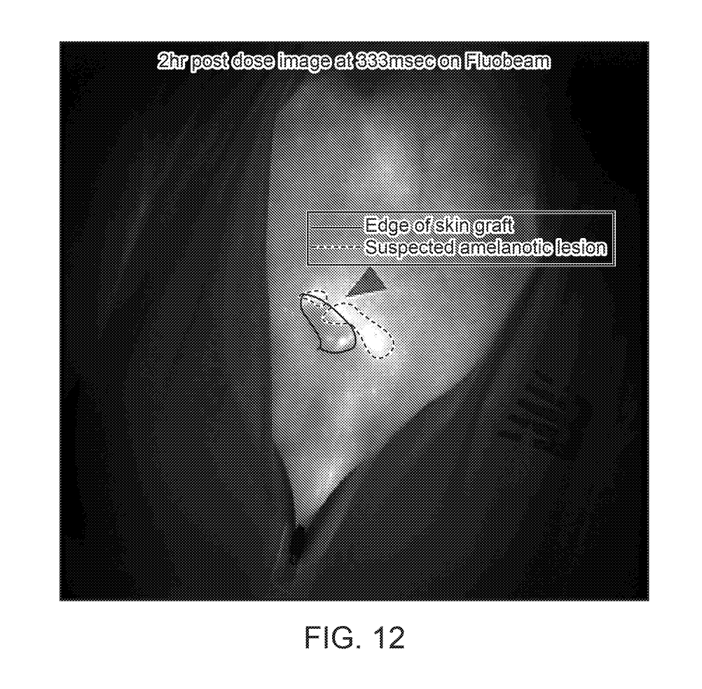

FIG. 12 further shows a FLUOBEAM 800 image collected at 333 msec exposure 2 hrs after the 12 mg Compound 76 dose. The solid line shows the edge of a skin graft and the dashed line show a the suspected amelanomic region. As evidenced by FIG. 12, the use of real-time tumor fluorescence enables surgical planning by highlighting the tumor margins to allow for the excision and inclusion of all of the tumor.

### Example 6

### In Situ Tumor Imaging Using Conjugate Fluorescence

This example describes in situ fluorescence imaging of Compound 76 after a single intravenous (IV) slow-bolus injection in human subjects with glioma undergoing surgical excision of their tumor(s).

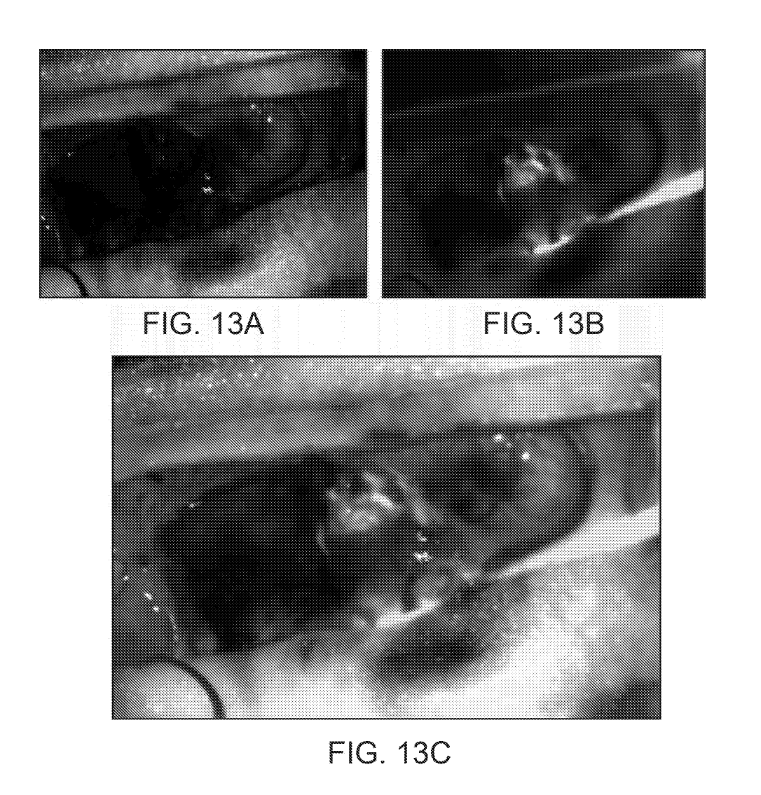

Each subject was administered a single slow-bolus IV injection of Compound 76. The single slow-bolus IV injection was given over the course of 3-4 minutes. Three human subjects were given a 3 mg dose. Four human subjects were given a 9 mg dose. Four human subjects were given an 18 mg dose. Four hours following the single slow-bolus IV injection, imaging of tumors in situ was performed using the Synchronized Infra-Red Imaging System (SIRIS). FIGS. 13A, 13B, and 13C show the in situ image from a human subject given 18 mg, who had a pathologically confirmed glioblastoma multiforme. In these images, in situ fluorescent contrast was seen in tumors of subjects with accessible tumors treated with either 9 mg or 18 mg of Compound 76. The in situ contrast ratios ranged from 1.6 to 2.5 in these subjects. FIG. 13A shows a white light image of exposed tumor. FIG. 13B shows a Near Infrared (NIR) tissue image. FIG. 13C shows the combined visible and NIR tissue images. The fluorescent signal from presumed areas of normal brain was measurable for all subjects and serves as a measure of biologic background intensity. There was no increase in normal brain fluorescence intensity seen with increasing dose.

### Example 7

### Ex Vivo Fluorescence Imaging

This example describes the ex vivo imaging fluorescence of Compound 76 after a single intravenous (IV) slow-bolus injection in human subjects with glioma undergoing surgical excision of their tumor(s).

Subjects were administered a single slow-bolus IV injection of Compound 76. Each single slow-bolus IV injection was given over the course of 3-4 minutes. Three human subjects were given a 3 mg dose. Four human subjects were given a 9 mg dose. Four human subjects were given an 18 mg dose. Four to twenty-nine hours following the administration of each single slow-bolus IV injection, Synchronized Infra-Red Imaging System (SIRIS) images of excised tumor samples were taken for two of the subjects given a 3 mg dose, three of the subjects given a 9 mg dose, and three of the subjects given an 18 mg dose. Additionally, Odyssey scans of ex vivo tissues were taken of these excised tumors. Areas of viable tumor were verified by histopathology, and mean fluorescence intensity was measured in these areas. The ex vivo tissues had an area of intense fluorescence and an adjacent area with little or no fluorescence signal in both types of images. The fluorescence intensity data in the Odyssey scans were concordant with the fluorescence intensity data in the SIRIS images, demonstrating a correlation between immediate ex vivo fluorescence imaging and Odyssey scan data.

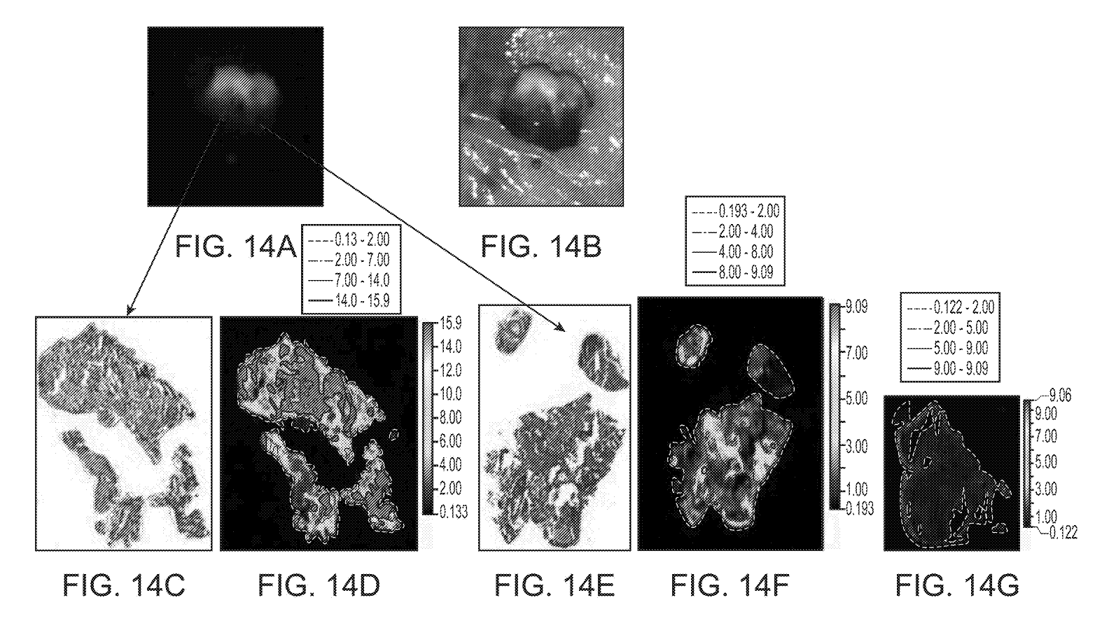

FIGS. 14A, 14B, 14C, 14D, 14E, 14F, and 14G show an example of ex vivo tissue images from a human subject 20 hours following an 18 mg dose of Compound 76. Freshly excised tissue was imaged using the SIRIS. The near-infrared (NIR) image is shown in FIG. 14A, and NIR overlaid on white light image is shown in FIG. 14B. Contrast between the bright and dim areas was approximately 8-fold.

FIG. 14C shows an H&E staining image of a tissue slice from the upper fluorescent region of the ex vivo tissue from a human subject given an 18 mg dose of Compound 76 corresponding to tissue area in FIG. 14A marked by an arrow from FIG. 14A to this Figure. The entire tissue slice shown in this figure contains tumor. FIG. 14D shows an Odyssey scan of the tissue slice shown in FIG. 14C, in which the tissue is ex vivo tissue from a human subject given an 18 mg dose of Compound 76 and the entire tissue slice is from the fluorescent tumor region in the upper portion of FIG. 14A. The entire tissue slice shown in this figure contains tumor. Fluorescence signal intensity varied in the tissue, but was overall the NIR signal intensity was high.

FIG. 14E shows an H&E staining image of the a tissue slice from the lower dark region of the ex vivo tissue from a human subject given an 18 mg dose of Compound 76 corresponding to tissue area in FIG. 14A marked by an arrow from FIG. 14A to this Figure. The entire tissue slice shown in this figure is mostly from necrotic tissue and has less viable tumor than FIG. 14C and FIG. 14D. FIG. 14F shows an Odyssey scan of the tissue slice shown in FIG. 14E, in which the tissue is ex vivo tissue from a human subject given an 18 mg dose of Compound 76 and the entire tissue slice is from the dark necrotic tissue region in the lower portion of FIG. 14A. The entire tissue slice shown in this figure is mostly from necrotic tissue and has less viable tumor than FIG. 14C and FIG. 14D. Fluorescence signal intensity has significantly less NIR fluorescence signal and is consistent with being sections from the dark region of FIG. 14A. This further indicated that the tissue has significantly less tumor and is largely necrotic tissue.

FIG. 14G shows an Odyssey scan of untreated cerebellum was used as a negative control. As shown by these images, the intensely fluorescent area is viable tumor, and the less-fluorescent area was necrotic tissue.

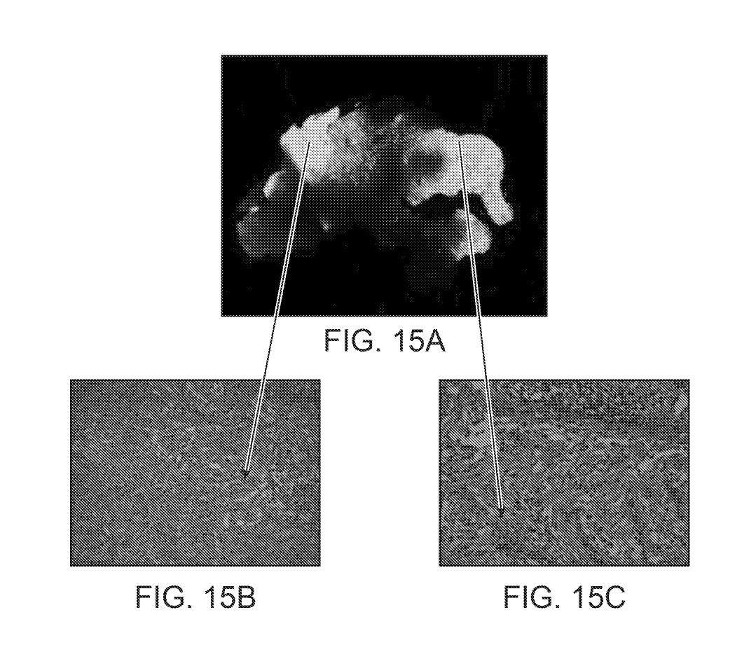

FIG. 15A depicts an ex vivo Odyssey scan compared to histopathology images in FIGS. 15B and 15C of a low-grade pleomorphic xanthocytoma tumor from a pediatric subject dosed with the equivalent of a 3 mg adult dose of Compound 76. The areas of fluorescence from the tumor in the Odyssey scan (FIG. 15A) depicts tumor pathology in the corresponding H&E stained panels (FIGS. 15B and 15C) as marked by the arrows from FIG. 15A pointing to either FIG. 15B or 15C. This Odyssey scan data demonstrates that the fluorescent signal following Compound 76 administration is correlated with areas containing tumor cells, whereas fluorescently dim areas have little tumor present.

### Example 8

### Ex Vivo Imaging

This example describes ex vivo imaging of tissues from five human subjects having breast cancer following administration of 12 mg Compound 76 at least two hours prior to surgery.

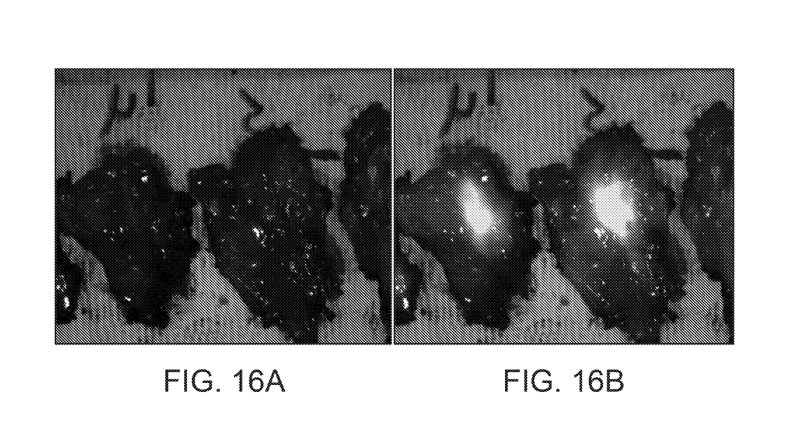

Fluorescent signal was observed in areas of suspected tumors in all cases. FIGS. 16A and 16B show an example of ex vivo Synchronized Infra-Red Imaging System (SIRIS) images from one subject. FIG. 16A shows the excised gross tissue specimens under white light. FIG. 16B shows the near-infrared overlay of the excised gross tissue specimens with the white light image in which the fluorescence indicates tumor.

### Example 9

### Predicting PK Profile Based on Compound 76 Administration

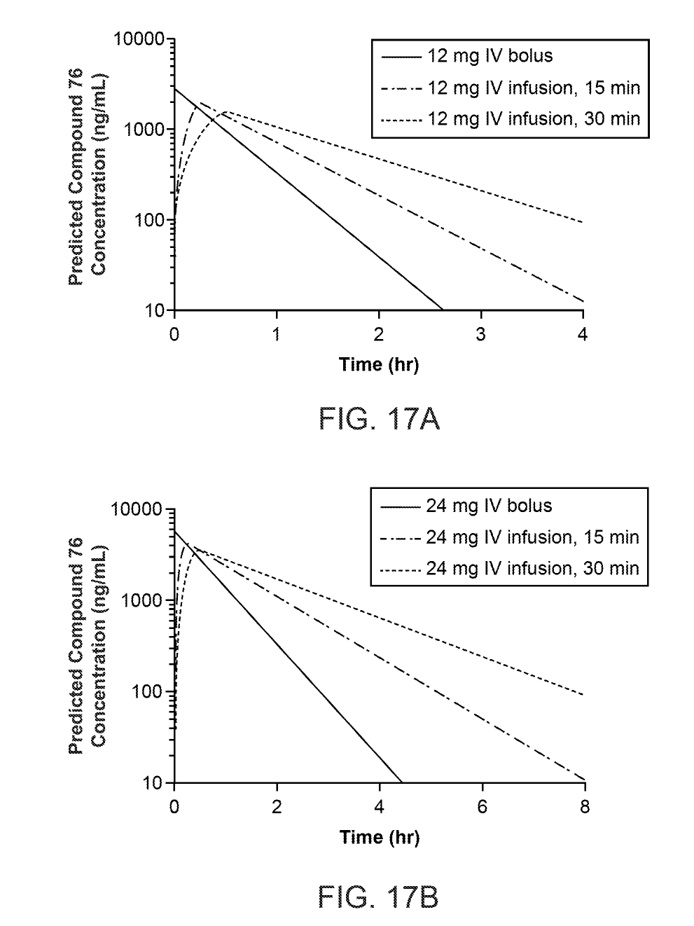

This example shows predicted the PK profiles of Compound 76 based on the rate of administration of a 12 mg dose of Compound 76. The effect of decreasing the rate of Compound 76 administration from an IV bolus dose to an IV infusion dose increases the volume of distribution and/or decreases the rate of clearance of Compound 76. This, in turn, leads to an increase in t_{1/2} and in the overall systemic exposure of Compound 76, as measured by AUC, for a given dose level. FIG. 17A shows a graph of predicted Compound 76 concentration versus time profiles after administration of 12 mg Compound 76 at different rates of administration. When a human subject is given a dose of 12 mg Compound 76, the volume of distribution is predicted to be 4200 mL and the clearance is predicted to be 9000 mL/hr when administered as an IV bolus. Based on these parameters, the t_{1/2} is predicted to be 0.32 hr and the AUC is predicted to be 1300 hr*ng/mL. In contrast, when Compound 76 is administered as a 30 minute IV infusion, the PK parameters for a 12 mg dose is predicted to be a volume of distribution equal to 6200 mL and a clearance equal to 5000 mL/hr. Based on these parameters the t_{1/2} is predicted to be 0.86 hr and the AUC would be 2400 hr*ng/mL. As another example, FIG. 17B shows a graph of predicted Compound 76 concentration versus time profiles after administration of 24 mg Compound 76 at different rates of administration.

### Example 10

### Pharmacokinetic Comparison Between Single Dose and Repeat Dose Administrations in Rats

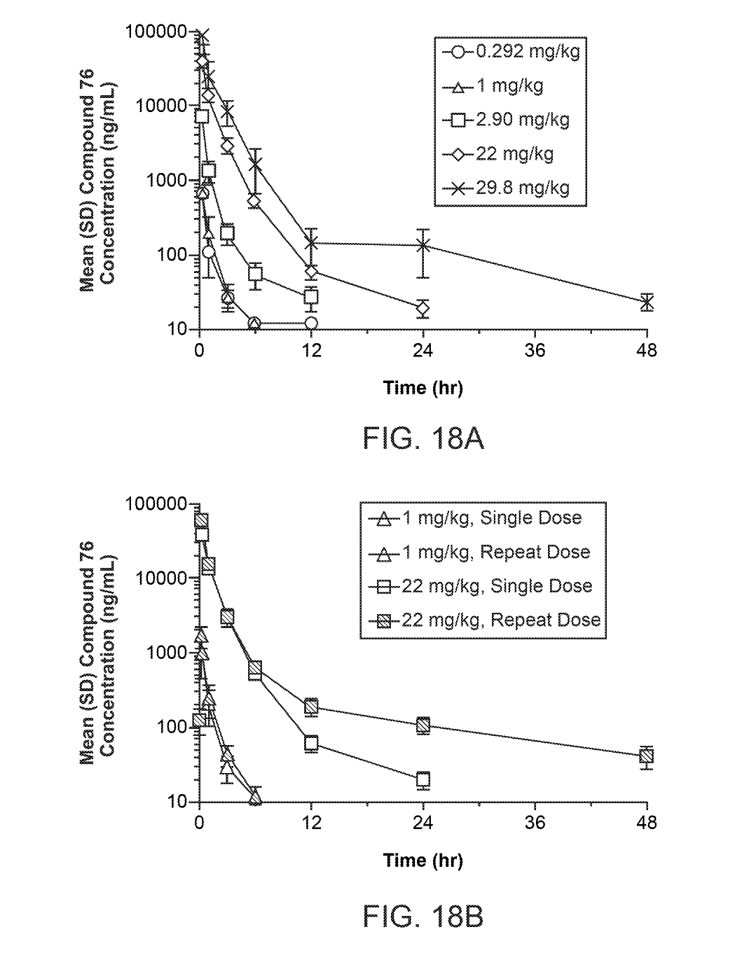

This example describes a pharmacokinetic comparison between single dose and repeat dose administrations in rats from Good Laboratory Practices (GLP) (21 C.F.R. § 58) compliant studies. Doses of 0.292 mg/kg, 1 mg/kg, 2.90 mg/kg, 22 mg/kg, or 29.8 mg/kg of Compound 76 was administered in rats by intravenous (IV) bolus injection. Rats either received a single dose administration (single dose) or were re-administered the dose (repeat dose) once daily for seven days total. IV bolus injections were administered over a period of about 2 minutes. Pharmacokinetic analysis was carried out using a non-compartmental analysis using the IV bolus model. FIG. 18 shows pharmacokinetic data from rats receiving single dose administrations at several dose levels or repeat dose administrations at two different dose levels. FIG. 18A shows single dose pharmacokinetic data at several dose levels including 0.292 mg/kg, 1 mg/kg, 2.90 mg/kg, 22 mg/kg, and 29.8 mg/kg. FIG. 18B shows a pharmacokinetic comparison between rats receiving single dose administration at doses of 1 mg/kg or 22 mg/kg versus repeat dose administration every day for 7 days at doses of 1 mg/kg or 22 mg/kg. Both FIG. 18A and FIG. 18B illustrate the pharmacokinetic nonlinearity of Compound 76 upon administration in rats, as evidenced by the graphical shape change of the line, resulting from changes in dose level or changes in number of administrations (single versus repeat).

### Example 11

### Pharmacokinetics of a Chlorotoxin Conjugate Following a Single Intravenous Slow-Bolus Injection

This example demonstrates the pharmacokinetic (PK) profile of Compound 76 after a single intravenous (IV) bolus injection in human subjects before surgical excision of breast cancer from the subjects (BB-005 clinical trial).

Each subject was given a single bolus IV injection of Compound 76. Each single bolus IV injection was given over the course of 3-4 minutes. Eleven subjects were given a 6 mg dose. Four subjects were given a 12 mg dose. Following the single bolus IV injection, blood samples were collected at 5, 15, and 30 minutes post-injection from each subject. Samples were analyzed for Compound 76 serum concentration using a validated liquid chromatography/mass spectrometry (LC/MS) method.

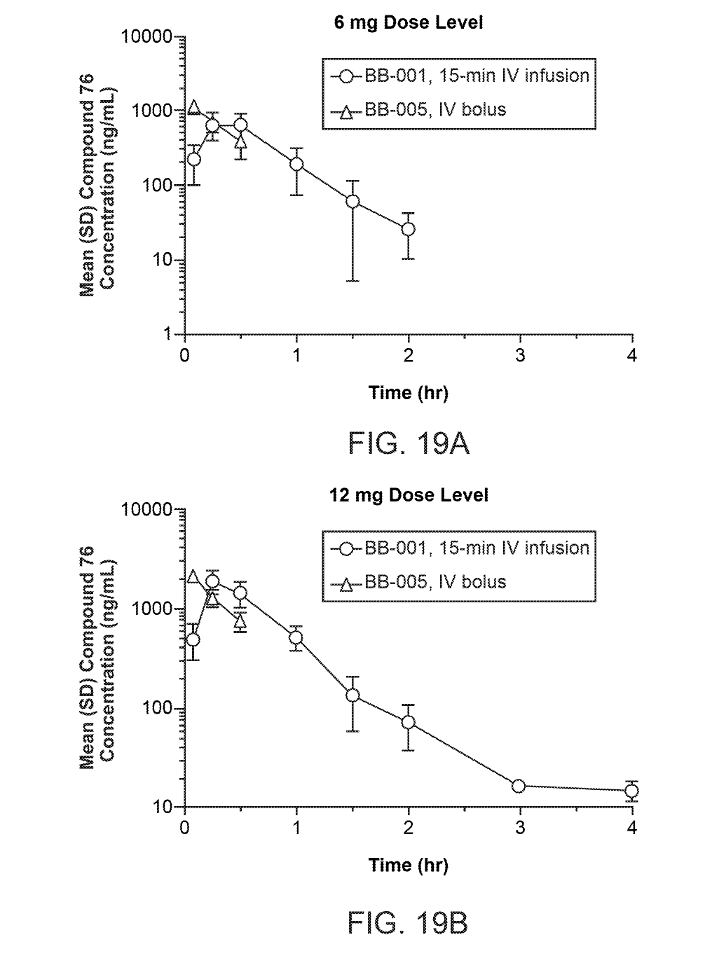

The mean serum concentration versus time profiles of Compound 76 after a single bolus injection are shown in FIG. 19 and compared to the pharmacokinetic results from BB-001 described in EXAMPLE 1. FIG. 19A illustrates a pharmacokinetic comparison of BB-001 (15-min IV infusion) and BB-005 (IV bolus administration) clinical trials at the 6 mg dose level. FIG. 19B illustrates a pharmacokinetic comparison of BB-001 (15-min IV infusion) and BB-005 (IV bolus administration) clinical trials at the 12 mg dose level. Following a single bolus administration, Compound 76 levels were sampled and measured out to 30 minutes post-dose at the 6 mg and 12 mg dose levels.

TABLE 7 shows a summary of pharmacokinetic parameters from the BB-001 clinical trial (EXAMPLE 1), the BB-002 (EXAMPLE 2) clinical trial, and the BB-005 clinical trial: Cₘₐₓ, AUC₀₋ₜ, and t_{1/2}. The Cₘₐₓ parameter is a measure of the maximum observed serum concentration. The AUC₀₋ₜ parameter is a measure of the area under the serum concentration-time curve from time zero (to) to the last measurable serum concentration, calculated by the linear up/log down trapezoidal rule. The t_{1/2} parameter is a measure of apparent terminal elimination half-life of Compound 76. Notable, the t_{1/2} at 12 mg from a 15 minute infusion is longer than from the same dose given by IV bolus (BB-001 verus BB-005 data). This trend was even more marked when comparing BB-001 versus BB-002 data at the 18 mg dose.

TABLE 7
Summarized Single Dose Compound 76 PK Parameters (Studies BB-001, BB-002, and BB-005)

| | | | BB-001 | | | BB-002 | | | BB-005 | | |
|---|---|---|---|---|---|---|---|---|---|---|---|
| Compound | | | (15-min IV Infusion) | | | (IV Bolus) | | | (IV Bolus)^{a} | | |
| 76 Dose | Parameter | Units | N | Mean | SD | N | Mean | SD | N | Mean | SD |

| 1 mg | Cₘₐₓ | ng/mL | 3 | 95.5 | 29.7 | dose not evaluated | | | dose not evaluated |
|---|---|---|---|---|---|---|---|---|---|
| | AUC₀₋ₜ | hr*ng/mL | 3 | 52.4 | 11.8 | | | | |
| | t_{1/2} | hr | 0 | NE | N/A | | | | |
| 3 mg | Cₘₐₓ | ng/mL | 3 | 315 | 89.5 | 3 | 350 | 115 | dose not evaluated |
| | AUC₀₋ₜ | hr*ng/mL | 3 | 184 | 77.5 | 3 | 134 | 36.9 | |
| | t_{1/2} | hr | 1 | 0.327 | N/A | 3 | 0.242 | 0.0466 | |

| | | | | | | | | | |
|---|---|---|---|---|---|---|---|---|---|
| 6 mg | Cₘₐₓ | ng/mL | 6 | 783 | 208 | dose not evaluated | 11 | 1130 | 209 |
| | AUC₀₋ₜ | hr*ng/mL | 6 | 548 | 197 | | 11 | 401 | 81.9 |
| | t_{1/2} | hr | 6 | 0.364 | 0.0697 | | 11 | 0.278 | 0.103 |

| | | | | | | | |
|---|---|---|---|---|---|---|---|
| 9 mg | Cₘₐₓ | ng/mL | dose not evaluated | 4 | 2260 | 321 | dose not evaluated |
| | AUC₀₋ₜ | hr*ng/mL | | 4 | 1150 | 125 | |
| | t_{1/2} | hr | | 4 | 0.326 | 0.0131 | |

| | | | | | | | | | |
|---|---|---|---|---|---|---|---|---|---|
| 12 mg | Cₘₐₓ | ng/mL | 6 | 1900 | 529 | dose not evaluated | 3 | 2180 | 193 |
| | AUC₀₋ₜ | hr*ng/mL | 6 | 1470 | 450 | | 3 | 752 | 75.0 |
| | t_{1/2} | hr | 6 | 0.498 | 0.115 | | 2 | 0.300 | 0.0728 |

| | | | | | | | | | |
|---|---|---|---|---|---|---|---|---|---|
| 18 mg | Cₘₐₓ | ng/mL | 3 | 3830 | 1230 | 4 | 3980 | 212 | dose not evaluated |
| | AUC₀₋ₜ | hr*ng/mL | 3 | 4430 | 1790 | 4 | 2370 | 363 | |
| | t_{1/2} | hr | 2 | 1.75 | N/A | 3 | 0.392 | 0.0838 | |

| | | | | | | | |
|---|---|---|---|---|---|---|---|
| 24 mg | Cₘₐₓ | ng/mL | dose not evaluated | 3 | 5130 | 1310 | dose not evaluated |
| | AUC₀₋ₜ | hr*ng/mL | | 3 | 4320 | 1320 | |
| | t_{1/2} | hr | | 3 | 0.521 | 0.0764 | |
| 30 mg | Cₘₐₓ | ng/mL | dose not evaluated | 3 | 7120 | 1310 | dose not evaluated |
| | AUC₀₋ₜ | hr*ng/mL | | 3 | 4630 | 789 | |
| | t_{1/2} | hr | | 3 | 0.485 | 0.0532 | |

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of administering a composition to a human subject, the method comprising:
intravenously administering to the human subject a compound comprising a polypeptide having at least 80%, at least 85%, at least 90%, or at least 95% sequence identity with MCMPCFTTDHQMARRCDDCCGGRGRGKCYGPQCLCR (SEQ ID NO: 9) or a fragment thereof, wherein the compound is administered at a dosage within a range from about 1 mg to about 100 mg over a time period within a range from about 1 minute to about 120 minutes; and
producing an average maximum blood plasma concentration (average Cₘₐₓ) in the human subject within a range from about 15 ng/mL to about 600 ng/mL per each 1 mg dosage of the compound administered.

2. A method of administering a composition to a human subject, the method comprising:
intravenously administering to the human subject a compound comprising a polypeptide having at least 80%, at least 85%, at least 90%, or at least 95% sequence identity with any one of SEQ ID NO: 1-SEQ ID NO: 481 or a fragment thereof, wherein the compound is administered at a dosage within a range from about 1 mg to about 100 mg over a time period within a range from about 1 minute to about 120 minutes; and
producing an average maximum blood plasma concentration (average Cₘₐₓ) in the human subject within a range from about 15 ng/mL to about 600 ng/mL per each 1 mg dosage of the compound administered.

3. A method of administering a composition to a human subject, the method comprising:
intravenously administering to the human subject a compound comprising a polypeptide of any one of SEQ ID NO: 482-SEQ ID NO: 485 or a fragment thereof, wherein the compound is administered at a dosage within a range from about 1 mg to about 100 mg over a time period within a range from about 1 minute to about 120 minutes; and
producing an average maximum blood plasma concentration (average Cₘₐₓ) in the human subject within a range from about 15 ng/mL to about 600 ng/mL per each 1 mg dosage of the compound administered.

4. The method of any one of claims 1-3, wherein the time period is greater than or equal to about 5 minutes, greater than or equal to about 10 minutes, greater than or equal to about 15 minutes, greater than or equal to about 20 minutes, greater than or equal to about 25 minutes, greater than or equal to about 30 minutes, greater than or equal to about 40 minutes, greater than or equal to about 50 minutes, greater than or equal to about 60 minutes, greater than or equal to about 70 minutes, greater than or equal to about 80 minutes, greater than or equal to about 90 minutes, greater than or equal to about 100 minutes, or greater than or equal to about 110 minutes.

5. The method of any one of claims 1-3, wherein the time period is less than or equal to about 5 minutes, less than or equal to about 10 minutes, less than or equal to about 15 minutes, less than or equal to about 20 minutes, less than or equal to about 25 minutes, less than or equal to about 30 minutes, less than or equal to about 40 minutes, less than or equal to about 50 minutes, less than or equal to about 60 minutes, less than or equal to about 70 minutes, less than or equal to about 80 minutes, less than or equal to about 90 minutes, less than or equal to about 100 minutes, or less than or equal to about 110 minutes.

6. The method of any one of claims 1-3, wherein the time period is within a range from about 1 minute to about 2 minutes, within range from about 2 minutes to about 5 minutes, or within a range from about 5 minutes to about 120 minutes.

7. The method of any one of claims 1-6, wherein the average Cₘₐₓ per each 1 mg dosage of the compound administered is greater than or equal to about 20 ng/mL, greater than or equal to about 30 ng/mL, greater than or equal to about 40 ng/mL, greater than or equal to about 50 ng/mL, greater than or equal to about 60 ng/mL, greater than or equal to about 70 ng/mL, greater than or equal to about 80 ng/mL, greater than or equal to about 90 ng/mL, greater than or equal to about 100 ng/mL, greater than or equal to about 150 ng/mL, greater than or equal to about 200 ng/mL, greater than or equal to about 250 ng/mL, greater than or equal to about 300 ng/mL, greater than or equal to about 350 ng/mL, greater than or equal to about 400 ng/mL, greater than or equal to about 450 ng/mL, greater than or equal to about 500 ng/mL, or greater than or equal to about 550 ng/mL.

8. The method of any one of claims 1-6, wherein the average Cₘₐₓ per each 1 mg dosage of the compound administered is less than or equal to about 20 ng/mL, less than or equal to about 30 ng/mL, less than or equal to about 40 ng/mL, less than or equal to about 50 ng/mL, less than or equal to about 60 ng/mL, less than or equal to about 70 ng/mL, less than or equal to about 80 ng/mL, less than or equal to about 90 ng/mL, less than or equal to about 100 ng/mL, less than or equal to about 150 ng/mL, less than or equal to about 200 ng/mL, less than or equal to about 250 ng/mL, less than or equal to about 300 ng/mL, less than or equal to about 350 ng/mL, less than or equal to about 400 ng/mL, less than or equal to about 450 ng/mL, less than or equal to about 500 ng/mL, or less than or equal to about 550 ng/mL.

9. The method of any one of claims 1-6, wherein the average Cₘₐₓ per each 1 mg dosage of the compound administered is within a range from about 50 ng/mL to about 300 ng/mL.

10. The method of any one of claims 1-9, wherein the average time (average Tₘₐₓ) at which the average Cₘₐₓ is reached is within a range from about 0.5 min to about 120 min following administration of the compound.

11. The method of any one of claims 1-10, wherein the average Cₘₐₓ increases non-linearly with increasing dosage.

12. The method of claim 11, wherein the average Cₘₐₓ/mg of the compound administered for dosages greater than 3 mg to 10 mg is up to 2 times, up to 3 times, up to 4 times, up to 5 times, up to 6 times, up to 7 times, up to 8 times, up to 9 times, or up to 10 times greater than the average Cₘₐₓ/mg of the compound administered for dosages of 0.1 mg to 3 mg.

13. The method of any one of claims 1-12, wherein the average Cₘₐₓ varies based on a rate of administration of the compound.

14. The method of claim 13, wherein the average Cₘₐₓ decreases non-linearly as the rate of administration of the compound decreases.

15. The method of claim 14, wherein the average Cₘₐₓ per each 1 mg dosage of the compound administered at a rate of greater than 0.2 mg/min to 120 mg/min is up to 1.5 times, up to 2 times, up to 2.5 times, or up to 3 times greater than the average Cₘₐₓ per each 1 mg dosage of the compound administered at a rate of 0.0007 mg/min to 0.2 mg/min.

16. The method of any one of claims 1-15, further comprising: producing an average area under the curve (average AUC) in the subject within a range from about 10 hr*ng/mL to about 750 hr*ng/mL per each 1 mg dosage of the compound administered.

17. The method of claim 16, wherein the average AUC per each 1 mg dosage of the compound administered is greater than or equal to about 20 hr*ng/mL, greater than or equal to about 30 hr*ng/mL, greater than or equal to about 40 hr*ng/mL, greater than or equal to about 50 hr*ng/mL, greater than or equal to about 60 hr*ng/mL, greater than or equal to about 70 hr*ng/mL, greater than or equal to about 80 hr*ng/mL, greater than or equal to about 90 hr*ng/mL, greater than or equal to about 100 hr*ng/mL, greater than or equal to about 150 hr*ng/mL, greater than or equal to about 200 hr*ng/mL, greater than or equal to about 250 hr*ng/mL, greater than or equal to about 300 hr*ng/mL, greater than or equal to about 350 hr*ng/mL, greater than or equal to about 400 hr*ng/mL, greater than or equal to about 450 hr*ng/mL, greater than or equal to about 500 hr*ng/mL, greater than or equal to about 550 hr*ng/mL, greater than or equal to about 600 hr*ng/mL, greater than or equal to about 650 hr*ng/mL, or greater than or equal to about 700 hr*ng/mL.

18. The method of claim 16, wherein the average AUC per each 1 mg dosage of the compound administered is less than or equal to about 20 hr*ng/mL, less than or equal to about 30 hr*ng/mL, less than or equal to about 40 hr*ng/mL, less than or equal to about 50 hr*ng/mL, less than or equal to about 60 hr*ng/mL, less than or equal to about 70 hr*ng/mL, less than or equal to about 80 hr*ng/mL, less than or equal to about 90 hr*ng/mL, less than or equal to about 100 hr*ng/mL, less than or equal to about 150 hr*ng/mL, less than or equal to about 200 hr*ng/mL, less than or equal to about 250 hr*ng/mL, less than or equal to about 300 hr*ng/mL, less than or equal to about 350 hr*ng/mL, less than or equal to about 400 hr*ng/mL, less than or equal to about 450 hr*ng/mL, less than or equal to about 500 hr*ng/mL, less than or equal to about 550 hr*ng/mL, less than or equal to about 600 hr*ng/mL, less than or equal to about 650 hr*ng/mL, or less than or equal to about 700 hr*ng/mL.

19. The method of claim 16, wherein the average AUC per each 1 mg dosage of the compound administered is within a range from about 15 hr*ng/mL to about 400 hr*ng/mL.

20. The method of any one of claims 16-19, wherein the average AUC increases non-linearly with increasing dosage.

21. The method of claim 20, wherein the average AUC/mg of the compound administered for dosages greater than 3 mg to 100 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average AUC/mg of the compound administered for dosages of 0.1 mg to 3 mg.

22. The method of any one of claims 16-21, wherein the average AUC varies based on a rate of administration of the compound.

23. The method of claim 22, wherein the average AUC increases non-linearly as the rate of administration of the compound decreases.

24. The method of claim 23, wherein the average AUC per each 1 mg dosage of the compound administered at a rate of 0.0007 mg/min to 0.2 mg/min is up to 2 times, up to 3 times, up to 4 times, or up to 5 times greater than the average AUC per each 1 mg dosage of the compound administered at a rate of greater than 0.2 mg/min to 120 mg/min.

25. The method of any one of claims 1-24, wherein the compound has an average elimination half-life (average t_{1/2}) in the human subject within a range from about 0.1 hr to about 10 hr.

26. The method of claim 25, wherein the average t_{1/2} is greater than or equal to about 0.2 hr, greater than or equal to about 0.3 hr, greater than or equal to about 0.4 hr, greater than or equal to about 0.5 hr, greater than or equal to about 0.6 hr, greater than or equal to about 0.7 hr, greater than or equal to about 0.8 hr, greater than or equal to about 0.9 hr, greater than or equal to about 1 hr, greater than or equal to about 1.5 hr, greater than or equal to about 2 hr, or greater than or equal to about 2.5 hr.

27. The method of claim 25, wherein the average t_{1/2} is less than or equal to about 0.2 hr, less than or equal to about 0.3 hr, less than or equal to about 0.4 hr, less than or equal to about 0.5 hr, less than or equal to about 0.6 hr, less than or equal to about 0.7 hr, less than or equal to about 0.8 hr, less than or equal to about 0.9 hr, less than or equal to about 1 hr, less than or equal to about 1.5 hr, less than or equal to about 2 hr, or less than or equal to about 2.5 hr.

28. The method of claim 25, wherein the average t_{1/2} is within a range from about 0.15 hr to about 3 hr.

29. The method of any one of claims 25-28, wherein the average t_{1/2} increases non-linearly with increasing dosage.

30. The method of claim 29, wherein the average t_{1/2} for dosages greater than 3 mg to 100 mg is up to 2 times, up to 3 times, up to 4 times, up to 5 times, up to 6 times, up to 7 times, up to 8 times, up to 9 times, up to 10 times, up to 15 times, or up to 20 times greater than the average t_{1/2} for dosages of 0.1 mg to 3 mg.

31. The method of any one of claims 25-30, wherein the average t_{1/2} varies based on a rate of administration of the compound.

32. The method of claim 31, wherein the average t_{1/2} increases non-linearly as the rate of administration of the compound decreases.

33. The method of claim 32, wherein the average t_{1/2} of a compound administered at a rate of 0.007 mg/min to 0.2 mg/min is up to 2 times, up to 3 times, up to 4 times, up to 5 times, up to 6 times, up to 7 times, up to 8 times, up to 9 times, or up to 10 times greater than the average t_{1/2} of a compound administered at a rate of greater than 0.2 mg/min to 120 mg/min.

34. The method of any one of claims 1-33, further comprising: producing an average clearance (average CL) in the subject within a range from about 2,000 mL/hr to about 100,000 mL/hr.

35. The method of claim 34, wherein the average CL of the compound administered is greater than or equal to 2,000 mL/hr, 4,000 mL/hr, 6,000 mL/hr, 8,000 mL/hr, 10,000 mL/hour, 15,000 mL/hr, 20,000 mL/hr, 25,000 mL/hr, 30,000 mL/hr, 35,000 mL/hr, 40,000 mL/hr, 45,000 mL/hr, or 50,000 mL/hr.

36. The method of claim 34, wherein the average CL per each 1 mg dosage of the compound administered is less than or equal to 60,000 mL/hr, 70,000 mL/hr, 80,000 mL/hr, 90,000 mL/hr, or 100,000 mL/hr.

37. The method of claim 34, wherein the average CL of the compound administered is within a range from 4,000 mL/hr to 46,000 mL/hr.

38. The method of any one of claims 34-37, wherein the average CL decreases non-linearly with increasing dosage.

39. The method of claim 38, the average CL of the compound administered for dosages of 0.1 mg to 3 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average CL of the compound administered for dosages greater than 3 mg to 100 mg.

40. The method of any one of claims 34-39, wherein the average CL varies based on a rate of administration of the compound.

41. The method of claim 40, wherein the average CL decreases non-linearly as the rate of administration of the compound decreases.

42. The method of claim 41, wherein the average CL of the compound administered at a rate of greater than 0.2 mg/min to 120 mg/min is up to 2 times, up to 3 times, up to 4 times, or up to 5 times greater than the average CL of the compound administered at a rate of 0.0007 mg/min to 0.2 mg/min.

43. The method of any one of claims 1-42, further comprising: producing an average volume of distribution (average V_{d}) in the subject within a range from about 200 mL to about 20,000 mL.

44. The method of claim 43, wherein the average V_{d} of the compound administered is greater than or equal to 200 mL, 300 mL, 400 mL, 500 mL, 1,000 mL, 1,500 mL, 2,000 mL, 2,500 mL, 3,000 mL, 4,000 mL, 5,000 mL, 6,000 mL, 7,000 mL, 8,000 mL, 9,000 mL, or 10,000 mL.

45. The method of claim 43, wherein the average V_{d} of the compound administered is less than or equal to 11,000 mL, 12,000 mL, 13,000 mL, 14,000 mL, 15,000 mL, 16,000 mL, 17,000 mL, 18,000 mL, 19,000 mL, or 20,000 mL.

46. The method of claim 43, wherein the average V_{d} of the compound administered is within a range from 3,000 mL to 10,000 mL.

47. The method of any one of claims 43-46, wherein the average V_{d} increases non-linearly with increasing dosage.

48. The method of claim 47, wherein the average V_{d} of the compound administered for dosages greater than 3 to 100 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average V_{d} of the compound administered for dosages of 0.1 mg to 3 mg.

49. The method of any one of claims 43-48, wherein the average V_{d} varies based on a rate of administration of the compound.

50. The method of claim 49, wherein the average V_{d} decreases non-linearly as the rate of administration of the compound decreases.

51. The method of claim 51, wherein the average V_{d} decreases as the rate of administration of the compound decreases. For example, the average V_{d} of the compound administered at a rate of greater than 0.2 mg/min to 120 mg/min is up to 2 times, up to 3 times, up to 4 times, or up to 5 times greater than the average V_{d} of the compound administered at a rate of 0.0007 mg/min to 0.2 mg/min.

52. The method of any one of claims 1-51, wherein the polypeptide has at least 80%, at least 85%, at least 90%, or at least 95% sequence identify with MCMPCFTTDHQMARRCDDCCGGRGRGKCYGPQCLCR (SEQ ID NO: 9) or a fragment thereof.

53. The method of any one of claims 1-51, wherein the polypeptide has at least 80%, at least 85%, at least 90%, or at least 95% sequence identify with any one of SEQ ID NO: 1-SEQ ID NO: 481 or a fragment thereof.

54. The method of any one of claims 1-51, wherein the polypeptide is any of SEQ ID NO: 482-SEQ ID NO: 485 or a fragment thereof.

55. The method of any one of claims 1-54, wherein the fragment of the polypeptide has a length of at least 25 residues.

56. The method of any one of claims 1-55, wherein each amino acid of the polypeptide is independently selected as an L- or D-enantiomer.

57. The method of any one of claims 1-56, wherein the polypeptide contains no lysine residues.

58. The method of any one of claims 1-57, wherein the polypeptide contains a single lysine residue.

59. The method of claim 58, wherein the single lysine residue is located at a position corresponding to K-27 of native chlorotoxin, K-23 of native chlorotoxin, or K-15 of native chlorotoxin.

60. The method of any one of claims 1-59, wherein one, two, or three methionine residues of the polypeptide are replaced with other amino acids.

61. The method of any one of claims 1-60, wherein the N-terminus of the polypeptide is blocked by acetylation or cyclization.

62. The method of any one of claims 1-61, wherein the polypeptide comprises at least 1, at least 2, at least 3, at least 4, at least 5, or at least 6 disulfide bonds.

63. The method of any one of claims 1-62, wherein the polypeptide comprises an isoelectric point of at least 6.0, at least 6.5, at least 7.0, at least 7.5, at least 8.0, at least 8.5, or at least 9.0.

64. The method of any one of claims 1-63, wherein the compound further comprises an agent.

65. The method of claim 64, wherein the polypeptide is conjugated to the agent.

66. The method of claim 65, wherein the polypeptide comprises a single lysine residue and the agent is conjugated to the polypeptide at the single lysine residue.

67. The method of claim 65, wherein the polypeptide comprises no lysine residues and the agent is conjugated to the polypeptide at the N-terminus of the polypeptide.

68. The method of any one of claims 1-67, wherein the compound has the structure of Formula (IV), or a pharmaceutically acceptable salt thereof: 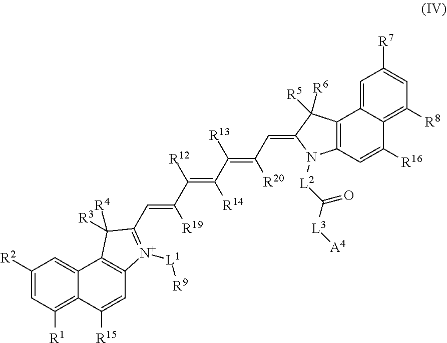
wherein:
R¹, R², R³, R⁴, R⁵, R⁶, R⁷, R⁸, R¹⁵, and R¹⁶ are each independently selected from hydrogen, C₁-C₆ alkyl, C₁-C₆ alkylene-COOH, sulfonate, C₁-C₆ alkylene-sulfonate, —COOH, —SO₂—NH₂, or C₁-C₆ alkoxy;
R⁹ is hydrogen, sulfonate, amine, or —COOH;
L¹ is C₃-C₆ alkylene;
L² is C₁-C₁₀ alkylene;
L³ is a bond, —O—, —NR¹⁰—, —NR¹⁰—C₁-C₆ alkylene-, —O—NR¹⁰—, —NR¹⁰—C₁-C₆ alkylene-(O—C₁-C₆ alkylene)ₙ-, —NR¹⁰-L⁴-, —NR¹⁰—C₁-C₆ alkylene-NR¹¹—(C(═O)—C₁-C₆ alkylene-O—)ₘ—, or —NR¹⁰—C₁-C₆ alkylene-NR¹⁰—C₁-C₆ alkylene-NR¹⁰—C₁-C₆ alkylene-;
L⁴ is a bond, -heterocyclyl-, or -heterocyclyl-CO—C₆ alkylene-;
R¹⁰ is hydrogen or C₁-C₆ alkyl;
R¹¹ is hydrogen or C₁-C₆ alkyl;
R¹² and R¹³ are independently selected from hydrogen, C₁-C₆ alkyl, or R¹² and R¹³ are joined together along with the other atoms to which they are attached to form a 5-membered or 6-membered carbocyclic or heterocyclic ring;
R¹⁴ is hydrogen or C₁-C₆ alkylene, (L⁵)-aryl; -(L⁵)-aryl-R²¹ (L⁵)-heteroaryl, -(L⁵)-heteroaryl-R²¹, —NR¹⁷R¹⁸, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
L⁵ is a bond, C₁-C₁₀ alkylene, —O—, —NR¹⁰—;
R¹⁷ and R¹⁸ are each independently hydrogen or aryl;
R¹⁹ and R²⁰ are independently selected from hydrogen, C₁-C₆ alkyl, R¹⁴ and R¹⁹ are joined together along with the other atoms to which they are attached to form a 5-membered or 6-membered carbocyclic or heterocyclic ring, or R¹⁴ and R²⁰ are joined together along with the other atoms to which they are attached to form a 5-membered or 6-membered carbocyclic or heterocyclic ring;
R²¹ is hydrogen, sulfonate, or —COOH;
n is 0, 1, 2, or 3;
m is 0, 1, 2, or 3;
p is 0, 1, 2, or 3;
q is 0, 1, 2, or 3; and
A⁴ is the polypeptide.

69. The method of claim 68, wherein:
R³, R⁴, R⁵, R⁶ are each independently methyl;
R¹, R², R⁷, R⁸, R¹⁵, and R¹⁶ are each independently hydrogen;
R¹², R¹³, R¹⁴, R¹⁹, and R²⁰ are each independently hydrogen;
R⁹ is sulfonate;
R¹⁰ is hydrogen;
L¹ is butylene;
L² is pentylene; or
L³ is selected from a bond, —O—, —NR¹⁰—, —NR¹⁰—C₁-C₆ alkylene-, —O—NR¹⁰—, or —NR¹⁰-L⁴-.

70. The method of any one of claims 1-69, wherein the compound has the structure of any one of Formulas (IX), (X), (XI), (XII), (XIII), (XIV), (XV), or (XVI), wherein A⁴ is the polypeptide:

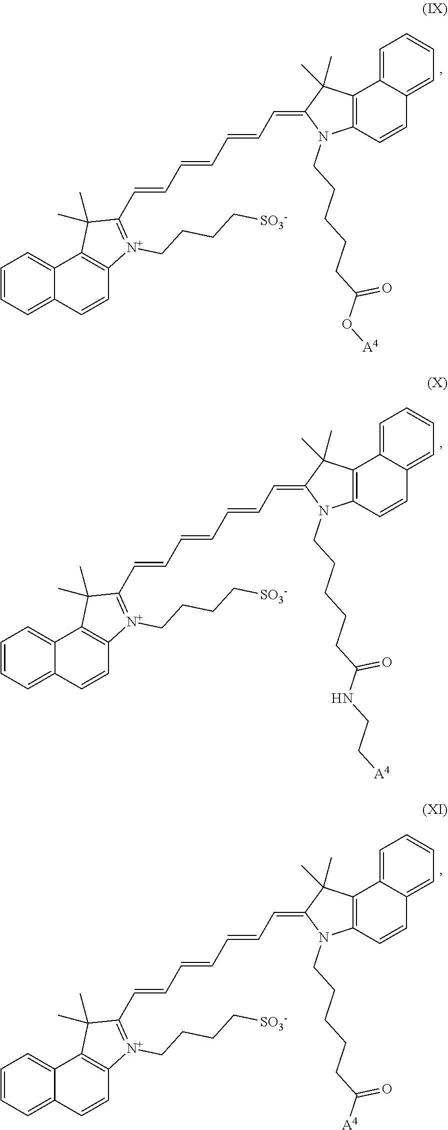

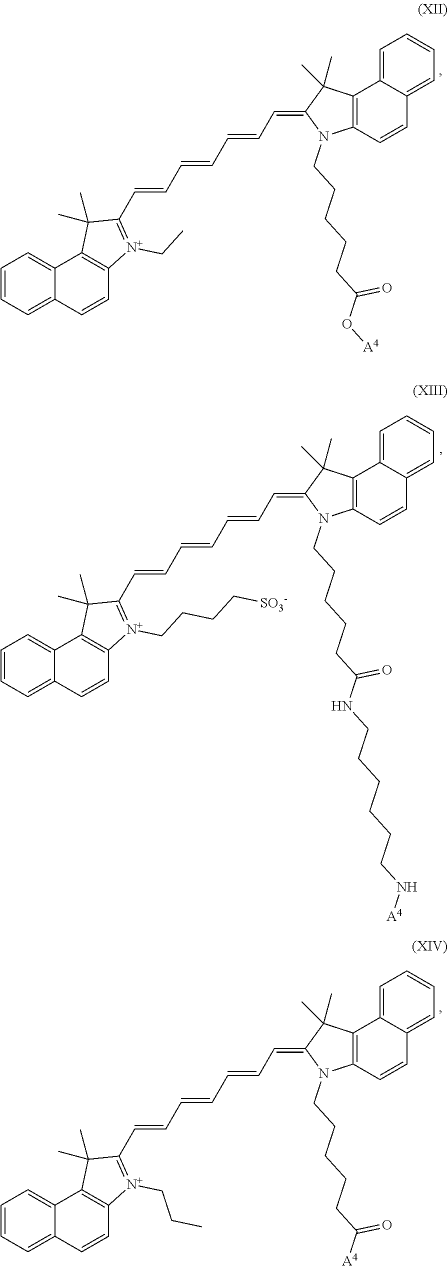

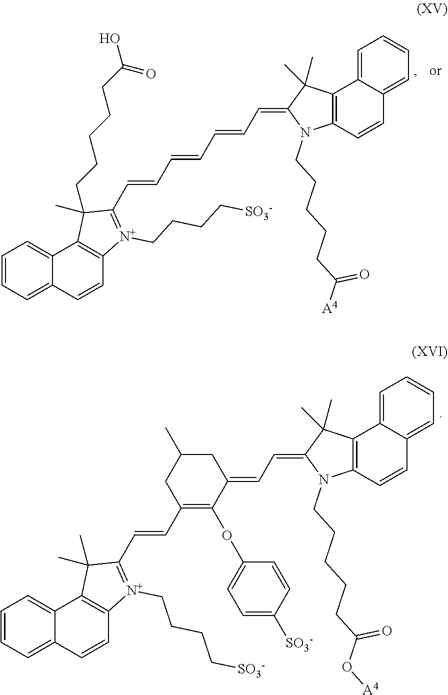

71. The method of any one of claims 1-70, wherein the compound comprises a detectable agent.

72. The method of claim 71, wherein the compound is conjugated to the detectable agent.

73. The method of any one of claims 71-72, wherein the detectable agent comprises a dye, a fluorophore, a fluorescent biotin compound, a luminescent compound, a chemiluminescent compound, a radioisotope, a paramagnetic metal ion, or a combination thereof.

74. The method of any one of claims 1-73, wherein the compound comprises a therapeutic agent.

75. The method of claim 74, wherein the polypeptide is conjugated to the therapeutic agent.

76. The method of any one of claims 74-75, wherein the therapeutic agent comprises a radioisotope, toxin, enzyme, sensitizing drug, radiosensitizer, nucleic acid, interfering RNA, antibody, antibody fragment, aptamer, anti-angiogenic agent, cisplatin, carboplatin, oxaliplatin, anti-metabolite, mitotic inhibitor, growth factor inhibitor, cytotoxin, microtubule disrupting agent, DNA modifying agent, maytansine derivative, auristatin derivative, dolostatin derivative, monomethyl auristatin E, monomethyl auristatin F, DM1, calicheamicin, duocarmycin derivative, campthotecin, pyrrolobenzodiazepine, paclitaxel, cyclophosphamide, chlorambucil, melphlan, bufulfan, carmustine, ifosfamide, temozolomide, topotecan, fluorouracil, vincristine, vinblastine, procarbazine, dacarbazine, altretamine, methotrexate, pemetrexed, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane, amifostine, lenalidomide, imatinib, abiraterone, erlotinib, enzalutimide, everolimus palbociclib, pomalidomide, sutininib, sorafenib, imatinib, gefitinib, afatinib, axitinib, crizotinib, vismoegib, dabrefenib, vemurafenib, or a combination thereof.

77. The method of any one of claims 1-76, wherein intravenously administering the compound comprises intravenously administering a composition comprising the compound and a pharmaceutically acceptable carrier.

78. The method of claim 77, wherein the composition comprises a pH within a range from about 6 to about 7.5.

79. The method of any one of claims 77-78, wherein the composition comprises an ionic strength less than or equal to about 50 mM.

80. The method of any one of claims 77-79, wherein the composition further comprises a buffer comprising histidine, tris, HEPES, ethylene diamine, or a combination thereof.

81. The method of any one of claims 77-80, wherein the composition further comprises a sugar alcohol.

82. The method of any one of claims 77-81, wherein the composition comprises from about 0 mM to about 50 mM histidine, from about 0 mM to about 20 mM tris, about 20 mM methionine, from about 3% to about 10% sugar alcohol, and a pH within a range from about 6 to about 7.5.

83. The method of any one of claims 1-82, further comprising detecting the presence or absence of the compound in a tissue or cell, wherein the presence of the compound in the tissue or cell indicates the presence of a cancerous tissue or cancer cell.

84. The method of claim 83, wherein the cancerous tissue or cancer cell is associated with one or more of: brain cancer, glioma, astrocytoma, medulloblastoma, oligiodendroglioma, choroids plexus carcinoma, ependymoma, pituitary cancer, neuroblastoma, basal cell carcinoma, cutaneous squamous cell carcinoma, melanoma, head and neck cancer, lung cancer, small cell lung cancer, non-small cell lung cancer, breast cancer, ductal carcinoma in situ, intestinal cancer, pancreatic cancer, liver cancer, kidney cancer, bladder cancer, carcinoma of unknown primary, sarcoma, osteosarcoma, rhabdomyosarcoma, Ewing's sarcoma, gastrointestinal stromal tumors, melanoma, ovarian cancer, cervical cancer, lymphoma, Hodgkin's lymphoma, non-Hodgkin's lymphoma, thyroid cancer, anal cancer, colo-rectal cancer, endometrial cancer, laryngeal cancer, multiple myeloma, prostate cancer, retinoblastoma, gastric cancer, esophageal cancer, testicular cancer, or Wilm's tumor.

85. The method of any one of claims 83-84, wherein the compound binds to the cancerous tissue or cancer cell.

86. The method of any one of claims 83-85, wherein the detecting is performed using fluorescence imaging.

87. The method of any one of claims 83-86, further comprising surgically removing the cancerous tissue or cancer cell from the human subject.

88. The method of any one of claims 1-87, wherein the compound is administered at a dosage sufficient to treat cancer in the human subject.

89. The method of claim 88, wherein the cancer comprises one or more of: brain cancer, glioma, astrocytoma, medulloblastoma, oligiodendroglioma, choroids plexus carcinoma, ependymoma, pituitary cancer, neuroblastoma, basal cell carcinoma, cutaneous squamous cell carcinoma, melanoma, head and neck cancer, lung cancer, small cell lung cancer, non-small cell lung cancer, breast cancer, ductal carcinoma in situ, intestinal cancer, pancreatic cancer, liver cancer, kidney cancer, bladder cancer, carcinoma of unknown primary, sarcoma, osteosarcoma, rhabdomyosarcoma, Ewing's sarcoma, gastrointestinal stromal tumors, melanoma, ovarian cancer, cervical cancer, lymphoma, Hodgkin's lymphoma, non-Hodgkin's lymphoma, thyroid cancer, anal cancer, colo-rectal cancer, endometrial cancer, laryngeal cancer, multiple myeloma, prostate cancer, retinoblastoma, gastric cancer, esophageal cancer, testicular cancer, or Wilm's tumor.

90. The method of any one of claims 88-89, wherein the compound binds to a cancerous tissue or cancer cell.

91. The method of any one of claims 1-90, wherein the compound is intravenously administered about 1 hr, about 2 hrs, about 3 hrs, about 4 hrs, about 5 hrs, about 6 hrs, about 7 hrs, about 8 hrs, about 9 hrs, about 10 hrs, about 11 hrs, about 12 hrs, about 13 hrs, about 14 hrs, about 15 hrs, about 16 hrs, about 17 hrs, about 18 hrs, about 19 hrs, about 20 hrs, about 21 hrs, about 22 hrs, about 23 hrs, about 24 hrs, about 36 hrs, about 48 hrs, about 60 hrs, or about 72 hrs prior to performing surgery on the human subject.

92. A method of administering a composition to a human subject, the method comprising:
determining a rate of administration of a compound to a human subject, the compound comprising a polypeptide having at least 80% sequence identity with MCMPCFTTDHQMARRCDDCCGGRGRGKCYGPQCLCR (SEQ ID NO: 9) or a fragment thereof, wherein a pharmacokinetic profile of the compound in the human subject varies according to the rate of administration of the compound; and
intravenously administering the compound to the human subject at the determined rate.

93. A method of administering a composition to a human subject, the method comprising:
determining a rate of administration of a compound to a human subject, the compound comprising a polypeptide having at least 80% sequence identity with any one of SEQ ID NO: 1-SEQ ID NO: 481 or a fragment thereof, wherein a pharmacokinetic profile of the compound in the human subject varies according to the rate of administration of the compound; and
intravenously administering the compound to the human subject at the determined rate.

94. A method of administering a composition to a human subject, the method comprising:
determining a rate of administration of a compound to a human subject, the compound comprising a polypeptide any one of SEQ ID NO: 482-SEQ ID NO: 485 or a fragment thereof, wherein a pharmacokinetic profile of the compound in the human subject varies according to the rate of administration of the compound; and
intravenously administering the compound to the human subject at the determined rate.

95. The method of any one of claims 92-94, wherein the rate of administration per 1 mg dosage is selected from 120 mg/min to 0.5 mg/min, 0.5 mg/min to 0.2 mg/min, or 0.2 mg/min to 0.0007 mg/min.

96. The method of any one of claims 92-95, wherein determining the rate of administration comprises determining a time period over which a predetermined dosage is to be intravenously administered to the human subject.

97. The method of claim 96, wherein the predetermined dosage is within a range from about 0.1 mg to about 100 mg.

98. The method of any one of claims 96-97, wherein the time period is selected from: less than or equal to about 2 minutes, within a range from about 2 minutes to about 5 minutes, or greater than or equal to about 5 minutes.

99. The method of any one of claims 92-98, wherein the rate of administration is determined based on one or more characteristics of a cancer in the human subject.

100. The method of claim 99, wherein the cancer comprises one or more of: brain cancer, glioma, astrocytoma, medulloblastoma, oligiodendroglioma, choroids plexus carcinoma, ependymoma, pituitary cancer, neuroblastoma, basal cell carcinoma, cutaneous squamous cell carcinoma, melanoma, head and neck cancer, lung cancer, small cell lung cancer, non-small cell lung cancer, breast cancer, ductal carcinoma in situ, intestinal cancer, pancreatic cancer, liver cancer, kidney cancer, bladder cancer, carcinoma of unknown primary, sarcoma, osteosarcoma, rhabdomyosarcoma, Ewing's sarcoma, gastrointestinal stromal tumors, melanoma, ovarian cancer, cervical cancer, lymphoma, Hodgkin's lymphoma, non-Hodgkin's lymphoma, thyroid cancer, anal cancer, colo-rectal cancer, endometrial cancer, laryngeal cancer, multiple myeloma, prostate cancer, retinoblastoma, gastric cancer, esophageal cancer, testicular cancer, or Wilm's tumor.

101. The method of any one of claims 99-100, wherein the one or more characteristics comprise a type of the cancer.

102. The method of any one of claims 99-101, wherein the one or more characteristics comprise an aggressiveness of the cancer.

103. The method of claim 100, wherein the determined rate of administration is higher when the cancer is more aggressive and lower when the cancer is less aggressive.

104. The method of any one of claims 99-103, wherein the one or more characteristics comprise a location of the cancer.

105. The method of claim 104, wherein the determined rate of administration is lower when the cancer is located in the brain and higher when the cancer is not located in the brain.

106. The method of any one of claims 99-105, wherein the one or more characteristics comprises a rate of uptake of the compound by cancerous tissue or cancer cells.

107. The method of claim 106, wherein the determined rate of administration is higher when the rate of uptake is higher and lower when the rate of uptake is lower.

108. The method of any one of claims 99-107, wherein the rate of administration is determined based on an amount of time between the administration of the compound and performing of a surgical procedure on the human subject.

109. The method of claim 108, wherein the determined rate is higher when the amount of time is shorter and lower when the amount of time is longer.

110. The method of any one of claims 108-109, wherein the rate of administration is determined based on a type of a surgical procedure to be performed on the human subject following the administration of the compound.

111. The method of any one of claims 108-110, further comprising performing the surgical procedure on the human subject, wherein the determined rate of administration results in an average blood plasma concentration of the compound greater than about 10 ng/mL when the surgical procedure is performed.

112. The method of claim 111, wherein the surgical procedure is performed to remove cancerous tissue or cancer cells from the human subject.

113. The method of any one of claims 99-112, wherein the rate of administration is determined based on a therapeutic usage of the compound.

114. The method of any one of claims 99-113, further comprising producing a pharmacokinetic profile in the human subject.

115. The method of claim 114, wherein the pharmacokinetic profile comprises an average maximum blood plasma concentration (average Cₘₐₓ) in the human subject within a range from about 15 ng/mL to about 600 ng/mL per each 1 mg dosage of the compound administered.

116. The method of claim 115, wherein the average Cₘₐₓ per each 1 mg dosage of the compound administered is greater than or equal to about 20 ng/mL, greater than or equal to about 30 ng/mL, greater than or equal to about 40 ng/mL, greater than or equal to about 50 ng/mL, greater than or equal to about 60 ng/mL, greater than or equal to about 70 ng/mL, greater than or equal to about 80 ng/mL, greater than or equal to about 90 ng/mL, greater than or equal to about 100 ng/mL, greater than or equal to about 150 ng/mL, greater than or equal to about 200 ng/mL, greater than or equal to about 250 ng/mL, greater than or equal to about 300 ng/mL, greater than or equal to about 350 ng/mL, greater than or equal to about 400 ng/mL, greater than or equal to about 450 ng/mL, greater than or equal to about 500 ng/mL, or greater than or equal to about 550 ng/mL.

117. The method of claim 116, wherein the average Cₘₐₓ per each 1 mg dosage of the compound administered is less than or equal to about 20 ng/mL, less than or equal to about 30 ng/mL, less than or equal to about 40 ng/mL, less than or equal to about 50 ng/mL, less than or equal to about 60 ng/mL, less than or equal to about 70 ng/mL, less than or equal to about 80 ng/mL, less than or equal to about 90 ng/mL, less than or equal to about 100 ng/mL, less than or equal to about 150 ng/mL, less than or equal to about 200 ng/mL, less than or equal to about 250 ng/mL, less than or equal to about 300 ng/mL, less than or equal to about 350 ng/mL, less than or equal to about 400 ng/mL, less than or equal to about 450 ng/mL, less than or equal to about 500 ng/mL, or less than or equal to about 550 ng/mL.

118. The method of claim 116, wherein the average Cₘₐₓ per each 1 mg dosage of the compound administered is within a range from about 50 ng/mL to about 300 ng/mL.

119. The method of any one of claims 115-118, wherein the average time (average Tₘₐₓ) at which the average Cₘₐₓ is reached is within a range from about 0.5 min to about 120 min following administration of the compound.

120. The method of any one of claims 115-119, wherein the average Cₘₐₓ increases non-linearly with increasing dosage.

121. The method of claim 120, wherein the average Cₘₐₓ/mg of the compound administered for dosages greater than 3 mg to 100 mg is up to 2 times, up to 3 times, up to 4 times, up to 5 times, up to 6 times, up to 7 times, up to 8 times, up to 9 times, or up to 10 times greater than the average Cₘₐₓ/mg of the compound administered for dosages of 0.1 mg to 3 mg.

122. The method of any one of claims 115-121, wherein the average Cₘₐₓ varies based on a rate of administration of the compound.

123. The method of claim 122, wherein the average Cₘₐₓ decreases non-linearly as the rate of administration of the compound decreases.

124. The method of claim 123, wherein the average Cₘₐₓ per each 1 mg dosage of the compound administered at a rate of greater than 0.2 mg/min to 120 mg/min is up to 1.5 times, up to 2 times, up to 2.5 times, or up to 3 times greater than the average Cₘₐₓ per each 1 mg dosage of the compound administered at a rate of 0.0007 mg/min to 0.2 mg/min.

125. The method of any one of claims 114-124, wherein the pharmacokinetic profile comprises an average area under the curve (average AUC) in the subject within a range from about 10 hr*ng/mL to about 750 hr*ng/mL per each 1 mg dosage of the compound administered.

126. The method of claim 125, wherein the average AUC per each 1 mg dosage of the compound administered is greater than or equal to about 20 hr*ng/mL, greater than or equal to about 30 hr*ng/mL, greater than or equal to about 40 hr*ng/mL, greater than or equal to about 50 hr*ng/mL, greater than or equal to about 60 hr*ng/mL, greater than or equal to about 70 hr*ng/mL, greater than or equal to about 80 hr*ng/mL, greater than or equal to about 90 hr*ng/mL, greater than or equal to about 100 hr*ng/mL, greater than or equal to about 150 hr*ng/mL, greater than or equal to about 200 hr*ng/mL, greater than or equal to about 250 hr*ng/mL, greater than or equal to about 300 hr*ng/mL, greater than or equal to about 350 hr*ng/mL, greater than or equal to about 400 hr*ng/mL, greater than or equal to about 450 hr*ng/mL, greater than or equal to about 500 hr*ng/mL, greater than or equal to about 550 hr*ng/mL, greater than or equal to about 600 hr*ng/mL, greater than or equal to about 650 hr*ng/mL, or greater than or equal to about 700 hr*ng/mL.

127. The method of claim 125, wherein the average AUC per each 1 mg dosage of the compound administered is less than or equal to about 20 hr*ng/mL, less than or equal to about 30 hr*ng/mL, less than or equal to about 40 hr*ng/mL, less than or equal to about 50 hr*ng/mL, less than or equal to about 60 hr*ng/mL, less than or equal to about 70 hr*ng/mL, less than or equal to about 80 hr*ng/mL, less than or equal to about 90 hr*ng/mL, less than or equal to about 100 hr*ng/mL, less than or equal to about 150 hr*ng/mL, less than or equal to about 200 hr*ng/mL, less than or equal to about 250 hr*ng/mL, less than or equal to about 300 hr*ng/mL, less than or equal to about 350 hr*ng/mL, less than or equal to about 400 hr*ng/mL, less than or equal to about 450 hr*ng/mL, less than or equal to about 500 hr*ng/mL, less than or equal to about 550 hr*ng/mL, less than or equal to about 600 hr*ng/mL, less than or equal to about 650 hr*ng/mL, or less than or equal to about 700.

128. The method of claim 125, wherein the average AUC per each 1 mg dosage of the compound administered is within a range from about 15 hr*ng/mL to about 400 hr*ng/mL.

129. The method of any one of claims 125-128 wherein the average AUC increases non-linearly with increasing dosage.

130. The method of claim 129, wherein the average AUC/mg of the compound administered for dosages greater than 3 mg to 100 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average AUC/mg of the compound administered for dosages of 0.1 mg to 3 mg.

131. The method of any one of claims 125-130, wherein the average AUC varies based on the rate of administration of the compound.

132. The method of claim 131, wherein the average AUC increases non-linearly as the rate of administration of the compound decreases.

133. The method of claim 132, wherein the average AUC per each 1 mg dosage of the compound administered at a rate of 0.0007 mg/min to 0.2 mg/min is up to 2 times, up to 3 times, up to 4 times, or up to 5 times greater than the average AUC per each 1 mg dosage of the compound administered at a rate of greater than 0.2 mg/min to 120 mg/min.

134. The method of any one of claims 114-133, wherein the pharmacokinetic profile comprises an average elimination half-life (average t_{1/2}) in the human subject within a range from about 0.1 hr to about 10 hr.

135. The method of claim 134, wherein the average t_{1/2} is greater than or equal to about 0.2 hr, greater than or equal to about 0.3 hr, greater than or equal to about 0.4 hr, greater than or equal to about 0.5 hr, greater than or equal to about 0.6 hr, greater than or equal to about 0.7 hr, greater than or equal to about 0.8 hr, greater than or equal to about 0.9 hr, greater than or equal to about 1 hr, greater than or equal to about 1.5 hr, greater than or equal to about 2 hr, or greater than or equal to about 2.5 hr.

136. The method of claim 134, wherein the average t_{1/2} is less than or equal to about 0.2 hr, less than or equal to about 0.3 hr, less than or equal to about 0.4 hr, less than or equal to about 0.5 hr, less than or equal to about 0.6 hr, less than or equal to about 0.7 hr, less than or equal to about 0.8 hr, less than or equal to about 0.9 hr, less than or equal to about 1 hr, less than or equal to about 1.5 hr, less than or equal to about 2 hr, or less than or equal to about 2.5 hr.

137. The method of claim 134, wherein the average t_{1/2} is within a range from about 0.15 hr to about 3 hr.

138. The method of any one of claims 134-137, wherein the average t_{1/2} increases non-linearly with increasing dosage.

139. The method of claim 138, wherein the average t_{1/2} for dosages greater than 3 mg to 100 mg is up to 2 times, up to 3 times, up to 4 times, up to 5 times, up to 6 times, up to 7 times, up to 8 times, up to 9 times, up to 10 times, up to 15 times, or up to 20 times greater than the average t_{1/2} for dosages of 0.1 mg to 3 mg.

140. The method of any one of claims 134-139, wherein the average t_{1/2} varies based on the rate of administration of the compound.

141. The method of claim 140, wherein the average t_{1/2} increases non-linearly as the rate of administration of the compound decreases.

142. The method of claim 141, wherein the average t_{1/2} of the compound administered at a rate of 0.0007 mg/min to 0.2 mg/min is up to 2 times, up to 3 times, up to 4 times, up to 5 times, up to 6 times, up to 7 times, up to 8 times, up to 9 times, or up to 10 times greater than the average t_{1/2} of the compound administered at a rate of greater than 0.2 mg/min to 120 mg/min.

143. The method of any one of claims 114-142, wherein the pharmacokinetic profile comprises an average clearance (average CL) in the subject within a range from about 2,000 mL/hr to about 100,000 mL/hr.

144. The method of claim 143, wherein the average CL of the compound administered is greater than or equal to 2,000 mL/hr, 4,000 mL/hr, 6,000 mL/hr, 8,000 mL/hr, 10,000 mL/hour, 15,000 mL/hr, 20,000 mL/hr, 25,000 mL/hr, 30,000 mL/hr, 35,000 mL/hr, 40,000 mL/hr, 45,000 mL/hr, or 50,000 mL/hr.

145. The method of claim 143, wherein the average CL of the compound administered is less than or equal to 60,000 mL/hr, 70,000 mL/hr, 80,000 mL/hr, 90,000 mL/hr, or 100,000 mL/hr.

146. The method of claim 143, wherein the average CL of the compound administered is within a range from 4,000 mL/hr to 46,000 mL/hr.

147. The method of any one of claims 143-146, wherein the average CL decreases non-linearly with increasing dosage.

148. The method of claim 147, wherein the average CL of the compound administered for dosages of 0.1 mg to 3 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average CL of the compound administered for dosages greater than 3 mg to 100 mg.

149. The method of any one of claims 143-148, wherein the average CL varies based on a rate of administration of the compound.

150. The method of claim 149, wherein the average CL decreases non-linearly as the rate of administration of the compound decreases.

151. The method of claim 150, wherein the average CL of the compound administered at a rate of greater than 0.2 mg/min to 120 mg/min is up to 2 times, up to 3 times, up to 4 times, or up to 5 times greater than the average CL of the compound administered at a rate of 0.0007 mg/min to 0.2 mg/min.

152. The method of any one of claims 114-151, wherein the pharmacokinetic profile comprises an average volume of distribution (average V_{d}) in the subject within a range from about 200 mL to about 20,000 mL.

153. The method of claim 152, wherein the average V_{d} of the compound administered is greater than or equal to 200 mL, 300 mL, 400 mL, 500 mL, 1,000 mL, 1,500 mL, 2,000 mL, 2,500 mL, 3,000 mL, 4,000 mL, 5,000 mL, 6,000 mL, 7,000 mL, 8,000 mL, 9,000 mL, or 10,000 mL.

154. The method of claim 152, wherein the average V_{d} of the compound administered is less than or equal to 11,000 mL, 12,000 mL, 13,000 mL, 14,000 mL, 15,000 mL, 16,000 mL, 17,000 mL, 18,000 mL, 19,000 mL, or 20,000 mL.

155. The method of claim 152, wherein the average V_{d} of the compound administered is within a range from 3,000 mL to 10,000 mL.

156. The method of any one of claims 152-155, wherein the average V_{d} increases non-linearly with increasing dosage.

157. The method of claim 156, wherein the average V_{d} of the compound administered for dosages greater than 3 to 100 mg is up to 2 times, up to 3 times, up to 4 times, up to 5 times, up to 6 times, up to 7 times, up to 8 times, up to 9 times, up to 10 times, up to 15 times, up to 20 times, or up to 25 times greater than the average V_{d} of the compound administered for dosages of 0.1 mg to 3 mg.

158. The method of any one of claims 152-157, wherein the average V_{d} varies based on a rate of administration of the compound.

159. The method of claim 158, wherein the average V_{d} decreases non-linearly as the rate of administration of the compound decreases.

160. The method of claim 159, wherein the average V_{d} decreases as the rate of administration of the compound decreases. For example, the average V_{d} of the compound administered at a rate of greater than 0.2 mg/min to 120 mg/min is up to 2 times, up to 3 times, up to 4 times, or up to 5 times greater than the average V_{d} of the compound administered at a rate of 0.0007 mg/min to 0.2 mg/min.


SEQUENCE LISTING (485 sequences)

SEQ ID NO: 1 - protein, 36 aa, Artificial Sequence
  Description of Artificial Sequence Synthetic polypeptide
MCMPCFTTDHQMARKCDDCCGGKGRGKCYGPQCLCR

SEQ ID NO: 2 - protein, 36 aa, Artificial Sequence
  Description of Artificial Sequence Synthetic polypeptide
MCMPCFTTDHQMARACDDCCGGKGRGKCYGPQCLCR

SEQ ID NO: 3 - protein, 36 aa, Artificial Sequence
  Description of Artificial Sequence Synthetic polypeptide
MCMPCFTTDHQMARRCDDCCGGKGRGKCYGPQCLCR

SEQ ID NO: 4 - protein, 36 aa, Artificial Sequence
  Description of Artificial Sequence Synthetic polypeptide
MCMPCFTTDHQMARKCDDCCGGAGRGKCYGPQCLCR

SEQ ID NO: 5 - protein, 36 aa, Artificial Sequence
  Description of Artificial Sequence Synthetic polypeptide
MCMPCFTTDHQMARACDDCCGGAGRGKCYGPQCLCR

SEQ ID NO: 6 - protein, 36 aa, Artificial Sequence
  Description of Artificial Sequence Synthetic polypeptide
MCMPCFTTDHQMARRCDDCCGGAGRGKCYGPQCLCR

SEQ ID NO: 7 - protein, 36 aa, Artificial Sequence
  Description of Artificial Sequence Synthetic polypeptide
MCMPCFTTDHQMARKCDDCCGGRGRGKCYGPQCLCR

SEQ ID NO: 8 - protein, 36 aa, Artificial Sequence
  Description of Artificial Sequence Synthetic polypeptide
MCMPCFTTDHQMARACDDCCGGRGRGKCYGPQCLCR

SEQ ID NO: 9 - protein, 36 aa, Artificial Sequence
  Description of Artificial Sequence Synthetic polypeptide
MCMPCFTTDHQMARRCDDCCGGRGRGKCYGPQCLCR

SEQ ID NO: 10 - protein, 36 aa, Artificial Sequence
  Description of Artificial Sequence Synthetic polypeptide
MCMPCFTTDHQMARKCDDCCGGKGRGACYGPQCLCR

SEQ ID NO: 11 - protein, 36 aa, Artificial Sequence
  Description of Artificial Sequence Synthetic polypeptide
MCMPCFTTDHQMARACDDCCGGKGRGACYGPQCLCR

SEQ ID NO: 12 - protein, 36 aa, Artificial Sequence
  Description of Artificial Sequence Synthetic polypeptide
MCMPCFTTDHQMARRCDDCCGGKGRGACYGPQCLCR

SEQ ID NO: 13 - protein, 36 aa, Artificial Sequence
  Description of Artificial Sequence Synthetic polypeptide
MCMPCFTTDHQMARKCDDCCGGAGRGACYGPQCLCR

SEQ ID NO: 14 - protein, 36 aa, Artificial Sequence
  Description of Artificial Sequence Synthetic polypeptide
MCMPCFTTDHQMARACDDCCGGAGRGACYGPQCLCR

SEQ ID NO: 15 - protein, 36 aa, Artificial Sequence
  Description of Artificial Sequence Synthetic polypeptide
MCMPCFTTDHQMARRCDDCCGGAGRGACYGPQCLCR

SEQ ID NO: 16 - protein, 36 aa, Artificial Sequence
  Description of Artificial Sequence Synthetic polypeptide
MCMPCFTTDHQMARKCDDCCGGRGRGACYGPQCLCR

SEQ ID NO: 17 - protein, 36 aa, Artificial Sequence
  Description of Artificial Sequence Synthetic polypeptide
MCMPCFTTDHQMARACDDCCGGRGRGACYGPQCLCR

SEQ ID NO: 18 - protein, 36 aa, Artificial Sequence
  Description of Artificial Sequence Synthetic polypeptide
MCMPCFTTDHQMARRCDDCCGGRGRGACYGPQCLCR

SEQ ID NO: 19 - protein, 36 aa, Artificial Sequence
  Description of Artificial Sequence Synthetic polypeptide
MCMPCFTTDHQMARKCDDCCGGKGRGRCYGPQCLCR

SEQ ID NO: 20 - protein, 36 aa, Artificial Sequence
  Description of Artificial Sequence Synthetic polypeptide
MCMPCFTTDHQMARACDDCCGGKGRGRCYGPQCLCR

SEQ ID NO: 21 - protein, 36 aa, Artificial Sequence
  Description of Artificial Sequence Synthetic polypeptide
MCMPCFTTDHQMARRCDDCCGGKGRGRCYGPQCLCR

SEQ ID NO: 22 - protein, 36 aa, Artificial Sequence
  Description of Artificial Sequence Synthetic polypeptide
MCMPCFTTDHQMARKCDDCCGGAGRGRCYGPQCLCR

SEQ ID NO: 23 - protein, 36 aa, Artificial Sequence
  Description of Artificial Sequence Synthetic polypeptide
MCMPCFTTDHQMARACDDCCGGAGRGRCYGPQCLCR

SEQ ID NO: 24 - protein, 36 aa, Artificial Sequence
  Description of Artificial Sequence Synthetic polypeptide
MCMPCFTTDHQMARRCDDCCGGAGRGRCYGPQCLCR

SEQ ID NO: 25 - protein, 36 aa, Artificial Sequence
  Description of Artificial Sequence Synthetic polypeptide
MCMPCFTTDHQMARKCDDCCGGRGRGRCYGPQCLCR

SEQ ID NO: 26 - protein, 36 aa, Artificial Sequence
  Description of Artificial Sequence Synthetic polypeptide
MCMPCFTTDHQMARACDDCCGGRGRGRCYGPQCLCR

SEQ ID NO: 27 - protein, 36 aa, Artificial Sequence
  Description of Artificial Sequence Synthetic polypeptide
MCMPCFTTDHQMARRCDDCCGGRGRGRCYGPQCLCR

SEQ ID NO: 28 - protein, 36 aa, Artificial Sequence; identical to SEQ ID NO: 27
  Description of Artificial Sequence Synthetic polypeptide

SEQ ID NO: 29 - protein, 36 aa, Artificial Sequence
  Description of Artificial Sequence Synthetic polypeptide
KCMPCFTTDHQMARRCDDCCGGRGRGRCYGPQCLCR

SEQ ID NO: 30 - protein, 36 aa, Artificial Sequence
  Description of Artificial Sequence Synthetic polypeptide
ACAPCFTTDHQAARRCDDCCGGRGRGRCYGPQCLCR

SEQ ID NO: 31 - protein, 36 aa, Artificial Sequence
  Description of Artificial Sequence Synthetic polypeptide
KCAPCFTTDHQAARRCDDCCGGRGRGRCYGPQCLCR

SEQ ID NO: 32 - protein, 36 aa, Artificial Sequence
  Description of Artificial Sequence Synthetic polypeptide
  MOD_RES (15)..(15): Citrulline
  MOD_RES (23)..(23): Citrulline
MCMPCFTTDHQMARXCDDCCGGXGRGKCYGPQCLCR

SEQ ID NO: 33 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
MCMPCFTTDHQMARXCDDCCGGXGRGXCYGPQCLCR

SEQ ID NO: 34 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCMPCFTTDHQMARXCDDCCGGXGRGXCYGPQCLCR

SEQ ID NO: 35 - protein, 36 aa, Artificial Sequence
  Description of Artificial Sequence Synthetic polypeptide
  MOD_RES (15)..(15): Citrulline
  MOD_RES (23)..(23): Citrulline
ACAPCFTTDHQAARXCDDCCGGXGRGKCYGPQCLCR

SEQ ID NO: 36 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
ACAPCFTTDHQAARXCDDCCGGXGRGXCYGPQCLCR

SEQ ID NO: 37 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCAPCFTTDHQAARXCDDCCGGXGRGXCYGPQCLCR

SEQ ID NO: 38 - protein, 43 aa, Artificial Sequence
  Description of Artificial Sequence Synthetic polypeptide
MCMPCFTTDHQMARKCDDCCGGKGRGKCYGPQCLCRGAGAAGG

SEQ ID NO: 39 - protein, 43 aa, Artificial Sequence
  Description of Artificial Sequence Synthetic polypeptide
MCMPCFTTDHQMARACDDCCGGKGRGKCYGPQCLCRGAGAAGG

SEQ ID NO: 40 - protein, 43 aa, Artificial Sequence
  Description of Artificial Sequence Synthetic polypeptide
MCMPCFTTDHQMARRCDDCCGGKGRGKCYGPQCLCRGAGAAGG

SEQ ID NO: 41 - protein, 43 aa, Artificial Sequence
  Description of Artificial Sequence Synthetic polypeptide
MCMPCFTTDHQMARKCDDCCGGAGRGKCYGPQCLCRGAGAAGG

SEQ ID NO: 42 - protein, 43 aa, Artificial Sequence
  Description of Artificial Sequence Synthetic polypeptide
MCMPCFTTDHQMARACDDCCGGAGRGKCYGPQCLCRGAGAAGG

SEQ ID NO: 43 - protein, 43 aa, Artificial Sequence
  Description of Artificial Sequence Synthetic polypeptide
MCMPCFTTDHQMARRCDDCCGGAGRGKCYGPQCLCRGAGAAGG

SEQ ID NO: 44 - protein, 43 aa, Artificial Sequence
  Description of Artificial Sequence Synthetic polypeptide
MCMPCFTTDHQMARKCDDCCGGRGRGKCYGPQCLCRGAGAAGG

SEQ ID NO: 45 - protein, 43 aa, Artificial Sequence
  Description of Artificial Sequence Synthetic polypeptide
MCMPCFTTDHQMARACDDCCGGRGRGKCYGPQCLCRGAGAAGG

SEQ ID NO: 46 - protein, 43 aa, Artificial Sequence
  Description of Artificial Sequence Synthetic polypeptide
MCMPCFTTDHQMARRCDDCCGGRGRGKCYGPQCLCRGAGAAGG

SEQ ID NO: 47 - protein, 43 aa, Artificial Sequence
  Description of Artificial Sequence Synthetic polypeptide
MCMPCFTTDHQMARKCDDCCGGKGRGACYGPQCLCRGAGAAGG

SEQ ID NO: 48 - protein, 43 aa, Artificial Sequence
  Description of Artificial Sequence Synthetic polypeptide
MCMPCFTTDHQMARACDDCCGGKGRGACYGPQCLCRGAGAAGG

SEQ ID NO: 49 - protein, 43 aa, Artificial Sequence
  Description of Artificial Sequence Synthetic polypeptide
MCMPCFTTDHQMARRCDDCCGGKGRGACYGPQCLCRGAGAAGG

SEQ ID NO: 50 - protein, 43 aa, Artificial Sequence
  Description of Artificial Sequence Synthetic polypeptide
MCMPCFTTDHQMARKCDDCCGGAGRGACYGPQCLCRGAGAAGG

SEQ ID NO: 51 - protein, 43 aa, Artificial Sequence
  Description of Artificial Sequence Synthetic polypeptide
MCMPCFTTDHQMARACDDCCGGAGRGACYGPQCLCRGAGAAGG

SEQ ID NO: 52 - protein, 43 aa, Artificial Sequence
  Description of Artificial Sequence Synthetic polypeptide
MCMPCFTTDHQMARRCDDCCGGAGRGACYGPQCLCRGAGAAGG

SEQ ID NO: 53 - protein, 43 aa, Artificial Sequence
  Description of Artificial Sequence Synthetic polypeptide
MCMPCFTTDHQMARKCDDCCGGRGRGACYGPQCLCRGAGAAGG

SEQ ID NO: 54 - protein, 43 aa, Artificial Sequence
  Description of Artificial Sequence Synthetic polypeptide
MCMPCFTTDHQMARACDDCCGGRGRGACYGPQCLCRGAGAAGG

SEQ ID NO: 55 - protein, 43 aa, Artificial Sequence
  Description of Artificial Sequence Synthetic polypeptide
MCMPCFTTDHQMARRCDDCCGGRGRGACYGPQCLCRGAGAAGG

SEQ ID NO: 56 - protein, 43 aa, Artificial Sequence
  Description of Artificial Sequence Synthetic polypeptide
MCMPCFTTDHQMARKCDDCCGGKGRGRCYGPQCLCRGAGAAGG

SEQ ID NO: 57 - protein, 43 aa, Artificial Sequence
  Description of Artificial Sequence Synthetic polypeptide
MCMPCFTTDHQMARACDDCCGGKGRGRCYGPQCLCRGAGAAGG

SEQ ID NO: 58 - protein, 43 aa, Artificial Sequence
  Description of Artificial Sequence Synthetic polypeptide
MCMPCFTTDHQMARRCDDCCGGKGRGRCYGPQCLCRGAGAAGG

SEQ ID NO: 59 - protein, 43 aa, Artificial Sequence
  Description of Artificial Sequence Synthetic polypeptide
MCMPCFTTDHQMARKCDDCCGGAGRGRCYGPQCLCRGAGAAGG

SEQ ID NO: 60 - protein, 43 aa, Artificial Sequence
  Description of Artificial Sequence Synthetic polypeptide
MCMPCFTTDHQMARACDDCCGGAGRGRCYGPQCLCRGAGAAGG

SEQ ID NO: 61 - protein, 43 aa, Artificial Sequence
  Description of Artificial Sequence Synthetic polypeptide
MCMPCFTTDHQMARRCDDCCGGAGRGRCYGPQCLCRGAGAAGG

SEQ ID NO: 62 - protein, 43 aa, Artificial Sequence
  Description of Artificial Sequence Synthetic polypeptide
MCMPCFTTDHQMARKCDDCCGGRGRGRCYGPQCLCRGAGAAGG

SEQ ID NO: 63 - protein, 43 aa, Artificial Sequence
  Description of Artificial Sequence Synthetic polypeptide
MCMPCFTTDHQMARACDDCCGGRGRGRCYGPQCLCRGAGAAGG

SEQ ID NO: 64 - protein, 43 aa, Artificial Sequence
  Description of Artificial Sequence Synthetic polypeptide
MCMPCFTTDHQMARRCDDCCGGRGRGRCYGPQCLCRGAGAAGG

SEQ ID NO: 65 - protein, 43 aa, Artificial Sequence; identical to SEQ ID NO: 64
  Description of Artificial Sequence Synthetic polypeptide

SEQ ID NO: 66 - protein, 43 aa, Artificial Sequence
  Description of Artificial Sequence Synthetic polypeptide
KCMPCFTTDHQMARRCDDCCGGRGRGRCYGPQCLCRGAGAAGG

SEQ ID NO: 67 - protein, 43 aa, Artificial Sequence
  Description of Artificial Sequence Synthetic polypeptide
ACAPCFTTDHQAARRCDDCCGGRGRGRCYGPQCLCRGAGAAGG

SEQ ID NO: 68 - protein, 43 aa, Artificial Sequence
  Description of Artificial Sequence Synthetic polypeptide
KCAPCFTTDHQAARRCDDCCGGRGRGRCYGPQCLCRGAGAAGG

SEQ ID NO: 69 - protein, 43 aa, Artificial Sequence
  Description of Artificial Sequence Synthetic polypeptide
  MOD_RES (15)..(15): Citrulline
  MOD_RES (23)..(23): Citrulline
MCMPCFTTDHQMARXCDDCCGGXGRGKCYGPQCLCRGAGAAGG

SEQ ID NO: 70 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
MCMPCFTTDHQMARXCDDCCGGXGRGXCYGPQCLCRGAGAAGG

SEQ ID NO: 71 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCMPCFTTDHQMARXCDDCCGGXGRGXCYGPQCLCRGAGAAGG

SEQ ID NO: 72 - protein, 43 aa, Artificial Sequence
  Description of Artificial Sequence Synthetic polypeptide
  MOD_RES (15)..(15): Citrulline
  MOD_RES (23)..(23): Citrulline
ACAPCFTTDHQAARXCDDCCGGXGRGKCYGPQCLCRGAGAAGG

SEQ ID NO: 73 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
ACAPCFTTDHQAARXCDDCCGGXGRGXCYGPQCLCRGAGAAGG

SEQ ID NO: 74 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCAPCFTTDHQAARXCDDCCGGXGRGXCYGPQCLCRGAGAAGG

SEQ ID NO: 75 - protein, 36 aa, Artificial Sequence
  Description of Artificial Sequence Synthetic polypeptide
MCMPCFTTDHQMVRKCDDCCGGKGRGKCYGPQCLCR

SEQ ID NO: 76 - protein, 36 aa, Artificial Sequence
  Description of Artificial Sequence Synthetic polypeptide
MCMPCFTTDHQMVRVCDDCCGGKGRGKCYGPQCLCR

SEQ ID NO: 77 - protein, 36 aa, Artificial Sequence
  Description of Artificial Sequence Synthetic polypeptide
MCMPCFTTDHQMVRRCDDCCGGKGRGKCYGPQCLCR

SEQ ID NO: 78 - protein, 36 aa, Artificial Sequence
  Description of Artificial Sequence Synthetic polypeptide
MCMPCFTTDHQMVRKCDDCCGGVGRGKCYGPQCLCR

SEQ ID NO: 79 - protein, 36 aa, Artificial Sequence
  Description of Artificial Sequence Synthetic polypeptide
MCMPCFTTDHQMVRVCDDCCGGVGRGKCYGPQCLCR

SEQ ID NO: 80 - protein, 36 aa, Artificial Sequence
  Description of Artificial Sequence Synthetic polypeptide
MCMPCFTTDHQMVRRCDDCCGGVGRGKCYGPQCLCR

SEQ ID NO: 81 - protein, 36 aa, Artificial Sequence
  Description of Artificial Sequence Synthetic polypeptide
MCMPCFTTDHQMVRKCDDCCGGRGRGKCYGPQCLCR

SEQ ID NO: 82 - protein, 36 aa, Artificial Sequence
  Description of Artificial Sequence Synthetic polypeptide
MCMPCFTTDHQMVRVCDDCCGGRGRGKCYGPQCLCR

SEQ ID NO: 83 - protein, 36 aa, Artificial Sequence
  Description of Artificial Sequence Synthetic polypeptide
MCMPCFTTDHQMVRRCDDCCGGRGRGKCYGPQCLCR

SEQ ID NO: 84 - protein, 36 aa, Artificial Sequence
  Description of Artificial Sequence Synthetic polypeptide
MCMPCFTTDHQMVRKCDDCCGGKGRGVCYGPQCLCR

SEQ ID NO: 85 - protein, 36 aa, Artificial Sequence
  Description of Artificial Sequence Synthetic polypeptide
MCMPCFTTDHQMVRVCDDCCGGKGRGVCYGPQCLCR

SEQ ID NO: 86 - protein, 36 aa, Artificial Sequence
  Description of Artificial Sequence Synthetic polypeptide
MCMPCFTTDHQMVRRCDDCCGGKGRGVCYGPQCLCR

SEQ ID NO: 87 - protein, 36 aa, Artificial Sequence
  Description of Artificial Sequence Synthetic polypeptide
MCMPCFTTDHQMVRKCDDCCGGVGRGVCYGPQCLCR

SEQ ID NO: 88 - protein, 36 aa, Artificial Sequence
  Description of Artificial Sequence Synthetic polypeptide
MCMPCFTTDHQMVRVCDDCCGGVGRGVCYGPQCLCR

SEQ ID NO: 89 - protein, 36 aa, Artificial Sequence
  Description of Artificial Sequence Synthetic polypeptide
MCMPCFTTDHQMVRRCDDCCGGVGRGVCYGPQCLCR

SEQ ID NO: 90 - protein, 36 aa, Artificial Sequence
  Description of Artificial Sequence Synthetic polypeptide
MCMPCFTTDHQMVRKCDDCCGGRGRGVCYGPQCLCR

SEQ ID NO: 91 - protein, 36 aa, Artificial Sequence
  Description of Artificial Sequence Synthetic polypeptide
MCMPCFTTDHQMVRVCDDCCGGRGRGVCYGPQCLCR

SEQ ID NO: 92 - protein, 36 aa, Artificial Sequence
  Description of Artificial Sequence Synthetic polypeptide
MCMPCFTTDHQMVRRCDDCCGGRGRGVCYGPQCLCR

SEQ ID NO: 93 - protein, 36 aa, Artificial Sequence
  Description of Artificial Sequence Synthetic polypeptide
MCMPCFTTDHQMVRKCDDCCGGKGRGRCYGPQCLCR

SEQ ID NO: 94 - protein, 36 aa, Artificial Sequence
  Description of Artificial Sequence Synthetic polypeptide
MCMPCFTTDHQMVRVCDDCCGGKGRGRCYGPQCLCR

SEQ ID NO: 95 - protein, 36 aa, Artificial Sequence
  Description of Artificial Sequence Synthetic polypeptide
MCMPCFTTDHQMVRRCDDCCGGKGRGRCYGPQCLCR

SEQ ID NO: 96 - protein, 36 aa, Artificial Sequence
  Description of Artificial Sequence Synthetic polypeptide
MCMPCFTTDHQMVRKCDDCCGGVGRGRCYGPQCLCR

SEQ ID NO: 97 - protein, 36 aa, Artificial Sequence
  Description of Artificial Sequence Synthetic polypeptide
MCMPCFTTDHQMVRVCDDCCGGVGRGRCYGPQCLCR

SEQ ID NO: 98 - protein, 36 aa, Artificial Sequence
  Description of Artificial Sequence Synthetic polypeptide
MCMPCFTTDHQMVRRCDDCCGGVGRGRCYGPQCLCR

SEQ ID NO: 99 - protein, 36 aa, Artificial Sequence
  Description of Artificial Sequence Synthetic polypeptide
MCMPCFTTDHQMVRKCDDCCGGRGRGRCYGPQCLCR

SEQ ID NO: 100 - protein, 36 aa, Artificial Sequence
  Description of Artificial Sequence Synthetic polypeptide
MCMPCFTTDHQMVRVCDDCCGGRGRGRCYGPQCLCR

SEQ ID NO: 101 - protein, 36 aa, Artificial Sequence
  Description of Artificial Sequence Synthetic polypeptide
MCMPCFTTDHQMVRRCDDCCGGRGRGRCYGPQCLCR

SEQ ID NO: 102 - protein, 36 aa, Artificial Sequence; identical to SEQ ID NO: 101
  Description of Artificial Sequence Synthetic polypeptide

SEQ ID NO: 103 - protein, 36 aa, Artificial Sequence
  Description of Artificial Sequence Synthetic polypeptide
KCMPCFTTDHQMVRRCDDCCGGRGRGRCYGPQCLCR

SEQ ID NO: 104 - protein, 36 aa, Artificial Sequence
  Description of Artificial Sequence Synthetic polypeptide
VCVPCFTTDHQVVRRCDDCCGGRGRGRCYGPQCLCR

SEQ ID NO: 105 - protein, 36 aa, Artificial Sequence
  Description of Artificial Sequence Synthetic polypeptide
KCVPCFTTDHQVVRRCDDCCGGRGRGRCYGPQCLCR

SEQ ID NO: 106 - protein, 36 aa, Artificial Sequence
  Description of Artificial Sequence Synthetic polypeptide
  MOD_RES (15)..(15): Citrulline
  MOD_RES (23)..(23): Citrulline
MCMPCFTTDHQMVRXCDDCCGGXGRGKCYGPQCLCR

SEQ ID NO: 107 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
MCMPCFTTDHQMVRXCDDCCGGXGRGXCYGPQCLCR

SEQ ID NO: 108 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCMPCFTTDHQMVRXCDDCCGGXGRGXCYGPQCLCR

SEQ ID NO: 109 - protein, 36 aa, Artificial Sequence
  Description of Artificial Sequence Synthetic polypeptide
  MOD_RES (15)..(15): Citrulline
  MOD_RES (23)..(23): Citrulline
VCVPCFTTDHQVVRXCDDCCGGXGRGKCYGPQCLCR

SEQ ID NO: 110 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
VCVPCFTTDHQVVRXCDDCCGGXGRGXCYGPQCLCR

SEQ ID NO: 111 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCVPCFTTDHQVVRXCDDCCGGXGRGXCYGPQCLCR

SEQ ID NO: 112 - protein, 43 aa, Artificial Sequence
  Description of Artificial Sequence Synthetic polypeptide
MCMPCFTTDHQMVRKCDDCCGGKGRGKCYGPQCLCRGAGAAGG

SEQ ID NO: 113 - protein, 43 aa, Artificial Sequence
  Description of Artificial Sequence Synthetic polypeptide
MCMPCFTTDHQMVRVCDDCCGGKGRGKCYGPQCLCRGAGAAGG

SEQ ID NO: 114 - protein, 43 aa, Artificial Sequence
  Description of Artificial Sequence Synthetic polypeptide
MCMPCFTTDHQMVRRCDDCCGGKGRGKCYGPQCLCRGAGAAGG

SEQ ID NO: 115 - protein, 43 aa, Artificial Sequence
  Description of Artificial Sequence Synthetic polypeptide
MCMPCFTTDHQMVRKCDDCCGGVGRGKCYGPQCLCRGAGAAGG

SEQ ID NO: 116 - protein, 43 aa, Artificial Sequence
  Description of Artificial Sequence Synthetic polypeptide
MCMPCFTTDHQMVRVCDDCCGGVGRGKCYGPQCLCRGAGAAGG

SEQ ID NO: 117 - protein, 43 aa, Artificial Sequence
  Description of Artificial Sequence Synthetic polypeptide
MCMPCFTTDHQMVRRCDDCCGGVGRGKCYGPQCLCRGAGAAGG

SEQ ID NO: 118 - protein, 43 aa, Artificial Sequence
  Description of Artificial Sequence Synthetic polypeptide
MCMPCFTTDHQMVRKCDDCCGGRGRGKCYGPQCLCRGAGAAGG

SEQ ID NO: 119 - protein, 43 aa, Artificial Sequence
  Description of Artificial Sequence Synthetic polypeptide
MCMPCFTTDHQMVRVCDDCCGGRGRGKCYGPQCLCRGAGAAGG

SEQ ID NO: 120 - protein, 43 aa, Artificial Sequence
  Description of Artificial Sequence Synthetic polypeptide
MCMPCFTTDHQMVRRCDDCCGGRGRGKCYGPQCLCRGAGAAGG

SEQ ID NO: 121 - protein, 43 aa, Artificial Sequence
  Description of Artificial Sequence Synthetic polypeptide
MCMPCFTTDHQMVRKCDDCCGGKGRGVCYGPQCLCRGAGAAGG

SEQ ID NO: 122 - protein, 43 aa, Artificial Sequence
  Description of Artificial Sequence Synthetic polypeptide
MCMPCFTTDHQMVRVCDDCCGGKGRGVCYGPQCLCRGAGAAGG

SEQ ID NO: 123 - protein, 43 aa, Artificial Sequence
  Description of Artificial Sequence Synthetic polypeptide
MCMPCFTTDHQMVRRCDDCCGGKGRGVCYGPQCLCRGAGAAGG

SEQ ID NO: 124 - protein, 43 aa, Artificial Sequence
  Description of Artificial Sequence Synthetic polypeptide
MCMPCFTTDHQMVRKCDDCCGGVGRGVCYGPQCLCRGAGAAGG

SEQ ID NO: 125 - protein, 43 aa, Artificial Sequence
  Description of Artificial Sequence Synthetic polypeptide
MCMPCFTTDHQMVRVCDDCCGGVGRGVCYGPQCLCRGAGAAGG

SEQ ID NO: 126 - protein, 43 aa, Artificial Sequence
  Description of Artificial Sequence Synthetic polypeptide
MCMPCFTTDHQMVRRCDDCCGGVGRGVCYGPQCLCRGAGAAGG

SEQ ID NO: 127 - protein, 43 aa, Artificial Sequence
  Description of Artificial Sequence Synthetic polypeptide
MCMPCFTTDHQMVRKCDDCCGGRGRGVCYGPQCLCRGAGAAGG

SEQ ID NO: 128 - protein, 43 aa, Artificial Sequence
  Description of Artificial Sequence Synthetic polypeptide
MCMPCFTTDHQMVRVCDDCCGGRGRGVCYGPQCLCRGAGAAGG

SEQ ID NO: 129 - protein, 43 aa, Artificial Sequence
  Description of Artificial Sequence Synthetic polypeptide
MCMPCFTTDHQMVRRCDDCCGGRGRGVCYGPQCLCRGAGAAGG

SEQ ID NO: 130 - protein, 43 aa, Artificial Sequence
  Description of Artificial Sequence Synthetic polypeptide
MCMPCFTTDHQMVRKCDDCCGGKGRGRCYGPQCLCRGAGAAGG

SEQ ID NO: 131 - protein, 43 aa, Artificial Sequence
  Description of Artificial Sequence Synthetic polypeptide
MCMPCFTTDHQMVRVCDDCCGGKGRGRCYGPQCLCRGAGAAGG

SEQ ID NO: 132 - protein, 43 aa, Artificial Sequence
  Description of Artificial Sequence Synthetic polypeptide
MCMPCFTTDHQMVRRCDDCCGGKGRGRCYGPQCLCRGAGAAGG

SEQ ID NO: 133 - protein, 43 aa, Artificial Sequence
  Description of Artificial Sequence Synthetic polypeptide
MCMPCFTTDHQMVRKCDDCCGGVGRGRCYGPQCLCRGAGAAGG

SEQ ID NO: 134 - protein, 43 aa, Artificial Sequence
  Description of Artificial Sequence Synthetic polypeptide
MCMPCFTTDHQMVRVCDDCCGGVGRGRCYGPQCLCRGAGAAGG

SEQ ID NO: 135 - protein, 43 aa, Artificial Sequence
  Description of Artificial Sequence Synthetic polypeptide
MCMPCFTTDHQMVRRCDDCCGGVGRGRCYGPQCLCRGAGAAGG

SEQ ID NO: 136 - protein, 43 aa, Artificial Sequence
  Description of Artificial Sequence Synthetic polypeptide
MCMPCFTTDHQMVRKCDDCCGGRGRGRCYGPQCLCRGAGAAGG

SEQ ID NO: 137 - protein, 43 aa, Artificial Sequence
  Description of Artificial Sequence Synthetic polypeptide
MCMPCFTTDHQMVRVCDDCCGGRGRGRCYGPQCLCRGAGAAGG

SEQ ID NO: 138 - protein, 43 aa, Artificial Sequence
  Description of Artificial Sequence Synthetic polypeptide
MCMPCFTTDHQMVRRCDDCCGGRGRGRCYGPQCLCRGAGAAGG

SEQ ID NO: 139 - protein, 43 aa, Artificial Sequence; identical to SEQ ID NO: 138
  Description of Artificial Sequence Synthetic polypeptide

SEQ ID NO: 140 - protein, 43 aa, Artificial Sequence
  Description of Artificial Sequence Synthetic polypeptide
KCMPCFTTDHQMVRRCDDCCGGRGRGRCYGPQCLCRGAGAAGG

SEQ ID NO: 141 - protein, 43 aa, Artificial Sequence
  Description of Artificial Sequence Synthetic polypeptide
VCVPCFTTDHQVVRRCDDCCGGRGRGRCYGPQCLCRGAGAAGG

SEQ ID NO: 142 - protein, 43 aa, Artificial Sequence
  Description of Artificial Sequence Synthetic polypeptide
KCVPCFTTDHQVVRRCDDCCGGRGRGRCYGPQCLCRGAGAAGG

SEQ ID NO: 143 - protein, 43 aa, Artificial Sequence
  Description of Artificial Sequence Synthetic polypeptide
  MOD_RES (15)..(15): Citrulline
  MOD_RES (23)..(23): Citrulline
MCMPCFTTDHQMVRXCDDCCGGXGRGKCYGPQCLCRGAGAAGG

SEQ ID NO: 144 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
MCMPCFTTDHQMVRXCDDCCGGXGRGXCYGPQCLCRGAGAAGG

SEQ ID NO: 145 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCMPCFTTDHQMVRXCDDCCGGXGRGXCYGPQCLCRGAGAAGG

SEQ ID NO: 146 - protein, 43 aa, Artificial Sequence
  Description of Artificial Sequence Synthetic polypeptide
  MOD_RES (15)..(15): Citrulline
  MOD_RES (23)..(23): Citrulline
VCVPCFTTDHQVVRXCDDCCGGXGRGKCYGPQCLCRGAGAAGG

SEQ ID NO: 147 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
VCVPCFTTDHQVVRXCDDCCGGXGRGXCYGPQCLCRGAGAAGG

SEQ ID NO: 148 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCVPCFTTDHQVVRXCDDCCGGXGRGXCYGPQCLCRGAGAAGG

SEQ ID NO: 149 - protein, 36 aa, Artificial Sequence
  Description of Artificial Sequence Synthetic polypeptide
MCMPCFTTDHQMLRKCDDCCGGKGRGKCYGPQCLCR

SEQ ID NO: 150 - protein, 36 aa, Artificial Sequence
  Description of Artificial Sequence Synthetic polypeptide
MCMPCFTTDHQMLRLCDDCCGGKGRGKCYGPQCLCR

SEQ ID NO: 151 - protein, 36 aa, Artificial Sequence
  Description of Artificial Sequence Synthetic polypeptide
MCMPCFTTDHQMLRRCDDCCGGKGRGKCYGPQCLCR

SEQ ID NO: 152 - protein, 36 aa, Artificial Sequence
  Description of Artificial Sequence Synthetic polypeptide
MCMPCFTTDHQMLRKCDDCCGGLGRGKCYGPQCLCR

SEQ ID NO: 153 - protein, 36 aa, Artificial Sequence
  Description of Artificial Sequence Synthetic polypeptide
MCMPCFTTDHQMLRLCDDCCGGLGRGKCYGPQCLCR

SEQ ID NO: 154 - protein, 36 aa, Artificial Sequence
  Description of Artificial Sequence Synthetic polypeptide
MCMPCFTTDHQMLRRCDDCCGGLGRGKCYGPQCLCR

SEQ ID NO: 155 - protein, 36 aa, Artificial Sequence
  Description of Artificial Sequence Synthetic polypeptide
MCMPCFTTDHQMLRKCDDCCGGRGRGKCYGPQCLCR

SEQ ID NO: 156 - protein, 36 aa, Artificial Sequence
  Description of Artificial Sequence Synthetic polypeptide
MCMPCFTTDHQMLRLCDDCCGGRGRGKCYGPQCLCR

SEQ ID NO: 157 - protein, 36 aa, Artificial Sequence
  Description of Artificial Sequence Synthetic polypeptide
MCMPCFTTDHQMLRRCDDCCGGRGRGKCYGPQCLCR

SEQ ID NO: 158 - protein, 36 aa, Artificial Sequence
  Description of Artificial Sequence Synthetic polypeptide
MCMPCFTTDHQMLRKCDDCCGGKGRGLCYGPQCLCR

SEQ ID NO: 159 - protein, 36 aa, Artificial Sequence
  Description of Artificial Sequence Synthetic polypeptide
MCMPCFTTDHQMLRLCDDCCGGKGRGLCYGPQCLCR

SEQ ID NO: 160 - protein, 36 aa, Artificial Sequence
  Description of Artificial Sequence Synthetic polypeptide
MCMPCFTTDHQMLRRCDDCCGGKGRGLCYGPQCLCR

SEQ ID NO: 161 - protein, 36 aa, Artificial Sequence
  Description of Artificial Sequence Synthetic polypeptide
MCMPCFTTDHQMLRKCDDCCGGLGRGLCYGPQCLCR

SEQ ID NO: 162 - protein, 36 aa, Artificial Sequence
  Description of Artificial Sequence Synthetic polypeptide
MCMPCFTTDHQMLRLCDDCCGGLGRGLCYGPQCLCR

SEQ ID NO: 163 - protein, 36 aa, Artificial Sequence
  Description of Artificial Sequence Synthetic polypeptide
MCMPCFTTDHQMLRRCDDCCGGLGRGLCYGPQCLCR

SEQ ID NO: 164 - protein, 36 aa, Artificial Sequence
  Description of Artificial Sequence Synthetic polypeptide
MCMPCFTTDHQMLRKCDDCCGGRGRGLCYGPQCLCR

SEQ ID NO: 165 - protein, 36 aa, Artificial Sequence
  Description of Artificial Sequence Synthetic polypeptide
MCMPCFTTDHQMLRLCDDCCGGRGRGLCYGPQCLCR

SEQ ID NO: 166 - protein, 36 aa, Artificial Sequence
  Description of Artificial Sequence Synthetic polypeptide
MCMPCFTTDHQMLRRCDDCCGGRGRGLCYGPQCLCR

SEQ ID NO: 167 - protein, 36 aa, Artificial Sequence
  Description of Artificial Sequence Synthetic polypeptide
MCMPCFTTDHQMLRKCDDCCGGKGRGRCYGPQCLCR

SEQ ID NO: 168 - protein, 36 aa, Artificial Sequence
  Description of Artificial Sequence Synthetic polypeptide
MCMPCFTTDHQMLRLCDDCCGGKGRGRCYGPQCLCR

SEQ ID NO: 169 - protein, 36 aa, Artificial Sequence
  Description of Artificial Sequence Synthetic polypeptide
MCMPCFTTDHQMLRRCDDCCGGKGRGRCYGPQCLCR

SEQ ID NO: 170 - protein, 36 aa, Artificial Sequence
  Description of Artificial Sequence Synthetic polypeptide
MCMPCFTTDHQMLRKCDDCCGGLGRGRCYGPQCLCR

SEQ ID NO: 171 - protein, 36 aa, Artificial Sequence
  Description of Artificial Sequence Synthetic polypeptide
MCMPCFTTDHQMLRLCDDCCGGLGRGRCYGPQCLCR

SEQ ID NO: 172 - protein, 36 aa, Artificial Sequence
  Description of Artificial Sequence Synthetic polypeptide
MCMPCFTTDHQMLRRCDDCCGGLGRGRCYGPQCLCR

SEQ ID NO: 173 - protein, 36 aa, Artificial Sequence
  Description of Artificial Sequence Synthetic polypeptide
MCMPCFTTDHQMLRKCDDCCGGRGRGRCYGPQCLCR

SEQ ID NO: 174 - protein, 36 aa, Artificial Sequence
  Description of Artificial Sequence Synthetic polypeptide
MCMPCFTTDHQMLRLCDDCCGGRGRGRCYGPQCLCR

SEQ ID NO: 175 - protein, 36 aa, Artificial Sequence
  Description of Artificial Sequence Synthetic polypeptide
MCMPCFTTDHQMLRRCDDCCGGRGRGRCYGPQCLCR

SEQ ID NO: 176 - protein, 36 aa, Artificial Sequence; identical to SEQ ID NO: 175
  Description of Artificial Sequence Synthetic polypeptide

SEQ ID NO: 177 - protein, 36 aa, Artificial Sequence
  Description of Artificial Sequence Synthetic polypeptide
KCMPCFTTDHQMLRRCDDCCGGRGRGRCYGPQCLCR

SEQ ID NO: 178 - protein, 36 aa, Artificial Sequence
  Description of Artificial Sequence Synthetic polypeptide
LCLPCFTTDHQLLRRCDDCCGGRGRGRCYGPQCLCR

SEQ ID NO: 179 - protein, 36 aa, Artificial Sequence
  Description of Artificial Sequence Synthetic polypeptide
KCLPCFTTDHQLLRRCDDCCGGRGRGRCYGPQCLCR

SEQ ID NO: 180 - protein, 36 aa, Artificial Sequence
  Description of Artificial Sequence Synthetic polypeptide
  MOD_RES (15)..(15): Citrulline
  MOD_RES (23)..(23): Citrulline
MCMPCFTTDHQMLRXCDDCCGGXGRGKCYGPQCLCR

SEQ ID NO: 181 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
MCMPCFTTDHQMLRXCDDCCGGXGRGXCYGPQCLCR

SEQ ID NO: 182 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCMPCFTTDHQMLRXCDDCCGGXGRGXCYGPQCLCR

SEQ ID NO: 183 - protein, 36 aa, Artificial Sequence
  Description of Artificial Sequence Synthetic polypeptide
  MOD_RES (15)..(15): Citrulline
  MOD_RES (23)..(23): Citrulline
LCLPCFTTDHQLLRXCDDCCGGXGRGKCYGPQCLCR

SEQ ID NO: 184 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
LCLPCFTTDHQLLRXCDDCCGGXGRGXCYGPQCLCR

SEQ ID NO: 185 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCLPCFTTDHQLLRXCDDCCGGXGRGXCYGPQCLCR

SEQ ID NO: 186 - protein, 43 aa, Artificial Sequence
  Description of Artificial Sequence Synthetic polypeptide
MCMPCFTTDHQMLRKCDDCCGGKGRGKCYGPQCLCRGAGAAGG

SEQ ID NO: 187 - protein, 43 aa, Artificial Sequence
  Description of Artificial Sequence Synthetic polypeptide
MCMPCFTTDHQMLRLCDDCCGGKGRGKCYGPQCLCRGAGAAGG

SEQ ID NO: 188 - protein, 43 aa, Artificial Sequence
  Description of Artificial Sequence Synthetic polypeptide
MCMPCFTTDHQMLRRCDDCCGGKGRGKCYGPQCLCRGAGAAGG

SEQ ID NO: 189 - protein, 43 aa, Artificial Sequence
  Description of Artificial Sequence Synthetic polypeptide
MCMPCFTTDHQMLRKCDDCCGGLGRGKCYGPQCLCRGAGAAGG

SEQ ID NO: 190 - protein, 43 aa, Artificial Sequence
  Description of Artificial Sequence Synthetic polypeptide
MCMPCFTTDHQMLRLCDDCCGGLGRGKCYGPQCLCRGAGAAGG

SEQ ID NO: 191 - protein, 43 aa, Artificial Sequence
  Description of Artificial Sequence Synthetic polypeptide
MCMPCFTTDHQMLRRCDDCCGGLGRGKCYGPQCLCRGAGAAGG

SEQ ID NO: 192 - protein, 43 aa, Artificial Sequence
  Description of Artificial Sequence Synthetic polypeptide
MCMPCFTTDHQMLRKCDDCCGGRGRGKCYGPQCLCRGAGAAGG

SEQ ID NO: 193 - protein, 43 aa, Artificial Sequence
  Description of Artificial Sequence Synthetic polypeptide
MCMPCFTTDHQMLRLCDDCCGGRGRGKCYGPQCLCRGAGAAGG

SEQ ID NO: 194 - protein, 43 aa, Artificial Sequence
  Description of Artificial Sequence Synthetic polypeptide
MCMPCFTTDHQMLRRCDDCCGGRGRGKCYGPQCLCRGAGAAGG

SEQ ID NO: 195 - protein, 43 aa, Artificial Sequence
  Description of Artificial Sequence Synthetic polypeptide
MCMPCFTTDHQMLRKCDDCCGGKGRGLCYGPQCLCRGAGAAGG

SEQ ID NO: 196 - protein, 43 aa, Artificial Sequence
  Description of Artificial Sequence Synthetic polypeptide
MCMPCFTTDHQMLRLCDDCCGGKGRGLCYGPQCLCRGAGAAGG

SEQ ID NO: 197 - protein, 43 aa, Artificial Sequence
  Description of Artificial Sequence Synthetic polypeptide
MCMPCFTTDHQMLRRCDDCCGGKGRGLCYGPQCLCRGAGAAGG

SEQ ID NO: 198 - protein, 43 aa, Artificial Sequence
  Description of Artificial Sequence Synthetic polypeptide
MCMPCFTTDHQMLRKCDDCCGGLGRGLCYGPQCLCRGAGAAGG

SEQ ID NO: 199 - protein, 43 aa, Artificial Sequence
  Description of Artificial Sequence Synthetic polypeptide
MCMPCFTTDHQMLRLCDDCCGGLGRGLCYGPQCLCRGAGAAGG

SEQ ID NO: 200 - protein, 43 aa, Artificial Sequence
  Description of Artificial Sequence Synthetic polypeptide
MCMPCFTTDHQMLRRCDDCCGGLGRGLCYGPQCLCRGAGAAGG

SEQ ID NO: 201 - protein, 43 aa, Artificial Sequence
  Description of Artificial Sequence Synthetic polypeptide
MCMPCFTTDHQMLRKCDDCCGGRGRGLCYGPQCLCRGAGAAGG

SEQ ID NO: 202 - protein, 43 aa, Artificial Sequence
  Description of Artificial Sequence Synthetic polypeptide
MCMPCFTTDHQMLRLCDDCCGGRGRGLCYGPQCLCRGAGAAGG

SEQ ID NO: 203 - protein, 43 aa, Artificial Sequence
  Description of Artificial Sequence Synthetic polypeptide
MCMPCFTTDHQMLRRCDDCCGGRGRGLCYGPQCLCRGAGAAGG

SEQ ID NO: 204 - protein, 43 aa, Artificial Sequence
  Description of Artificial Sequence Synthetic polypeptide
MCMPCFTTDHQMLRKCDDCCGGKGRGRCYGPQCLCRGAGAAGG

SEQ ID NO: 205 - protein, 43 aa, Artificial Sequence
  Description of Artificial Sequence Synthetic polypeptide
MCMPCFTTDHQMLRLCDDCCGGKGRGRCYGPQCLCRGAGAAGG

SEQ ID NO: 206 - protein, 43 aa, Artificial Sequence
  Description of Artificial Sequence Synthetic polypeptide
MCMPCFTTDHQMLRRCDDCCGGKGRGRCYGPQCLCRGAGAAGG

SEQ ID NO: 207 - protein, 43 aa, Artificial Sequence
  Description of Artificial Sequence Synthetic polypeptide
MCMPCFTTDHQMLRKCDDCCGGLGRGRCYGPQCLCRGAGAAGG

SEQ ID NO: 208 - protein, 43 aa, Artificial Sequence
  Description of Artificial Sequence Synthetic polypeptide
MCMPCFTTDHQMLRLCDDCCGGLGRGRCYGPQCLCRGAGAAGG

SEQ ID NO: 209 - protein, 43 aa, Artificial Sequence
  Description of Artificial Sequence Synthetic polypeptide
MCMPCFTTDHQMLRRCDDCCGGLGRGRCYGPQCLCRGAGAAGG

SEQ ID NO: 210 - protein, 43 aa, Artificial Sequence
  Description of Artificial Sequence Synthetic polypeptide
MCMPCFTTDHQMLRKCDDCCGGRGRGRCYGPQCLCRGAGAAGG

SEQ ID NO: 211 - protein, 43 aa, Artificial Sequence
  Description of Artificial Sequence Synthetic polypeptide
MCMPCFTTDHQMLRLCDDCCGGRGRGRCYGPQCLCRGAGAAGG

SEQ ID NO: 212 - protein, 43 aa, Artificial Sequence
  Description of Artificial Sequence Synthetic polypeptide
MCMPCFTTDHQMLRRCDDCCGGRGRGRCYGPQCLCRGAGAAGG

SEQ ID NO: 213 - protein, 43 aa, Artificial Sequence; identical to SEQ ID NO: 212
  Description of Artificial Sequence Synthetic polypeptide

SEQ ID NO: 214 - protein, 43 aa, Artificial Sequence
  Description of Artificial Sequence Synthetic polypeptide
KCMPCFTTDHQMLRRCDDCCGGRGRGRCYGPQCLCRGAGAAGG

SEQ ID NO: 215 - protein, 43 aa, Artificial Sequence
  Description of Artificial Sequence Synthetic polypeptide
LCLPCFTTDHQLLRRCDDCCGGRGRGRCYGPQCLCRGAGAAGG

SEQ ID NO: 216 - protein, 43 aa, Artificial Sequence
  Description of Artificial Sequence Synthetic polypeptide
KCLPCFTTDHQLLRRCDDCCGGRGRGRCYGPQCLCRGAGAAGG

SEQ ID NO: 217 - protein, 43 aa, Artificial Sequence
  Description of Artificial Sequence Synthetic polypeptide
  MOD_RES (15)..(15): Citrulline
  MOD_RES (23)..(23): Citrulline
MCMPCFTTDHQMLRXCDDCCGGXGRGKCYGPQCLCRGAGAAGG

SEQ ID NO: 218 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
MCMPCFTTDHQMLRXCDDCCGGXGRGXCYGPQCLCRGAGAAGG

SEQ ID NO: 219 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCMPCFTTDHQMLRXCDDCCGGXGRGXCYGPQCLCRGAGAAGG

SEQ ID NO: 220 - protein, 43 aa, Artificial Sequence
  Description of Artificial Sequence Synthetic polypeptide
  MOD_RES (15)..(15): Citrulline
  MOD_RES (23)..(23): Citrulline
LCLPCFTTDHQLLRXCDDCCGGXGRGKCYGPQCLCRGAGAAGG

SEQ ID NO: 221 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
LCLPCFTTDHQLLRXCDDCCGGXGRGXCYGPQCLCRGAGAAGG

SEQ ID NO: 222 - protein, 43 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCLPCFTTDHQLLRXCDDCCGGXGRGXCYGPQCLCRGAGAAGG

SEQ ID NO: 223 - protein, 36 aa, Artificial Sequence
  Description of Artificial Sequence Synthetic polypeptide
GCGPCFTTDHQGARKCDDCCGGKGRGKCYGPQCLCR

SEQ ID NO: 224 - protein, 36 aa, Artificial Sequence
  Description of Artificial Sequence Synthetic polypeptide
GCGPCFTTDHQGARACDDCCGGKGRGKCYGPQCLCR

SEQ ID NO: 225 - protein, 36 aa, Artificial Sequence
  Description of Artificial Sequence Synthetic polypeptide
GCGPCFTTDHQGARRCDDCCGGKGRGKCYGPQCLCR

SEQ ID NO: 226 - protein, 36 aa, Artificial Sequence
  Description of Artificial Sequence Synthetic polypeptide
GCGPCFTTDHQGARKCDDCCGGAGRGKCYGPQCLCR

SEQ ID NO: 227 - protein, 36 aa, Artificial Sequence
  Description of Artificial Sequence Synthetic polypeptide
GCGPCFTTDHQGARACDDCCGGAGRGKCYGPQCLCR

SEQ ID NO: 228 - protein, 36 aa, Artificial Sequence
  Description of Artificial Sequence Synthetic polypeptide
GCGPCFTTDHQGARRCDDCCGGAGRGKCYGPQCLCR

SEQ ID NO: 229 - protein, 36 aa, Artificial Sequence
  Description of Artificial Sequence Synthetic polypeptide
GCGPCFTTDHQGARKCDDCCGGRGRGKCYGPQCLCR

SEQ ID NO: 230 - protein, 36 aa, Artificial Sequence
  Description of Artificial Sequence Synthetic polypeptide
GCGPCFTTDHQGARACDDCCGGRGRGKCYGPQCLCR

SEQ ID NO: 231 - protein, 36 aa, Artificial Sequence
  Description of Artificial Sequence Synthetic polypeptide
GCGPCFTTDHQGARRCDDCCGGRGRGKCYGPQCLCR

SEQ ID NO: 232 - protein, 36 aa, Artificial Sequence
  Description of Artificial Sequence Synthetic polypeptide
GCGPCFTTDHQGARKCDDCCGGKGRGACYGPQCLCR

SEQ ID NO: 233 - protein, 36 aa, Artificial Sequence
  Description of Artificial Sequence Synthetic polypeptide
GCGPCFTTDHQGARACDDCCGGKGRGACYGPQCLCR

SEQ ID NO: 234 - protein, 36 aa, Artificial Sequence
  Description of Artificial Sequence Synthetic polypeptide
GCGPCFTTDHQGARRCDDCCGGKGRGACYGPQCLCR

SEQ ID NO: 235 - protein, 36 aa, Artificial Sequence
  Description of Artificial Sequence Synthetic polypeptide
GCGPCFTTDHQGARKCDDCCGGAGRGACYGPQCLCR

SEQ ID NO: 236 - protein, 36 aa, Artificial Sequence
  Description of Artificial Sequence Synthetic polypeptide
GCGPCFTTDHQGARACDDCCGGAGRGACYGPQCLCR

SEQ ID NO: 237 - protein, 36 aa, Artificial Sequence
  Description of Artificial Sequence Synthetic polypeptide
GCGPCFTTDHQGARRCDDCCGGAGRGACYGPQCLCR

SEQ ID NO: 238 - protein, 36 aa, Artificial Sequence
  Description of Artificial Sequence Synthetic polypeptide
GCGPCFTTDHQGARKCDDCCGGRGRGACYGPQCLCR

SEQ ID NO: 239 - protein, 36 aa, Artificial Sequence
  Description of Artificial Sequence Synthetic polypeptide
GCGPCFTTDHQGARACDDCCGGRGRGACYGPQCLCR

SEQ ID NO: 240 - protein, 36 aa, Artificial Sequence
  Description of Artificial Sequence Synthetic polypeptide
GCGPCFTTDHQGARRCDDCCGGRGRGACYGPQCLCR

SEQ ID NO: 241 - protein, 36 aa, Artificial Sequence
  Description of Artificial Sequence Synthetic polypeptide
GCGPCFTTDHQGARKCDDCCGGKGRGRCYGPQCLCR

SEQ ID NO: 242 - protein, 36 aa, Artificial Sequence
  Description of Artificial Sequence Synthetic polypeptide
GCGPCFTTDHQGARACDDCCGGKGRGRCYGPQCLCR

SEQ ID NO: 243 - protein, 36 aa, Artificial Sequence
  Description of Artificial Sequence Synthetic polypeptide
GCGPCFTTDHQGARRCDDCCGGKGRGRCYGPQCLCR

SEQ ID NO: 244 - protein, 36 aa, Artificial Sequence
  Description of Artificial Sequence Synthetic polypeptide
GCGPCFTTDHQGARKCDDCCGGAGRGRCYGPQCLCR

SEQ ID NO: 245 - protein, 36 aa, Artificial Sequence
  Description of Artificial Sequence Synthetic polypeptide
GCGPCFTTDHQGARACDDCCGGAGRGRCYGPQCLCR

SEQ ID NO: 246 - protein, 36 aa, Artificial Sequence
  Description of Artificial Sequence Synthetic polypeptide
GCGPCFTTDHQGARRCDDCCGGAGRGRCYGPQCLCR

SEQ ID NO: 247 - protein, 36 aa, Artificial Sequence
  Description of Artificial Sequence Synthetic polypeptide
GCGPCFTTDHQGARKCDDCCGGRGRGRCYGPQCLCR

SEQ ID NO: 248 - protein, 36 aa, Artificial Sequence
  Description of Artificial Sequence Synthetic polypeptide
GCGPCFTTDHQGARACDDCCGGRGRGRCYGPQCLCR

SEQ ID NO: 249 - protein, 36 aa, Artificial Sequence
  Description of Artificial Sequence Synthetic polypeptide
GCGPCFTTDHQGARRCDDCCGGRGRGRCYGPQCLCR

SEQ ID NO: 250 - protein, 36 aa, Artificial Sequence; identical to SEQ ID NO: 249
  Description of Artificial Sequence Synthetic polypeptide

SEQ ID NO: 251 - protein, 36 aa, Artificial Sequence
  Description of Artificial Sequence Synthetic polypeptide
KCGPCFTTDHQGARRCDDCCGGRGRGRCYGPQCLCR

SEQ ID NO: 252 - protein, 36 aa, Artificial Sequence; identical to SEQ ID NO: 30
  Description of Artificial Sequence Synthetic polypeptide

SEQ ID NO: 253 - protein, 36 aa, Artificial Sequence; identical to SEQ ID NO: 31
  Description of Artificial Sequence Synthetic polypeptide

SEQ ID NO: 254 - protein, 36 aa, Artificial Sequence
  Description of Artificial Sequence Synthetic polypeptide
  MOD_RES (15)..(15): Citrulline
  MOD_RES (23)..(23): Citrulline
GCGPCFTTDHQGARXCDDCCGGXGRGKCYGPQCLCR

SEQ ID NO: 255 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
GCGPCFTTDHQGARXCDDCCGGXGRGXCYGPQCLCR

SEQ ID NO: 256 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCGPCFTTDHQGARXCDDCCGGXGRGXCYGPQCLCR

SEQ ID NO: 257 - protein, 36 aa, Artificial Sequence; identical to SEQ ID NO: 35
  Description of Artificial Sequence Synthetic polypeptide
  MOD_RES (15)..(15): Citrulline
  MOD_RES (23)..(23): Citrulline

SEQ ID NO: 258 - protein, 36 aa, Artificial Sequence; identical to SEQ ID NO: 36
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 259 - protein, 36 aa, Artificial Sequence; identical to SEQ ID NO: 37
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 260 - protein, 36 aa, Artificial Sequence
  Description of Artificial Sequence Synthetic polypeptide
ACAPCFTTDHQAARKCDDCCGGKGRGKCYGPQCLCR

SEQ ID NO: 261 - protein, 36 aa, Artificial Sequence
  Description of Artificial Sequence Synthetic polypeptide
ACAPCFTTDHQAARACDDCCGGKGRGKCYGPQCLCR

SEQ ID NO: 262 - protein, 36 aa, Artificial Sequence
  Description of Artificial Sequence Synthetic polypeptide
ACAPCFTTDHQAARRCDDCCGGKGRGKCYGPQCLCR

SEQ ID NO: 263 - protein, 36 aa, Artificial Sequence
  Description of Artificial Sequence Synthetic polypeptide
ACAPCFTTDHQAARKCDDCCGGAGRGKCYGPQCLCR

SEQ ID NO: 264 - protein, 36 aa, Artificial Sequence
  Description of Artificial Sequence Synthetic polypeptide
ACAPCFTTDHQAARACDDCCGGAGRGKCYGPQCLCR

SEQ ID NO: 265 - protein, 36 aa, Artificial Sequence
  Description of Artificial Sequence Synthetic polypeptide
ACAPCFTTDHQAARRCDDCCGGAGRGKCYGPQCLCR

SEQ ID NO: 266 - protein, 36 aa, Artificial Sequence
  Description of Artificial Sequence Synthetic polypeptide
ACAPCFTTDHQAARKCDDCCGGRGRGKCYGPQCLCR

SEQ ID NO: 267 - protein, 36 aa, Artificial Sequence
  Description of Artificial Sequence Synthetic polypeptide
ACAPCFTTDHQAARACDDCCGGRGRGKCYGPQCLCR

SEQ ID NO: 268 - protein, 36 aa, Artificial Sequence
  Description of Artificial Sequence Synthetic polypeptide
ACAPCFTTDHQAARRCDDCCGGRGRGKCYGPQCLCR

SEQ ID NO: 269 - protein, 36 aa, Artificial Sequence
  Description of Artificial Sequence Synthetic polypeptide
ACAPCFTTDHQAARKCDDCCGGKGRGACYGPQCLCR

SEQ ID NO: 270 - protein, 36 aa, Artificial Sequence
  Description of Artificial Sequence Synthetic polypeptide
ACAPCFTTDHQAARACDDCCGGKGRGACYGPQCLCR

SEQ ID NO: 271 - protein, 36 aa, Artificial Sequence
  Description of Artificial Sequence Synthetic polypeptide
ACAPCFTTDHQAARRCDDCCGGKGRGACYGPQCLCR

SEQ ID NO: 272 - protein, 36 aa, Artificial Sequence
  Description of Artificial Sequence Synthetic polypeptide
ACAPCFTTDHQAARKCDDCCGGAGRGACYGPQCLCR

SEQ ID NO: 273 - protein, 36 aa, Artificial Sequence
  Description of Artificial Sequence Synthetic polypeptide
ACAPCFTTDHQAARACDDCCGGAGRGACYGPQCLCR

SEQ ID NO: 274 - protein, 36 aa, Artificial Sequence
  Description of Artificial Sequence Synthetic polypeptide
ACAPCFTTDHQAARRCDDCCGGAGRGACYGPQCLCR

SEQ ID NO: 275 - protein, 36 aa, Artificial Sequence
  Description of Artificial Sequence Synthetic polypeptide
ACAPCFTTDHQAARKCDDCCGGRGRGACYGPQCLCR

SEQ ID NO: 276 - protein, 36 aa, Artificial Sequence
  Description of Artificial Sequence Synthetic polypeptide
ACAPCFTTDHQAARACDDCCGGRGRGACYGPQCLCR

SEQ ID NO: 277 - protein, 36 aa, Artificial Sequence
  Description of Artificial Sequence Synthetic polypeptide
ACAPCFTTDHQAARRCDDCCGGRGRGACYGPQCLCR

SEQ ID NO: 278 - protein, 36 aa, Artificial Sequence
  Description of Artificial Sequence Synthetic polypeptide
ACAPCFTTDHQAARKCDDCCGGKGRGRCYGPQCLCR

SEQ ID NO: 279 - protein, 36 aa, Artificial Sequence
  Description of Artificial Sequence Synthetic polypeptide
ACAPCFTTDHQAARACDDCCGGKGRGRCYGPQCLCR

SEQ ID NO: 280 - protein, 36 aa, Artificial Sequence
  Description of Artificial Sequence Synthetic polypeptide
ACAPCFTTDHQAARRCDDCCGGKGRGRCYGPQCLCR

SEQ ID NO: 281 - protein, 36 aa, Artificial Sequence
  Description of Artificial Sequence Synthetic polypeptide
ACAPCFTTDHQAARKCDDCCGGAGRGRCYGPQCLCR

SEQ ID NO: 282 - protein, 36 aa, Artificial Sequence
  Description of Artificial Sequence Synthetic polypeptide
ACAPCFTTDHQAARACDDCCGGAGRGRCYGPQCLCR

SEQ ID NO: 283 - protein, 36 aa, Artificial Sequence
  Description of Artificial Sequence Synthetic polypeptide
ACAPCFTTDHQAARRCDDCCGGAGRGRCYGPQCLCR

SEQ ID NO: 284 - protein, 36 aa, Artificial Sequence
  Description of Artificial Sequence Synthetic polypeptide
ACAPCFTTDHQAARKCDDCCGGRGRGRCYGPQCLCR

SEQ ID NO: 285 - protein, 36 aa, Artificial Sequence
  Description of Artificial Sequence Synthetic polypeptide
ACAPCFTTDHQAARACDDCCGGRGRGRCYGPQCLCR

SEQ ID NO: 286 - protein, 36 aa, Artificial Sequence; identical to SEQ ID NO: 30
  Description of Artificial Sequence Synthetic polypeptide

SEQ ID NO: 287 - protein, 36 aa, Artificial Sequence; identical to SEQ ID NO: 30
  Description of Artificial Sequence Synthetic polypeptide

SEQ ID NO: 288 - protein, 36 aa, Artificial Sequence; identical to SEQ ID NO: 31
  Description of Artificial Sequence Synthetic polypeptide

SEQ ID NO: 289 - protein, 36 aa, Artificial Sequence; identical to SEQ ID NO: 30
  Description of Artificial Sequence Synthetic polypeptide

SEQ ID NO: 290 - protein, 36 aa, Artificial Sequence; identical to SEQ ID NO: 31
  Description of Artificial Sequence Synthetic polypeptide

SEQ ID NO: 291 - protein, 36 aa, Artificial Sequence; identical to SEQ ID NO: 35
  Description of Artificial Sequence Synthetic polypeptide
  MOD_RES (15)..(15): Citrulline
  MOD_RES (23)..(23): Citrulline

SEQ ID NO: 292 - protein, 36 aa, Artificial Sequence; identical to SEQ ID NO: 36
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 293 - protein, 36 aa, Artificial Sequence; identical to SEQ ID NO: 37
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 294 - protein, 36 aa, Artificial Sequence; identical to SEQ ID NO: 35
  Description of Artificial Sequence Synthetic polypeptide
  MOD_RES (15)..(15): Citrulline
  MOD_RES (23)..(23): Citrulline

SEQ ID NO: 295 - protein, 36 aa, Artificial Sequence; identical to SEQ ID NO: 36
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 296 - protein, 36 aa, Artificial Sequence; identical to SEQ ID NO: 37
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 297 - protein, 36 aa, Artificial Sequence
  Description of Artificial Sequence Synthetic polypeptide
ICIPCFTTDHQIARKCDDCCGGKGRGKCYGPQCLCR

SEQ ID NO: 298 - protein, 36 aa, Artificial Sequence
  Description of Artificial Sequence Synthetic polypeptide
ICIPCFTTDHQIARACDDCCGGKGRGKCYGPQCLCR

SEQ ID NO: 299 - protein, 36 aa, Artificial Sequence
  Description of Artificial Sequence Synthetic polypeptide
ICIPCFTTDHQIARRCDDCCGGKGRGKCYGPQCLCR

SEQ ID NO: 300 - protein, 36 aa, Artificial Sequence
  Description of Artificial Sequence Synthetic polypeptide
ICIPCFTTDHQIARKCDDCCGGAGRGKCYGPQCLCR

SEQ ID NO: 301 - protein, 36 aa, Artificial Sequence
  Description of Artificial Sequence Synthetic polypeptide
ICIPCFTTDHQIARACDDCCGGAGRGKCYGPQCLCR

SEQ ID NO: 302 - protein, 36 aa, Artificial Sequence
  Description of Artificial Sequence Synthetic polypeptide
ICIPCFTTDHQIARRCDDCCGGAGRGKCYGPQCLCR

SEQ ID NO: 303 - protein, 36 aa, Artificial Sequence
  Description of Artificial Sequence Synthetic polypeptide
ICIPCFTTDHQIARKCDDCCGGRGRGKCYGPQCLCR

SEQ ID NO: 304 - protein, 36 aa, Artificial Sequence
  Description of Artificial Sequence Synthetic polypeptide
ICIPCFTTDHQIARACDDCCGGRGRGKCYGPQCLCR

SEQ ID NO: 305 - protein, 36 aa, Artificial Sequence
  Description of Artificial Sequence Synthetic polypeptide
ICIPCFTTDHQIARRCDDCCGGRGRGKCYGPQCLCR

SEQ ID NO: 306 - protein, 36 aa, Artificial Sequence
  Description of Artificial Sequence Synthetic polypeptide
ICIPCFTTDHQIARKCDDCCGGKGRGACYGPQCLCR

SEQ ID NO: 307 - protein, 36 aa, Artificial Sequence
  Description of Artificial Sequence Synthetic polypeptide
ICIPCFTTDHQIARACDDCCGGKGRGACYGPQCLCR

SEQ ID NO: 308 - protein, 36 aa, Artificial Sequence
  Description of Artificial Sequence Synthetic polypeptide
ICIPCFTTDHQIARRCDDCCGGKGRGACYGPQCLCR

SEQ ID NO: 309 - protein, 36 aa, Artificial Sequence
  Description of Artificial Sequence Synthetic polypeptide
ICIPCFTTDHQIARKCDDCCGGAGRGACYGPQCLCR

SEQ ID NO: 310 - protein, 36 aa, Artificial Sequence
  Description of Artificial Sequence Synthetic polypeptide
ICIPCFTTDHQIARACDDCCGGAGRGACYGPQCLCR

SEQ ID NO: 311 - protein, 36 aa, Artificial Sequence
  Description of Artificial Sequence Synthetic polypeptide
ICIPCFTTDHQIARRCDDCCGGAGRGACYGPQCLCR

SEQ ID NO: 312 - protein, 36 aa, Artificial Sequence
  Description of Artificial Sequence Synthetic polypeptide
ICIPCFTTDHQIARKCDDCCGGRGRGACYGPQCLCR

SEQ ID NO: 313 - protein, 36 aa, Artificial Sequence
  Description of Artificial Sequence Synthetic polypeptide
ICIPCFTTDHQIARACDDCCGGRGRGACYGPQCLCR

SEQ ID NO: 314 - protein, 36 aa, Artificial Sequence
  Description of Artificial Sequence Synthetic polypeptide
ICIPCFTTDHQIARRCDDCCGGRGRGACYGPQCLCR

SEQ ID NO: 315 - protein, 36 aa, Artificial Sequence
  Description of Artificial Sequence Synthetic polypeptide
ICIPCFTTDHQIARKCDDCCGGKGRGRCYGPQCLCR

SEQ ID NO: 316 - protein, 36 aa, Artificial Sequence
  Description of Artificial Sequence Synthetic polypeptide
ICIPCFTTDHQIARACDDCCGGKGRGRCYGPQCLCR

SEQ ID NO: 317 - protein, 36 aa, Artificial Sequence
  Description of Artificial Sequence Synthetic polypeptide
ICIPCFTTDHQIARRCDDCCGGKGRGRCYGPQCLCR

SEQ ID NO: 318 - protein, 36 aa, Artificial Sequence
  Description of Artificial Sequence Synthetic polypeptide
ICIPCFTTDHQIARKCDDCCGGAGRGRCYGPQCLCR

SEQ ID NO: 319 - protein, 36 aa, Artificial Sequence
  Description of Artificial Sequence Synthetic polypeptide
ICIPCFTTDHQIARACDDCCGGAGRGRCYGPQCLCR

SEQ ID NO: 320 - protein, 36 aa, Artificial Sequence
  Description of Artificial Sequence Synthetic polypeptide
ICIPCFTTDHQIARRCDDCCGGAGRGRCYGPQCLCR

SEQ ID NO: 321 - protein, 36 aa, Artificial Sequence
  Description of Artificial Sequence Synthetic polypeptide
ICIPCFTTDHQIARKCDDCCGGRGRGRCYGPQCLCR

SEQ ID NO: 322 - protein, 36 aa, Artificial Sequence
  Description of Artificial Sequence Synthetic polypeptide
ICIPCFTTDHQIARACDDCCGGRGRGRCYGPQCLCR

SEQ ID NO: 323 - protein, 36 aa, Artificial Sequence
  Description of Artificial Sequence Synthetic polypeptide
ICIPCFTTDHQIARRCDDCCGGRGRGRCYGPQCLCR

SEQ ID NO: 324 - protein, 36 aa, Artificial Sequence; identical to SEQ ID NO: 323
  Description of Artificial Sequence Synthetic polypeptide

SEQ ID NO: 325 - protein, 36 aa, Artificial Sequence
  Description of Artificial Sequence Synthetic polypeptide
KCIPCFTTDHQIARRCDDCCGGRGRGRCYGPQCLCR

SEQ ID NO: 326 - protein, 36 aa, Artificial Sequence; identical to SEQ ID NO: 30
  Description of Artificial Sequence Synthetic polypeptide

SEQ ID NO: 327 - protein, 36 aa, Artificial Sequence; identical to SEQ ID NO: 31
  Description of Artificial Sequence Synthetic polypeptide

SEQ ID NO: 328 - protein, 36 aa, Artificial Sequence
  Description of Artificial Sequence Synthetic polypeptide
  MOD_RES (15)..(15): Citrulline
  MOD_RES (23)..(23): Citrulline
ICIPCFTTDHQIARXCDDCCGGXGRGKCYGPQCLCR

SEQ ID NO: 329 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
ICIPCFTTDHQIARXCDDCCGGXGRGXCYGPQCLCR

SEQ ID NO: 330 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCIPCFTTDHQIARXCDDCCGGXGRGXCYGPQCLCR

SEQ ID NO: 331 - protein, 36 aa, Artificial Sequence; identical to SEQ ID NO: 35
  Description of Artificial Sequence Synthetic polypeptide
  MOD_RES (15)..(15): Citrulline
  MOD_RES (23)..(23): Citrulline

SEQ ID NO: 332 - protein, 36 aa, Artificial Sequence; identical to SEQ ID NO: 36
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 333 - protein, 36 aa, Artificial Sequence; identical to SEQ ID NO: 37
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 334 - protein, 36 aa, Artificial Sequence
  Description of Artificial Sequence Synthetic polypeptide
TCTPCFTTDHQTARKCDDCCGGKGRGKCYGPQCLCR

SEQ ID NO: 335 - protein, 36 aa, Artificial Sequence
  Description of Artificial Sequence Synthetic polypeptide
TCTPCFTTDHQTARACDDCCGGKGRGKCYGPQCLCR

SEQ ID NO: 336 - protein, 36 aa, Artificial Sequence
  Description of Artificial Sequence Synthetic polypeptide
TCTPCFTTDHQTARRCDDCCGGKGRGKCYGPQCLCR

SEQ ID NO: 337 - protein, 36 aa, Artificial Sequence
  Description of Artificial Sequence Synthetic polypeptide
TCTPCFTTDHQTARKCDDCCGGAGRGKCYGPQCLCR

SEQ ID NO: 338 - protein, 36 aa, Artificial Sequence
  Description of Artificial Sequence Synthetic polypeptide
TCTPCFTTDHQTARACDDCCGGAGRGKCYGPQCLCR

SEQ ID NO: 339 - protein, 36 aa, Artificial Sequence
  Description of Artificial Sequence Synthetic polypeptide
TCTPCFTTDHQTARRCDDCCGGAGRGKCYGPQCLCR

SEQ ID NO: 340 - protein, 36 aa, Artificial Sequence
  Description of Artificial Sequence Synthetic polypeptide
TCTPCFTTDHQTARKCDDCCGGRGRGKCYGPQCLCR

SEQ ID NO: 341 - protein, 36 aa, Artificial Sequence
  Description of Artificial Sequence Synthetic polypeptide
TCTPCFTTDHQTARACDDCCGGRGRGKCYGPQCLCR

SEQ ID NO: 342 - protein, 36 aa, Artificial Sequence
  Description of Artificial Sequence Synthetic polypeptide
TCTPCFTTDHQTARRCDDCCGGRGRGKCYGPQCLCR

SEQ ID NO: 343 - protein, 36 aa, Artificial Sequence
  Description of Artificial Sequence Synthetic polypeptide
TCTPCFTTDHQTARKCDDCCGGKGRGACYGPQCLCR

SEQ ID NO: 344 - protein, 36 aa, Artificial Sequence
  Description of Artificial Sequence Synthetic polypeptide
TCTPCFTTDHQTARACDDCCGGKGRGACYGPQCLCR

SEQ ID NO: 345 - protein, 36 aa, Artificial Sequence
  Description of Artificial Sequence Synthetic polypeptide
TCTPCFTTDHQTARRCDDCCGGKGRGACYGPQCLCR

SEQ ID NO: 346 - protein, 36 aa, Artificial Sequence
  Description of Artificial Sequence Synthetic polypeptide
TCTPCFTTDHQTARKCDDCCGGAGRGACYGPQCLCR

SEQ ID NO: 347 - protein, 36 aa, Artificial Sequence
  Description of Artificial Sequence Synthetic polypeptide
TCTPCFTTDHQTARACDDCCGGAGRGACYGPQCLCR

SEQ ID NO: 348 - protein, 36 aa, Artificial Sequence
  Description of Artificial Sequence Synthetic polypeptide
TCTPCFTTDHQTARRCDDCCGGAGRGACYGPQCLCR

SEQ ID NO: 349 - protein, 36 aa, Artificial Sequence
  Description of Artificial Sequence Synthetic polypeptide
TCTPCFTTDHQTARKCDDCCGGRGRGACYGPQCLCR

SEQ ID NO: 350 - protein, 36 aa, Artificial Sequence
  Description of Artificial Sequence Synthetic polypeptide
TCTPCFTTDHQTARACDDCCGGRGRGACYGPQCLCR

SEQ ID NO: 351 - protein, 36 aa, Artificial Sequence
  Description of Artificial Sequence Synthetic polypeptide
TCTPCFTTDHQTARRCDDCCGGRGRGACYGPQCLCR

SEQ ID NO: 352 - protein, 36 aa, Artificial Sequence
  Description of Artificial Sequence Synthetic polypeptide
TCTPCFTTDHQTARKCDDCCGGKGRGRCYGPQCLCR

SEQ ID NO: 353 - protein, 36 aa, Artificial Sequence
  Description of Artificial Sequence Synthetic polypeptide
TCTPCFTTDHQTARACDDCCGGKGRGRCYGPQCLCR

SEQ ID NO: 354 - protein, 36 aa, Artificial Sequence
  Description of Artificial Sequence Synthetic polypeptide
TCTPCFTTDHQTARRCDDCCGGKGRGRCYGPQCLCR

SEQ ID NO: 355 - protein, 36 aa, Artificial Sequence
  Description of Artificial Sequence Synthetic polypeptide
TCTPCFTTDHQTARKCDDCCGGAGRGRCYGPQCLCR

SEQ ID NO: 356 - protein, 36 aa, Artificial Sequence
  Description of Artificial Sequence Synthetic polypeptide
TCTPCFTTDHQTARACDDCCGGAGRGRCYGPQCLCR

SEQ ID NO: 357 - protein, 36 aa, Artificial Sequence
  Description of Artificial Sequence Synthetic polypeptide
TCTPCFTTDHQTARRCDDCCGGAGRGRCYGPQCLCR

SEQ ID NO: 358 - protein, 36 aa, Artificial Sequence
  Description of Artificial Sequence Synthetic polypeptide
TCTPCFTTDHQTARKCDDCCGGRGRGRCYGPQCLCR

SEQ ID NO: 359 - protein, 36 aa, Artificial Sequence
  Description of Artificial Sequence Synthetic polypeptide
TCTPCFTTDHQTARACDDCCGGRGRGRCYGPQCLCR

SEQ ID NO: 360 - protein, 36 aa, Artificial Sequence
  Description of Artificial Sequence Synthetic polypeptide
TCTPCFTTDHQTARRCDDCCGGRGRGRCYGPQCLCR

SEQ ID NO: 361 - protein, 36 aa, Artificial Sequence; identical to SEQ ID NO: 360
  Description of Artificial Sequence Synthetic polypeptide

SEQ ID NO: 362 - protein, 36 aa, Artificial Sequence
  Description of Artificial Sequence Synthetic polypeptide
KCTPCFTTDHQTARRCDDCCGGRGRGRCYGPQCLCR

SEQ ID NO: 363 - protein, 36 aa, Artificial Sequence; identical to SEQ ID NO: 30
  Description of Artificial Sequence Synthetic polypeptide

SEQ ID NO: 364 - protein, 36 aa, Artificial Sequence; identical to SEQ ID NO: 31
  Description of Artificial Sequence Synthetic polypeptide

SEQ ID NO: 365 - protein, 36 aa, Artificial Sequence
  Description of Artificial Sequence Synthetic polypeptide
  MOD_RES (15)..(15): Citrulline
  MOD_RES (23)..(23): Citrulline
TCTPCFTTDHQTARXCDDCCGGXGRGKCYGPQCLCR

SEQ ID NO: 366 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
TCTPCFTTDHQTARXCDDCCGGXGRGXCYGPQCLCR

SEQ ID NO: 367 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCTPCFTTDHQTARXCDDCCGGXGRGXCYGPQCLCR

SEQ ID NO: 368 - protein, 36 aa, Artificial Sequence; identical to SEQ ID NO: 35
  Description of Artificial Sequence Synthetic polypeptide
  MOD_RES (15)..(15): Citrulline
  MOD_RES (23)..(23): Citrulline

SEQ ID NO: 369 - protein, 36 aa, Artificial Sequence; identical to SEQ ID NO: 36
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 370 - protein, 36 aa, Artificial Sequence; identical to SEQ ID NO: 37
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 371 - protein, 36 aa, Artificial Sequence
  Description of Artificial Sequence Synthetic polypeptide
VCVPCFTTDHQVARKCDDCCGGKGRGKCYGPQCLCR

SEQ ID NO: 372 - protein, 36 aa, Artificial Sequence
  Description of Artificial Sequence Synthetic polypeptide
VCVPCFTTDHQVARACDDCCGGKGRGKCYGPQCLCR

SEQ ID NO: 373 - protein, 36 aa, Artificial Sequence
  Description of Artificial Sequence Synthetic polypeptide
VCVPCFTTDHQVARRCDDCCGGKGRGKCYGPQCLCR

SEQ ID NO: 374 - protein, 36 aa, Artificial Sequence
  Description of Artificial Sequence Synthetic polypeptide
VCVPCFTTDHQVARKCDDCCGGAGRGKCYGPQCLCR

SEQ ID NO: 375 - protein, 36 aa, Artificial Sequence
  Description of Artificial Sequence Synthetic polypeptide
VCVPCFTTDHQVARACDDCCGGAGRGKCYGPQCLCR

SEQ ID NO: 376 - protein, 36 aa, Artificial Sequence
  Description of Artificial Sequence Synthetic polypeptide
VCVPCFTTDHQVARRCDDCCGGAGRGKCYGPQCLCR

SEQ ID NO: 377 - protein, 36 aa, Artificial Sequence
  Description of Artificial Sequence Synthetic polypeptide
VCVPCFTTDHQVARKCDDCCGGRGRGKCYGPQCLCR

SEQ ID NO: 378 - protein, 36 aa, Artificial Sequence
  Description of Artificial Sequence Synthetic polypeptide
VCVPCFTTDHQVARACDDCCGGRGRGKCYGPQCLCR

SEQ ID NO: 379 - protein, 36 aa, Artificial Sequence
  Description of Artificial Sequence Synthetic polypeptide
VCVPCFTTDHQVARRCDDCCGGRGRGKCYGPQCLCR

SEQ ID NO: 380 - protein, 36 aa, Artificial Sequence
  Description of Artificial Sequence Synthetic polypeptide
VCVPCFTTDHQVARKCDDCCGGKGRGACYGPQCLCR

SEQ ID NO: 381 - protein, 36 aa, Artificial Sequence
  Description of Artificial Sequence Synthetic polypeptide
VCVPCFTTDHQVARACDDCCGGKGRGACYGPQCLCR

SEQ ID NO: 382 - protein, 36 aa, Artificial Sequence
  Description of Artificial Sequence Synthetic polypeptide
VCVPCFTTDHQVARRCDDCCGGKGRGACYGPQCLCR

SEQ ID NO: 383 - protein, 36 aa, Artificial Sequence
  Description of Artificial Sequence Synthetic polypeptide
VCVPCFTTDHQVARKCDDCCGGAGRGACYGPQCLCR

SEQ ID NO: 384 - protein, 36 aa, Artificial Sequence
  Description of Artificial Sequence Synthetic polypeptide
VCVPCFTTDHQVARACDDCCGGAGRGACYGPQCLCR

SEQ ID NO: 385 - protein, 36 aa, Artificial Sequence
  Description of Artificial Sequence Synthetic polypeptide
VCVPCFTTDHQVARRCDDCCGGAGRGACYGPQCLCR

SEQ ID NO: 386 - protein, 36 aa, Artificial Sequence
  Description of Artificial Sequence Synthetic polypeptide
VCVPCFTTDHQVARKCDDCCGGRGRGACYGPQCLCR

SEQ ID NO: 387 - protein, 36 aa, Artificial Sequence
  Description of Artificial Sequence Synthetic polypeptide
VCVPCFTTDHQVARACDDCCGGRGRGACYGPQCLCR

SEQ ID NO: 388 - protein, 36 aa, Artificial Sequence
  Description of Artificial Sequence Synthetic polypeptide
VCVPCFTTDHQVARRCDDCCGGRGRGACYGPQCLCR

SEQ ID NO: 389 - protein, 36 aa, Artificial Sequence
  Description of Artificial Sequence Synthetic polypeptide
VCVPCFTTDHQVARKCDDCCGGKGRGRCYGPQCLCR

SEQ ID NO: 390 - protein, 36 aa, Artificial Sequence
  Description of Artificial Sequence Synthetic polypeptide
VCVPCFTTDHQVARACDDCCGGKGRGRCYGPQCLCR

SEQ ID NO: 391 - protein, 36 aa, Artificial Sequence
  Description of Artificial Sequence Synthetic polypeptide
VCVPCFTTDHQVARRCDDCCGGKGRGRCYGPQCLCR

SEQ ID NO: 392 - protein, 36 aa, Artificial Sequence
  Description of Artificial Sequence Synthetic polypeptide
VCVPCFTTDHQVARKCDDCCGGAGRGRCYGPQCLCR

SEQ ID NO: 393 - protein, 36 aa, Artificial Sequence
  Description of Artificial Sequence Synthetic polypeptide
VCVPCFTTDHQVARACDDCCGGAGRGRCYGPQCLCR

SEQ ID NO: 394 - protein, 36 aa, Artificial Sequence
  Description of Artificial Sequence Synthetic polypeptide
VCVPCFTTDHQVARRCDDCCGGAGRGRCYGPQCLCR

SEQ ID NO: 395 - protein, 36 aa, Artificial Sequence
  Description of Artificial Sequence Synthetic polypeptide
VCVPCFTTDHQVARKCDDCCGGRGRGRCYGPQCLCR

SEQ ID NO: 396 - protein, 36 aa, Artificial Sequence
  Description of Artificial Sequence Synthetic polypeptide
VCVPCFTTDHQVARACDDCCGGRGRGRCYGPQCLCR

SEQ ID NO: 397 - protein, 36 aa, Artificial Sequence
  Description of Artificial Sequence Synthetic polypeptide
VCVPCFTTDHQVARRCDDCCGGRGRGRCYGPQCLCR

SEQ ID NO: 398 - protein, 36 aa, Artificial Sequence; identical to SEQ ID NO: 397
  Description of Artificial Sequence Synthetic polypeptide

SEQ ID NO: 399 - protein, 36 aa, Artificial Sequence
  Description of Artificial Sequence Synthetic polypeptide
KCVPCFTTDHQVARRCDDCCGGRGRGRCYGPQCLCR

SEQ ID NO: 400 - protein, 36 aa, Artificial Sequence; identical to SEQ ID NO: 30
  Description of Artificial Sequence Synthetic polypeptide

SEQ ID NO: 401 - protein, 36 aa, Artificial Sequence; identical to SEQ ID NO: 31
  Description of Artificial Sequence Synthetic polypeptide

SEQ ID NO: 402 - protein, 36 aa, Artificial Sequence
  Description of Artificial Sequence Synthetic polypeptide
  MOD_RES (15)..(15): Citrulline
  MOD_RES (23)..(23): Citrulline
VCVPCFTTDHQVARXCDDCCGGXGRGKCYGPQCLCR

SEQ ID NO: 403 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
VCVPCFTTDHQVARXCDDCCGGXGRGXCYGPQCLCR

SEQ ID NO: 404 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCVPCFTTDHQVARXCDDCCGGXGRGXCYGPQCLCR

SEQ ID NO: 405 - protein, 36 aa, Artificial Sequence; identical to SEQ ID NO: 35
  Description of Artificial Sequence Synthetic polypeptide
  MOD_RES (15)..(15): Citrulline
  MOD_RES (23)..(23): Citrulline

SEQ ID NO: 406 - protein, 36 aa, Artificial Sequence; identical to SEQ ID NO: 36
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 407 - protein, 36 aa, Artificial Sequence; identical to SEQ ID NO: 37
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 408 - protein, 36 aa, Artificial Sequence
  Description of Artificial Sequence Synthetic polypeptide
LCLPCFTTDHQLARKCDDCCGGKGRGKCYGPQCLCR

SEQ ID NO: 409 - protein, 36 aa, Artificial Sequence
  Description of Artificial Sequence Synthetic polypeptide
LCLPCFTTDHQLARACDDCCGGKGRGKCYGPQCLCR

SEQ ID NO: 410 - protein, 36 aa, Artificial Sequence
  Description of Artificial Sequence Synthetic polypeptide
LCLPCFTTDHQLARRCDDCCGGKGRGKCYGPQCLCR

SEQ ID NO: 411 - protein, 36 aa, Artificial Sequence
  Description of Artificial Sequence Synthetic polypeptide
LCLPCFTTDHQLARKCDDCCGGAGRGKCYGPQCLCR

SEQ ID NO: 412 - protein, 36 aa, Artificial Sequence
  Description of Artificial Sequence Synthetic polypeptide
LCLPCFTTDHQLARACDDCCGGAGRGKCYGPQCLCR

SEQ ID NO: 413 - protein, 36 aa, Artificial Sequence
  Description of Artificial Sequence Synthetic polypeptide
LCLPCFTTDHQLARRCDDCCGGAGRGKCYGPQCLCR

SEQ ID NO: 414 - protein, 36 aa, Artificial Sequence
  Description of Artificial Sequence Synthetic polypeptide
LCLPCFTTDHQLARKCDDCCGGRGRGKCYGPQCLCR

SEQ ID NO: 415 - protein, 36 aa, Artificial Sequence
  Description of Artificial Sequence Synthetic polypeptide
LCLPCFTTDHQLARACDDCCGGRGRGKCYGPQCLCR

SEQ ID NO: 416 - protein, 36 aa, Artificial Sequence
  Description of Artificial Sequence Synthetic polypeptide
LCLPCFTTDHQLARRCDDCCGGRGRGKCYGPQCLCR

SEQ ID NO: 417 - protein, 36 aa, Artificial Sequence
  Description of Artificial Sequence Synthetic polypeptide
LCLPCFTTDHQLARKCDDCCGGKGRGACYGPQCLCR

SEQ ID NO: 418 - protein, 36 aa, Artificial Sequence
  Description of Artificial Sequence Synthetic polypeptide
LCLPCFTTDHQLARACDDCCGGKGRGACYGPQCLCR

SEQ ID NO: 419 - protein, 36 aa, Artificial Sequence
  Description of Artificial Sequence Synthetic polypeptide
LCLPCFTTDHQLARRCDDCCGGKGRGACYGPQCLCR

SEQ ID NO: 420 - protein, 36 aa, Artificial Sequence
  Description of Artificial Sequence Synthetic polypeptide
LCLPCFTTDHQLARKCDDCCGGAGRGACYGPQCLCR

SEQ ID NO: 421 - protein, 36 aa, Artificial Sequence
  Description of Artificial Sequence Synthetic polypeptide
LCLPCFTTDHQLARACDDCCGGAGRGACYGPQCLCR

SEQ ID NO: 422 - protein, 36 aa, Artificial Sequence
  Description of Artificial Sequence Synthetic polypeptide
LCLPCFTTDHQLARRCDDCCGGAGRGACYGPQCLCR

SEQ ID NO: 423 - protein, 36 aa, Artificial Sequence
  Description of Artificial Sequence Synthetic polypeptide
LCLPCFTTDHQLARKCDDCCGGRGRGACYGPQCLCR

SEQ ID NO: 424 - protein, 36 aa, Artificial Sequence
  Description of Artificial Sequence Synthetic polypeptide
LCLPCFTTDHQLARACDDCCGGRGRGACYGPQCLCR

SEQ ID NO: 425 - protein, 36 aa, Artificial Sequence
  Description of Artificial Sequence Synthetic polypeptide
LCLPCFTTDHQLARRCDDCCGGRGRGACYGPQCLCR

SEQ ID NO: 426 - protein, 36 aa, Artificial Sequence
  Description of Artificial Sequence Synthetic polypeptide
LCLPCFTTDHQLARKCDDCCGGKGRGRCYGPQCLCR

SEQ ID NO: 427 - protein, 36 aa, Artificial Sequence
  Description of Artificial Sequence Synthetic polypeptide
LCLPCFTTDHQLARACDDCCGGKGRGRCYGPQCLCR

SEQ ID NO: 428 - protein, 36 aa, Artificial Sequence
  Description of Artificial Sequence Synthetic polypeptide
LCLPCFTTDHQLARRCDDCCGGKGRGRCYGPQCLCR

SEQ ID NO: 429 - protein, 36 aa, Artificial Sequence
  Description of Artificial Sequence Synthetic polypeptide
LCLPCFTTDHQLARKCDDCCGGAGRGRCYGPQCLCR

SEQ ID NO: 430 - protein, 36 aa, Artificial Sequence
  Description of Artificial Sequence Synthetic polypeptide
LCLPCFTTDHQLARACDDCCGGAGRGRCYGPQCLCR

SEQ ID NO: 431 - protein, 36 aa, Artificial Sequence
  Description of Artificial Sequence Synthetic polypeptide
LCLPCFTTDHQLARRCDDCCGGAGRGRCYGPQCLCR

SEQ ID NO: 432 - protein, 36 aa, Artificial Sequence
  Description of Artificial Sequence Synthetic polypeptide
LCLPCFTTDHQLARKCDDCCGGRGRGRCYGPQCLCR

SEQ ID NO: 433 - protein, 36 aa, Artificial Sequence
  Description of Artificial Sequence Synthetic polypeptide
LCLPCFTTDHQLARACDDCCGGRGRGRCYGPQCLCR

SEQ ID NO: 434 - protein, 36 aa, Artificial Sequence
  Description of Artificial Sequence Synthetic polypeptide
LCLPCFTTDHQLARRCDDCCGGRGRGRCYGPQCLCR

SEQ ID NO: 435 - protein, 36 aa, Artificial Sequence; identical to SEQ ID NO: 434
  Description of Artificial Sequence Synthetic polypeptide

SEQ ID NO: 436 - protein, 36 aa, Artificial Sequence
  Description of Artificial Sequence Synthetic polypeptide
KCLPCFTTDHQLARRCDDCCGGRGRGRCYGPQCLCR

SEQ ID NO: 437 - protein, 36 aa, Artificial Sequence; identical to SEQ ID NO: 30
  Description of Artificial Sequence Synthetic polypeptide

SEQ ID NO: 438 - protein, 36 aa, Artificial Sequence; identical to SEQ ID NO: 31
  Description of Artificial Sequence Synthetic polypeptide

SEQ ID NO: 439 - protein, 36 aa, Artificial Sequence
  Description of Artificial Sequence Synthetic polypeptide
  MOD_RES (15)..(15): Citrulline
  MOD_RES (23)..(23): Citrulline
LCLPCFTTDHQLARXCDDCCGGXGRGKCYGPQCLCR

SEQ ID NO: 440 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
LCLPCFTTDHQLARXCDDCCGGXGRGXCYGPQCLCR

SEQ ID NO: 441 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCLPCFTTDHQLARXCDDCCGGXGRGXCYGPQCLCR

SEQ ID NO: 442 - protein, 36 aa, Artificial Sequence; identical to SEQ ID NO: 35
  Description of Artificial Sequence Synthetic polypeptide
  MOD_RES (15)..(15): Citrulline
  MOD_RES (23)..(23): Citrulline

SEQ ID NO: 443 - protein, 36 aa, Artificial Sequence; identical to SEQ ID NO: 36
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 444 - protein, 36 aa, Artificial Sequence; identical to SEQ ID NO: 37
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 445 - protein, 36 aa, Artificial Sequence
  Description of Artificial Sequence Synthetic polypeptide
SCSPCFTTDHQSARKCDDCCGGKGRGKCYGPQCLCR

SEQ ID NO: 446 - protein, 36 aa, Artificial Sequence
  Description of Artificial Sequence Synthetic polypeptide
SCSPCFTTDHQSARACDDCCGGKGRGKCYGPQCLCR

SEQ ID NO: 447 - protein, 36 aa, Artificial Sequence
  Description of Artificial Sequence Synthetic polypeptide
SCSPCFTTDHQSARRCDDCCGGKGRGKCYGPQCLCR

SEQ ID NO: 448 - protein, 36 aa, Artificial Sequence
  Description of Artificial Sequence Synthetic polypeptide
SCSPCFTTDHQSARKCDDCCGGAGRGKCYGPQCLCR

SEQ ID NO: 449 - protein, 36 aa, Artificial Sequence
  Description of Artificial Sequence Synthetic polypeptide
SCSPCFTTDHQSARACDDCCGGAGRGKCYGPQCLCR

SEQ ID NO: 450 - protein, 36 aa, Artificial Sequence
  Description of Artificial Sequence Synthetic polypeptide
SCSPCFTTDHQSARRCDDCCGGAGRGKCYGPQCLCR

SEQ ID NO: 451 - protein, 36 aa, Artificial Sequence
  Description of Artificial Sequence Synthetic polypeptide
SCSPCFTTDHQSARKCDDCCGGRGRGKCYGPQCLCR

SEQ ID NO: 452 - protein, 36 aa, Artificial Sequence
  Description of Artificial Sequence Synthetic polypeptide
SCSPCFTTDHQSARACDDCCGGRGRGKCYGPQCLCR

SEQ ID NO: 453 - protein, 36 aa, Artificial Sequence
  Description of Artificial Sequence Synthetic polypeptide
SCSPCFTTDHQSARRCDDCCGGRGRGKCYGPQCLCR

SEQ ID NO: 454 - protein, 36 aa, Artificial Sequence
  Description of Artificial Sequence Synthetic polypeptide
SCSPCFTTDHQSARKCDDCCGGKGRGACYGPQCLCR

SEQ ID NO: 455 - protein, 36 aa, Artificial Sequence
  Description of Artificial Sequence Synthetic polypeptide
SCSPCFTTDHQSARACDDCCGGKGRGACYGPQCLCR

SEQ ID NO: 456 - protein, 36 aa, Artificial Sequence
  Description of Artificial Sequence Synthetic polypeptide
SCSPCFTTDHQSARRCDDCCGGKGRGACYGPQCLCR

SEQ ID NO: 457 - protein, 36 aa, Artificial Sequence
  Description of Artificial Sequence Synthetic polypeptide
SCSPCFTTDHQSARKCDDCCGGAGRGACYGPQCLCR

SEQ ID NO: 458 - protein, 36 aa, Artificial Sequence
  Description of Artificial Sequence Synthetic polypeptide
SCSPCFTTDHQSARACDDCCGGAGRGACYGPQCLCR

SEQ ID NO: 459 - protein, 36 aa, Artificial Sequence
  Description of Artificial Sequence Synthetic polypeptide
SCSPCFTTDHQSARRCDDCCGGAGRGACYGPQCLCR

SEQ ID NO: 460 - protein, 36 aa, Artificial Sequence
  Description of Artificial Sequence Synthetic polypeptide
SCSPCFTTDHQSARKCDDCCGGRGRGACYGPQCLCR

SEQ ID NO: 461 - protein, 36 aa, Artificial Sequence
  Description of Artificial Sequence Synthetic polypeptide
SCSPCFTTDHQSARACDDCCGGRGRGACYGPQCLCR

SEQ ID NO: 462 - protein, 36 aa, Artificial Sequence
  Description of Artificial Sequence Synthetic polypeptide
SCSPCFTTDHQSARRCDDCCGGRGRGACYGPQCLCR

SEQ ID NO: 463 - protein, 36 aa, Artificial Sequence
  Description of Artificial Sequence Synthetic polypeptide
SCSPCFTTDHQSARKCDDCCGGKGRGRCYGPQCLCR

SEQ ID NO: 464 - protein, 36 aa, Artificial Sequence
  Description of Artificial Sequence Synthetic polypeptide
SCSPCFTTDHQSARACDDCCGGKGRGRCYGPQCLCR

SEQ ID NO: 465 - protein, 36 aa, Artificial Sequence
  Description of Artificial Sequence Synthetic polypeptide
SCSPCFTTDHQSARRCDDCCGGKGRGRCYGPQCLCR

SEQ ID NO: 466 - protein, 36 aa, Artificial Sequence
  Description of Artificial Sequence Synthetic polypeptide
SCSPCFTTDHQSARKCDDCCGGAGRGRCYGPQCLCR

SEQ ID NO: 467 - protein, 36 aa, Artificial Sequence
  Description of Artificial Sequence Synthetic polypeptide
SCSPCFTTDHQSARACDDCCGGAGRGRCYGPQCLCR

SEQ ID NO: 468 - protein, 36 aa, Artificial Sequence
  Description of Artificial Sequence Synthetic polypeptide
SCSPCFTTDHQSARRCDDCCGGAGRGRCYGPQCLCR

SEQ ID NO: 469 - protein, 36 aa, Artificial Sequence
  Description of Artificial Sequence Synthetic polypeptide
SCSPCFTTDHQSARKCDDCCGGRGRGRCYGPQCLCR

SEQ ID NO: 470 - protein, 36 aa, Artificial Sequence
  Description of Artificial Sequence Synthetic polypeptide
SCSPCFTTDHQSARACDDCCGGRGRGRCYGPQCLCR

SEQ ID NO: 471 - protein, 36 aa, Artificial Sequence
  Description of Artificial Sequence Synthetic polypeptide
SCSPCFTTDHQSARRCDDCCGGRGRGRCYGPQCLCR

SEQ ID NO: 472 - protein, 36 aa, Artificial Sequence; identical to SEQ ID NO: 471
  Description of Artificial Sequence Synthetic polypeptide

SEQ ID NO: 473 - protein, 36 aa, Artificial Sequence
  Description of Artificial Sequence Synthetic polypeptide
KCSPCFTTDHQSARRCDDCCGGRGRGRCYGPQCLCR

SEQ ID NO: 474 - protein, 36 aa, Artificial Sequence; identical to SEQ ID NO: 30
  Description of Artificial Sequence Synthetic polypeptide

SEQ ID NO: 475 - protein, 36 aa, Artificial Sequence; identical to SEQ ID NO: 31
  Description of Artificial Sequence Synthetic polypeptide

SEQ ID NO: 476 - protein, 36 aa, Artificial Sequence
  Description of Artificial Sequence Synthetic polypeptide
  MOD_RES (15)..(15): Citrulline
  MOD_RES (23)..(23): Citrulline
SCSPCFTTDHQSARXCDDCCGGXGRGKCYGPQCLCR

SEQ ID NO: 477 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
SCSPCFTTDHQSARXCDDCCGGXGRGXCYGPQCLCR

SEQ ID NO: 478 - protein, 36 aa, Artificial Sequence
  Description of Artificial Sequence Synthetic polypeptide
  MOD_RES (15)..(15): Citrulline
  MOD_RES (23)..(23): Citrulline
  MOD_RES (27)..(27): Citrulline
KCSPCFTTDHQSARXCDDCCGGXGRGXCYGPQCLCR

SEQ ID NO: 479 - protein, 36 aa, Artificial Sequence; identical to SEQ ID NO: 35
  Description of Artificial Sequence Synthetic polypeptide
  MOD_RES (15)..(15): Citrulline
  MOD_RES (23)..(23): Citrulline

SEQ ID NO: 480 - protein, 36 aa, Artificial Sequence; identical to SEQ ID NO: 36
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 481 - protein, 36 aa, Artificial Sequence; identical to SEQ ID NO: 37
  Description of Artificial Sequence Synthetic polypeptide
  MOD_RES (15)..(15): Citrulline
  MOD_RES (23)..(23): Citrulline
  MOD_RES (27)..(27): Citrulline

SEQ ID NO: 482 - protein, 36 aa, Artificial Sequence; identical to SEQ ID NO: 33
  Description of Artificial Sequence Synthetic polypeptide
  MOD_RES (15)..(15): Any amino acid
  MOD_RES (23)..(23): Any amino acid
  MOD_RES (27)..(27): Any amino acid

SEQ ID NO: 483 - protein, 36 aa, Artificial Sequence; identical to SEQ ID NO: 33
  Description of Artificial Sequence Synthetic polypeptide
  MOD_RES (15)..(15): Lys, Ala or Arg
  MOD_RES (23)..(23): Lys, Ala or Arg
  MOD_RES (27)..(27): Lys, Ala or Arg

SEQ ID NO: 484 - protein, 36 aa, Artificial Sequence; identical to SEQ ID NO: 33
  Description of Artificial Sequence Synthetic polypeptide
  MOD_RES (15)..(15): Ala or Arg
  MOD_RES (23)..(23): Ala or Arg
  MOD_RES (27)..(27): Ala or Arg

SEQ ID NO: 485 - protein, 36 aa, Artificial Sequence; identical to SEQ ID NO: 32
  Description of Artificial Sequence Synthetic polypeptide
  MOD_RES (15)..(15): Ala or Arg
  MOD_RES (23)..(23): Ala or Arg